SEQUENCE LISTING (75 sequences)

SEQ ID NO: 1 - protein, 16 aa, Artificial Sequence
  Description of Artificial Sequence Synthetic peptide
AKTTPKLEEGEFSEAR

SEQ ID NO: 2 - protein, 17 aa, Artificial Sequence
  Description of Artificial Sequence Synthetic peptide
AKTTPKLEEGEFSEARV

SEQ ID NO: 3 - protein, 9 aa, Artificial Sequence
  Description of Artificial Sequence Synthetic peptide
AKTTPKLGG

SEQ ID NO: 4 - protein, 10 aa, Artificial Sequence
  Description of Artificial Sequence Synthetic peptide
SAKTTPKLGG

SEQ ID NO: 5 - protein, 6 aa, Artificial Sequence
  Description of Artificial Sequence Synthetic peptide
SAKTTP

SEQ ID NO: 6 - protein, 6 aa, Artificial Sequence
  Description of Artificial Sequence Synthetic peptide
RADAAP

SEQ ID NO: 7 - protein, 9 aa, Artificial Sequence
  Description of Artificial Sequence Synthetic peptide
RADAAPTVS

SEQ ID NO: 8 - protein, 12 aa, Artificial Sequence
  Description of Artificial Sequence Synthetic peptide
RADAAAAGGPGS

SEQ ID NO: 9 - protein, 27 aa, Artificial Sequence
  Description of Artificial Sequence Synthetic peptide
RADAAAAGGGGSGGGGSGGGGSGGGGS

SEQ ID NO: 10 - protein, 18 aa, Artificial Sequence
  Description of Artificial Sequence Synthetic peptide
SAKTTPKLEEGEFSEARV

SEQ ID NO: 11 - protein, 5 aa, Artificial Sequence
  Description of Artificial Sequence Synthetic peptide
ADAAP

SEQ ID NO: 12 - protein, 12 aa, Artificial Sequence
  Description of Artificial Sequence Synthetic peptide
ADAAPTVSIFPP

SEQ ID NO: 13 - protein, 5 aa, Artificial Sequence
  Description of Artificial Sequence Synthetic peptide
TVAAP

SEQ ID NO: 14 - protein, 12 aa, Artificial Sequence
  Description of Artificial Sequence Synthetic peptide
TVAAPSVFIFPP

SEQ ID NO: 15 - protein, 6 aa, Artificial Sequence
  Description of Artificial Sequence Synthetic peptide
QPKAAP

SEQ ID NO: 16 - protein, 13 aa, Artificial Sequence
  Description of Artificial Sequence Synthetic peptide
QPKAAPSVTLFPP

SEQ ID NO: 17 - protein, 6 aa, Artificial Sequence
  Description of Artificial Sequence Synthetic peptide
AKTTPP

SEQ ID NO: 18 - protein, 13 aa, Artificial Sequence
  Description of Artificial Sequence Synthetic peptide
AKTTPPSVTPLAP

SEQ ID NO: 19 - protein, 6 aa, Artificial Sequence
  Description of Artificial Sequence Synthetic peptide
AKTTAP

SEQ ID NO: 20 - protein, 13 aa, Artificial Sequence
  Description of Artificial Sequence Synthetic peptide
AKTTAPSVYPLAP

SEQ ID NO: 21 - protein, 6 aa, Artificial Sequence
  Description of Artificial Sequence Synthetic peptide
ASTKGP

SEQ ID NO: 22 - protein, 13 aa, Artificial Sequence
  Description of Artificial Sequence Synthetic peptide
ASTKGPSVFPLAP

SEQ ID NO: 23 - protein, 15 aa, Artificial Sequence
  Description of Artificial Sequence Synthetic peptide
GGGGSGGGGSGGGGS

SEQ ID NO: 24 - protein, 15 aa, Artificial Sequence
  Description of Artificial Sequence Synthetic peptide
GENKVEYAPALMALS

SEQ ID NO: 25 - protein, 15 aa, Artificial Sequence
  Description of Artificial Sequence Synthetic peptide
GPAKELTPLKEAKVS

SEQ ID NO: 26 - protein, 15 aa, Artificial Sequence
  Description of Artificial Sequence Synthetic peptide
GHEAAAVMQVQYPAS

SEQ ID NO: 27 - protein, 10 aa, Artificial Sequence
  Description of Artificial Sequence Synthetic peptide
GGGGSGGGGS

SEQ ID NO: 28 - protein, 334 aa, Artificial Sequence
  Description of Artificial Sequence Synthetic polypeptide
DIQMTQSPSSLSASVGDRVTITCRASEDIYSNLAWYQQKPGKAPKLLIYDTNNLADGVPSRFSGSGSGTDFTLTISSLQPEDFATYYCQQYNNYPPTFGQGTKLEIKRTVAAPSVFIFPPDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 577 aa, Artificial Sequence
  Description of Artificial Sequence Synthetic polypeptide
EVQLVESGGGLVQPGGSLRLSCAASGFTFSNFPMAWVRQAPGKGLEWVATISSSDGTTYYRDSVKGRFTISRDNAKNSLYLQMNSLRAEDTAVYYCARGYYNSPFAYWGQGTLVTVSSASTKGPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 584 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPQVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 329 aa, Artificial Sequence
  Description of Artificial Sequence Synthetic polypeptide
QIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPESALTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 584 aa, Artificial Sequence
  Description of Artificial Sequence Synthetic polypeptide
QVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSASTKGPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 329 aa, Artificial Sequence
  Description of Artificial Sequence Synthetic polypeptide
ESALTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLGQPKAAPQIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 579 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 327 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 579 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 327 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 579 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 328 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 579 aa, Artificial Sequence
  Description of Artificial Sequence Synthetic polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 328 aa, Artificial Sequence
  Description of Artificial Sequence Synthetic polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 577 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 327 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 577 aa, Artificial Sequence
  Description of Artificial Sequence Synthetic polypeptide
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 327 aa, Artificial Sequence
  Description of Artificial Sequence Synthetic polypeptide
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRTVAAPDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 579 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 327 aa, Artificial Sequence
  Description of Artificial Sequence Synthetic polypeptide
QIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 579 aa, Artificial Sequence
  Description of Artificial Sequence Synthetic polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 327 aa, Artificial Sequence
  Description of Artificial Sequence Synthetic polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPQIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 240 aa, Artificial Sequence
  Description of Artificial Sequence Synthetic polypeptide
EVQLVESGGGLVQPGGSLRLSCAASGFTFTDNWISWVRQAPGKGLEWVGYISPNSGFTYYADSVKGRFTISADTSKNTAYLQMNSLRAEDTAVYYCARDNFGGYFDYWGQGTLVTVSSASTKGPQVQLQQSGAELVKPGASVKISCKASGYTFTDYYINWVKLAPGQGLEWIGWIYPGSGNTKYNEKFKGKATLTIDTSSSTAYMQLSSLTSEDTAVYFCVRDSPFFDYWGQGTLLTVSS

SEQ ID NO: 51 - protein, 227 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATTYYCQQSYTGTVTFGQGTKVEIKRTVAAPDIVLTQSPDSLAVSLGERVTMNCKSSQSLLNSGMRKSFLAWYQQKPGQSPKLLIYWASTRESGVPDRFTGSGSGTDFTLTISSVQAEDVAVYYCKQSYHLFTFGSGTKLEIKR

SEQ ID NO: 52 - protein, 240 aa, Artificial Sequence
  Description of Artificial Sequence Synthetic polypeptide
QVQLQQSGAELVKPGASVKISCKASGYTFTDYYINWVKLAPGQGLEWIGWIYPGSGNTKYNEKFKGKATLTIDTSSSTAYMQLSSLTSEDTAVYFCVRDSPFFDYWGQGTLLTVSSASTKGPEVQLVESGGGLVQPGGSLRLSCAASGFTFTDNWISWVRQAPGKGLEWVGYISPNSGFTYYADSVKGRFTISADTSKNTAYLQMNSLRAEDTAVYYCARDNFGGYFDYWGQGTLVTVSS

SEQ ID NO: 53 - protein, 227 aa, Artificial Sequence
  Description of Artificial Sequence Synthetic polypeptide
DIVLTQSPDSLAVSLGERVTMNCKSSQSLLNSGMRKSFLAWYQQKPGQSPKLLIYWASTRESGVPDRFTGSGSGTDFTLTISSVQAEDVAVYYCKQSYHLFTFGSGTKLEIKRTVAAPDIQMTQSPSSLSASVGDRVTITCRASQDVSTAVAWYQQKPGKAPKLLIYSASFLYSGVPSRFSGSGSGTDFTLTISSLQPEDFATTYYCQQSYTGTVTFGQGTKVEIKR

SEQ ID NO: 54 - protein, 580 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPEVQLQQSGPELMKPGASVKMSCKASGYTFTDYNMHWMKQNQGKSLEWIGEINPNSGGSGYNQKFKGKATLTVDKSSSTAYMELRSLTSEDSAVYYCARLGYYGNYEDWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 327 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPDLQMTQTTSSLSASLGDRVTISCRASQDISNYLNWYQQKPDGTVKLLIFYTSTLQSGVPSRFSGSGSGTNYSLTITNLEQDDAATYFCQQGDTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 580 aa, Artificial Sequence
  Description of Artificial Sequence Synthetic polypeptide
EVQLQQSGPELMKPGASVKMSCKASGYTFTDYNMHWMKQNQGKSLEWIGEINPNSGGSGYNQKFKGKATLTVDKSSSTAYMELRSLTSEDSAVYYCARLGYYGNYEDWYFDVWGAGTTVTVSSASTKGPEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 327 aa, Artificial Sequence
  Description of Artificial Sequence Synthetic polypeptide
DLQMTQTTSSLSASLGDRVTISCRASQDISNYLNWYQQKPDGTVKLLIFYTSTLQSGVPSRFSGSGSGTNYSLTITNLEQDDAATYFCQQGDTLPYTFGGGTKLEIKRTVAAPDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 577 aa, Artificial Sequence
  Description of Artificial Sequence Synthetic polypeptide
QVQLQQSGAELMKPGASVKLSCKATGYTFTGSWIEWIKQRPGHGLEWIGQILPGSGSAYYNEKFKGKATFTADTSSKTVYIQLISLTTEDSAIYYCAREDNYGSSSLAYWGQGTLLTVSAASTKGPEVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 331 aa, Artificial Sequence
  Description of Artificial Sequence Synthetic polypeptide
DILLTQSPAILSVSPGERVSFSCRASQSIGTNIHWYQQRTNGSPRLLIKYASESISGIPSRFSGGGSGTDFTLSINSVESEDIADYYCQQSNNWPLTFGAGTKLELKRTVAAPDIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 577 aa, Artificial Sequence
  Description of Artificial Sequence Synthetic polypeptide
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPQVQLQQSGAELMKPGASVKLSCKATGYTFTGSWIEWIKQRPGHGLEWIGQILPGSGSAYYNEKFKGKATFTADTSSKTVYIQLISLTTEDSAIYYCAREDNYGSSSLAYWGQGTLLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 331 aa, Artificial Sequence
  Description of Artificial Sequence Synthetic polypeptide
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPDILLTQSPAILSVSPGERVSFSCRASQSIGTNIHWYQQRTNGSPRLLIKYASESISGIPSRFSGGGSGTDFTLSINSVESEDIADYYCQQSNNWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 587 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSEVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 332 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGSGGGGSGSEIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 573 aa, Artificial Sequence [sequence omitted]

SEQ ID NO: 65 - protein, 332 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPDVLMTQTPLSLPVTPGEPASISCTSSQNIVHSNGNTYLEWYLQKPGQSPQLLIYKVSNRFSGVPDRFSGSGSGTDFTLKISRVEAEDVGVYYCFQVSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 577 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 327 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 584 aa, Artificial Sequence
  Description of Artificial Sequence Synthetic polypeptide
EVQLVESGGGLVQPGGSLRLSCAASGFTFRHFPMAWVRQAPGKGLEWVATISSSDAWPSYRDSVKGRFTISRDNAKNSLYLQMNSLRAEDTAVYYCSRGYYNSPFAYWGQGTLVTVSSASTKGPSVFPLAPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 327 aa, Artificial Sequence
  Description of Artificial Sequence Synthetic polypeptide
DIQMTQSPSSLSASVGDRVTITCRASEDIYSNLAWYQQKPGKAPKLLIYDTNNLADGVPSRFSGSGSGTDFTLTISSLQPEDFATYYCQQYNNYPPTFGQGTKLEIKRTVAAPDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 576 aa, Artificial Sequence
  Description of Artificial Sequence Synthetic polypeptide
EVTLRESGPALVKPTQTLTLTCTFSGFSLSKSVMGVSWIRQPPGKALEWLAHIYWDDDKYYNPSLKSRLTISKDTSKNQVVLTMTNMDPVDTATYYCARRGIRSAMDYWGQGTTVTVSSASTKGPEVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 328 aa, Artificial Sequence
  Description of Artificial Sequence Synthetic polypeptide
DIVMTQSPDSLAVSLGERATINCKASQSVSNDVAWYQQKPGQPPKLLIYYASNRYTGVPDRFSGSGSGTDFTLTISSLQAEDVAVYYCQQDYNSPWTFGGGTKVEIKRTVAAPEIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 584 aa, Artificial Sequence
  Description of Artificial Sequence Synthetic polypeptide
EVQLVESGGGLVQPGGSLRLSCAASGFTFSNFPMAWVRQAPGKGLEWVATISSSDGTTYYRDSVKGRFTISRDNAKNSLYLQMNSLRAEDTAVYYCARGYYNSPFAYWGQGTLVTVSSASTKGPSVFPLAPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 327 aa, Artificial Sequence; identical to SEQ ID NO: 69
  Description of Artificial Sequence Synthetic polypeptide

SEQ ID NO: 74 - protein, 577 aa, Artificial Sequence
  Description of Artificial Sequence Synthetic polypeptide
EVQLVESGGGLVQPGGSLRLSCAASGFTFRHFPMAWVRQAPGKGLEWVATISSSDAWPSYRDSVKGRFTISRDNAKNSLYLQMNSLRAEDTAVYYCSRGYYNSPFAYWGQGTLVTVSSASTKGPEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 334 aa, Artificial Sequence; identical to SEQ ID NO: 28
  Description of Artificial Sequence Synthetic polypeptide

(19) United States
(12) Patent Application Publication
(10) Pub. No.: US 2017/0210796 A1
(43) Pub. Date: Jul. 27, 2017

(54) Stable Dual Variable Domain Immunoglobulin Protein Formulations

(71) Applicant: AbbVie, Inc., North Chicago, IL (US)

(72) Inventors: Michael SIEDLER, Heidelberg (DE); Vineet KUMAR, Storrs, CT (US); Ravi CHARI, Worcester, MA (US); Sonal SALUJA, Shrewsbury, MA (US); Vishwesh PATIL, Arlington, MA (US)

(21) Appl. No.: 15/343,327

(22) Filed: Nov. 4, 2016

Related U.S. Application Data
(62) Division of application No. 14/070,155, filed on Nov. 1, 2013, now abandoned.
(60) Provisional application No. 61/794,231, filed on Mar. 15, 2013.
(60) Provisional application No. 61/721,364, filed on Nov. 1, 2012.

(51) Int. Cl. C07K 16/24; C07K 16/18; C07K 16/22; A61K 39/395; A61K 47/22; A61K 47/10; A61K 47/26; A61K 9/08; C07K 16/26; A61K 9/19

(52) U.S. Cl. CPC C07K 16/245; C07K 16/244; C07K 16/241; C07K 16/26; C07K 16/18; C07K 16/22; A61K 9/19; A61K 47/22; A61K 47/10; A61K 47/26; A61K 9/08; A61K 39/39591; C07K 2317/56; C07K 2317/31; C07K 2317/94; A61K 2039/505

(57) ABSTRACT

The invention provides stable aqueous formulations comprising an Aqueous Stable Dual Variable Domain Immunoglobulin (AS-DVD-Ig) protein. The invention also provides stable lyophilized formulations comprising a Lyophilized Stable Dual Variable Domain Immunoglobulin (LS-DVD-Ig) protein.

### RELATED APPLICATIONS

This application claims the benefit of priority to U.S. Provisional Appln. No. 61/721,364, filed on Nov. 1, 2012. This application also claims the benefit of priority to U.S. Provisional Appln. No. 61/794,231, filed on Mar. 15, 2013. The contents of both the priority applications are hereby incorporated by reference.

### BACKGROUND OF THE INVENTION

A basic principle of pharmaceutical protein formulation is that certain instabilities, e.g., chemical instability and physical instability, must be overcome. Chemical instabilities often lead to the modification of the protein through bond formation or cleavage. Examples of problems associated with chemical instability include deamidation, racemization, hydrolysis, oxidation, beta elimination, and disulfide exchange. While physical instabilities do not lead to covalent changes in proteins, they are just as problematic and difficult to overcome. Physical instabilities involve changes in the higher order structure (secondary and above) of proteins, which can result in denaturation, adsorption to surfaces, aggregation, and/or precipitation (Manning et al. (1989) Pharm. Res. 6:903). For therapeutic proteins, chemical and physical instabilities can create significant challenges in formulating the protein for delivery to a patient. Aggregation is often considered the most common type of physical instability. For example, exposure to hydrophobic interfaces fosters physical instability phenomena, which can result by alignment of protein molecules at the interface, unfolding the protein and maximizing exposure of hydrophobic residues to air and initiating aggregation.

Highly concentrated protein formulations, especially those in liquid form, are often desirable for therapeutic purposes since they allow for dosages with smaller volumes, and provide for the possibility of subcutaneous delivery. The development of high protein concentration formulations, however, presents many challenges. For example, a high protein concentration often results in increased protein aggregation, insolubility and degradation (for review, see Shire et al. (2004) J. Pharm. Sci. 93:1390).

To date, the majority of approved therapeutic proteins are antibodies. The development of commercially viable antibody pharmaceutical formulations has not, however, been straightforward despite the fact that antibodies generally have the same structure (see Wang et al. (2007) J. Pharm. Sci. 96:1). Concentration dependent aggregation is considered one of the greatest challenges in formulating antibodies (see Shire et al. (2004) J. Pharm. Sci. 93:1390).

Dual Variable Domain Immunoglobulins (DVD-Ig™s) are multivalent binding proteins that are engineered to combine the function and specificity of two monoclonal antibodies into one molecular entity (See Wu et al., U.S. Pat. No. 7,612,181). Given the multivalent nature of DVD-Ig proteins, these molecules hold tremendous promise as therapeutics. However, DVD-Ig proteins present a formulation challenge given their large size (approximately 200 kDa) and complexity, compared to antibodies.

### SUMMARY OF THE INVENTION

The invention is based, in part, on the surprising discovery that while the majority of Dual Variable Domain Immunoglobulin (DVD-Ig™) proteins are prone to destabilization (e.g., aggregation) in aqueous formulations, a subset of DVD-Ig proteins can be stably formulated. Such stable DVD-Ig proteins are referred to herein as Aqueous Stable Dual Variable Domain-Immunoglobulin proteins or AS-DVD-Ig proteins.

In one embodiment, the invention provides stable aqueous formulations comprising AS-DVD-Ig proteins, including high concentration AS-DVD-Ig formulations. AS-DVD-Ig proteins are a subpopulation of DVD-Ig proteins characterized by their ability to remain stable in concentrations 50 mg/ml or greater during storage (e.g., exhibit an aggregation increase of less than 3% following accelerated storage (40° C.) in an aqueous formulation at a concentration of at least 50 mg/ml). In one embodiment, an AS-DVD-Ig protein is characterized as having less than 10% aggregation (determined by SEC) when formulated in a citrate phosphate buffer at a concentration of at least 50 mg/ml following 14 days of storage at 40° C. In one embodiment, the AS-DVD-Ig protein is characterized as having 6% or less aggregation as determined by SEC following 14 days of storage at 40° C., wherein the AS-DVD-Ig protein at a concentration of at least 50 mg/ml is stored in a citrate phosphate buffer or a histidine buffer. In another embodiment, the AS-DVD-Ig protein is characterized as having 5% or less aggregation as determined by SEC following 14 days of storage at 40° C., wherein the AS-DVD-Ig protein at a concentration of at least 50 mg/ml is stored in a citrate phosphate buffer or a histidine buffer. In one embodiment, the AS-DVD-Ig protein is characterized as having 4% or less aggregation as determined by SEC following 14 days of storage at 40° C., wherein the AS-DVD-Ig protein at a concentration of at least 50 mg/ml is stored in a citrate phosphate buffer or a histidine buffer. In one embodiment, the AS-DVD-Ig protein is characterized as having 3% or less aggregation as determined by SEC following 14 days of storage at 40° C., wherein the AS-DVD-Ig protein at a concentration of at least 50 mg/ml is stored in a citrate phosphate buffer or a histidine buffer. In one embodiment, the AS-DVD-Ig protein is characterized as having 2% or less aggregation as determined by SEC following 14 days of storage at 40° C., wherein the AS-DVD-Ig protein at a concentration of at least 50 mg/ml is stored in a citrate phosphate buffer or a histidine buffer. In one embodiment, the AS-DVD-Ig protein is characterized as having 1% or less aggregation as determined by SEC following 14 days of storage at 40° C., wherein the AS-DVD-Ig protein at a concentration of at least 50 mg/ml is stored in a citrate phosphate buffer or a histidine buffer. Examples of AS-DVD-Ig proteins that may be included in the formulations of the invention include, but are not limited to, an AS-DVD-Ig protein having binding specificity for IL4 and IL13; IL1α and IL1β; and TNFα and IL17.

In one embodiment, the invention provides an aqueous formulation comprising an AS-DVD-Ig protein, a buffer, a polyol, and a surfactant. For example, the invention provides an aqueous formulation comprising an AS-DVD-Ig, a buffer having a molarity of about 5 to about 50 mM, a surfactant, and a polyol, wherein the formulation has a pH of about 4.5 to about 7.5. In one embodiment, the formulation comprises 1-250 mg/ml, 10-230 mg/ml, 20-210 mg/ml, 30-190 mg/ml, 40-170 mg/ml, 50-150 mg/ml, 60-130 mg/ml, 70-110 mg/ml, or 80-105 mg/ml of the AS-DVD-Ig. In one embodiment, the polyol is sorbitol at a concentration, for example, of about 20 to about 60 mg/ml sorbitol, about 25 to about 55 mg/ml, about 30 to about 50 mg/ml, or about 35 to about 45 mg/ml. In one embodiment, the polyol is sucrose at a concentration, for example of about 60 to about 100 mg/ml, about 65 to about 95 mg/ml, about 70 to about 90 mg/ml, or about 75 to about 85 mg/ml. In one embodiment, the polyol is mannitol and is at a concentration, for example, of about 10 to about 100 mg/ml, or about 20 to about 80, about 20 to about 70, about 30 to about 60, or about 30 to about 50 mg/ml. In one embodiment, the surfactant is a polysorbate at, for example, a concentration of 0.001% to 1%, 0.005% to 0.05%, 0.01% to 0.05%, or about 0.1%.

In another embodiment, the invention provides an aqueous formulation comprising an AS-DVD-Ig, a buffer and a surfactant. For example, the invention provides an aqueous formulation comprising an AS-DVD-Ig, a buffer having a molarity of about 5 to about 50 mM, and a surfactant, wherein the formulation has a pH of about 4.5 to about 7.5. In one embodiment, the formulation comprises 1-250 mg/ml, 10-230 mg/ml, 20-210 mg/ml, 30-190 mg/ml, 40-170 mg/ml, 50-150 mg/ml, 60-130 mg/ml, 70-110 mg/ml, or 80-105 mg/ml of the AS-DVD-Ig. In one embodiment, the surfactant is a polysorbate at, for example, a concentration of 0.001% to 1%, 0.005% to 0.05%, 0.01% to 0.05%, or about 0.1%.

In another embodiment, the invention includes an aqueous formulation comprising an AS-DVD-Ig, a buffer, and a polyol. For example, the invention provides an aqueous formulation comprising an AS-DVD-Ig, a buffer having a molarity of about 5 to about 50 mM, and a polyol, wherein the formulation has a pH of about 4.5 to about 7.5. In one embodiment, the formulation comprises 1-250 mg/ml, 10-230 mg/ml, 20-210 mg/ml, 30-190 mg/ml, 40-170 mg/ml, 50-150 mg/ml, 60-130 mg/ml, 70-110 mg/ml, or 80-105 mg/ml of the AS-DVD-Ig. In one embodiment, the polyol is sorbitol at a concentration, for example, of about 20 to about 60 mg/ml sorbitol, about 25 to about 55 mg/ml, about 30 to about 50 mg/ml, or about 35 to about 45 mg/ml. In another embodiment, the polyol is sucrose in a concentration, for example of about 60 to about 100 mg/ml, about 65 to about 95 mg/ml, about 70 to about 90 mg/ml, or about 75 to about 85 mg/ml. In one embodiment, the polyol is mannitol and is at a concentration, for example, of about 10 to about 100 mg/ml, or about 20 to about 80, about 20 to about 70, about 30 to about 60, or about 30 to about 50 mg/ml.

In a further embodiment, the invention includes an aqueous formulation comprising an AS-DVD-Ig protein and a buffer. For example, the invention includes an aqueous formulation comprising an AS-DVD-Ig protein and a buffer having a molarity of about 5 to about 50 mM, wherein the formulation has a pH of about 4.5 to about 7.5. In one embodiment, the formulation comprises 1-250 mg/ml, 10-230 mg/ml, 20-210 mg/ml, 30-190 mg/ml, 40-170 mg/ml, 50-150 mg/ml, 60-130 mg/ml, 70-110 mg/ml, or 80-105 mg/ml of the AS-DVD-Ig.

In one embodiment, the invention provides a formulation comprising a DVD-Ig protein, a polyol, histidine buffer, and a polysorbate, wherein said formulation has a pH of about 5-7, and wherein the DVD-Ig protein is characterized as having 15% aggregation or less as determined by SEC, where the DVD-Ig protein is formulated in a citrate phosphate buffer or histidine buffer at a concentration of at least 60 mg/ml, following 14 days storage at 40° C. Such formulations may be either in a lyophilized or an aqueous state, as DVD-Ig proteins identified as having 15% aggregation or less as determined by SEC, where the DVD-Ig protein is formulated in a citrate phosphate buffer or a histidine buffer at a concentration of at least 60 mg/ml, following 14 days storage at 40° C. In one embodiment, the invention provides a formulation comprising a DVD-Ig protein, a polyol, histidine buffer, and a polysorbate, wherein said formulation has a pH of about 5-7, and wherein the DVD-Ig protein is characterized as having 6% aggregation or less as determined by SEC, where the DVD-Ig protein is formulated in a citrate phosphate buffer or histidine buffer at a concentration of at least 60 mg/ml, following 14 days storage at 40° C. In one embodiment, the DVD-Ig protein is stable in the formulations of the invention in either the aqueous or lyophilized state.

The invention is also based, in part, on the surprising discovery that while the majority of DVD-Ig™ proteins are prone to destabilization in a lyophilized state, a subset of DVD-Ig proteins are able to be stably formulated in a lyophilized form. Such stable DVD-Ig proteins are referred to herein as Lyophilized Stable Dual Variable Domain-Ig proteins or LS-DVD-Ig proteins.

Another aspect of the invention is a lyophilized formulation comprising a Lyophilized-Stable DVD-Ig (LS-DVD-Ig) protein, wherein when said formulation is reconstituted said formulation comprises about 1-100 mg/ml of the LS-DVD-Ig protein, about 10-50 mM of a buffer, a polyol, about 0.01-0.2 mg/ml of a polysorbate, and has a pH of about 5-7.

Another aspect of the invention is a lyophilized formulation prepared by lyophilizing an aqueous formulation comprising a buffer have a molarity of 5 to 50 mM, a surfactant, and a polyol, wherein the formulation has a pH of 4.5 to 7.5.

One aspect of the invention is that AS-DVD-Ig proteins are stable in formulations having a pH of about 4.5 to about 7.5. In one embodiment, the formulation has a pH of 5 to 6.5. In another embodiment, the formulation has a pH of about 5.7 to about 6.3. In one embodiment, the formulation the formulation of the invention has a pH of about 5.5 to 6.5. In one embodiment, the formulation of the invention has a pH of 5.8 to 6.2, or a pH of 6.

Examples of buffers that may be used in the aqueous formulations of the invention include, but are not limited to, acetate, histidine, glycine, arginine, phosphate, and citrate. In one embodiment, the molarity of the buffer in the formulation is about 5 to about 50 mM. In another embodiment, the buffer molarity is about 10 mM to about 20 mM.

Examples of polyols that may be used in the formulations of the invention include, but are not limited to, sorbitol, mannitol, and sucrose. In one embodiment, the polyol is sorbitol. In another embodiment, about 30 to about 50 mg/ml of sorbitol is used in the formulation. In another embodiment, the polyol is sucrose. In another embodiment, about 70 to about 90 mg/ml of sucrose is used in the formulation. In a further embodiment, the polyol is mannitol. In another embodiment, about 30 to about 50 mg/ml of mannitol is used in the formulation.

Examples of surfactants that may be used in the formulations of the invention include, but are not limited to, polysorbates and poloxamers. In one embodiment, the surfactant is a polysorbate, examples of which are polysorbate 80 and polysorbate 20. Other examples include poloxamer Pluronic F-68, albumin, lecithin, cyclodextrins. In another embodiment, the polysorbate has a concentration of about 0.05 mg/ml to about 2 mg/ml. In a further embodiment, the polysorbate has a concentration of about 0.01 to about 0.2 mg/ml.

One advantage of the compositions of the invention is that the AS-DVD-Ig or LS-DVD-Ig protein can be stably formulated in liquid form at a high concentration. In one embodiment, the AS-DVD-Ig or LS-DVD-Ig protein has a concentration of about 1 to about 200 mg/ml. In another embodiment, the AS-DVD-Ig or LS-DVD-Ig protein has a concentration of about 20 to about 100 mg/ml. In one embodiment, the formulation comprises 1-250 mg/ml, 10-230 mg/ml, 20-210 mg/ml, 30-190 mg/ml, 40-170 mg/ml, 50-150 mg/ml, 60-130 mg/ml, 70-110 mg/ml, or 80-105 mg/ml of the AS-DVD-Ig or LS-DVD-Ig.

In one embodiment, the AS-DVD-Ig protein or LS-DVD-Ig protein comprises a polypeptide chain comprising VD1-(X1)n-VD2-C-(X2)n, wherein VD1 is a first variable domain, VD2 is a second variable domain, C is a constant domain, X1 represents an amino acid or polypeptide, X2 represents an Fc region and n is 0 or 1. In another embodiment, the AS-DVD-Ig or LS-DVD-Ig protein used in the compositions and methods of the invention comprises four polypeptide chains, wherein two polypeptide chains comprise VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, and X2 is an Fc region; and two polypeptide chains comprise VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1; and wherein said four polypeptide chains of said binding protein form four functional antigen binding sites.

In one embodiment, the AS-DVD-Ig protein or LS-DVD-Ig protein comprises a polypeptide chain wherein the polypeptide chain comprises VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; and n is 0 or 1. In one embodiment, the AS-DVD-Ig protein or LS-DVD-Ig protein comprises a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not a CH1 or CL; X2 does not comprise an Fc region; and n is 0 or 1. In a further embodiment, (X1)n on the heavy and/or light chain is (X1)0 and/or (X2)n on the heavy and/or light chain is (X2)0.

In one embodiment, the AS-DVD-Ig protein or LS-DVD-Ig protein comprises first and second polypeptide chains, wherein the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a first linker with the proviso that it is not CH2; X2 is an Fc region; n is 0 or 1; and wherein the second polypeptide chain comprises a second VD1-(X1)n-VD2-C-(X2)n, wherein VD1 is a first light chain variable domain; VD2 is a second light chain variable domain; C is a light chain constant domain; X1 is a second linker with the proviso that it is not CH1 or CL; X2 does not comprise an Fc region; and n is 0 or 1.

In one embodiment, the VD1 of the first polypeptide chain and the VD1 of the second polypeptide chain are from different first and second parent antibodies, respectively, or binding portions thereof. In one embodiment, the VD2 of the first polypeptide chain and the VD2 of the second polypeptide chain are from different first and second parent antibodies, respectively, or binding portions thereof. In one embodiment, the first and the second parent antibodies bind different epitopes on the same target or different targets. In one embodiment, the first parent antibody or binding portion thereof binds the first target with a potency different from the potency with which the second parent antibody or binding portion thereof binds the second target. In one embodiment, the first parent antibody or binding portion thereof binds the first target with an affinity different from the affinity with which the second parent antibody or binding portion thereof binds the second target.

In one embodiment, the AS-DVD-Ig protein or LS-DVD-Ig protein comprise, two first polypeptide chains and two second polypeptide chains.

In one embodiment, the AS-DVD-Ig protein or LS-DVD-Ig protein comprises first and second polypeptide chains, each independently comprising VD1-(X1)n-VD2-C-(X2)n, wherein VD1 is a first variable domain; VD2 is a second variable domain; C is a constant domain; X1 is a linker with the proviso that it is not CH1; X2 is an Fc region; n is 0 or 1, wherein the VD1 domains on the first and second polypeptide chains form a first functional target binding site and the VD2 domains on the first and second polypeptide chains form a second functional target binding site. IN a further embodiment, the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; n is 0 or 1, and wherein the second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1; X2 does not comprise an Fc region; n is 0 or 1, wherein the VD1 domains on the first and second polypeptide chains form a first functional target binding site and the VD2 domains on the first and second polypeptide chains form a second functional target binding site.

In one embodiment, the AS-DVD-Ig protein or LS-DVD-Ig protein comprises first and second polypeptide chains, each independently comprising VD1-(X1)n-VD2-C-(X2)n, wherein VD1 is a first variable domain; VD2 is a second variable domain; C is a constant domain; X1 is a linker with the proviso that it is not CH1; X2 is an Fc region; n is 0 or 1, and wherein the VD1 domains on the first and second polypeptide chains form a first functional target binding site and the VD2 domains on the first and second polypeptide chains form a second functional target binding site. IN a further embodiment, the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; n is 0 or 1, and wherein the second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1; X2 does not comprise an Fc region; n is 0 or 1, wherein the VD1 domains on the first and second polypeptide chains form a first functional target binding site and the VD2 domains on the first and second polypeptide chains form a second functional target binding site.

In one embodiment, the formulation of the invention comprises a DVD-Ig protein comprising a heavy or light chain amino acid sequence as set forth in Table 61 or 66.

In one embodiment, the formulation of the invention comprises a DVD-Ig protein comprising a heavy or light chain variable region amino acid sequence as set forth in Table 61 or 66 (SEQ ID NOs: 28-75). Alternatively, the formulation of the invention comprises a DVD-Ig protein comprising CDRs as set forth in the heavy or light chain variable region amino acid sequences as set forth in Table 61 or 66 (SEQ ID NOs: 28-75).

In one embodiment, the formulation of the invention comprises an anti-TNF/IL-17 DVD-Ig having a heavy and light chain sequences having an amino acid sequence as set forth in SEQ ID NOs: 62 and 63, respectively.

In one embodiment, the formulation of the invention comprises an anti-IL1α/IL1β DVD-Ig having a heavy and light chain sequences having an amino acid sequence as set forth in SEQ ID NOs: 66 and 67, respectively.

In one embodiment, the DVD-Ig protein used in the formulation of the invention binds one of the following target combinations (in either target order): CD20/CD80, VEGF/Her2, TNF/RANKL, TNF/DKK, CD20/RANKL, DLL4/PLGF, TNF/SOST (S2), IL-9 (S2)/IgE, IL-12/IL-18, TNF/IL-17, TNF/PGE2, IL1α/IL1β, or DLL4/VEGF.

One advantage of the aqueous and lyophilized formulations of the invention is that they are stable despite being aqueous and having high concentrations of AS-DVD-Ig or LS-DVD-Ig proteins. In an embodiment, the formulations of the invention have low levels of aggregation of AS-DVD-Igs or LS-DVD-Ig proteins. In one embodiment, the formulation comprises less than 10% aggregate AS-DVD-Ig or LS-DVD-Ig proteins. In one embodiment, the formulation comprises less than 9% aggregate AS-DVD-Ig or LS-DVD-Ig proteins. In one embodiment, the formulation comprises less than 8% aggregate AS-DVD-Ig or LS-DVD-Ig proteins. In one embodiment, the formulation comprises less than 7% aggregate AS-DVD-Ig or LS-DVD-Ig proteins. In one embodiment, the formulation comprises less than 6% aggregate AS-DVD-Ig or LS-DVD-Ig proteins. In another embodiment, the formulation comprises less than 5% aggregate AS-DVD-Ig or LS-DVD-Ig proteins. In one embodiment, the formulation comprises less than 4% aggregate AS-DVD-Ig or LS-DVD-Ig proteins. In a further embodiment, the formulation comprises less than 3% aggregate AS-DVD-Ig or LS-DVD-Ig proteins. Aggregation can be determined, for example, by SEC analysis. Other examples of stability are provided in the examples below.

In one embodiment, the AS-DVD-Ig protein is characterized as a DVD-Ig protein having a 10% relative (rel.) peak area or less change in monomers at about 40° C. after 21 days of storage at a concentration of 100 mg/ml in an aqueous formulation at a pH between about 5.5 to 6.5. In another embodiment, the AS-DVD-Ig protein is characterized as a DVD-Ig protein having a 1% rel. peak area or less change in monomers at about 5° C. after 21 days of storage at a concentration of 100 mg/ml at a pH between about 5.5 to 6.5 in an aqueous formulation.

In one embodiment, the LS-DVD-Ig protein has more than 10% rel. peak area change in monomers observed, following accelerated storage at a pH between 5.5-6.5 in an aqueous formulation for 21 days at 40° C., when formulated at a concentration over 100 mg/ml.

In one embodiment, the formulation of the invention is a pharmaceutical formulation.

Also included in the invention are methods of making and using AS-DVD-Ig protein or LS-DVD-Ig protein formulations. In one embodiment, the formulations are used for treating a disorder in a subject.

A further embodiment of the invention is a method of identifying either an AS-DVD-Ig protein or an LS-DVD-Ig protein. Such methods include aggregation testing (e.g., by SEC analysis) following accelerated storage (e.g., 14 days at 40 degrees C.) of a liquid formulation comprising the DVD-Ig protein, a citrate/phosphate buffer, and a high concentration of DVD-Ig protein (e.g., 50 mg/ml or greater).

### BRIEF DESCRIPTION OF DRAWINGS

FIG. 1 shows a graphic description of a comparison of DSC profiles of an IgG1 antibody (Briakinumab) to that of a DVD-Ig protein (TNF/PGE2) showing the difference in three vs. four domain unfolding, respectively. The sample composition of the DVD-Ig solution used was 1 mg/ml DVD-Ig protein, 1 mM ionic strength Histidine pH 6, 1° C./minute scan rate.

FIG. 2A shows a graphic description of serum stability of various DVD-Ig proteins.

FIG. 2B shows the domain orientation concept for different variable domain combinations.

FIG. 3 shows a graphic description of the correlation between pharmacokinetic parameters of different DVD-Ig proteins and high molecular weight (HMW) aggregate formation.

### DETAILED DESCRIPTION

### I. Definitions

The term “multivalent binding protein” is used to denote a binding protein comprising two or more target binding sites. The multivalent binding protein may be engineered to have the three or more antigen binding sites, and is generally not a naturally occurring antibody.

The term “multispecific binding protein” refers to a binding protein capable of binding two or more related or unrelated targets. An example of a multivalent binding protein is a Dual Variable Domain (DVD) binding protein, such as a DVD-Ig™. In an embodiment, DVD binding proteins comprise two or more antigen binding sites and are tetravalent or multivalent binding proteins. DVDs may be monospecific, i.e., capable of binding one target, or multispecific, i.e., capable of binding two or more targets.

The term “Dual Variable Domain Immunoglobulin” or “DVD-Ig™” or “DVD-Ig protein” refers to a DVD binding protein comprising two heavy chain DVD polypeptides and two light chain DVD polypeptides. Each half of a DVD-Ig comprises a heavy chain DVD polypeptide and a light chain DVD polypeptide, and two target binding sites. Each binding site comprises a heavy chain variable domain and a light chain variable domain with a total of 6 CDRs involved in target binding. Each variable domain (VD) in a DVD-Ig protein may be obtained from one or more “parent” monoclonal antibodies (mAbs) that bind one or more desired antigens or epitopes. In an embodiment, the resulting DVD-Ig molecule retains activities of both parental mAbs. The term “DVD-Ig” is inclusive of the terms AS-DVD-Ig protein and LS-DVD-Ig proteins described below.

The term “Aqueous Stable Dual Variable Domain Immunoglubulin” or “AS-DVD-Ig” or “AS-DVD-Ig protein” refers to a subset of DVD-Igs that have low aggregation or a low change in monomer content due to physical degradation, such as aggregation, following stability tests at either 5° C. or 40° C. for 14 to 21 days at a concentration ranging from 1 to 100 mg/ml and at a pH between about 5.5 to about 6.5. Different stability tests may be used to define an AS-DVD-Ig. In one embodiment, an AS-DVD-Ig is a DVD-Ig protein that has 10% relative (rel.) peak area or less change in monomers at about 40° C. after 21 days of storage at a concentration of 100 mg/ml in an aqueous formulation or, alternatively, a DVD-Ig protein that has 1% rel. peak area or less change in monomers at about 5° C. after 21 days of storage at a concentration of 100 mg/ml and at a pH between about 5.5 to 6.5 in an aqueous formulation. Alternatively, an AS-DVD-Ig is a DVD-Ig protein that has 1.5% rel. peak area or less change in monomers at about 5° C. after 21 days of storage at a concentration of 1 mg/ml in an aqueous formulation or 3% rel. peak area or less change in monomers at about 40° C. after 21 days of storage at a concentration of 1 mg/ml and at a pH between about 5.5 to 6.5 in an aqueous formulation. In another embodiment, an AS-DVD-Ig protein is defined as a DVD-Ig protein that has a change in monomers less than 10% rel. peak area following accelerated storage after 14 days at about 40° C., when formulated at a concentration over 50 mg/ml and at a pH between 5.5-6.5 in an aqueous formulation. In another embodiment, an AS-DVD-Ig protein is defined as a DVD-Ig protein that has less than 8% rel. peak area change in monomers following 14 days of accelerated storage (at, for example, about 40° C.) when formulated at a concentration over 60 mg/ml and at a pH between 5.5-6.5 in an aqueous formulation. In one embodiment, an AS-DVD-Ig protein is defined as a DVD-Ig protein that has 6% rel. peak area or less change in monomers following 14 days of accelerated storage (at, for example, about 40° C.). In a further embodiment, an AS-DVD-Ig protein is defined as a DVD-Ig protein that has less than 5% rel. peak area change in monomers following 14 days of accelerated storage (at, for example, about 40° C.). In one embodiment, the accelerated storage conditions include storing the DVD-Ig protein in the absence of light at 40° C. DVD-Ig proteins may be tested in aqueous formulations containing citrate and phosphate buffer, or histidine buffer at a pH between 5.5-6.5. In one embodiment, an AS-DVD-Ig protein has 10% rel. peak area or less change in monomers as determined by SEC analysis following accelerated storage for 21 days at about 40° C., where the AS-DVD-Ig protein is formulated at a concentration of at least 100 mg/ml in a citrate phosphate buffer or histidine buffer at a pH between 5.5-6.5 in an aqueous formulation. In one embodiment, an AS-DVD-Ig protein has less than 6% rel. peak area change in monomers as determined by SEC analysis following accelerated storage for 14 days at about 40° C., where the AS-DVD-Ig protein is formulated at a concentration of at least 50 mg/ml in a citrate phosphate buffer or histidine buffer in an aqueous formulation.

In one embodiment, an AS-DVD-Ig is defined as a DVD-Ig protein that has 10% or less aggregation at about 40° C. after 21 days of storage at a concentration of 100 mg/ml in an aqueous formulation or, alternatively, a DVD-Ig protein that has 1% or less aggregation at about 5° C. after 21 days of storage at a concentration of 100 mg/ml in an aqueous formulation. Alternatively, an AS-DVD-Ig is a DVD-Ig protein that has 1.5% or less aggregation at about 5° C. after 21 days of storage at a concentration of 1 mg/ml in an aqueous formulation or 3% or less aggregation at about 40° C. after 21 days of storage at a concentration of 1 mg/ml in an aqueous formulation. In another embodiment, an AS-DVD-Ig protein is defined as a DVD-Ig protein that has less than 10% aggregate formed following accelerated storage after 14 days at about 40° C., when formulated at a concentration over 50 mg/ml in an aqueous formulation. In one embodiment, an AS-DVD-Ig protein is defined as a DVD-Ig protein that has less than 10% aggregation following 14 days of accelerated storage (at, for example, about 40° C.). In another embodiment, an AS-DVD-Ig protein is defined as a DVD-Ig protein that has less than 8% aggregation following 14 days of accelerated storage (at, for example, about 40° C.). In one embodiment, an AS-DVD-Ig protein is defined as a DVD-Ig protein that has 6% or less aggregation following 14 days of accelerated storage (at, for example, about 40° C.). In a further embodiment, an AS-DVD-Ig protein is defined as a DVD-Ig protein that has less than 5% aggregation following 14 days of accelerated storage (at, for example, about 40° C.). Aggregation can be determined according to methods known in the art, including, but not limited to, size exclusion chromatography (SEC). In one embodiment, the accelerated storage conditions include storing the DVD-Ig protein in the absence of light at 40° C. DVD-Ig proteins may be tested in aqueous formulations containing citrate and phosphate buffer, or histidine buffer. In one embodiment, an AS-DVD-Ig protein has 10% or less aggregation as determined by SEC analysis following accelerated storage for 21 days at about 40° C., where the AS-DVD-Ig protein is formulated at a concentration of at least 100 mg/ml in a citrate phosphate buffer or histidine buffer in an aqueous formulation. In one embodiment, an AS-DVD-Ig protein has less than 6% aggregation as determined by SEC analysis following accelerated storage for 14 days at about 40° C., where the AS-DVD-Ig protein is formulated at a concentration of at least 50 mg/ml in a citrate phosphate buffer or histidine buffer in an aqueous formulation.

The term “aqueous formulation” refers to a liquid solution in which the solvent is water. In another embodiment, the term “aqueous formulation” refers to a liquid formulation in which the solvent is water wherein the formulation was not previously lyophilized, (i.e., does not result from reconstitution of a lyophilized formulation).

The term “Lyophilized Stable Dual Variable Domain Immunoglubulin” or “LS-DVD-Ig” or “LS-DVD-Ig protein” refers to a subset of DVD-Ig proteins that have low aggregation or elevated levels of change in monomers in the liquid state. Different stability tests may be used to define an LS-DVD-Ig. In one embodiment, an LS-DVD-Ig protein has more than 10% rel. peak area change in monomers observed, following accelerated storage, e.g., 21 days of accelerated storage at 40° C., when formulated at a concentration over 100 mg/ml at a pH between 5.5-6.5 in an aqueous formulation. In one embodiment, an LS-DVD-Ig protein has 20% rel. peak area or less change in monomers following 21 days of accelerated storage at 40° C., when formulated at a concentration over 100 mg/ml at a pH between 5.5-6.5 in an aqueous formulation. In another embodiment, an LS-DVD-Ig protein has less than 18% rel. peak area change in monomers following 14 days of accelerated storage, at, for example, about 40° C. In a further embodiment, an LS-DVD-Ig protein has less than 13% rel. peak area change in monomers following 14 days of accelerated storage, at, for example, about 40° C. Alternatively, an LS-DVD-Ig protein is defined as a DVD-Ig protein that has 1% rel. peak area or less change in monomers following 4 freeze thaw cycles cycles. Alternatively, an LS-DVD-Ig protein is defined as a DVD-Ig protein that has 4% rel. peak area or less change in monomers following 7 days at 25° C. at a concentration between 1-100 mg/mL in aqueous solution at the most stable pH. Alternatively, an LS-DVD-Ig protein is defined as a DVD-Ig protein that has 1% rel. peak area or less change in monomers following 7 days at 5° C. in aqueous solution at the most stable pH. DVD-Ig proteins may be tested in aqueous formulations containing citrate and phosphate buffer, or histidine buffer. Change in monomers can be determined according to methods known in the art, including, but not limited to, SEC. In one embodiment, the accelerated storage conditions include storing the DVD-Ig protein in the absence of light at 40° C. In one embodiment, an LS-DVD-Ig protein has 20% rel. peak area or less change in monomers as determined by SEC analysis following accelerated storage for 14 days at about 40° C., where the LS-DVD-Ig protein is formulated at a concentration of at least 50 mg/ml in a citrate phosphate buffer in an aqueous formulation.

In one embodiment, an LS-DVD-Ig protein has less than 15% aggregate formed, following accelerated storage, e.g., 14 days of accelerated storage at 40° C., when formulated at a concentration over 50 mg/ml in an aqueous formulation. In one embodiment, an LS-DVD-Ig protein has 15% or less aggregation following 14 days of accelerated storage at, for example, 40° C. In another embodiment, an LS-DVD-Ig protein has less than 14% aggregation following 14 days of accelerated storage, at, for example, about 40° C. In a further embodiment, an LS-DVD-Ig protein has less than 13% aggregation following 14 days of accelerated storage, at, for example, about 40° C. Alternatively, an LS-DVD-Ig protein is defined as a DVD-Ig protein that has 1% or less aggregation following 4 freeze thaw cycles cycles. DVD-Ig proteins may be tested in aqueous formulations containing citrate and phosphate buffer, or histidine buffer. Aggregation can be determined according to methods known in the art, including, but not limited to, SEC. In one embodiment, the accelerated storage conditions include storing the DVD-Ig protein in the absence of light at 40° C. In one embodiment, an LS-DVD-Ig protein has 15% or less aggregation as determined by SEC analysis following accelerated storage for 14 days at about 40° C., where the LS-DVD-Ig protein is formulated at a concentration of at least 50 mg/ml in a citrate phosphate buffer in an aqueous formulation.

The term “pharmaceutical formulation” refers to preparations that are in such a form as to permit the biological activity of the active ingredients to be effective and, therefore, may be administered to a subject for therapeutic use.

A “stable” formulation is one in which the DVD-Ig protein therein essentially retains its physical stability and/or chemical stability and/or biological activity upon storage. Various analytical techniques for measuring protein stability are available in the art and are reviewed in, e.g., Peptide and Protein Drug Delivery, pp. 247-301, Vincent Lee Ed., Marcel Dekker, Inc., New York, N.Y., Pubs. (1991) and Jones (1993) Adv. Drug Delivery Rev. 10: 29-90. In one embodiment, the stability of the DVD-Ig protein is determined according to the percentage of monomer protein in the solution, with a low percentage of degraded (e.g., fragmented) and/or aggregated protein. For example, an aqueous formulation comprising a stable DVD-Ig protein may include at least 95% monomer DVD-Ig protein, e.g., AS-DVD-Ig protein. Alternatively, an aqueous formulation of the invention may include no more than 5% aggregate and/or degraded DVD-Ig protein, e.g., AS-DVD-Ig protein.

A DVD-Ig protein “retains its physical stability” in a pharmaceutical formulation if it shows substantially no signs of aggregation, precipitation and/or denaturation upon visual examination of color and/or clarity, or as measured by UV light scattering or by size exclusion chromatography. In one aspect of the invention, a stable aqueous formulation is a formulation having less than about 10% aggregation, and less than about 5% AS-DVD-Ig protein aggregation in the formulation.

A DVD-Ig protein “retains it chemical stability” in a pharmaceutical formulation if the chemical stability at a given time is such that the DVD-Ig protein is considered to still retain its biological activity as defined below. Chemical stability can be assessed by detecting and quantifying chemically altered forms of the DVD-Ig. Chemical alteration may involve size modifications (e.g., clipping) which can be evaluated using size exclusion chromatography, SDS-PAGE and/or matrix-assisted laser desorption ionization/time of flight mass spectrometry (MALDI/TOF MS), for example. Other types of chemical alternation include charge alteration (e.g., occurring as a result of deamidation), which can be evaluated by, e.g., ion-exchange chromatography.

A DVD-Ig protein “retains its biological activity” in a pharmaceutical formulation, if the protein in a pharmaceutical formulation is biologically active for its intended purpose. For example, biological activity of a DVD-Ig protein is retained if the biological activity of the DVD-Ig protein in the pharmaceutical formulation is within about 30%, about 20%, or about 10% (within the errors of the assay) of the biological activity exhibited at the time the pharmaceutical formulation was prepared (e.g., as determined in an antigen binding assay).

The term “surfactant”, as used herein, refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Examples of suitable surfactants include, but are not limited to, sodium lauryl sulfate, polysorbates such as polyoxyethylene sorbitan monooleate, monolaurate, monopalmitate, monstearate or another ester of polyoxyethylene sorbitan (e.g., the commercially available Tweens™, such as, Tween™ 20 and Tween™ 80 (ICI Speciality Chemicals)), sodium dioctylsulfosuccinate (DOSS), lecithin, stearylic alcohol, cetostearylic alcohol, cholesterol, polyoxyethylene ricin oil, polyoxyethylene fatty acid glycerides, poloxamers (e.g., Pluronics F68™ and F108™, which are block copolymers of ethylene oxide and propylene oxide); polyoxyethylene castor oil derivatives or mixtures thereof. In one embodiment, a formulation of the disclosure comprises Polysorbate 20, Polysorbate 40, Polysorbate 60, or Polysorbate 80.

The term “tonicity modifier” or “tonicity agent” refers to a compound that can be used to adjust the tonicity of a liquid formulation. Examples of tonicity modifiers include glycerin, lactose, mannitol, dextrose, sodium chloride, magnesium sulfate, magnesium chloride, sodium sulfate, sorbitol, trehalose, sucrose, raffinose, maltose and others known to those or ordinary skill in the art.

The term “polyol” refers to a substance with multiple hydroxyl groups, and includes sugars (reducing and nonreducing sugars), sugar alcohols and sugar acids. In one embodiment, polyols have a molecular weight that is less than about 600 kD (e.g., in the range from about 120 to about 400 kD). A “reducing sugar” is one that contains a free aldehyde or ketone group and can reduce metal ions or react covalently with lysine and other amino groups in proteins. A “nonreducing sugar” is one that lacks a free aldehyde or ketonic group and is not oxidised by mild oxidising agents such as Fehling's or Benedict's solutions. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof. The polyol may also act as a tonicity agent. In one embodiment of the invention, one ingredient of the formulation is sorbitol in a concentration of about 10 to about 70 mg/ml. In a particular embodiment of the invention, the concentration of sorbitol is about 30 to about 50 mg/ml. In another embodiment, the concentration of sucrose is about 60 to about 100 mg/ml. In a particular embodiment of the invention, the concentration of sucrose is about 70 to about 90 mg/ml.

The term “buffer” refers to a buffered solution that resists changes in pH by the action of its acid-base conjugate components. A buffer used in this invention has a pH in the range from about 4.5 to about 7.5. Examples of buffers that will control the pH in this range include acetate (e.g., sodium acetate), succinate (such as sodium succinate), gluconate, methionine, imidazole, histidine, glycine, arginine, citrate, phosphate, citrate and phosphate, Tris, and other organic acid buffers. In one embodiment, the buffer used in the formulation of the invention is histidine, glycine, arginine, acetate, citrate, and/or phosphate buffered saline (PBS).

A “reconstituted” formulation is one which has been prepared by dissolving a lyophilized protein formulation in a diluent such that the protein is dispersed in the reconstituted formulation. The reconstituted formulation is suitable for administration (e.g. parenteral administration) to a patient to be treated with the protein of interest (e.g., LS-DVD-Ig).

A “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “therapeutically effective amount” or “effective amount” of a binding protein refers to an amount effective in the prevention or treatment of a disorder for the treatment of which the antibody is effective.

The term “disorder” refers to any condition that would benefit from treatment with the formulations of the invention. This includes chronic and acute disorders or diseases including those pathological conditions that predispose the subject to the disorder in question.

The term “treatment” refers to both therapeutic treatment and prophylactic or preventative measures. Those patients in need of treatment include those already with the disorder as well as those in which the disorder is to be prevented.

The terms “parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. The phrases “systemic administration,” “administered systemically,” “peripheral administration” and “administered peripherally” mean the administration of a compound, drug or other material other than directly into the central nervous system, such that it enters the patient's system and is subject to metabolism and other like processes, for example, subcutaneous administration.

The term “antibody” broadly refers to an immunoglobulin (Ig) molecule, generally comprised of four polypeptide chains, two heavy (H) chains and two light (L) chains, or any functional fragment, mutant, variant, or derivative thereof, that retains the essential target binding features of an Ig molecule.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY) and class (e.g., IgG1, IgG2, IgG 3, IgG4, IgA1 and IgA2) or subclass.

The term “Fc region” means the C-terminal region of an immunoglobulin heavy chain, which may be generated by papain digestion of an intact antibody. The Fc region may be a native sequence Fc region or a variant sequence Fc region. The Fc region of an immunoglobulin generally comprises two constant domains, a CH2 domain and a CH3 domain, and optionally comprises a CH4 domain.

The term “antigen-binding portion” refers to one or more fragments of a binding proteinthat specifically binds to a target or an antigen. Such embodiments may be monospecific, or may be bispecific, dual specific, or multi-specific (may specifically bind two or more different antigens).

A “functional antigen binding site” of a binding protein is one that is capable of binding a target antigen. The antigen binding affinity of the functional antigen binding site is not necessarily as strong as the parent antibody from which the antigen binding site is derived, but the ability to bind antigen must be measurable using a known method for evaluating antibody binding to an antigen. Moreover, the antigen binding affinity of each of the functional antigen binding sites of a multivalent binding protein need not be quantitatively the same.

The term “linker” denotes polypeptides comprising two or more amino acid residues joined by peptide bonds that are used to link one or more antigen binding portions. Such linker polypeptides are well known in the art (see, e.g., Holliger et al. (1993) Proc. Natl. Acad. Sci. USA 90:6444-6448; Poljak et al. (1994) Structure 2:1121-1123).

An “immunoglobulin constant domain” refers to a heavy or light chain constant domain. Human heavy chain and light chain (e.g., IgG) constant domain amino acid sequences are known in the art.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different epitopes, each monoclonal antibody is directed against a single epitope on the antigen. The modifier “monoclonal” is not to be construed as requiring production of the antibody by any particular method.

The term “human antibody” includes antibodies that have variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody” is not intended to include antibodies in which CDR sequences derived from the germline of another mammalian species, such as a mouse, have been grafted onto human framework sequences.

The term “chimeric antibody” means antibodies that comprise heavy and light chain variable region sequences from one species and constant region sequences from another species, such as antibodies having murine heavy and light chain variable regions linked to human constant regions.

The term “CDR-grafted antibody” means antibodies that comprise heavy and light chain variable region sequences from one species but in which the sequences of one or more of the CDR regions of their VH and/or VL are replaced with the CDR sequences of another species, such as antibodies having human heavy and light chain variable regions in which one or more of the murine CDRs (e.g., CDR3) has been replaced with murine CDR sequences.

The term “humanized antibody” means an antibody that comprises heavy and light chain variable region sequences from a non-human species (e.g., a mouse) but in which at least a portion of the VH and/or VL sequence has been altered to be more “human-like”, i.e., more similar to human germline variable sequences. One type of humanized antibody comprises non-human CDR sequences and human framework sequences.

The term “CDR” means the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the target. The exact boundaries of these CDRs have been defined differently according to different systems.

The terms “Kabat numbering”, “Kabat definitions and “Kabat labeling” are used interchangeably herein. These terms refer to a system of numbering amino acid residues that are more variable (i.e., hypervariable) than other amino acid residues in the heavy and light chain variable regions of an antibody, or an antigen binding portion thereof (Kabat et al. (1971) Ann. NY Acad Sci. 190:382-391 and Kabat et al. (1991) Sequences of Proteins of Immunological Interest, Fifth Edition, U.S. Department of Health and Human Services, NIH Publication No. 91-3242). For the heavy chain variable region, the hypervariable region generally ranges from amino acid positions 31 to 35 for CDR1, amino acid positions 50 to 65 for CDR2, and amino acid positions 95 to 102 for CDR3. For the light chain variable region, the hypervariable region generally ranges from amino acid positions 24 to 34 for CDR1, amino acid positions 50 to 56 for CDR2, and amino acid positions 89 to 97 for CDR3. Chothia and coworkers (Chothia and Lesk (1987) J. Mol. Biol. 196:901-917 and Chothia et al. (1989) Nature 342:877-883 found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although embodiments use Kabat or Chothia defined CDRs.

The term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-H1, -H2, and -H3 of the heavy chain and CDR-L1, -L2, and -L3 of the light chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. The term “activity” includes activities such as the binding specificity and binding affinity of a DVD-Ig protein for two or more antigens.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope is a region of an antigen that is bound by an antibody or multispecific binding protein.

The term “surface plasmon resonance” or “SPR”, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson et al. (1993) Ann. Biol. Clin. 51:19-26; Jonsson et al. (1991) Biotechniques 11:620-627; Johnsson et al. (1995) J. Mol. Recognit. 8:125-131; and Johnnson et al. (1991) Anal. Biochem. 198:268-277.

The term “Kₒₙ” refers to the on rate constant for association of a binding protein to the antigen to form the binding protein/antigen complex as is known in the art.

The term “K_{off}” refers to the off rate constant for dissociation of a binding protein from the binding protein/antigen complex as is known in the art.

The term “K_{d}” refers to the dissociation constant of a particular antibody-antigen interaction as is known in the art.

### II. Dual Variable Domain Immunoglobulin (DVD-Ig) Proteins for Use in Formulations of the Invention

The invention pertains to formulations, and uses thereof, of DVD-Ig proteins, particularly those identified as AS-DVD-Ig proteins or LS-DVD-Ig proteins (described in more detail below).

In one embodiment, the DVD-Ig protein used in the formulations and methods of the invention comprises a polypeptide chain, wherein said polypeptide chain comprises VD1-(X1)n-VD2-C-(X2)n, wherein VD1 is a first variable domain, VD2 is a second variable domain, C is a constant domain, X1 represents an amino acid or polypeptide, X2 represents an Fc region and n is 0 or 1.

Examples of DVD-Ig proteins that may be used in the method and compositions of the invention are provided in Tables 61 and 66 and described in SEQ ID NOs: 28 to 75.

In one embodiment, a DVD-Ig protein contains two polypeptide chains, wherein a first polypeptide chain comprises VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, and X2 is an Fc region; and a second polypeptide chain comprises VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1; and wherein said two polypeptide chains of said binding protein form two functional antigen binding sites.

In one embodiment, a DVD-Ig protein contains four polypeptide chains, wherein two polypeptide chains comprise VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, and X2 is an Fc region; and two polypeptide chains comprise VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1; and wherein said four polypeptide chains of said binding protein form four functional antigen binding sites.

In one embodiment, the AS-DVD-Ig protein or LS-DVD-Ig protein comprises a polypeptide chain wherein the polypeptide chain comprises VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; and n is 0 or 1. In one embodiment, the AS-DVD-Ig protein or LS-DVD-Ig protein comprises a polypeptide chain, wherein the polypeptide chain comprises VD1-(X1)n-VD2-C-(X2)n, wherein VD1 is a first light chain variable domain; VD2 is a second light chain variable domain; C is a light chain constant domain; X1 is a linker with the proviso that it is not a CH1 or CL; X2 does not comprise an Fc region; and n is 0 or 1. In a further embodiment, (X1)n on the heavy and/or light chain is (X1)0 and/or (X2)n on the heavy and/or light chain is (X2)0.

In one embodiment, the AS-DVD-Ig protein or LS-DVD-Ig protein comprises first and second polypeptide chains, wherein the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a first linker with the proviso that it is not CH2; X2 is an Fc region; n is 0 or 1; and wherein the second polypeptide chain comprises a second VD1-(X1)n-VD2-C-(X2)n, wherein VD1 is a first light chain variable domain; VD2 is a second light chain variable domain; C is a light chain constant domain; X1 is a second linker with the proviso that it is not CH1 or CL; X2 does not comprise an Fc region; and n is 0 or 1.

In one embodiment, the VD1 of the first polypeptide chain and the VD1 of the second polypeptide chain are from different first and second parent antibodies, respectively, or binding portions thereof. In one embodiment, the VD2 of the first polypeptide chain and the VD2 of the second polypeptide chain are from different first and second parent antibodies, respectively, or binding portions thereof. In one embodiment, the first and the second parent antibodies bind different epitopes on the same target or different targets. In one embodiment, the first parent antibody or binding portion thereof binds the first target with a potency different from the potency with which the second parent antibody or binding portion thereof binds the second target. In one embodiment, the first parent antibody or binding portion thereof binds the first target with an affinity different from the affinity with which the second parent antibody or binding portion thereof binds the second target.

In one embodiment, the AS-DVD-Ig protein or LS-DVD-Ig protein comprise, two first polypeptide chains and two second polypeptide chains.

In one embodiment, the AS-DVD-Ig protein or LS-DVD-Ig protein comprises first and second polypeptide chains, each independently comprising VD1-(X1)n-VD2-C-(X2)n, wherein VD1 is a first variable domain; VD2 is a second variable domain; C is a constant domain; X1 is a linker with the proviso that it is not CH1; X2 is an Fc region; n is 0 or 1, wherein the VD1 domains on the first and second polypeptide chains form a first functional target binding site and the VD2 domains on the first and second polypeptide chains form a second functional target binding site. IN a further embodiment, the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; n is 0 or 1, and wherein the second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1; X2 does not comprise an Fc region; n is 0 or 1, wherein the VD1 domains on the first and second polypeptide chains form a first functional target binding site and the VD2 domains on the first and second polypeptide chains form a second functional target binding site.

In one embodiment, the AS-DVD-Ig protein or LS-DVD-Ig protein comprises first and second polypeptide chains, each independently comprising VD1-(X1)n-VD2-C-(X2)n, wherein VD1 is a first variable domain; VD2 is a second variable domain; C is a constant domain; X1 is a linker with the proviso that it is not CH1; X2 is an Fc region; n is 0 or 1, and wherein the VD1 domains on the first and second polypeptide chains form a first functional target binding site and the VD2 domains on the first and second polypeptide chains form a second functional target binding site. IN a further embodiment, the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; n is 0 or 1, and wherein the second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1; X2 does not comprise an Fc region; n is 0 or 1, wherein the VD1 domains on the first and second polypeptide chains form a first functional target binding site and the VD2 domains on the first and second polypeptide chains form a second functional target binding site.

Examples of DVD-Ig proteins are described in U.S. Pat. No. 7,612,181, which is incorporated by reference herein.

### Generation of DVD-Ig Proteins

The variable domains of a DVD-Ig protein can be obtained from parent antibodies, including polyclonal and monoclonal antibodies capable of binding targets of interest. These antibodies may be naturally occurring or may be generated by recombinant technology. Examples of antibodies that may be used in making DVD-Ig proteins include chimeric antibodies, human antibodies, and humanized antibodies. Monoclonal antibodies can be prepared using a wide variety of techniques known in the art including, for example, the use of hybridoma, recombinant, and phage display technologies, or any combination thereof. Monoclonal antibodies may also be produced by immunizing a non-human animal comprising some, or all, of the human immunoglobulin locus with an antigen of interest, such as, for example, XENOMOUSE™ transgenic mouse, an engineered mouse strain that comprises large fragments of the human immunoglobulin loci and is deficient in mouse antibody production. Methods of generating DVD-Ig proteins are described in U.S. Pat. No. 7,612,181, the teachings of which are incorporated by reference herein. DVD-Ig proteins used in the compositions and methods of the invention may be made from antibodies capable of binding specific targets and well known in the art. These include, but are not limited to an anti-TNF antibody (U.S. Pat. No. 6,258,562), anti-IL-12 and or anti-IL-12p40 antibody (U.S. Pat. No. 6,914,128); anti-IL-18 antibody (US Patent Publication No. 20050147610), as well as anti-05, anti-CBL, anti-CD147, anti-gp120, anti-VLA4, anti-CD11a, anti-CD18, anti-VEGF, anti-CD40L, anti-Id, anti-ICAM-1, anti-CXCL13, anti-CD2, anti-EGFR, anti-TGF-beta 2, anti-E-selectin, anti-Fact VII, anti-Her2/neu, anti-F gp, anti-CD11/18, anti-CD14, anti-ICAM-3, anti-CD80, anti-CD4, anti-CD3, anti-CD23, anti-beta2-integrin, anti-alpha4beta7, anti-CD52, anti-HLA DR, anti-CD22, anti-CD20, anti-MIF, anti-CD64 (FcR), anti-TCR alpha beta, anti-CD2, anti-Hep B, anti-CA 125, anti-EpCAM, anti-gp120, anti-CMV, anti-gpIIbIIIa, anti-IgE, anti-CD25, anti-CD33, anti-HLA, anti-VNRintegrin, anti-IL-1alpha, anti-IL-1beta, anti-IL-1 receptor, anti-IL-2 receptor, anti-IL-4, anti-IL4 receptor, anti-IL5, anti-IL-5 receptor, anti-IL-6, anti-IL-8, anti-IL-9, anti-IL-13, anti-IL-13 receptor, anti-IL-17, and anti-IL-23 antibodies (see Presta (2005) J. Allergy Clin. Immunol. 116:731-6 and Clark “Antibodies for Therapeutic Applications,” Department of Pathology, Cambridge University, UK (2000), published online at M. Clark's home page at the website for the Department of Pathology, Cambridge University.

Parent monoclonal antibodies may also be selected from various therapeutic antibodies approved for use, in clinical trials, or in development for clinical use. Such therapeutic antibodies include, but are not limited to: rituzimab (RITUXAN™ Biogen Idec, Genentech/Roche) (see for example U.S. Pat. No. 5,736,137) a chimeric anti-CD20 antibody approved to treat non-Hodgkin's lymphoma; ofatumumab (HUMAX-CD20™ Genmab, GlaxoSmithKlein) (described in U.S. Pat. No. 5,500,362) an anti-CD20 antibody approved to treat chronic lymphocytic leukemia that is refractory to fludarabine and alemtuzumab; AME-133v (Mentrik Biotech) an anti-CD20 antibody; veltuzumab (hA20) (Immunomedics) an anti-CD20 antibody; HumaLYM (Intracel); PRO70769 (Genentech/Roche) (PCT/US2003/040426) an anti-CD20 antibody; trastuzumab (HERCEPTIN™ Genentech/Roche) (described in U.S. Pat. No. 5,677,171) a humanized anti-Her2/neu antibody approved to treat breast cancer; pertuzumab (rhuMab-2C4, OMNITARG™ Genentech/Roche) (described in U.S. Pat. No. 4,753,894); cetuximab (ERBITUX™ Imclone) (described in U.S. Pat. No. 4,943,533; PCT WO 96/40210) a chimeric anti-EGFR antibody approved to treat colorectal and head and neck cancer; panitumumab (ABX-EGF VECTIBIX® Amgen) (described in U.S. Pat. No. 6,235,883) an anti-EGFR antibody approved to treat colorectal cancer; zalutumumab (HUMAX-EGFR™ Genmab) (described in U.S. patent application Ser. No. 10/172,317) an anti-EGFR antibody; EMD55900 (Mab 425 Merck) an anti-EGFR antibody; EMD62000 and EMD72000 (Mab 425 Merck) anti-EGFR antibodies (described in U.S. Pat. No. 5,558,864; Murthy et al. (1987) Arch. Biochem. Biophys. 252(2):549-60; Rodeck et al. (1987) J. Cell. Biochem. 35(4):315-20; Kettleborough et al. (1991) Protein Eng. 4(7):773-83; ICR62 (Institute of Cancer Research) an anti-EGFR antibody (described in PCT Publication No. WO 95/20045; Modjtahedi et al. (1993) J. Cell. Biophys. 22(1-3):129-46; Modjtahedi et al. (1993) Br. J. Cancer 67(2):247-53; Modjtahedi et al. (1996) Br. J. Cancer 73(2):228-35; Modjtahedi et al. (2003) Int. J. Cancer 105(2):273-80); nimotuzumab (TheraCIM hR3, THERALOC® YM Biosciences, Oncoscience AG) (described in U.S. Pat. No. 5,891,996; U.S. Pat. No. 6,506,883; Mateo et al. (1997) Immunotechnol. 3(1):71-81) an anti-EGFR antibody; ABT-806 (Ludwig Institute for Cancer Research, Memorial Sloan-Kettering) (Jungbluth et al. (2003) Proc. Natl. Acad. Sci. USA 100(2):639-44) an anti-EGFR antibody; KSB-102 (KS Biomedix); MR1-1 (IVAX, National Cancer Institute) (PCT Publication No. WO 0162931A2) an anti-EGFRvIII antibody; SC100 (Scancell) (PCT Publication No. WO 01/88138) an anti-EGFR antibody; alemtuzumab (CAMPATH™ Genzyme/Sanofi) an anti-CD52 antibody approved to treat B-cell chronic lymphocytic leukemia; muromonab-CD3 (Orthoclone OKT3™ Johnson and Johnson) an anti-CD3 antibody approved to treat organ transplant rejection; ibritumomab tiuxetan (ZEVALIN™ Spectrum Pharmaceuticals) an anti-CD20 antibody approved to treat non-Hogkins Lymphoma; gemtuzumab ozogamicin (hP67.6 MYLOTARG™ Pfizer) an anti-CD33 antibody conjugated to calicheamicin; alefacept (AMEVIVE™ Astellas Pharma) an anti-CD2 LFA-3 Fc fusion; abciximab (REOPRO™ Centocor Ortho Biotech Products, Lilly) a chimeric human-mouse anti-glycoprotein IIb/IIIa receptor and anti-vitronectic αᵥβ₃ receptor antibody approved as an adjunct to percutaneous coronary intervention to prevent cardiac ishemia; basiliximab (SIMULECT™ Novartis) an anti-CF25 antibody approved to treat organ transplant rejection; palivizumab (SYNAGIS™ Medimmune) an antibody to the A antigenic site of F protein of RSV approved to treat RSV invection; infliximab (REMICADE™ Janssen Biotech) an anti-TNFalphaα antibody approved to treat Crohn's disease, ulcerative colitis, arthritis, ankylosing spondylitis, psoriatic arthritis, and plaque psoriasis; adalimumab (HUMIRA™ Abbott) an anti-TNFα antibody approved to treat rheumatoid arthritis, juvenile idiopathic arthritis, psoriatic arthritis, ankylosing spondylitis, Crohn's disease, ulcerative colitis, plaque psoriasis; CDP571 (HUMICADE™ Celltech, Biogen IDEC) an anti-TNFα antibody; etanercept (ENBREL™ Amgen, Pfizer) an anti-TNFα Fc fusion antibody approved to treat rheumatoid arthritis, juvenile idiopathic arthritis, psoriatic arthritis, ankylosing spondylitis, plaque psoriasis; certolizumab pegol (CIMZIA)UCB Pharma) an anti-TNFα antibody approved to treat rheumatoid arthritis and Crohn's disease; ustekinumab (STELARA Janssen Biotech) a human anti-p40 subunit of IL-12 and IL-23 antibody approved to treat plaque psoriasis; galilimomab (ABX-CBL Abgenix) a mouse anti-CD147 antibody; ABX-IL8 (Abgenix) an anti-IL8 antibody; ABX-MA1 (Abgenix) an anti-MUC18 antibody; pemtumomab (Theragyn, R1549, 90Y-muHMFGlAntisoma) a mouse anti-MUC1-Yttrium 90 antibody conjugate; Therex (R1550 Antisoma) an anti-MUC1 antibody; AngioMab (muBC-1, AS1405 Antisoma) f; HuBC-1 (Antisoma); Thioplatin (AS1407 Antisoma); natalizumab (TYSABRI® Biogen Idec, Elan) an anti-α4 integrin antibody approved to treat multiple sclerosis and Crohn's disease; VLA-1 (Santarus) a humanized anti-VLA-1 antibody; LTBR mAb (Biogen Idec) an anti-lymphotoxin β receptor antibody; lerdelimumab (CAT-152 Cambridge Antibody Technology/Abbott) an anti-TGF-β2 antibody; briakinumab (Abbott) an anti-IL-12 and 23 antibody; metelimumab (CAT-192 Cambridge Antibody Technology, Genzyme) an anti-TGFβ1 antibody; bertilimumab (CAT-213, iCO-008 Cambridge Antibody Technology, iCo Therapeutics, Immune Pharmaceuticals) an anti-eotaxin1 antibody; belimumab (BENLYSTA® Human Genome Science, GlaxoSmithKline) an anti-B lymphocyte stimulator protein antibody approved to treat systemic lupus erythematosus; maputumumab (HGS-ETR1 Cambridge Antibody Technology, Human Genome Sciences) an anti-TRAIL-R1 antibody; bevacizumab (AVASTIN™ Genentech/Roche) an anti-VEGF antibody approved to treat metastatic colorectal cancer, non-squamous non-small cell lung cancer, glioblastoma, metastatic renal cell cancer; anti-HER3/EGFR antibody (Genentech/Roche); an Anti-Tissue Factor antibody (Genentech/Roche); omalizumab (XOLAIR™ Genentech/Roche, Novartis) an anti-IgE antibody approved to treat severe allergic asthma; efalizumab (RAPTIVA™ Genentech/Roche, Merck Serono) an anti-CD11a antibody; MLN-02 (Millenium, Genentech/Roche) an anti-α4β7 integrin antibody; zanolimumab (HUMAX CD4™ Emergent BioSolutions) an anti-CD4 antibody; HUMAX-IL15™ (AMG-714 Genmab, Amgen) an anti-IL15 antibody; HuMax-IL8 (HUMAX-Inflam™, MDX-018 Genmab, Cormorant Pharmaceuticals) an anti-IL8 antibody; HUMAX™-Cancer, (Genmab, Medarex, Oxford GlycoSciences) an anti-Heparanase I antibody; HUMAX™-Lymphoma (Genmab) an anti-IL8 antibody; HUMAX™-TAC (Genmab) an anti-IL-2Rα, CD25 antibody; daratumumab (HuMax®-CD38, Genmab, Janssen Biotech) an anti-CD38 antibody; toralizumab (IDEC-131 Biogen Idec) an anti-CD40L antibody; clenolimimab (IDEC-151 Biogen Idec) an anti-CD4 antibody; glaiximab (IDEC-114 Biogen Idec) an anti-CD80 antibody; lumilixmab (IDEC-152 Biogen Idec) an anti-CD23; anti-macrophage migration factor (MIF) antibodies (Biogen Idec, Taisho Pharmaceutical); mitumomab (BEC2 Imclone) a mouse anti-idiotypic antibody; IMC-1C11 (Imclone) a chimeric anti-VEGFR2 antibody; DC101 (Imclone) murine anti-VEGFR2 antibody; anti-VE cadherin antibody (Imclone); labetuzumab (CEA-CIDE™ Immunomedics) an anti-carcinoembryonic antigen antibody; epratuzumab (LYMPHOCIDE™ Immunomedics) an anti-CD22 antibody; yttrium (⁹⁰Y) tacatuzumab tetraxetan (AFP-Cide® Immunomedics) an anti-αfetoprotein antibody; milatuzumab (MyelomaCide® Immunomedics) an anti-CF74 antibody; LeukoCide® (Immunomedics); ProstaCide® (Immunomedics); ipilimumab (Yervoy™, MDX-010 Bristol-Myers Squibb) an anti-CTLA4 antibody approved to treat melanoma; iratumumab (MDX-060 Medarex) an anti-CD30 antibody; MDX-070 (Medarex) an anti-prostate specific membrane antigen; OSIDEM™ (IDM-1 Medarex, Immuno-Designed Molecules) an anti-Her2 antibody; HUMAX™-CD4, an anti-CD4 antibody being developed by Medarex and Genmab; HuMax-IL15, an anti-IL15 antibody being developed by Medarex and Genmab; golimumab (SIMPONI™ Janssen Biotech) an anti-TNFα antibody approved to treat rheumatoid arthritis, psoriatic arthritis, ankylosing spondylitis; ustekinumab (STELARA®, CNTO 1275 Janssen Biotech) an anti-IL-12 antibody approved to treat plaque psoriasis; MOR101 and MOR102 (MorphoSys) anti-intercellular adhesion molecule-1 (ICAM-1) (CD54) antibodies; MOR201 (MorphoSys) an anti-fibroblast growth factor receptor 3 antibody; visilizumab (NUVION™ PDL BioPharma) an anti-CD3 antibody; fontolizumab (HUZAF™ PDL BioPharma) an anti-INFγ antibody; volociximab (M200 PDL BioPharma, Biogen Idec) an anti-α5β1 integrin antibody; SMART® IL-12 (PDL BioPharma) an anti-IL-12; ING-1 (Xoma) an anti-Ep-CAM antibody; omalizumab (XOLAIR™ Genentech/Roche, Novartis) an anti-IgE antibody approved to treat allergic asthma; MLN01 (Xoma) an anti-β integrin antibody; and tocilizumab (ACTEMRA™ Genentech/Roche) an anti-IL6 antibody approved to treat rhemuatoid arthritis and systemic juvenile idiopathic arthritis.

### Construction of DVD-Ig Proteins

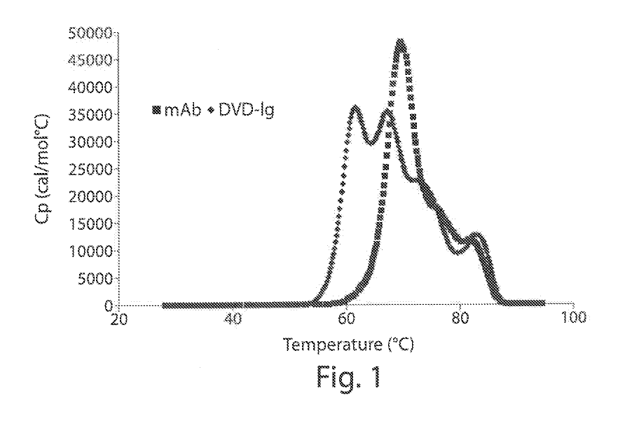

A DVD-Ig protein is formed by combining two heavy chain DVD polypeptides and two light chain DVD polypeptides. The dual variable domain immunoglobulin (DVD-Ig) heavy chain comprises two heavy chain variable domains (VH) linked in tandem, directly or by a linker, followed by the constant domain CH1 and Fc region. The dual variable domain immunoglobulin (DVD-Ig) light chain is designed such that two light chain variable domains (VL) from the two parent mAbs are linked in tandem, directly or via a linker, followed by the light chain constant domain (CL). (see FIG. 1A of U.S. Pat. No. 7,612,181, incorporated by reference herein). Methods of making DVD-Ig proteins are also described in U.S. Pat. No. 7,612,181, incorporated by reference herein.

The variable domains of the DVD-Ig protein can be obtained using recombinant DNA techniques from a parent antibody generated by any one of the methods described above. In one embodiment, the variable domain is a CDR grafted or a humanized variable heavy or light chain domain. In another embodiment, the variable domain is a human heavy or light chain variable domain. The linker sequence may be a single amino acid or a polypeptide sequence. Examples of linker sequences that may be used to link variable domains include, but are not limited to, AKTTPKLEEGEFSEAR (SEQ ID NO:1); AKTTPKLEEGEFSEARV (SEQ ID NO:2); AKTTPKLGG (SEQ ID NO:3); SAKTTPKLGG (SEQ ID NO:4); SAKTTP (SEQ ID NO:5); RADAAP (SEQ ID NO:6); RADAAPTVS (SEQ ID NO:7); RADAAAAGGPGS (SEQ ID NO:8); RADAAAA(G₄ₛ).₄ (SEQ ID NO:9), SAKTTPKLEEGEFSEARV (SEQ ID NO:10); ADAAP (SEQ ID NO:11); ADAAPTVSIFPP (SEQ ID NO:12); TVAAP (SEQ ID NO:13); TVAAPSVFIFPP (SEQ ID NO:14); QPKAAP (SEQ ID NO:15); QPKAAPSVTLFPP (SEQ ID NO:16); AKTTPP (SEQ ID NO:17); AKTTPPSVTPLAP (SEQ ID NO:18); AKTTAP (SEQ ID NO:19); AKTTAPSVYPLAP (SEQ ID NO:20); ASTKGP (SEQ ID NO:21); ASTKGPSVFPLAP (SEQ ID NO:22); GGGGSGGGGSGGGGS (SEQ ID NO:23); GENKVEYAPALMALS (SEQ ID NO:24); GPAKELTPLKEAKVS (SEQ ID NO:25); GHEAAAVMQVQYPAS (SEQ ID NO:26); and GGGGSGGGGS (SEQ ID NO: 27). Other examples of linkers are described in U.S. Patent Publication No. 20100226923. The choice of linker sequences may be determined based on crystal structure analysis of several antibody Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of the CL or CH1 domain. DVD Igs of the invention were generated using N-terminal 5-6 amino acid residues, or 11-12 amino acid residues, of CL or CH1 as the linker in the light chain and the heavy chain of the DVD-Ig proteins, respectively. The N-terminal residues of the CL or the CH1 domains, particularly the first 5-6 amino acid residues, adopt a loop conformation without strong secondary structure, and therefore can act as flexible linkers between the two variable domains. The N-terminal residues of the CL or CH1 domains are natural extensions of the variable domains, as they are part of the Ig sequences, and therefore immunogenicity potentially arising from the linkers or junctions is minimized.

Other linker sequences may include a sequence of any length of the CL or CH1 domain but not all residues of a CL/CH1 domain; for example the first 5-12 amino acid residues of the CL or CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins, (e.g., TCR, FcR, KIR); G/S based sequences (e.g., G4S repeats); hinge region-derived sequences; and other natural sequences from other proteins.

In an embodiment, a constant domain is linked to the two linked variable domains using recombinant DNA techniques. For example, a sequence comprising linked heavy chain variable domains is linked to a heavy chain constant domain and sequence comprising linked light chain variable domains is linked to a light chain constant domain. In an embodiment, the constant domains are a human heavy chain constant domain and a human light chain constant domain, respectively. In another embodiment, the DVD-Ig heavy chain is further linked to an Fc region. The Fc region may comprise a native Fc region sequence, or a variant Fc region sequence. In an embodiment, the Fc region is a human Fc region. For example, the Fc region comprises an Fc region from an IgG1, IgG2, IgG3, IgG4, IgA, IgM, IgE, or IgD.

In an embodiment, the DVD-Ig protein is a dual-specific tetravalent binding protein. In one embodiment, the DVD-Ig protein binds CD20 and CD80 In another embodiment, the DVD-Ig protein binds VEGF and HER2. In another embodiment, the DVD-Ig protein binds TNF and RANKL. In another embodiment, the DVD-Ig protein binds TNF and DKK. In another embodiment, the DVD-Ig protein binds CD20 and RANKL. In another embodiment, the DVD-Ig protein binds DLL4 and PLGF. In another embodiment, the DVD-Ig protein binds DLL4 and VEGF. In another embodiment, the DVD-Ig protein binds TNF and SOST. In another embodiment, the DVD-Ig protein binds IL-9 and IgE. In another embodiment, the DVD-Ig protein binds IL-12 and IL-18. An example of an IL-12 and IL-18 DVD-Ig protein is described in U.S. Pat. No. 7,612,181. In another embodiment, the DVD-Ig protein binds TNF and IL-17. In another embodiment, the DVD-Ig protein binds TNF and PGE2. Examples of PGE2 DVD-Ig proteins are provided in U.S. Patent Publication No. 20100074900. In another embodiment, the DVD-Ig protein binds IL-1α and IL-1β. An example of an IL-1α and IL-1β DVD-Ig protein is described in U.S. Pat. No. 7,612,181. In another embodiment, the DVD-Ig protein binds IL-4 and IL-1. An example of an IL-4 and IL-13 DVD-Ig protein is described in U.S. Publication No. 20100226923. The amino acid and nucleic acid sequences described in the aforementioned patents and patent applications are incoroporated by reference herein. Sequences of DVD-Ig proteins that may be used in the methods and compositions of the invention are described in SEQ ID NOs: 28-75.

### Expression of DVD-Igs Proteins

DVD-Ig proteins of the present invention may be produced by any of a number of techniques known in the art. For example, expression from host cells, wherein expression vector(s) encoding the DVD heavy and DVD light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Mammalian host cells for expressing the recombinant antibodies of the invention include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) J. Mol. Biol. 159:601-621) and DG44 or DUXB11 cells (Urlaub et al. (1986) Som. Cell Molec. Genet. 12:555; Haynes et al. (1983) Nuc. Acid. Res. 11:687-706; Lau et al. (1984) Mol. Cell. Biol. 4:1469-1475), NS0 myeloma cells, monkey kidney line (e.g., CVI and COS, such as a COS 7 cell), SP2 cells, human embryonic kidney (HEK) cells, such as a HEK-293 cell, Chinese hamster fibroblast (e.g., R1610), human cervical carcinoma (e.g., HELA), murine fibroblast (e.g., BALBc/3T3), murine myeloma (P3x63-Ag3.653; NS0; SP2/O), hamster kidney line (e.g., HAK), murine L cell (e.g., L-929), human lymphocyte (e.g., RAJI), human kidney (e.g., 293 and 293T). Host cell lines are typically commercially available (e.g., from BD Biosciences, Lexington, Ky.; Promega, Madison, Wis.; Life Technologies, Gaithersburg, Md.) or from the American Type Culture Collection (ATCC, Manassas, Va.).

When recombinant expression vectors encoding DVD-Ig proteins are introduced into mammalian host cells, the DVD-Ig proteins are produced by culturing the host cells for a period of time sufficient to allow for expression of the DVD-Ig proteins in the host cells or secretion of the DVD-Ig proteins into the culture medium in which the host cells are grown. DVD-Ig proteins can be recovered from the culture medium using standard protein purification methods.

In an exemplary system for recombinant expression of DVD-Ig proteins, a recombinant expression vector encoding both the DVD-Ig heavy chain and the DVD-Ig light chain is introduced into dhfr-CHO cells by calcium phosphate-mediated transfection. Within the recombinant expression vector, the DVD-Ig heavy and light chain cDNAs are each operatively linked to CMV enhancer/AdMLP promoter regulatory elements to drive high levels of transcription of the cDNAs. The recombinant expression vector also carries cDNA encoding DHFR, which allows for selection of CHO cells that have been transfected with the vector using methotrexate selection/amplification. The selected transformant host cells are cultured to allow for expression of the DVD-Ig heavy and light chains and intact DVD-Ig protein is recovered from the culture medium. Standard molecular biology techniques are used to prepare the recombinant expression vector, transfect the host cells, select for transformants, culture the host cells and recover the DVD-Ig protein from the culture medium. Still further, the invention provides a method of synthesizing a DVD-Ig protein of the invention by culturing a host cell of the invention in a suitable culture medium until a DVD-Ig protein of the invention is synthesized. The method can further comprise isolating the DVD-Ig protein from the culture medium. An important feature of DVD-Ig protein is that it can be produced and purified in a similar way as a conventional antibody.

### Methods for Identifying Aqueous Stable DVD-Igs (AS-DVD-Igs) Proteins and Lyophilized Stable DVD-Ig (LS-DVD-Ig) Proteins

An unexpected and surprising finding is that a certain subset of DVD-Ig proteins (referred to as AS-DVD-Ig protein and LS-DVD-Ig proteins) are stable—even at high concentrations—in aqueous formulations, while a large number of DVD-Ig proteins are unstable and prone to aggregation. In addition, while the majority of DVD-Ig proteins have been found not to be stable in a lyophilized state, a certain subset of DVD-Ig proteins (referred to as LS-DVD-Ig proteins) are stable and can be successfully lyophilized using the formulations of the invention. Notably, DVD-Ig proteins identified as AS-DVD-Ig proteins are also LS-DVD-Ig proteins. The distinction between the two subpopulations is based on the level of aggregation, as described in the below assays.

Thus, in one embodiment, the invention comprises a method for distinguishing between AS-DVD-Ig proteins and non-AS-DVD-Igs. The invention also comprises a method for distinguishing between LS-DVD-Ig proteins and non-LS-DVD-Ig proteins. Following identification, AS-DVD-Ig and LS-DVD-Ig proteins may be successfully formulated in the compositions of the invention, while non-AS-DVD-Ig and non-LS-DVD-Ig proteins fail to remain stable in such formulations and are prone to aggregation.

In order to determine whether a DVD-Ig protein is an AS-DVD-Ig protein or an LS-DVD-Ig protein, accelerated storage testing can be performed. For example, accelerated storage testing may be performed at 5° C. or 40° C. for 14 to 21 days at a DVD-Ig protein concentration ranging from 1 to 100 mg/ml. In one embodiment, testing is based on a solution's DVD-Ig protein aggregation levels at a high temperature (e.g., 40° C.) and a high concentration (e.g., 50 mg/ml) as determined by SEC. For example, the DVD-Ig protein may be formulated at a concentration of at least about 50 mg/ml in an aqueous formulation using a citrate phosphate buffer or a histidine buffer, and stored under accelerated conditions. Accelerated conditions may include temperatures higher than room temperature, e.g., storage temperatures of about 35 to about 45° Celsius (C), for extended periods of time, e.g., about 10 to about 21 days. In another embodiment, the accelerated storage conditions used to screen for an AS-DVD-Ig or LS-DVD-Ig protein are 14 days of storage at a temperature of 40° C. at a DVD-Ig protein concentration of 50 mg/ml or greater, e.g., about 60 mg/ml or 50-100 mg/ml. Following accelerated storage testing at a concentration of 50 mg/ml or greater, e.g. 50-100 mg/ml, the solution may be tested for signs of DVD-Ig protein aggregation.

Notably, lower levels of DVD-Ig protein concentration (e.g., 1 mg/ml) may also be used to test the protein, wherein lower levels of aggregate would be expected for an AS-DVD-Ig protein or an LS-DVD-Ig protein. For example, an AS-DVD-Ig protein is a DVD-Ig protein that has 3% or less aggregation when stored at about 40° C. after 21 days at a concentration of 1 mg/ml in an aqueous formulation.

Protein aggregation may be determined according to methods known in the art, including, but not limited to, Size Exclusion Chromatography (SEC).

In one embodiment, the DVD-Ig protein is considered an AS-DVD-Ig protein if the solution has 10% or less aggregation of the DVD-Ig protein as determined by Size Exclusion Chromatography (SEC) analysis following accelerated storage at a concentration of 1-100 mg/ml. In one embodiment, the DVD-Ig protein is considered an AS-DVD-Ig protein if the solution has 6% or less aggregation of the DVD-Ig protein as determined by SEC analysis following accelerated storage at a concentration of 1-100 mg/ml. In one embodiment, the DVD-Ig protein is considered an AS-DVD-Ig protein if the DVD-Ig protein has less than 10%, alternatively less than 9%, less than 8%, less than 7%, less than 6%, less than 5%, less than 4%, less than 3%, less than 2%, or less than 1% aggregation as determined by SEC analysis following accelerated storage at a concentration of 1-100 mg/ml. In another embodiment, an AS-DVD-Ig protein is defined as a DVD-Ig that has less than 8% aggregation following 14 days of accelerated storage (at, for example, about 40° C.). In one embodiment, an AS-DVD-Ig protein is defined as a DVD-Ig that has 6% or less aggregation following 14 days of accelerated storage (at, for example, about 40° C.). In a further embodiment, an AS-DVD-Ig protein is defined as a DVD-Ig that has less than 5% aggregation following 14 days of accelerated storage (at, for example, about 40° C.). In one embodiment, an AS-DVD-Ig protein is defined as a DVD-Ig that has 10% or less aggregation at about 40° C. after 21 days of storage at a concentration of 100 mg/ml in an aqueous formulation or has 10% or less aggregate following accelerated storage after 14 days at about 40° C., when formulated at a concentration over 50 mg/ml in an aqueous formulation.

While percent aggregation may be used to determine whether aggregation is present following accelerated storage, monomer content of the DVD-Ig protein may also be used. Alternatively, a DVD-Ig protein may be considered an AS-DVD-Ig protein if the protein has 6% or less monomer loss (determined by SEC) after 14 days at 40° C. or 3% or less monomer loss (determined by SEC) after 7 days at 40° C. in a solution having a concentration of 50 mg/ml DVD-Ig protein at pH 5.5 to 6.0 in 15 mM histidine. Monomer content may be used under any testing conditions, including, but not limited to, storage at 40° C. and/or at a pH of 5.5 to 6.5.

In another alternative, AS-DVD-Ig proteins are identified based on a solution's stability aggregation at a low temperature (e.g., 5° C.) and a high concentration (e.g., 50 mg/ml) of DVD-Ig as determined by SEC. For example, a solution containing 50 mg/ml of an AS-DVD-Ig protein at a pH of 5.5 to 6.0 in 15 mM histidine may have 1% or less monomer (determined by SEC) loss after 7 days at 5° C. (determined by SEC). In another example, a solution containing 50 mg/ml of an AS-DVD-Ig protein at a pH of 5.5 to 6.0 in 15 mM histidine may have 2% or less monomer loss after 14 days at 5° C. Alternatively, an AS-DVD-Ig has 1% or less aggregation at about 5° C. after 21 days of storage at a concentration of 100 mg/ml in an aqueous formulation, or 1.5% or less aggregation at about 5° C. after 21 days of storage at a concentration of 1 mg/ml in an aqueous formulation. In one embodiment, monomer loss is determined at a pH of 5.5 to 6.5.

In another alternative, freeze/thaw (e.g., −80° C./30° C.) is used as a means to determine whether a DVD-Ig protein is an AS-DVD-Ig protein. Such a method relies upon determining the percentage of high molecular weight (HMW) species in a solution having a high concentration of DVD-Ig protein (e.g., 100 mg/ml). An AS-DVD-Ig protein would show 1% or less HMW species in such conditions.

In one embodiment, the test solution conditions described herein also contain 0.02% (w/v) sodium azide as a bacteriostatic.

In one embodiment, the DVD-Ig protein is considered an LS-DVD-Ig protein if the solution has 15% or less aggregation of the DVD-Ig protein as determined by Size Exclusion Chromatography (SEC) analysis. In one embodiment, the LS-DVD-Ig protein is considered an LS-DVD-Ig protein if the DVD-Ig protein has 15% or less, alternatively less than 14%, less than 13%, less than 12%, less than 11%, less than aggregation as determined by SEC analysis. Once the DVD-Ig protein is identified as being an AS-DVD-Ig or LS-DVD-Ig protein according to the aforementioned test, the AS-DVD-Ig or LS-DVD-Ig protein can be stably formulated. Further identification of AS-DVD-Ig and LS-DVD-Ig proteins is described below in Example 4.

DVD-Igs may be tested in aqueous formulations containing, for example, citrate and phosphate buffer, or histidine buffer. In one embodiment, an AS-DVD-Ig protein has 10% or less aggregation as determined by SEC analysis following accelerated storage for 21 days at about 40° C., where the AS-DVD-Ig protein is formulated at a concentration of at least 100 mg/ml in a citrate phosphate buffer or histidine buffer in an aqueous formulation. In one embodiment, an AS-DVD-Ig protein has less than 6% aggregation as determined by SEC analysis following accelerated storage for 14 days at about 40° C., where the AS-DVD-Ig protein is formulated at a concentration of 50 mg/ml in a citrate phosphate buffer or histidine buffer in an aqueous formulation. Formulations for testing AS-DVD-Ig proteins may also include a sugar, such as, but not limited to, sucrose.

### III. Aqueous Stable Dual Variable Domain Immunoglobulin (AS-DVD-Ig) Formulations of the Invention

The invention provides stable aqueous formulations comprising AS-DVD-Igs. The present invention features formulations having improved properties as compared to art-recognized formulations, in that AS-DVD-Ig proteins can be stably formulated, even at high concentrations.

Thus, the invention is based, at least in part, on the discovery that a subpopulation of DVD-Ig proteins can be stably formulated in an aqueous formulation having a pH of about 4.5 to about 7.5, and containing a buffer, a surfactant, and/or a polyol. These “Aqueous Stable DVD-Ig proteins” are referred to as AS-DVD-Ig proteins and can be identified using an accelerated storage assay where the DVD-Ig protein is formulated in a liquid form at a concentration greater than 50 mg/ml.

In one embodiment, the AS-DVD-Ig protein is an anti-TNF/IL-17 DVD-Ig protein having a heavy and light chain sequences having an amino acid sequence as set forth in SEQ ID NOs: 62 and 63, respectively.

In one embodiment, the AS-DVD-Ig protein is an anti-IL1α/IL-1β DVD-Ig protein comprising an anti-IL1a/IL1B DVD-Ig protein having a heavy and light chain sequences having an amino acid sequence as set forth in SEQ ID NOs: 66 and 67, respectively.

In one aspect, the formulation of the invention has a pH of about 4.5 to about 7.5. As described in the working examples, pH was found to have an impact on the stability of the AS-DVD-Ig protein in a buffered formulation. In one embodiment, the pH of the formulation containing the AS-DVD-Ig protein ranges from about 4.5 to about 7.5; alternatively, the pH of the AS-DVD-Ig protein formulation ranges from about 5.0 to about 7.0; alternatively the pH may range from about 5 to about 6.5; alternatively the pH of the formulation may range from about 5.5 to about 6.5. In a further embodiment, the pH ranges from about 5.8 to about 6.2. The ranges intermediate to the aforementioned pH values, e.g., about 5.6 to about 6.4, are also intended to be part of the invention. Ranges of values using a combination of any of the aforementioned values as upper/lower limits are also intended to be included, e.g., a pH range of about 5.5 to about 6.2. In one embodiment, the pH of the formulation of the invention is about 6.0.

In one embodiment, the formulation of the invention includes an AS-DVD-Ig protein and a buffer. Examples of buffers that may be used in the formulation of the invention include, but are not limited to, acetate, histidine, glycine, arginine, phosphate, Tris, and citrate. The molarity of the buffer used in the formulation of the invention may range from about 1 to about 50 mM. In one embodiment, the aqueous formulation of the invention has a buffer with a molarity of about 5 to about 50 mM. Alternatively, the molarity of the buffer is about 10 to about 20 mM.

In one embodiment of the invention, the buffer system comprises about 1 to about 200 mM histidine (e.g., about 2 to about 100 mM; about 5 to about 70 mM; about 5 to about 60 mM; about 5 to about 50 mM; about 10 to about 40 mM, about 10 to about 30 mM, or about 10 to about 20 mM) with a pH of about 4.5 to about 7.5, e.g., a pH of about 5 to about 7, or a pH of about 5.5 to about 6.5. In one embodiment, the buffer system of the invention comprises about 15 mM histidine with a pH of about 4.5 to about 7.5, e.g., a pH of about 5 to about 7, or a pH of about 5.5 to about 6.5.

In one embodiment, the buffer system comprises about 1 to about 50 mM (e.g., about 5 to about 40 mM) glycine with a pH of about 4.5 to about 7.5. In a particular embodiment, the buffer system comprises glycine at a concentration of about 20 mM. In a more particular embodiment, the buffer system comprises glycine at a concentration of about 20 mM, and glycerol at a concentration of about 20 to about 30 mg/ml, e.g., about 26 mg/ml, with a pH of about 4.5 to about 7.5, e.g., a pH of about 5 to about 7, or a pH of about 5.5 to about 6.5.

In another embodiment, the buffer system comprises about 1 to about 50 mM acetate (e.g., about 5 to about 50 mM, about 2 to about 40 mM; about 5 to about 30 mM; or about 2 to about 15 mM) with a pH of about 4.5 to about 7.5, e.g., a pH of about 5 to about 7, or a pH of about 5.5 to about 6.5. In a particular embodiment, the buffer system comprises acetate at a concentration of about 2 to about 15 mM.

In another embodiment of the invention, the buffer system comprises about 1 to about 50 mM (e.g., about 5 to about 50 mM, about 2 to about 40 mM; about 5 to about 30 mM; or about 2 to about 15 mM) arginine with a pH of about 4.5 to about 7.5, e.g., a pH of about 5 to about 7, or a pH of about 5.5 to about 6.5. In a particular embodiment, the buffer system comprises arginine at a concentration of about 15 mM.

In still another embodiment of the invention, the buffer system comprises about 1 to about 50 mM (e.g., about 5 to about 50 mM) citrate with a pH of about 4.5 to about 7.5, e.g., a pH of about 5 to about 7, or a pH of about 5.5 to about 6.5. In a particular embodiment, the buffer system comprises citrate at a concentration of about 15 mM.

In still another embodiment of the invention, the buffer system comprises about 1 to about 50 mM (e.g., about 5 to about 50 mM) phosphate with a pH of about 4.5 to about 7.5, e.g., a pH of about 5 to about 7, or a pH of about 5.5 to about 6.5. In a particular embodiment, the buffer system comprises phosphate at a concentration of about 10 mM. In a one embodiment, the buffer system comprises phosphate at a concentration of about 10 mM, and sodium chloride at a concentration of about 125 mM.

In one embodiment, the buffer system comprises about 1 to about 50 mM (e.g., about 5 to about 50 mM) Tris with a pH of about 4.5 to about 7.5, e.g., a pH of about 5 to about 7, or a pH of about 5.5 to about 6.5. In a particular embodiment, the buffer system comprises Tris at a concentration of about 2 to about 10 mM.

In yet another embodiment, the buffer system comprises phosphate and citrate, e.g., phosphate (e.g., sodium hydrogen phosphate) at a concentration of about 1 to about 50 mM (e.g., about 5 to about 50 mM, about 5 to about 10 mM), and citrate (citric acid) at a concentration of about 1 to about 50 mM (e.g., about 5 to about 10 mM). In a particular embodiment, the buffer system comprises phosphate at a concentration of about 5 mM and citrate (citric acid) at a concentration of about 5 mM. In one embodiment, the buffer system comprises phosphate at a concentration of about 10 mM and citrate (citric acid) at a concentration of about 10 mM.

In addition to the buffer, a polyol may be added to the formulation, e.g., for added stability. The polyol may be added to the formulation in an amount that may vary with respect to the desired isotonicity of the formulation. In an embodiment, the aqueous formulation is isotonic.

Examples of polyols that may be used in the formulations of the invention include, but are not limited to, sorbitol, mannitol, and sucrose fructose, mannose, maltose, lactose, arabinose, xylose, ribose, rhamnose, galactose and glucose. Nonreducing sugars include sucrose, trehalose, sorbose, melezitose and raffinose. mannitol, xylitol, erythritol, threitol, sorbitol and glycerol. The amount of polyol added may also vary with respect to the molecular weight of the polyol. For example, a lower amount of a monosaccharide (e.g., mannitol) may be added, compared to a disaccharide (e.g., trehalose).

In one embodiment, the concentration of a polyol such as sorbitol is about 30 to about 50 mg/ml. In a one embodiment, the composition comprises about 20 to about 60 mg/ml sorbitol, about 25 to about 55 mg/ml, about 30 to about 50 mg/ml, about 35 to about 45 mg/ml, and ranges inbetween, e.g., about 33 to about 48 mg/ml of sorbitol.

In another embodiment, sucrose has a concentration of about 70 to about 90 mg/ml. In an embodiment, the composition comprises about 60 to about 100 mg/ml sucrose, about 65 to about 95 mg/ml, about 70 to about 90 mg/ml, about 75 to about 85 mg/ml, and ranges inbetween, e.g., about 72 to about 84 mg/ml of sucrose.

In another embodiment, the polyol is mannitol. In an embodiment, the composition comprises about 10 to about 100 mg/ml, or about 20 to about 80, about 20 to about 70, about 30 to about 60, about 30 to about 50 mg/ml of mannitol, for example, about 10, about 20, about 30, about 40, about 50, about 60, about 70, about 80, about 90, or about 100 mg/ml.

In one embodiment, the formulation of the invention includes an AS-DVD-Ig protein, a buffer having a molarity of about 5 to about 50 mM, and a polyol, wherein the formulation has a pH of about 4.5 to about 7.5.

In addition to the buffer, a surfactant may be added to the formulations, e.g., for added stability. Exemplary surfactants include nonionic detergents such as polysorbates (e.g., polysorbates 20, 80) or poloxamers (e.g., poloxamer 188). In an embodiment, the amount of surfactant added is such that it reduces aggregation of the formulated AS-DVD-Ig protein and/or minimizes the formation of particulates in the formulation and/or reduces adsorption.

In an embodiment, the formulation contains the detergent polysorbate 80 or Tween 80. Tween 80 is a term used to describe polyoxyethylene (20) sorbitan monooleate. In one embodiment, the formulation contains about 0.001 to about 1% polysorbate 80, or about 0.005 and about 0.05% polysorbate 80, for example, about 0.001%, about 0.005, about 0.01%, about 0.05%, or about 0.1% polysorbate 80. In one embodiment, about 0.01% polysorbate 80 is found in the formulation of the invention.

In one embodiment, the formulation of the invention includes an AS-DVD-Ig protein, a buffer having a molarity of about 5 to about 50 mM, and a surfactant, wherein the formulation has a pH of about 4.5 to about 7.5. In one embodiment, the surfactant is a polysorbate, e.g., polysorbate 80 or polysorbate 20. In one embodiment, the polysorbate has a concentration of about 0.005% to about 0.02%.

In one embodiment, the formulation of the invention includes an AS-DVD-Ig protein, a buffer having a molarity of about 5 to about 50 mM, a surfactant, and a polyol, wherein the formulation has a pH of about 4.5 to about 7.5. In one embodiment, the formulation includes an AS-DVD-Ig protein, a buffer (e.g., histidine), a polysorbate, e.g., polysorbate 80, and a sugar alcohol, e.g., mannitol or sorbitol. In another embodiment, the formulation includes an AS-DVD-Ig protein, a buffer (e.g., histidine), a polysorbate, e.g., polysorbate 80, and a non-reducing sugar, e.g., sucrose.

One advantage of the formulation of the invention is that high concentrations of AS-DVD-Ig proteins may be stably maintained in an aqueous solution. Thus, in an aspect, the formulations of the invention comprise a high protein concentration, including, for example, a protein concentration greater than about 10 mg/ml, greater than about 20 mg/ml, greater than about 30 mg/ml, greater than about 40 mg/ml, greater than about 50 mg/ml, greater than about 100 mg/ml, greater than about 110 mg/ml, greater than about 120 mg/ml, greater than about 130 mg/ml, greater than about 140 mg/ml, greater than about 150 mg/ml, greater than about 160 mg/ml, greater than about 170 mg/ml, greater than about 180 mg/ml, greater than about 190 mg/ml, or greater than about 200 mg/ml.

In various embodiments of the invention, the concentration of the AS-DVD-Ig protein in the formulation is about 0.1-250 mg/ml, about 0.5-220 mg/ml, about 1-210 mg/ml, about 5-200 mg/ml, about 10-195 mg/ml, about 15-190 mg/ml, about 20-185 mg/ml, about 25-180 mg/ml, about 30-175 mg/ml, about 35-170 mg/ml, about 40-165 mg/ml, about 45-160 mg/ml, about 50-155 mg/ml, about 55-150 mg/ml, about 60-145 mg/ml, about 65-140 mg/ml, about 70-135 mg/ml, about 75-130 mg/ml, about 80-125 mg/ml, about 85-120 mg/ml, about 90-H5 mg/ml, about 95-110 mg/ml, about 95-105 mg/ml, or about 100 mg/ml. Ranges intermediate to the above recited concentrations, e.g., about 31-174 mg/ml, are also intended to be part of this invention. For example, ranges of values using a combination of any of the above recited values as upper and/or lower limits are intended to be included.

The present invention features formulations having improved properties as compared to art-recognized formulations. For example, the formulations of the invention have an AS-DVD-Ig protein aggregation level of less than 7% aggregate, less than 6% aggregate, or less than 5% aggregate.

### IV. Lyophilized Stable Dual Variable Domain Immunoglobulin (LS-DVD-Ig) Protein Formulations of the Invention

The invention further provides stable lyophilized formulations comprising LS-DVD-Ig proteins. Thus, the invention is based, at least in part, on the discovery that a subpopulation of DVD-Ig proteins can be stably formulated in a lyophilized formulation having a pH of about 4.5 to about 7.5, and containing a buffer, a surfactant, and/or a polyol. These “Lyophilized Stable DVD-Ig proteins” or “LS-DVD-Ig proteins” can be identified using an accelerated storage assay (described above) where the DVD-Ig protein is formulated in a liquid form at a concentration greater than 50 mg/ml.

In one aspect, the formulation of the invention has a pH of about 4.5 to about 7.5. In one embodiment, the pH of the formulation containing the LS-DVD-Ig protein ranges from about 4.5 to about 7.5; alternatively, the pH of the LS-DVD-Ig protein formulation ranges from about 5.0 to about 7.0; alternatively the pH may range from about 5 to about 6.5; alternatively the pH of the formulation may range from about 5.5 to about 6.5. In a further embodiment, the pH ranges from about 5.8 to about 6.2. The ranges intermediate to the aforementioned pH values, e.g., about 5.6 to about 6.4, are also intended to be part of the invention. Ranges of values using a combination of any of the aforementioned values as upper/lower limits are also intended to be included, e.g., a pH range of about 5.5 to about 6.2. In one embodiment, the pH of the formulation of the invention is about 6.0.

In one embodiment, the formulation of the invention includes an LS-DVD-Ig protein and a buffer. Examples of buffers that may be used in the formulation of the invention include, but are not limited to, acetate, histidine, glycine, arginine, phosphate, Tris, and citrate. The molarity of the buffer used in the formulation of the invention may range from about 1 to about 50 mM. In one embodiment, the aqueous formulation of the invention has a buffer with a molarity of about 5 to about 50 mM. Alternatively, the molarity of the buffer is about 10 to about 20 mM.

In one embodiment of the invention, the buffer system comprises about 1 to about 200 mM histidine (e.g., about 2 to about 100 mM; about 5 to about 70 mM; about 5 to about 60 mM; about 5 to about 50 mM; about 10 to about 40 mM, about 10 to about 30 mM, or about 10 to about 20 mM) with a pH of about 4.5 to about 7.5, e.g., a pH of about 5 to about 7, or a pH of about 5.5 to about 6.5. In one embodiment, the buffer system of the invention comprises about 15 mM histidine with a pH of about 4.5 to about 7.5, e.g., a pH of about 5 to about 7, or a pH of about 5.5 to about 6.5.

In one embodiment, the buffer system comprises about 1 to about 50 mM (e.g., about 5 to about 40 mM) glycine with a pH of about 4.5 to about 7.5. In a particular embodiment, the buffer system comprises glycine at a concentration of about 20 mM. In a more particular embodiment, the buffer system comprises glycine at a concentration of about 20 mM, and glycerol at a concentration of about 20 to about 30 mg/ml, e.g., about 26 mg/ml, with a pH of about 4.5 to about 7.5, e.g., a pH of about 5 to about 7, or a pH of about 5.5 to about 6.5.

In another embodiment, the buffer system comprises about 1 to about 50 mM acetate (e.g., about 5 to about 50 mM, about 2 to about 40 mM; about 5 to about 30 mM; or about 2 to about 15 mM) with a pH of about 4.5 to about 7.5, e.g., a pH of about 5 to about 7, or a pH of about 5.5 to about 6.5. In a particular embodiment, the buffer system comprises acetate at a concentration of about 2 to about 15 mM.

In another embodiment of the invention, the buffer system comprises about 1 to about 50 mM (e.g., about 5 to about 50 mM, about 2 to about 40 mM; about 5 to about 30 mM; or about 2 to about 15 mM) arginine with a pH of about 4.5 to about 7.5, e.g., a pH of about 5 to about 7, or a pH of about 5.5 to about 6.5. In a particular embodiment, the buffer system comprises arginine at a concentration of about 15 mM.

In still another embodiment of the invention, the buffer system comprises about 1 to about 50 mM (e.g., about 5 to about 50 mM) citrate with a pH of about 4.5 to about 7.5, e.g., a pH of about 5 to about 7, or a pH of about 5.5 to about 6.5. In a particular embodiment, the buffer system comprises citrate at a concentration of about 15 mM.

In still another embodiment of the invention, the buffer system comprises about 1 to about 50 mM (e.g., about 5 to about 50 mM) phosphate with a pH of about 4.5 to about 7.5, e.g., a pH of about 5 to about 7, or a pH of about 5.5 to about 6.5. In a particular embodiment, the buffer system comprises phosphate at a concentration of about 10 mM. In a one embodiment, the buffer system comprises phosphate at a concentration of about 10 mM, and sodium chloride at a concentration of about 125 mM.

In one embodiment, the buffer system comprises about 1 to about 50 mM (e.g., about 5 to about 50 mM) Tris with a pH of about 4.5 to about 7.5, e.g., a pH of about 5 to about 7, or a pH of about 5.5 to about 6.5. In a particular embodiment, the buffer system comprises Tris at a concentration of about 2 to about 10 mM.

In yet another embodiment, the buffer system comprises phosphate and citrate, e.g., phosphate (e.g., sodium hydrogen phosphate) at a concentration of about 1 to about 50 mM (e.g., about 5 to about 50 mM, about 5 to about 10 mM), and citrate (citric acid) at a concentration of about 1 to about 50 mM (e.g., about 5 to about 10 mM). In a particular embodiment, the buffer system comprises phosphate at a concentration of about 5 mM and citrate (citric acid) at a concentration of about 5 mM. In one embodiment, the buffer system comprises phosphate at a concentration of about 10 mM and citrate (citric acid) at a concentration of about 10 mM.

In addition to the buffer, a polyol may be added to the formulation, e.g., for added stability. The polyol may be added to the formulation in an amount that may vary with respect to the desired isotonicity of the formulation. In an embodiment, the lyophilized formulation is isotonic upon reconstitution.

Examples of polyols that may be used in the formulations of the invention include, but are not limited to, mannitol, sucrose, trehalose and raffinose. The amount of polyol added may also vary with respect to the molecular weight of the polyol. For example, a lower amount of a monosaccharide (e.g., mannitol) may be added, compared to a disaccharide (e.g., trehalose).

In one embodiment, the concentration of a polyol such as sorbitol is about 30 to about 50 mg/ml. In a one embodiment, the composition comprises about 20 to about 60 mg/ml sorbitol, about 25 to about 55 mg/ml, about 30 to about 50 mg/ml, about 35 to about 45 mg/ml, and ranges inbetween, e.g., about 33 to about 48 mg/ml of sorbitol.

In another embodiment, sucrose has a concentration of about 70 to about 90 mg/ml. In an embodiment, the composition comprises about 60 to about 100 mg/ml sucrose, about 65 to about 95 mg/ml, about 70 to about 90 mg/ml, about 75 to about 85 mg/ml, and ranges inbetween, e.g., about 72 to about 84 mg/ml of sucrose.

In another embodiment, the polyol is mannitol. In an embodiment, the composition comprises about 10 to about 100 mg/ml, or about 20 to about 80, about 20 to about 70, about 30 to about 60, about 30 to about 50 mg/ml of mannitol, for example, about 10, about 20, about 30, about 40, about 50, about 60, about 70, about 80, about 90, or about 100 mg/ml.

In one embodiment, the formulation of the invention includes an AS-DVD-Ig, a buffer having a molarity of about 5 to about 50 mM, and a polyol, wherein the formulation has a pH of about 4.5 to about 7.5.

In addition to the buffer, a surfactant may be added to the formulations, e.g., for added stability. Exemplary surfactants include nonionic detergents such as polysorbates (e.g., polysorbates 20, 60, 80,) or poloxamers (e.g., poloxamer 188). In an embodiment, the amount of surfactant added is such that it reduces aggregation of the formulated LS-DVD-Ig protein and/or minimizes the formation of particulates in the formulation and/or reduces adsorption.

In an embodiment, the formulation contains the detergent polysorbate 80 or Tween 80. Tween 80 is a term used to describe polyoxyethylene (20) sorbitanmonooleate. In one embodiment, the formulation contains about 0.001 to about 0.1% polysorbate 80, or about 0.005 and about 0.05%, 20 polysorbate 80, for example, about 0.001, about 0.005, about 0.01, about 0.05, or about 0.1% polysorbate 80. In one embodiment, about 0.01% polysorbate 80 is found in the formulation of the invention.

In one embodiment, the formulation of the invention includes an LS-DVD-Ig protein, a buffer having a molarity of about 5 to about 50 mM, and a surfactant, wherein the formulation has a pH of about 4.5 to about 7.5. In one embodiment, the surfactant is a polysorbate, e.g., polysorbate 80 or polysorbate 20. In one embodiment, the polysorbate has a concentration of about 0.005% to about 0.02%.

In one embodiment, the formulation of the invention includes an LS-DVD-Ig protein, a buffer having a molarity of about 5 to about 50 mM, a surfactant, and a polyol, wherein the formulation has a pH of about 4.5 to about 7.5. In one embodiment, the formulation includes an LS-DVD-Ig protein, a buffer (e.g., histidine), a polysorbate (e.g., polysorbate 80), and a sugar alcohol (e.g., mannitol or sorbitol). In another embodiment, the formulation includes an LS-DVD-Ig protein, a buffer (e.g., histidine), a polysorbate, e.g., polysorbate 80, and a non-reducing sugar, e.g., sucrose.

The lyophilized formulation described herein is initially made as a “pre-lyophilized formulation,” which is the formulation prior to the lyophilzation process. The amount of protein present in the pre-lyophilized formulation is determined taking into account the desired dose volumes, mode(s) of administration etc. For example, the starting concentration of an LS-DVD-Ig protein can be from about 2 mg/ml to about 50 mg/ml.

Lyophilization may be performed according to methods known in the art. Many different freeze-dryers are available for this purpose such as Hu1150™ (Hull, USA) or GT20™ (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hrs). Optionally, a secondary drying stage may also be performed depending upon the desired residual moisture level in the product. The temperature at which the secondary drying is carried out ranges from about 0-40° C., depending primarily on the type and size of container and the type of protein employed. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Freeze-drying conditions can be varied depending on the formulation and vial size.

Prior to administration to the patient, the lyophilized formulation is reconstituted with a pharmaceutically acceptable diluent such that the protein concentration in the reconstituted formulation is at least about 2 mg/ml, for example from about 2 mg/ml to about 100 mg/ml, alternatively from about 10 mg/ml to about 90 mg/ml, alternatively from about 30 mg/ml to about 50 mg/ml. Such high protein concentrations in the reconstituted formulation are considered to be particularly useful where subcutaneous delivery of the reconstituted formulation is intended. However, for other routes of administration, such as intravenous administration, lower concentrations of the protein in the reconstituted formulation may be desired (for example from about 2-50 mg/ml, or from about 3-40 mg/ml protein in the reconstituted formulation). In certain embodiments, the protein concentration in the reconstituted formulation is significantly higher than that in the pre-lyophilized formulation. Reconstitution generally takes place at a temperature of about 25.degree C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%.

### V. Uses of the Invention

The formulations of the invention may be used both therapeutically, i.e., in vivo, or as reagents for in vitro or in situ purposes. The methods of the invention may also be used to make a water-based formulation having characteristics that are advantageous for therapeutic use. The aqueous formulation may be used as a pharmaceutical formulation to treat a disorder in a subject.

The formulation of the invention may be used to treat any disorder for which the therapeutic protein is appropriate for treating. A “disorder” is any condition that would benefit from treatment with the protein. This includes chronic and acute disorders or diseases including those pathological conditions which predispose the mammal to the disorder in question. In the case of an anti-TNF DVD-Ig protein, a therapeutically effective amount of the DVD-Ig protein may be administered to treat an autoimmune disease, such as rheumatoid arthritis, an intestinal disorder, such as Crohn's disease, a spondyloarthropathy, such as ankylosing spondylitis, or a skin disorder, such as psoriasis. In the case of an anti-IL-12 DVD-Ig, a therapeutically effective amount of the DVD-Ig protein may be administered to treat a neurological disorder, such as multiple sclerosis, or a skin disorder, such as psoriasis. Other examples of disorders in which the formulation of the invention may be used to treat include cancer, including breast cancer, leukemia, lymphoma, and colon cancer.

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

The term “treatment” refers to both therapeutic treatment and prophylactic or preventative measures. Those in need of treatment include those already with the disorder, as well as those in which the disorder is to be prevented.

The aqueous formulation may be administered to a mammal, including a human, in need of treatment in accordance with known methods of administration. Examples of methods of administration include parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. The protein is administered to the patient at one time or over a series of treatments and may be administered to the patient at any time from diagnosis onwards. The protein may be administered as the sole treatment or in conjunction with other drugs or therapies useful in treating the condition in question.

Actual dosage levels of the AS-DVD-Ig or LS-DVD-Ig protein, the active ingredient, in the pharmaceutical formulation of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the AS-DVD-Ig protein or LS-DVD-Ig protein found in the formulation, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

In an embodiment of the invention, the dosage of the AS-DVD-Ig protein in the formulation is about 1 to about 250 mg. In an embodiment, the dosage of the AS-DVD-Ig protein in the formulation is about 30 to about 140 mg, about 40 to about 120 mg, about 50 to about 110 mg, about 60 to about 100 mg, or about 70 to about 90 mg. In a further embodiment, the composition includes an AS-DVD-Ig protein dosage of about 1, 10, 20, 30, 40, 50, 60, 70, 80, 90, 100, 110, 120, 130, 140, 150, 160, 170, 180, 190, 200, 210, 220, 230, 240 or 250 mg.

In one embodiment of the invention, the dosage of the LS-DVD-Ig protein in the formulation (upon reconstitution) is about 1 to about 250 mg. In a further embodiment, the dosage of the LS-DVD-Ig protein in the formulation is about 30 to about 140 mg, about 40 to about 120 mg, about 50 to about 110 mg, about 60 to about 100 mg, or about 70 to about 90 mg. In a further embodiment, the composition includes an LS-DVD-Ig protein dosage of about 1, 10, 20, 30, 40, 50, 60, 70, 80, 90, 100, 110, 120, 130, 140, 150, 160, 170, 180, 190, 200, 210, 220, 230, 240 or 250 mg.

The formulations of the invention overcome common problems known in formulation development, including the problem of protein aggregation often associated with high concentrations of protein, particularly complex proteins such as DVD-Ig proteins. Thus, in one embodiment, the formulations of the invention provide a new means by which high levels of this new therapeutic protein format may be administered to a patient.

### Examples

The Examples presented herein describe formulations containing dual variable domain immunoglobulin (DVD-Ig) proteins that have unexpected stability characteristics. The experiments were surprising in that certain DVD-Ig proteins, referred to as Aqueous Stable DVD-Ig (AS-DVD-Ig) proteins or Lyophilized Stable DVD-Ig (LS-DVD-Ig) proteins, were stable in aqueous or lyophilized formulations, respectively, whereas other DVD-Ig proteins showed aggregation and instability when similarly formulated. The experiments exemplify methods for identifying AS-DVD-Ig proteins and LS-DVD-Ig proteins, as well as stable formulations thereof.

### Materials and Methods

The methods described herein were used in experiments performed to assess and monitor the stability of DVD-Ig proteins in aqueous and lyophilized formulations.

### General Methods

DVD-Ig protein formulations were tested for general quality parameters (e.g., pH), parameters of physical stability (e.g., clarity, color, particle contamination and purity), and parameters of chemical stability (e.g., deamidation, oxidation, and general chemical stability). Exemplary tests included tests for visible particulate contamination, light obscuration particle count tests for subvisible particles, and tests for purity such as size exclusion high pressure liquid chromatography (also referred to herein as size exclusion chromatography (SEC)) and ion exchange chromatography (IEC).

Particulate contamination (e.g., visible particles) was determined by visual inspection. Subvisible particles were monitored by light obscuration assays according to the United States Pharmacopeia (USP). The physicochemical stability of formulations was assessed by SEC, which allows for the detection of fragments and aggregates. To monitor chemical stability, SEC (for the detection of fragments and hydrolysis) and IEC were performed.

### DVD-Ig Proteins Tested

The DVD-Ig proteins that were tested in the Examples provided herein are listed in Table 1. The sequences of the DVD-Ig proteins described in Table 1 are provided in Table 66.

TABLE 1
Dual Variable Domain Immunoglobulin
(DVD-Ig) Proteins and Their Targets

| DVD-Ig Protein Name | Targets |
|---|---|
| DVD 5 | CD20/CD80 |
| DVD 6 | CD80/CD20 |
| DVD 37 | VEGF/HER2 |
| DVD 38 | HER2/VEGF |
| DVD 53 | TNF/RANKL |
| DVD 54 | RANKL/TNF |
| DVD 65 | TNF/DKK |
| DVD 66 | DKK/TNF |
| DVD 165 | CD20/RANKL |
| DVD 166 | RANKL/CD20 |
| DVD 257 | DLL4/PLGF |
| DVD 258 | PLGF/DLL4 |
| DVD 277 | TNF/SOST (S2) |
| DVD 278 | SOST(S2)/TNF |
| DVD 281 | IL-9(S2)/IgE |
| DVD 282 | IgE/IL-9(S2) |
| IL12IL18 | IL-12/IL-18 |
| DVD A | TNF/IL-17 |
| DVD B | TNF/PGE2 |
| DVD C | IL-1α/IL-1β |

DVD-Ig protein as starting material was provided following purification and was >95% monomeric form.

### Cation Exchange HPLC Methods

Cation exchange HPLC, a form of IEC, was used to determine the identity and purity of the DVD-Ig protein formulations. The assay was performed with the parameters detailed below.

A Dionex ProPac® WCX-10 analytical column (Dionex Corp., Sunnyvale, Calif.), combined with a Dionex WCX-10G guard column (Dionex Corp., Sunnyvale, Calif.), was run with upper column pressure being limited to 210 bar. The mobile phase A consisted of 10 mM Na₂HPO₄, pH 6.0. This buffer was created by dissolving 4.97 g anhydrous disodium hydrogen phosphate in approximately 3300 mL Milli-Q water, adjusting the pH to 7.0 using 1 M phosphoric acid, increasing buffer volume to 3500 mL with Milli-Q water and filtering the solution through a membrane filter. The mobile phase B consisted of 10 mM Na₂HPO₄, 500 mM NaCl, pH 6.0. This buffer was created by dissolving 2.56 g anhydrous disodium hydrogen phosphate in approximately 1500 ml Milli-Q water, adjusting the pH to 6.0 using 1 M phosphoric acid, increasing buffer volume to 1800 ml with Milli-Q water and filtering the solution through a membrane filter. A summary of the cation exchange HPLC methods is described in Table 2.

TABLE 2
Summary of Cation Exchange HPLC Methods

| Item | Description/Operating Conditions | |
|---|---|---|
| Guard column | ProPac WCX-10G, 4.0 × 50 mm | |
| Column | ProPac WCX-10, 4.0 × 250 mm | |
| Mobile phase A* | 10 mM disodium hydrogen phosphate, | |
| | pH 6.0 | |
| Mobile phase B* | 10 mM disodium hydrogen phosphate/500 | |
| | mM sodium chloride, pH 6.0 | |
| | Binary Gradient | |
| | Time (minute) | Mobile Phase B % |
| Gradient | 0.01 | 15.0 |
| | 30.00 | 30.0 |
| | 32.00 | 100.0 |
| | 37.00 | 100.0 |
| | 39.00 | 15.0 |
| | 44.00 | 15.0 |
| | 44.10 | Stop |

| | |
|---|---|
| Flow rate | 1.0 ml/minute |
| Detector wavelength | 280 nm |
| Autosampler temperature | Nominal 4° C. |
| Column oven temperature | 35° C. |
| Sample load | 100 μL/100 μg |
| Run time | 44.0 minutes |

For the IL12IL18 and DVD 66 DVD-Ig proteins, the mobile phases used were changed as follows:

Mobile phase A: 10 mM MES, pH 5.6; and
Mobile phase B: 10 mM MES+500 mM NaCl, pH 5.6.

For IL1αILβ, the mobile phases used were changed as follows:

Mobile phase A: 20 mM Phosphate, pH 8.0; and
Mobile phase B: 20 mM Phosphate+500 mM NaCl, pH 8.0.

The gradient for IL1αILβ was as follows:

TABLE 3
Gradient for IL1αILβ

| Time (minutes) | % B |
|---|---|
| 0 | 7 |
| 5 | 7 |
| 25 | 25 |
| 27 | 100 |
| 32 | 100 |
| 34 | 7 |
| 37 | 7 |

Similar versions of IEC were used for various other DVD-Ig proteins.

### Size Exclusion HPLC Methods

Size exclusion HPLC was used to determine the purity of DVD-Ig protein solutions. The assay was performed as outlined below.

A TSK gel guard (VWR Scientific, Bridgeport, N.J.; cat. no. 08543, 6.0 mm×4.0 cm, 7 μm), was combined with a TSK gel G3000SW (VWR Scientific, Bridgeport, N.J.; cat. no. 08541, 7.8 mm×30 cm, 5 μm) and run with an upper column pressure limit of 70 bar. The mobile phase consisted of 100 mM Na₂HPO₄/200 mM Na₂SO₄, pH 7.0. This buffer was created by dissolving 49.68 g anhydrous disodium hydrogen phosphate and 99.44 g anhydrous sodium sulfate in approximately 3300 ml Milli-Q water, adjusting the pH to 7.0 using 1 M phosphoric acid, increasing the buffer volume to 3500 ml with Milli-Q water and filtering the solution through a membrane filter.

The experimental parameters were listed as follows:

Flow rate: 0.3 ml/minute;
Injection volume (equivalent to 20 μg sample): 20 μl;
Column temperature: room temperature;
Autosampler temperature: 2 to 8° C.;
Run time: 50 minute;
Elution: isocratic gradient.

Detection was performed using a diode array detector using a 214 nm wavelength (>0.1 min peak width and 8 nm band width) and a 360 nm reference wavelength (100 nm band width).

Test samples were injected in duplicate. Purity was determined by comparing the area of DVD-Ig protein peak to the total area of all 214 nm absorbing components in the sample, excluding buffer-related peaks. High molecular weight aggregates and antibody fragments were resolved from intact DVD-Ig protein using this method.

### Freeze/Thaw Assays

The stability of DVD-Ig protein solutions was measured using repeated freeze/thaw assays. The DVD-Ig proteins were frozen at −80° C. and then thawed at 30° C. in a water bath and the resulting solutions were analyzed for aggregation by SEC and/or for subvisible particle formation by light obscuration assays.

### Accelerated and Real Time Storage Stability Studies

The pH and storage temperature of formulations are two important factors influencing protein stability during long-term storage of protein liquid and lyophilisate formulations. To assess the impact of these factors, the protein formulations were exposed to short-term storage at elevated temperatures, e.g., 40° C., (accelerated storage) in order to mimic long term storage and quickly gain insight in the formulation feasibility for long-term storage at lower temperatures (e.g., 2-8° C.). To assess the real time storage stability, the samples were also kept at 2-8° C.

### Light Obscuration Assays

Light obscuration assays were performed to measure the insoluble particulate content of aggregating DVD-Ig protein solutions. Light obscuration measurement equipment (particle counter, model syringe, Klotz Bad Liebenzell, Germany, series S20037) was equipped with laminar air hood (Thermo Electron Corp., Asheville, N.C.; Model No. ULT2586-9-A40) to minimize foreign particle contamination during measurements. Light obscuration analysis was performed as follows. A 3.5 ml sample was placed in a 5 ml round-bottom tube under laminar air flow conditions. Measurements were performed according to manufacturer's specifications in n=3 mode (0.8 mL per single measurement), after an initial 0.8 ml rinse.

### Differential Scanning Calorimetry (DSC)

Prior to DSC analysis, DVD-Ig proteins were dialyzed into a suitable buffer system using Slide-A-Lyzer Cassettes (Thermo Fisher Scientific, Waltham, Mass.). This buffer system (e.g., 5 mM phosphate/5 mM citrate was also used as a reference/blank for the DSC measurement. The antibody was analyzed at 1-2 mg/ml. An automated VP-Capillary DSC System (MicroCal, Piscataway, N.J.) was used. Unfolding of the molecules was studied by applying a 1° C./minute scan rate over a 25° C.-95° C. temperature range. Other measurement parameters were as follows: fitting period: 16 seconds; pre-scan wait: 10 minutes; feedback mode: none.

### Air/Liquid Interface Shaking Studies

Shaking studies were conducted at a concentration of 1 mg/ml in 6R glass vials on an HS 260 IKA shaker (Wilmington, N.C.) at a speed of 150 rpms (revolutions per minute). The optical density of samples was evaluated following shaking for various periods. Similarly, SEC was also done for samples pulled at various time points.

### PEG Solubility

PEG 3000 was used for solubility studies. A 50% w/v solution of PEG 3000 was prepared in water. Small aliquots of this solution were added to a stock solution of protein in buffer at 0.5 mg/ml concentration. The total volume required at the time first signs of precipitation originated was noted down.

### Real Solubility

For real solubility evaluations, the DVD-Ig protein was concentrated and stored overnight at 5° C. The solution was visually inspected for precipitates, phase separation, turbidity, etc. The supernatant (or both phases) was checked for dissolved concentration.

### Near UV-CD

The near UV-CD scans were taken at 1 mg/ml concentrations using 2 ml vial fill on Jasco spectrometer (JASCO Analytical Instruments, Easton, Md.) between 250 and 320 nm. The scan rate was 50 nm/minute and an average of 3 scans was taken. The spectrometer was allowed to equilibrate by turning on the lamp before data acquisition.

### ATR-FTIR Spectrometry

FTIR scans were taken at 1 mg/ml concentrations using 10 μl solutions on a Bruker ATR-FTIR (Bruker Optics, Billerica, Mass.). The scans were collected between 400-4000 cm⁻¹ and area normalized and second derivatized before being curve fitted using Origin software (OriginLab, Northampton, Mass.).

### Light Scattering

Light scattering studies were done on a Malvern zetasizer (Malvern Instruments Ltd., Worcestershire, UK) using a backscattering angle of 173°. Toluene was used as a standard and a buffer (e.g., acetate, histidine, and Tris) was used as a blank. An automatic mode was used.

### Dynamic Scanning Fluorimetry (DSF)

DSF was employed to assess the propensity of a protein to unfold. The technique involved the addition of dye Sypro Orange to the protein samples. This fluorescent dye is sensitive to hydrophobic surfaces and shows increased fluorescence in such environments. The sample with dye was then heated and the fluorescence signal as a function of temperature was monitored. As the temperature increased, the protein started to unfold and exposed its hydrophobic interior. This lead to dye binding to this region and a greater fluorescence signal. The temperature at which the signal begins to increase is the onset temperature (Tₒₙ). Proteins that have less intrinsic stability are more prone to unfold and have lower Tₒₙ values than proteins with greater intrinsic stability. DSF also provided a high throughput tool for rapid screening of clones in a 96 well format and eliminated potential limitations of larger quantities of samples and longer run times in DSC. 6 μl of the 0.4× SYPRO Orange dye (Invitrogen, Carlsbad, Calif.) was added to 27 μl of the DVD-Ig protein solution. The scan rate was 1° C./minute and scans were taken from 25-75° C.

### Lyophilization Methods

The DVD-Ig proteins were lyophilized according to standard methods and the stability of the resulting lyophilisates were investigated.

### Sample Preparation and Lyophilization

The vials were stoppered with autoclaved and dried lyo stoppers. Afterwards the vials were lyophilized with a freeze dryer. A typical cycle is shown below in Table 4. The samples were reconstituted to a 100 mg/ml DVD-Ig protein solution.

TABLE 4
Typical Freeze-Dry Cycle Used To Lyophilize DVD-Ig Proteins

| | | Shelf | Time/ | Step | |
|---|---|---|---|---|---|
| | | temperature | Step | time | Pressure |
| Step | | [° C.] | [hh:min] | [min] | [mbar] |
| 0 | Start | 20 | 0:00:00 | 0 | 1000 |
| 1 | Loading | 20 | 0:00:00 | 0 | 1000 |
| 2 | Freezing I (ramp) | 0 | 0:20:00 | 20 | 1000 |
| 3 | Freezing I | 0 | 2:10:00 | 130 | 1000 |
| 4 | Freezing II (ramp) | −45 | 1:20:00 | 80 | 1000 |
| 5 | Freezing II | −45 | 3:00:00 | 180 | 1000 |
| 6 | Adjust vacuum | −45 | 1:00:00 | 60 | 0.066 |
| 7 | Primary drying I | −25 | 1:00:00 | 60 | 0.066 |
| | (ramp) | | | | |
| 8 | Primary drying I | −25 | 70:00:00 | 4200 | 0.066 |
| 9 | Secondary drying II | 25 | 2:00:00 | 120 | 0.066 |
| | (ramp) | | | | |
| 10 | Secondary drying II | 25 | 0:15:00 | 15 | 0.036 |
| 11 | Secondary drying II | 25 | 8:00:00 | 480 | 0.036 |
| 12 | Holding step (ramp) | 5 | 0:30:00 | 30 | 0.036 |
| 13 | Holding step | 5 | 0:00:00 | 0 | 0.036 |
| 14 | Venting N2 | 5 | 0:00:00 | 0 | 500 |
| | atmosphere | | | | |
| | Total | | | 5375 | |

### I. Stability of Dvd-Ig Proteins

Examples 1-3 demonstrate that DVD-Ig proteins are less stable, e.g., aggregating more easily and having a lower melting temperature, than antibodies due to the increased structural complexity of DVD-Ig proteins.

### Example 1. Thermodynamic Comparison of DVD-Ig Proteins and Antibodies

An experiment was performed to determine the stability of a DVD-Ig protein in comparison to an antibody. Differential scanning calorimetry (DSC) of an IgG1 molecule (a monoclonal antibody, mAb) and a DVD-Ig, was performed to determine the differences in the thermodynamic properties of the two molecules. Specifically, DSC was performed to compare the thermodynamic properties of an exemplary antibody (Briakinumab, an anti-IL12 monoclonal antibody) to those of a DVD-Ig protein (TNF/PGE2). Formulation information and DSC conditions are provided below in Example 2. Comparison of the DSC profiles of Briakinumab to those of TNF/PGE2 shows the differences in three versus four domain unfolding (FIG. 1). It is also clear from FIG. 1 that the thermal unfolding of the DVD-Ig protein started earlier than that of the antibody, which indicates that the overall thermodynamic stability (or the intrinsic stability) of the DVD-Ig molecule is lower than that of the antibody. The thermodynamic stability of mAbs was generally higher than that of DVD-Ig proteins.

### Example 2. Impact of pH on the Stability of DVD-Ig Proteins in Solution

The thermodynamic stability (intrinsic stability) of DVD-Ig proteins formulated in solutions having a pH of 4, 6, and 8 was evaluated using differential scanning calorimetry (DSC). All formulations had a DVD-Ig protein concentration of 1 mg/ml in 5 mM citrate/5 mM phosphate buffer. Heating was performed at a scan rate of 1° C./minute. Results showing the impact of pH on the stability of multiple DVD-Ig proteins are provided in Table 5 below. Of note, Tm1-Tm4 described in Table 5 represent the thermal melting/unfolding temperatures of various domains, e.g., domain 1, domain 2, etc. The stability of two antibodies (Adalimumab and Briakinumab) is also described for comparison.

TABLE 5
Impact Of pH On Stability Of DVD-Ig Protein Formulations
With pH Of 4, 6, And 8, As Assessed By Differential
Scanning Calorimetry (DSC)

| | | | | | | Onset |
|---|---|---|---|---|---|---|
| DVD-Ig | pH | Tm1 ° C. | Tm2 ° C. | Tm3 ° C. | Tm4 ° C. | ° C. |
| 5 | 4 | 62.3 | 63.9 | 73.9 | 80.7 | 45 |
| 6 | 4 | 61.5 | 69.5 | 75.4 | 82.2 | 45 |
| 37 | 4 | 61.4 | 66.7 | 77.1 | 82.7 | 52 |
| 38 | 4 | 68.4 | 70.4 | 75.8 | 81.6 | 56 |
| 53 | 4 | 58.6 | 67.9 | 76.5 | 82.7 | 46 |
| 54 | 4 | 66 | 67.9 | 75.2 | 82.6 | 52.5 |
| 65 | 4 | 61.3 | 69 | 73.9 | 81.3 | 52.5 |
| 66 | 4 | 65.5 | 67.6 | 74.7 | 81.9 | 55 |
| 165 | 4 | 63 | 67.4 | 75.4 | 82.1 | 47.5 |
| 166 | 4 | 67.3 | 71.7 | 74.9 | 82.4 | 55 |
| 257 | 4 | 66.2 | 69.2 | 76.6 | 83.5 | 52.5 |
| 258 | 4 | 68.2 | 69.6 | 78.8 | 83.7 | 55 |
| 277 | 4 | 61.4 | 64.8 | 75.3 | 82.6 | 50 |
| 278 | 4 | 55.8 | 67.5 | 76.1 | 82.8 | 45 |
| 281 | 4 | 65.8 | 68.4 | 79.1 | 83.1 | 55 |
| 282 | 4 | 73.1 | 75 | 77.4 | 83.8 | 57.5 |
| 5 | 6 | 61.9 | 65.1 | 76 | 81.8 | 50 |
| 6 | 6 | 61.6 | 69.8 | 76.2 | 81.9 | 48 |
| 37 | 6 | 60.4 | 67.5 | 77.2 | 83.1 | 51 |
| 38 | 6 | 69.07 | 70.3 | 75.9 | 82 | 57.5 |
| 53 | 6 | 58.3 | 67.05 | 76.4 | 82 | 49 |
| 54 | 6 | 65.8 | 67.6 | 74.9 | 82 | 56 |
| 65 | 6 | 61.2 | 67.3 | 73.4 | 82.4 | 51.5 |
| 66 | 6 | 65.3 | 67.5 | 74.8 | 81.9 | 55 |
| 165 | 6 | 62.6 | 67.3 | 75.7 | 82.1 | 50 |
| 166 | 6 | 67.3 | 71 | 74 | 82 | 54 |
| 257 | 6 | 66.6 | 69.2 | 75.8 | 82.9 | 57 |
| 258 | 6 | 67.8 | 70 | 77.5 | 79.5 | 55 |
| 277 | 6 | 61.4 | 65.8 | 74.7 | 82.23 | 52.5 |
| 278 | 6 | 56.6 | 66.7 | 75.8 | 82.3 | 45 |
| 281 | 6 | 65.8 | 67.8 | 78.6 | 81.7 | 53 |
| 282 | 6 | 69.8 | 75 | 77.9 | 83.4 | 57 |
| 5 | 8 | 65.16 | 68.35 | 77 | 82.48 | 45 |
| 6 | 8 | 62 | 69.07 | 73.08 | 82.61 | 45 |
| 38 | 8 | 68.4 | 70.7 | 74.8 | 82.8 | 50 |
| 53 | 8 | 57.6 | 68.7 | 75 | 83 | 40 |
| 54 | 8 | 65.5 | 67.6 | 74.5 | 82.8 | 52.5 |
| 65 | 8 | 61 | 69.6 | 72.5 | 82.8 | 48 |
| 66 | 8 | 64.8 | 67.1 | 72.4 | 82.9 | 52.5 |
| 165 | 8 | 63.9 | 68.6 | 74.1 | 82.6 | 47.5 |
| 166 | 8 | 64.3 | 69.3 | 74 | 82.7 | 45 |
| 257 | 8 | 67.2 | 69.3 | 76.7 | 83.6 | 55 |
| 258 | 8 | 69.5 | 71.4 | 78.1 | 83.9 | 53 |
| 277 | 8 | 60.18 | 68.98 | 74.5 | 83 | 50 |
| 278 | 8 | 52.8 | 68.8 | 75.8 | 83.02 | 44 |
| 281 | 8 | 65.2 | 67.1 | 78.3 | 83.2 | 51 |
| 282 | 8 | 71.9 | 74.5 | 77.4 | 83.9 | 55 |
| mAbs | | | | | | |
| Adalimumab | 6 | 72 | 76 | 84 | NA | 62 |
| Briakinumab | 6 | 69 | 76 | 83 | NA | 59 |

As shown in Table 5, DVD-Ig proteins in general have unfolding onset temperatures of greater than 50° C., and the melting temperatures are therefore slightly lower than those of antibodies and other stable proteins. Example 2 shows that the onset temperature for Briakinumab and Adalimumab is around 60° C. at pH 6, whereas for DVD-Ig proteins the average is around 53° C.

The data also shows that the melting temperatures of DVD-Ig proteins are higher at pH 6 and pH 8 than at pH 4. Thus, pH affects the physico-chemical stability of DVD-Ig proteins, and stability appears best at approximately pH 6.

To further assess the impact of solution pH on the stability of DVD-Ig protein formulations during long-term storage, DVD-Ig protein formulations were analyzed using SEC before being subjected to storage (T0) or after being subjected to 3 months of accelerated storage (T3 m). Storage stability of the DVD-Ig proteins in solutions (1 mg/ml DVD-Ig protein in 5 mM citrate/5 mM phosphate buffer with the presence of 80 mg/ml sucrose) formulated at pH of 4, 6, or 8 was evaluated. For accelerated storage, samples were filled into sterile vials (approx. 500 μl each) and stored under controlled conditions (in temperature chambers and in the absence of light) at 40° C. The percentage of DVD-Ig protein monomers (Mon), aggregates (Agg), and fragments (Frag) was determined using SEC and the results are presented in Table 6.

TABLE 6
Impact Of pH On The Stability Of 1 mg/ml Concentration DVD-Ig
Protein Solutions Before And After Accelerated Storage

| | | Mon/ | Agg/ | Frag/ | Mon/ | Agg/ | Frag/ |
|---|---|---|---|---|---|---|---|
| DVD-Ig | pH | T0 | T0 | T0 | T3 m | T3 m | T3 m |
| 5 | 4 | 91.14 | 5 | 3.85 | 57.51 | 0.79 | 41.6 |
| 6 | 4 | 97.38 | 2.29 | 0.31 | 62.46 | 4.11 | 33.4 |
| 38 | 4 | 94.9 | 3.58 | 1.5 | 56.52 | 22.02 | 21.44 |
| 53 | 4 | 97.61 | 0.99 | 1.38 | 28.11 | 53.94 | 17.93 |
| 54 | 4 | 96.57 | 1.93 | 1.48 | 53.89 | 15.2 | 30.89 |
| 65 | 4 | 94.46 | 1.33 | 4.21 | 50.35 | 26.21 | 23.42 |
| 66 | 4 | 97.99 | 0.9 | 1.1 | 58.39 | 17.72 | 23.87 |
| 165 | 4 | 96.2 | 2.25 | 1.53 | 68.38 | 3.18 | 28.42 |
| 166 | 4 | 97.09 | 0.76 | 2.14 | 66.3 | 0.66 | 30.55 |
| 258 | 4 | 98.61 | 0.46 | 0.91 | 45.42 | 30.56 | 24 |
| 277 | 4 | 97.05 | 1.55 | 1.38 | 61.26 | 11.48 | 27.23 |
| 278 | 4 | 98.33 | 0.9 | 0.76 | 45.58 | 28.13 | 26.27 |
| 5 | 6 | 90.46 | 7.02 | 2.51 | 81.75 | 3.04 | 12.77 |
| 6 | 6 | 97.02 | 2.78 | 0.18 | 85.44 | 2.71 | 11.82 |
| 38 | 6 | 95.31 | 3.48 | 1.2 | 87.89 | 5.48 | 6.61 |
| 53 | 6 | 97.75 | 1.02 | 1.22 | 86.68 | 6.21 | 7.08 |
| 54 | 6 | 96.48 | 2.07 | 1.43 | 88.03 | 5.37 | 6.59 |
| 65 | 6 | 94.89 | 0.89 | 4.21 | 87.13 | 5.78 | 7.06 |
| 66 | 6 | 97.99 | 0.9 | 1.09 | 88.93 | 5.13 | 5.91 |
| 165 | 6 | 96.21 | 2.24 | 1.53 | 86.31 | 5.84 | 7.82 |
| 166 | 6 | 98.6 | 1.39 | 0 | 89.37 | 0.92 | 7.38 |
| 258 | 6 | 98.98 | 0.2 | 0.81 | 84.76 | 9.41 | 5.81 |
| 277 | 6 | 97.48 | 1.48 | 1.03 | 83.96 | 5.11 | 10.91 |
| 278 | 6 | 98.65 | 0.78 | 0.56 | 79.81 | 9.52 | 10.65 |
| 5 | 8 | 90.21 | 7.26 | 2.51 | 67.86 | 4.51 | 22.75 |
| 6 | 8 | 97.36 | 2.44 | 0.18 | 76.33 | 8.89 | 14.76 |
| 38 | 8 | 95.09 | 3.47 | 1.42 | 80.11 | 9.37 | 10.5 |
| 53 | 8 | 97.81 | 0.96 | 1.22 | 79.63 | 9.82 | 10.53 |
| 54 | 8 | 96.69 | 1.93 | 1.37 | 81.13 | 9.44 | 9.41 |
| 65 | 8 | 93.12 | 1.27 | 5.59 | 79.5 | 9.88 | 10.59 |
| 66 | 8 | 97.69 | 0.99 | 1.31 | 81.37 | 9.17 | 9.44 |
| 165 | 8 | 96.57 | 2.03 | 1.39 | 71.02 | 18.85 | 10.11 |
| 166 | 8 | 97.23 | 0.76 | 1.99 | 79.76 | 1.92 | 11.3 |
| 258 | 8 | 98.67 | 0.24 | 1.07 | 84.22 | 5.91 | 9.84 |
| 277 | 8 | 97.06 | 1.72 | 1.2 | 70.08 | 16.13 | 13.78 |
| 278 | 8 | 98.18 | 0.98 | 0.83 | 43.34 | 45.59 | 11.04 |

The results in Table 6 show that following accelerated storage, the DVD-Ig proteins generally were stable in the pH range 4-8. The DVD-Ig proteins had the greatest stability at around pH 6.

All of the DVD-Ig proteins tested (including DVD 5, DVD 6, DVD 38, DVD 53, DVD 54, DVD 65, DVD 66, DVD 165, DVD 166, DVD 258, DVD 277, and DVD 278) had a greater percentage of monomers and a lower percentage of fragments at pH 6 than at pH 4 or at pH 8. Nine of the twelve DVD-Ig proteins tested showed a lower percentage of aggregates at pH 6 than at pH 4, and for the three DVD-Ig proteins that showed the reverse pattern, the difference in the percentage of aggregates at pH 6 and pH 4 was very small (difference of less than 2.7%). Also, eleven of the twelve DVD-Ig proteins tested showed a lower percentage of aggregates at pH 6 than at pH 8. Thus, accelerated storage resulted in increased aggregate formation. However, the increase was less than anticipated, particularly at pH 6.

### Impact of Solution pH on the Storage Stability of IL12IL18 DVD-Ig Protein Formulations

To assess the impact of solution pH on the stability of DVD-Ig protein formulations during storage, DVD-Ig protein formulations with solution pH of 4, 6, or 8 were analyzed using SEC and IEC before being subjected to storage (T0) or after being subjected to 4 days (4 d), 7 days (7 d), or 21 days (21 d) of storage at 5° C., 40° C., or 50° C. (See Tables 7 and 8). The solutions evaluated had an IL12-IL18 DVD-Ig protein concentration of 2 mg/ml and were in a buffer of 10 mM citrate and 10 mM phosphate. Samples were filled into sterile vials (approx. 500 μl each) and stored under controlled conditions (in temperature chambers and in the absence of light). The percentage of DVD-Ig protein monomers, aggregates, and fragments was determined using SEC (see Table 7) and the numbers of main, acidic and basic species were assessed using IEC (see Table 8).

TABLE 7
Storage Stability of IL12-IL18 DVD-Ig Protein Formulations
with pH 4, 6, or 8, as Measured Using SEC

| | Monomer | | | Aggregates | | | Fragments | | |
|---|---|---|---|---|---|---|---|---|---|
| | pH 4 | pH 6 | pH 8 | pH 4 | pH 6 | pH 8 | pH 4 | pH 6 | pH 8 |
| T0 | 97.64 | 97.61 | 97.84 | 1.35 | 1.36 | 1.23 | 1.01 | 1.03 | 0.94 |
| 4 d, 5° C. | 97.98 | 97.64 | 97.95 | 1.07 | 1.31 | 1.19 | 0.95 | 1.06 | 0.87 |
| 7 d, 5° C. | 97.68 | 98.02 | 97.8 | 1.25 | 1.17 | 1.08 | 1.07 | 0.81 | 1.11 |
| 21 d, 5° C. | 98.13 | 97.81 | 97.64 | 1.23 | 1.17 | 1.08 | 0.88 | 0.96 | 1.28 |
| 4 d, 40° C. | 97.21 | 97.32 | 96 | 0.56 | 1.4 | 1.68 | 2.24 | 1.28 | 2.32 |
| 7 d, 40° C. | 96.15 | 97.32 | 94.57 | 0.71 | 1.35 | 1.96 | 3.15 | 1.33 | 3.48 |
| 21 d, 40° C. | 91.99 | 96.39 | 91.31 | 1.16 | 1.49 | 3.15 | 6.85 | 2.12 | 5.54 |
| 4 d, 50° C. | 90.2 | 97.07 | 91.73 | 5.54 | 1.46 | 2.78 | 4.26 | 1.47 | 5.49 |
| 7 d, 50° C. | 83.19 | 96.35 | 81.32 | 9.66 | 1.42 | 10.68 | 7.15 | 2.24 | 8.2 |
| 21 d, 50° C. | 52.98 | 91.36 | 46.57 | 33.1 | 4.17 | 33.35 | 13.91 | 4.47 | 17.18 |

The SEC data show that the IL12IL18 DVD-Ig protein formulations were stable at pH 6. At pH 6, the stored IL12IL18 DVD-Ig protein formulations generally showed >95% monomers and <2% aggregates. Even under accelerated storage conditions of 50° C. for 21 days, the formulation retained >90% monomers and <5% aggregates. IL12IL18 DVD-Ig protein formulations were more stable at pH 6 than pH 4 and pH 8, particularly in the longer duration and higher temperature storage conditions (e.g., in the 21 day, 50° C. condition).

TABLE 8
Storage Stability of IL12IL18 DVD-Ig Protein Formulations
with pH 4, 6, or 8, as Measured Using IEC

| | Main Species | | | Acidic | | | Basic | | |
|---|---|---|---|---|---|---|---|---|---|
| | pH 4 | pH 6 | pH 8 | pH 4 | pH 6 | pH 8 | pH 4 | pH 6 | pH 8 |
| T0 | 69.98 | 71.6 | 69.13 | 14.96 | 15.21 | 18.12 | 15.06 | 13.19 | 12.75 |
| 4 d, 5° C. | 70.32 | 71 | 68.46 | 15.08 | 15.54 | 18.84 | 14.6 | 13.46 | 12.75 |
| 7 d, 5° C. | 69.74 | 70.69 | 67.14 | 15.43 | 15.91 | 19.71 | 14.83 | 13.41 | 13.15 |
| 21 d, 5° C. | 69.67 | 71.32 | 66.78 | 15.65 | 16.26 | 20.95 | 14.68 | 12.42 | 12.26 |
| 4 d, 40° C. | 56.43 | 68.09 | 43.52 | 21.14 | 18.73 | 45.51 | 22.33 | 13.19 | 10.97 |
| 7 d, 40° C. | 49.1 | 65.11 | 26.55 | 26.08 | 22.03 | 59.93 | 24.83 | 12.86 | 13.53 |
| 4 d, 50° C. | 36.58 | 57.09 | 25.88 | 29.47 | 29.48 | 48.23 | 33.95 | 13.43 | 25.89 |

### Example 3. DVD-Ig Proteins Aggregate More Easily than Monoclonal Antibodies

The impact of shaking on the aggregation of antibodies versus DVD-Ig proteins was examined. Shaking is a stress that can lead to the aggregation of molecules. The susceptibility of a DVD-Ig protein, TNF/PGE2 (DVD B), to aggregation following shaking was compared with a monoclonal antibody, Briakinumab, using solutions having a protein concentration of 1 mg/ml (solutions at pH 6, 10 mM citrate/10 mM phosphate) in 6R vials. The 6R vials were filled with samples of 5 ml of the protein solution and shaken on an IKA shaker for varying durations of time (0, 5, 24, 48, or 96 hours). The samples were checked for optical density at 500 nm, which provides a measurement of the turbidity of the solutions. Higher turbidity indicates greater aggregation and less stability. The results are shown in Table 9, revealing that the DVD-Ig protein aggregates more readily than the monoclonal antibody.

TABLE 9
Impact Of Shaking on the OD₅₀₀ of 1 mg/ml Solutions
of TNF/PGE2 DVD-Ig Protein and Briakinumab

| | | TNF/ |
|---|---|---|
| | | PGE2 |
| Time (h) | Briakinumab | (DVD-B) |
| 0 | 0 | −0.0095 |
| 5 | 0.001 | 0.01175 |
| 24 | 0.01 | 0.0949 |
| 48 | 0.025 | 0.295 |
| 96 | 0.045 | 0.58 |

After 48 hours of shaking, the DVD-Ig protein was less stable than the monoclonal antibody. As indicated by OD₅₀₀ measurements which show turbidity of the solution, shaking caused the DVD-Ig protein to form more visible aggregates than the monoclonal antibody. The greater formation of visible aggregates of DVD-Ig protein compared with monoclonal antibody indicates that the DVD-Ig protein is less stable than the monoclonal antibody. Also, these results suggest that not all DVD-Ig molecules are stable at pH 6 as a monoclonal antibody.

### II. Assay for Identifying an Aqueous Stable DVD-Ig Protein (AS-DVD-Ig)

### Example 4. DVD-Ig Proteins can be Characterized as “Aqueous Stable” or “Aqueous Non-Stable”

The following example describes an SEC study showing that, surprisingly, DVD-Ig proteins can be characterized as either aqueous stable, e.g., the DVD-Ig protein shows low aggregation, or aqueous non-stable, e.g., the DVD-Ig protein is prone to aggregation. Notably, many of the DVD-Ig proteins tested were found to be aqueous/lyophilized non-stable. Due to the structural complexity of DVD-Ig proteins and the prominence of hydrophobic interactions at high concentrations, it was not expected that DVD-Ig proteins would be stable in formulations at high concentrations.

To assess the impact of storage temperature during accelerated or long-term storage of protein liquid formulations on protein stability, various DVD-Ig proteins were exposed to short-term storage at elevated temperatures in order to quickly gain insight in the formulation feasibility for long-term storage at lower temperatures (e.g., 2-8° C.).

### Stability Screen at a High Concentration for 14 Days

DVD-Ig protein formulations with concentrations of 60 mg/ml were analyzed using SEC before being subjected to storage (T=0) or after being subjected to 14 days of accelerated storage (T=14 days) (Table 10). Storage stability of the DVD-Ig proteins in solution (60 mg/ml, 10 mM citrate/10 mM phosphate buffer with 80 mg/ml sucrose) was evaluated at 40° C. After defined storage periods, samples were pulled and the impact of storage time on DVD-Ig protein stability was evaluated. Briefly, samples were filled into sterile vials (approx. 500 μL each) and stored under controlled conditions (in temperature chambers and in the absence of light) at 40° C. At predefined points of time, samples of prepared solutions were pulled for analysis according to the sample pull scheme. The percentages of DVD-Ig protein monomers (Mon), aggregates (Agg), and fragments (Frag) were determined using SEC, and the results are presented in Table 10.

TABLE 10
Impact of High Concentration on the Storage
Stability of DVD-Ig Protein Solutions

| | Mon/ | Agg/ | Frag/ | Mon/ | Agg/ | Frag/ |
|---|---|---|---|---|---|---|
| DVD-Ig | T0 | T0 | T0 | T14 d | T14 d | T14 d |
| 5 | 79.1 | 19.12 | 1.77 | 73.28 | 24.26 | 2.44 |
| 6 | 84.01 | 12.69 | 3.29 | 89.46 | 9.81 | 0.71 |
| 37 | 92.85 | 6.34 | 0.8 | 74.35 | 23.42 | 2.21 |
| 65 | 95.4 | 1.31 | 3.28 | 92.79 | 5.39 | 1.79 |
| 66 | 96.56 | 1.08 | 2.35 | 94.49 | 3.88 | 1.6 |
| 165 | 90.17 | 8.99 | 0.83 | 64.09 | 33.44 | 2.45 |
| 166 | 98.17 | 1.09 | 0.72 | 94.93 | 3.11 | 1.93 |
| 257 | 94.83 | 4.77 | 0.39 | 76.47 | 21.78 | 1.72 |
| 277 | 97.46 | 1.77 | 0.76 | 85.06 | 13.19 | 1.73 |
| 278 | 98.45 | 1.06 | 0.48 | 73.01 | 25.69 | 1.27 |
| 281 | 93.92 | 2.78 | 3.29 | 68.01 | 30.5 | 1.46 |
| 282 | 98.34 | 1.03 | 0.62 | 95.39 | 3.05 | 1.54 |

Surprisingly, as shown in Table 10, a subset of the DVD-Ig proteins tested was stable. Ten of the twelve DVD-Ig proteins tested (DVD 5, DVD 6, DVD 37, DVD 65, DVD 66, DVD 166, DVD 257, DVD 277, DVD 278, and DVD 282) showed less than 26% aggregate formation and had greater than 73% monomers following 14 days of accelerated storage. Five of the DVD-Ig proteins tested (DVD 6, DVD 65, DVD 66, DVD 166, and DVD 282) showed aggregate formation of less than 10%, and three of these (DVD 66, DVD 166, and DVD 282) showed aggregate formation of less than 5%.

As described above, certain DVD-Ig proteins (“Aqueous Stable DVD-Ig” proteins or “AS-DVD-Ig” proteins) remain stable (e.g., less than 6% aggregate formation or less than 10% aggregate formation) following accelerated storage in 14 days at 40° C., even when formulated at high concentration (e.g., concentrations of 60 mg/ml, or higher). The majority of DVD-Ig proteins (non-AS-DVD-Ig proteins) tended to aggregate during accelerated storage, as would be expected based on the general structure of DVD-Ig proteins and the stability studies described in Examples 1-3. Thus, in one embodiment, the cutoff for separating the AS-DVD-Ig proteins and the non-AS-DVD-Ig proteins was taken as the formation of 6% net aggregates or less in 14 days at 40° C. when stored at >50 mg/ml at pH 6, as four of the twelve DVD-Ig proteins tested showed aggregate formation at this level.

Overall the majority of DVD-Ig proteins do not show low aggregation, e.g., 1% or less aggregation at 5° C. after 21 days or 10% or less aggregation at 40° C. following 21 days of storage. For example, in an assay which examined monomer loss after 7 days in a solution having a TNF/IL13 DVD-Ig protein concentration of 50 mg/ml at either 4° C. or 40° C., the majority of DVD-Ig proteins showed an increase in monomer loss as determined by SEC. In some cases the amount of monomer loss was negative as the monomer level increased in these cases (e.g., some of the aggregates dissociated and formed back monomer and hence the apparent decrease in loss). A third experiment tested TNF/SOST DVD-Ig proteins in a solution having a DVD-Ig protein concentration of 50 mg/ml at 4° C. As in the experiment relating to TNF/IL13 DVD-Ig proteins, the majority of DVD-Ig proteins showed an increase in monomer loss (determined by SEC).

Notably, the above assays can also be used to distinguish Lyophilized-Stable DVD-Immunoglubulin (LS-DVD-Ig) proteins. The cutoff for separating the LS-DVD-Ig proteins and the non-LS-DVD-Ig proteins was taken as the formation of 15% net or less aggregates in 14 days at 40° C. when stored at >50 mg/ml at pH 6. Thus, DVD-Ig proteins tested in the above assay that result in less than 6% aggregation are considered both AS-DVD-Ig and LS-DVD-Ig proteins, and DVD-Ig proteins resulting in less than 15% aggregation are considered LS-DVD Ig proteins. Both AS-DVD-Ig and LS-DVD-Ig proteins represent only a small percentage of the overall DVD-Ig proteins tested.

### III. Stability of Non-AS-DVD-Ig Proteins in Formulations

The following examples provide data showing the stability of non-AS-DVD-Ig proteins (which fail the aggregation test, i.e., show more than, for example, 6% aggregation, described in Example 4 above), in formulations, in comparison to AS-DVD-Ig protein molecules (described in Sections IV to VIII).

### Example 5: Impact of Concentration on the Storage Stability of an Exemplary Non-AS-DVD-Ig Protein

To assess the impact of protein concentration on long term storage stability, formulations of an exemplary non-AS-DVD-Ig protein with concentrations of 1, 2, 5, 10, 25, 50, and 75 mg/ml were subjected to storage for 14 days at 40° C. The formulations had a pH of 6 and were in 15 mM histidine buffer alone. The samples were filled into sterile vials (approx. 500 μl each) and stored under controlled conditions (in temperature chambers and in the absence of light). The samples were analyzed using SEC to determine the percentage of aggregates following storage. The resulting data is provided in Table 11.

TABLE 11
The Total Percent Aggregates in the DVD-B protein as Measured
Using SEC Following Storage at 40° C. for 14 Days

| Concentration of | Aggregates |
|---|---|
| DVD B | % |
| 1 mg/ml | 0 |
| 2 mg/ml | 0.2 |
| 5 mg/ml | 3.64 |
| 10 mg/ml | 6.76 |
| 25 mg/ml | 18.02 |
| 50 mg/ml | 32.82 |
| 75 mg/ml | 48.28 |

The data in Table 11 indicate that under the tested conditions (i.e., pH 6, 15 mM histidine buffer) DVD B becomes unstable, namely, a high proportion of aggregates form after 14 days of storage at 40° C. when high concentrations are reached. The percentage of aggregates formed exceeded 18% at a concentration of 25 mg/ml or more. Thus, DVD B, a non-AS-DVD-Ig protein, was not stable in histidine buffer as evidenced by increased aggregation during storage.

### Example 6: Impact of pH, Ionic Strength, and Concentration on the Storage Stability of an Exemplary Non-AS-DVD-Ig Protein

To assess the impact of pH, ionic strength, and concentration on the storage stability of a DVD-Ig protein in solution, various formulations of DVD B (5 mg/ml and 100 mg/ml) were evaluated at 40° C. and 5° C. After defined storage periods, samples were pulled and the impact of storage time on DVD-Ig protein stability was evaluated. The following buffers were used: acetate for pH 4.5, histidine for pH 6 and Tris for pH 8. A 2 mM concentration of buffer was used for 1 mM ionic strength solutions and a 10 mM concentration of buffer for 20 and 100 mM ionic strength solutions (sodium chloride was used to further maintain ionic strength). Samples were filled into sterile vials, approx. 500 μl each, and stored under controlled conditions in a temperature chamber and in the absence of light. After 3 months at 5° C. (5 C, 3 m) or 21 days at 40° C. (40 C, 21 d), samples of prepared solutions were analyzed using SEC. The numbers of net aggregates measured using SEC are presented in Table 12. Tables 13 and 14 further show that the addition of different stabilizers/excipients did not result in the improvement in percent monomer remaining after defined time points (results were obtained using the methodology described above).

TABLE 12
Impact of Storage Of DVD B Protein Under Various Formulation
Conditions on the Amount Of Aggregates Formed as Measured By SEC

| | | Added | |
|---|---|---|---|
| DVD-Ig protein concentration | | Ionic | |
| and storage condition | pH | strength | Aggregate (Net) |
| DVD-B, 100 mg/ml, 5° C., 3 m | 4.5 | 1 | 49.51 |
| | | 20 | 54.79 |
| | | 100 | 56.64 |
| | 6 | 0 | 11.68 |
| | | 1 | 6.6 |
| | | 20 | 9.4 |
| | | 100 | 7.85 |
| | 8 | 1 | 5.66 |
| | | 20 | 8.08 |
| | | 100 | 7.87 |
| DVD-B, 5 mg/ml, 40° C., 21 d | 6 | 0 | −6.56 |
| | | 1 | −3.3 |
| | | 20 | 10.79 |
| | | 100 | 9.99 |
| DVD-B, 100 mg/ml, 40° C., 21 d | 6 | 0 | 73.37 |
| | | 1 | 70.39 |
| | | 20 | 77.39 |
| | | 100 | 65.4 |

TABLE 13
Polyol And Polysorbate Have Little to No Effect On Stability
Of DVD-B at 1 mg/ml in Histidine Buffer, pH 6

| | Sample | | | | |
|---|---|---|---|---|---|
| 1 mg/ml | No. | Monomer | Aggregate | Fragment | AUC |
| Histidine | | | | | |
| Buffer, | | | | | |
| T0 | | 98.97 | 0.34 | 0.67 | 79787 |
| T4, 40° C. | 1 | 98.13 | 0.82 | 1.04 | 78552 |
| | 2 | 97.98 | 0.87 | 1.13 | 78622 |
| | Average | 98.055 | 0.845 | 1.085 | 78587 |
| T7, 40° C. | 1 | 97.45 | 1.15 | 1.39 | 77836 |
| | 2 | 97.42 | 1.2 | 1.36 | 78137 |
| | Average | 97.435 | 1.175 | 1.375 | 77986.5 |
| T21, 40° C. | 1 | 92.21 | 4.34 | 3.44 | 170875 |
| | 2 | 93.19 | 3.7 | 3.1 | 72149 |
| | Average | 92.7 | 4.02 | 3.27 | 121512 |
| T4, 50° C. | 1 | 94.06 | 4.15 | 1.77 | 39698 |
| | 2 | 93.37 | 4.44 | 2.18 | 43002 |
| | Average | 93.715 | 4.295 | 1.975 | 41350 |
| T7, 50° C. | 1 | 93.09 | 3.9 | 2.99 | 26451 |
| | 2 | 91.95 | 4.81 | 3.22 | 30158 |
| | Average | 92.52 | 4.355 | 3.105 | 28304.5 |
| 30 mM | | | | | |
| Histidine, | | | | | |
| 80 mg/ml | | | | | |
| Sucrose, 0.02% | | | | | |
| Tween 80 pH 6 | | | | | |
| T0 | | 97.5 | 1.61 | 0.88 | 62902 |
| T21, 40° C. | 1 | 95.19 | 1.47 | 2.95 | 79362 |
| | 2 | 95.56 | 1.67 | 3.13 | 79445 |
| | Average | 95.375 | 1.57 | 3.04 | 79403.5 |
| T4, 50° C. | 1 | 94.94 | 3.38 | 1.66 | 80742 |
| | 2 | 94.98 | 3.3 | 1.62 | 79436 |
| | Average | 94.96 | 3.34 | 1.64 | 80089 |
| T7, 50° C. | 1 | 91.06 | 6.71 | 2.21 | 79672 |
| | 2 | 91.01 | 6.6 | 2.37 | 79820 |
| | Average | 91.035 | 6.655 | 2.29 | 79746 |

TABLE 14
Polysorbate Has Little to No Effect Oon Stability of DVD-B
at 100 mg/ml in Histidine Buffer, pH 6

| 100 mg/ml | | Monomer | Aggregate | Fragment | AUC |
|---|---|---|---|---|---|
| pH 6 | T0 | 96.26 | 2.43 | 1.3 | 73681 |
| (15 mM | T7 (Vial1) | 41.4 | 56.1 | 2.34 | 64692 |
| Histidine) | T7 (Vial2) | 42.5 | 55.2 | 2.14 | 63246 |
| | T7 (Avg.) | 41.95 | 55.65 | 2.24 | 63969 |
| | T21(Vial1) | 37.2 | 60.03 | 2.76 | 52389 |
| | T21(Vial2) | 38.8 | 58.05 | 3.13 | 50722 |
| | T21 (Avg) | 38 | 59.04 | 2.945 | 51555.5 |
| pH 6 | T0 | 96.22 | 2.43 | 1.33 | 72007 |
| (15 mM | T7 (Vial1) | 42.9 | 54.8 | 2.2 | 65403 |
| Histidine) + | T7 (Vial2) | 47 | 50.8 | 2.09 | 58048 |
| 0.02% | T7 (Avg.) | 44.95 | 52.8 | 2.145 | 61725.5 |
| Tween 80 | T21 (Vial1) | 40.32 | 54.54 | 5.12 | 32321 |
| | T21 (Vial2) | 38.38 | 55.9 | 5.7 | 30927 |
| | T21 (Avg) | 39.35 | 55.22 | 5.41 | 31624 |

TABLE 15
Polyol Or Surfactant Does Not Improve Stability (1 mg/ml) Of DVD-B

| | Monomer (%) | | | Aggregate (%) | | | Fragment (%) | | |
|---|---|---|---|---|---|---|---|---|---|
| pH 6 Formulation | T0 | 1M | 3M | T0 | 1M | 3M | T0 | 1M | 3M |
| 15 mM Na Phos. | 96.45 | 90.03 | 80.83 | 1.41 | 5 | 9.09 | 2.12 | 4.95 | 10.06 |
| 15 mM Na Cit. | 96.51 | 90.72 | 85.3 | 1.44 | 5.3 | 6.57 | 2.04 | 3.97 | 8.11 |
| 15 mM Na Succ. | 96.06 | 87.41 | 78.17 | 1.53 | 5.46 | 8.24 | 2.39 | 7.11 | 13.57 |
| 15 mM Na Acet. | 96.14 | 89.62 | 81.8 | 1.48 | 4.76 | 7.52 | 2.36 | 5.6 | 10.66 |
| 15 mM Arg. | 96.12 | 92.48 | 85.72 | 1.65 | 3.39 | 5.59 | 2.21 | 4.11 | 8.68 |
| 15 mM Hist. | 96.42 | 91.82 | 81.6 | 1.29 | 3.85 | 6.39 | 2.28 | 4.32 | 12 |
| Self Buff. | 96.03 | 88.79 | 81.44 | 1.57 | 3.91 | 4.18 | 2.38 | 7.29 | 14.37 |
| UB 10 mg/ml Mannitol | 95.86 | 90.18 | 84.08 | 2.01 | 5.74 | 8.09 | 2.11 | 4.07 | 7.81 |
| UB 10 mg/ml Sorbitol | 96.49 | 89.36 | 84.09 | 1.38 | 6.56 | 7.68 | 2.11 | 4.07 | 8.21 |
| UB 10 mg/ml Sucrose | 96.12 | 90.03 | 84.26 | 1.58 | 5.92 | 7.62 | 2.28 | 4.03 | 8.11 |
| UB 10 mg/ml Trehalose | 96.34 | 89.97 | 84.31 | 1.5 | 5.98 | 7.59 | 2.14 | 4.03 | 8.09 |
| UB 2.5% Gly | 96.43 | 87.22 | | 1.42 | 8.59 | | 2.13 | 4.17 | |
| UB 15 mM (NH₄)₂SO₄ | 96.69 | 90.92 | 85.76 | 1.22 | 4.97 | 5.68 | 2 | 4.1 | 8.55 |
| UB 20 mM NaCl | 96.44 | 90.35 | 84.36 | 1.41 | 5.5 | 6.74 | 2.13 | 4.13 | 8.88 |
| UB 200 mM NaCl | 96.37 | 91.85 | 85.2 | 1.52 | 3.24 | 3.62 | 2.09 | 4.89 | 11.16 |

### UB=Citrate/Phosphate Buffer

The data show that although various solution conditions were analyzed, DVD B was not very stable even at pH 6 (see, for example Table 12). Also, at pH 6, the ionic strength did not show a consistent relationship with net aggregate formation. The poor stability is indicated by the formation of high amounts of aggregates even in the 5° C. storage condition. Furthermore, the addition of a polyol and/or a surfactant did not improve aggregation of DVD B (see Tables 13, 14, and 15).

As described above in Examples 1 to 3, many DVD-Ig proteins are intrinsically unstable. However, surprisingly, certain DVD-Ig proteins can be characterized as being stable, as described in Example 4. The experiments described in the Examples below demonstrate that AS-DVD-Ig proteins can unexpectedly be stably formulated, even at high concentrations, despite the differences in amino acid sequence. The below examples stand in contrast to Examples 5 and 6, which show the failure of non-AS-DVD-Ig proteins to be formulated.

### IV. AS-DVD-Ig Proteins are Stable in Formulations Containing a Buffer at pH Range of 4.5-7.5

### Example 7: Effect of Buffer Concentrations on the Stability of DVD-Ig Proteins

The concentration of a buffer, e.g., histidine, is one of the important factors that may influence protein stability during accelerated/long-term storage of protein liquid formulations. To assess the impact, the protein was exposed to short-term storage at elevated and real time temperatures in order to quickly gain insight into stable formulations for long-term storage at lower temperatures (e.g., 2-8° C.).

Storage stability of DVD-Ig proteins in solution was evaluated at 40° C. and 5° C. After defined storage periods, samples were pulled and the impact of storage time on DVD-Ig protein stability was evaluated. The concentrations of histidine that were evaluated include 0, 5, 15, 50, and 200 mM.

Samples were filled into sterile vials (approx. 500 μl each) and stored under controlled conditions (in temperature chambers and in the absence of light). After 7 days and 21 days, samples of the prepared solutions were analyzed using SEC and IEC.

TABLE 16
Impact Of Storage Of Various DVD-Ig Proteins At Low And High
Concentrations Under Various Histidine Concentrations On SEC

| | | | | | | | | | |
|---|---|---|---|---|---|---|---|---|---|
| DVD-A, pH 5.2, | | | | | DVD-A, pH 5.2, | | | | |
| 1 mg/ml | Agg | Mon | Frag | Precipitation* | 75 mg/ml | Agg | Mon | Frag | Precipitation* |

T0

| | | | | | | | | | |
|---|---|---|---|---|---|---|---|---|---|
| 0 Histidine | 1.89 | 97.21 | 0.88 | N | 0 Histidine | 2.63 | 96.39 | 0.97 | N |
| 5 mM Histidine | 0.93 | 98.19 | 0.86 | N | 5 mM Histidine | 0.52 | 98.27 | 1.19 | N |
| 15 mM Histidine | 1.41 | 97.64 | 0.94 | N | 15 mM Histidine | 1.69 | 97.38 | 0.92 | N |
| 50 mM Histidine | 1.83 | 96.84 | 1.31 | N | 50 mM Histidine | 2.01 | 96.86 | 1.11 | N |
| 200 mM Histidine | 2.09 | 96.96 | 0.94 | N | 200 mM Histidine | 2.27 | 96.78 | 0.94 | N |

40 C., 7 d

| | | | | | | | | | |
|---|---|---|---|---|---|---|---|---|---|
| 0 Histidine | 1.55 | 96.4 | 2.03 | N | 0 Histidine | 10.52 | 87.77 | 1.7 | N |
| 5 mM Histidine | 1.19 | 97.17 | 1.62 | N | 5 mM Histidine | 1.26 | 95.01 | 3.71 | N |
| 15 mM Histidine | 2.47 | 95.49 | 2.03 | N | 15 mM Histidine | 13.76 | 83.35 | 2.88 | N |
| 50 mM Histidine | 2.31 | 95.42 | 2.25 | N | 50 mM Histidine | 16.62 | 80.82 | 2.54 | N |
| 200 mM Histidine | 2.14 | 95.92 | 1.92 | N | 200 mM Histidine | 7.47 | 89.68 | 2.83 | Y |

40 C., 21 d

| | | | | | | | | | |
|---|---|---|---|---|---|---|---|---|---|
| 0 Histidine | | | | | 0 Histidine | 15.77 | 82.2 | 2.02 | Y |
| 5 mM Histidine | 2.52 | 95.3 | 2.16 | N | 5 mM Histidine | 14.07 | 83.8 | 2.12 | N |
| 15 mM Histidine | 3.39 | 93.85 | 2.74 | N | 15 mM Histidine | 18.07 | 79.49 | 2.42 | N |
| 50 mM Histidine | 2.74 | 94.4 | 2.84 | N | 50 mM Histidine | 13.41 | 83.71 | 2.86 | Y |
| 200 mM Histidine | 1.9 | 94.91 | 3.18 | N | 200 mM Histidine | 8.35 | 87.66 | 3.97 | Y |

5 C., 21 d

| 0 Histidine | 1.24 | 97.62 | 1.12 | N | 0 Histidine | 1.78 | 97.16 | 1.04 | N |
|---|---|---|---|---|---|---|---|---|---|
| 5 mM Histidine | 1 | 97.86 | 1.12 | N | 5 mM Histidine | 1.74 | 97.33 | 0.91 | N |
| 15 mM Histidine | 1.08 | 97.43 | 1.47 | N | 15 mM Histidine | 2.01 | 96.77 | 1.21 | N |
| 50 mM Histidine | 1.25 | 97.36 | 1.37 | N | 50 mM Histidine | 2.31 | 96.45 | 1.22 | N |
| 200 mM Histidine | 1.61 | 97.08 | 1.3 | N | 200 mM Histidine | | | | N |
| DVD-C, pH 5.4, | | | | | DVD-C, pH 5.4, | | | | |
| 1 mg/ml | Agg | Mon | Frag | Precipitation* | 100 mg/ml | Agg | Mon | Frag | Precipitation* |

T0

| | | | | | | | | | |
|---|---|---|---|---|---|---|---|---|---|
| 0 Histidine | 2.31 | 96.35 | 1.33 | N | 0 Histidine | 3.05 | 95.18 | 1.75 | N |
| 5 mM Histidine | 1.82 | 96.64 | 1.52 | N | 5 mM Histidine | 2.55 | 95.84 | 1.6 | N |
| 15 mM Histidine | 1.83 | 96.53 | 1.63 | N | 15 mM Histidine | 2.2 | 96.1 | 1.68 | N |
| 50 mM Histidine | 1.87 | 96.67 | 1.44 | N | 50 mM Histidine | 1.8 | 96.54 | 1.64 | N |
| 200 mM Histidine | 2.17 | 96.14 | 1.68 | N | 200 mM Histidine | 1.7 | 96.49 | 1.8 | N |

40 C., 7 d

| | | | | | | | | | |
|---|---|---|---|---|---|---|---|---|---|
| 0 Histidine | 2.41 | 92.11 | 5.47 | N | 0 Histidine | 3.18 | 94.49 | 2.31 | N |
| 5 mM Histidine | 1.62 | 95.93 | 2.43 | N | 5 mM Histidine | 2.65 | 95.2 | 2.13 | N |
| 15 mM Histidine | 1.49 | 95.9 | 2.59 | N | 15 mM Histidine | 2.46 | 94.96 | 2.57 | N |
| 50 mM Histidine | 1.38 | 96.16 | 2.45 | N | 50 mM Histidine | 2.33 | 95.16 | 2.49 | N |
| 200 mM Histidine | 1.47 | 96.12 | 2.39 | N | 200 mM Histidine | 1.82 | 95.19 | 2.97 | N |

40 C., 21 d

| | | | | | | | | | |
|---|---|---|---|---|---|---|---|---|---|
| 0 Histidine | 1.38 | 95.5 | 3.11 | N | 0 Histidine | 3.45 | 93.64 | 2.89 | N |
| 5 mM Histidine | 1.5 | 95.39 | 3.1 | N | 5 mM Histidine | 2.8 | 94.27 | 2.91 | N |
| 15 mM Histidine | 1.26 | 96.01 | 2.71 | N | 15 mM Histidine | 2.55 | 94.56 | 2.87 | N |
| 50 mM Histidine | 1.34 | 95.83 | 2.82 | N | 50 mM Histidine | 2.26 | 94.9 | 2.83 | N |
| 200 mM Histidine | 1.38 | 95.72 | 2.88 | N | 200 mM Histidine | 2.84 | 93.29 | 3.86 | N |

5 C., 21 d

| | | | | | | | | | |
|---|---|---|---|---|---|---|---|---|---|
| 0 Histidine | 2.08 | 96.12 | 1.78 | N | 0 Histidine | 2.77 | 94.77 | 2.44 | N |
| 5 mM Histidine | 1.56 | 96.72 | 1.71 | N | 5 mM Histidine | 2.35 | 95.43 | 2.2 | N |
| 15 mM Histidine | 1.27 | 97.08 | 1.63 | N | 15 mM Histidine | 2.18 | 95.42 | 2.38 | N |
| 50 mM Histidine | 1.4 | 96.67 | 1.91 | N | 50 mM Histidine | 1.95 | 96.07 | 1.96 | N |
| 200 mM Histidine | 1.59 | 96.62 | 1.77 | N | 200 mM Histidine | 1.95 | 96.1 | 1.94 | N |

| | | | | | | | | | |
|---|---|---|---|---|---|---|---|---|---|
| IL12IL18, pH | | | | | IL12IL18, pH 5.4, | | | | |
| 5.4, 1 mg/ml | Agg | Mon | Frag | Precipitation* | 150 mg/ml | Agg | Mon | Frag | Precipitation* |

T0

| | | | | | | | | | |
|---|---|---|---|---|---|---|---|---|---|
| 0 Histidine | 2.95 | 95.23 | 1.8 | N | 0 Histidine | 3.48 | 94.22 | 2.29 | N |
| 5 mM Histidine | 1.92 | 96.28 | 1.79 | N | 5 mM Histidine | 4.64 | 93.26 | 2.09 | N |
| 15 mM Histidine | 2.16 | 95.71 | 2.12 | N | 15 mM Histidine | 4.33 | 93.53 | 2.12 | N |
| 50 mM Histidine | 2.07 | 96 | 1.91 | N | 50 mM Histidine | 3.93 | 94.2 | 1.88 | N |
| 200 mM Histidine | 2.46 | 95.65 | 1.87 | N | 200 mM Histidine | 3.55 | 94.48 | 1.96 | N |

40 C., 7 d

| | | | | | | | | | |
|---|---|---|---|---|---|---|---|---|---|
| 0 Histidine | 2.88 | 93.82 | 3.29 | N | 0 Histidine | 4.71 | 91.46 | 3.82 | N |
| 5 mM Histidine | 1.32 | 95.82 | 2.85 | N | 5 mM Histidine | 5.79 | 90.44 | 3.75 | N |
| 15 mM Histidine | 1.02 | 96.12 | 2.85 | N | 15 mM Histidine | 4.87 | 91.81 | 3.3 | N |
| 50 mM Histidine | 1.57 | 95.23 | 3.19 | N | 50 mM Histidine | 8.58 | 87.7 | 3.71 | N |
| 200 mM Histidine | 1.43 | 95.56 | 3 | N | 200 mM Histidine | 6.78 | 89.81 | 3.4 | N |

40 C., 21 d

| | | | | | | | | | |
|---|---|---|---|---|---|---|---|---|---|
| 0 Histidine | 1.51 | 94.69 | 3.79 | N | 0 Histidine | 5.18 | 91.21 | 3.6 | N |
| 5 mM Histidine | 1.08 | 95.87 | 3.04 | N | 5 mM Histidine | 6.76 | 89.67 | 3.56 | N |
| 15 mM Histidine | 1.07 | 95.85 | 3.06 | N | 15 mM Histidine | 5.7 | 91.19 | 3.09 | N |
| 50 mM Histidine | 1.2 | 95.56 | 3.32 | N | 50 mM Histidine | 12.87 | 83.35 | 3.76 | N |
| 200 mM Histidine | 1.42 | 95.42 | 3.15 | N | 200 mM Histidine | 7.21 | 89.88 | 2.89 | N |

5 C., 21d

| | | | | | | | | | |
|---|---|---|---|---|---|---|---|---|---|
| 0 Histidine | 1.71 | 96.03 | 2.24 | N | 0 Histidine | 5.96 | 91.35 | 2.67 | N |
| 5 mM Histidine | 1.25 | 96.44 | 2.3 | N | 5 mM Histidine | 6.94 | 90.97 | 2.07 | N |
| 15 mM Histidine | 1.4 | 96.13 | 2.46 | N | 15 mM Histidine | 5.69 | 92.19 | 2.1 | N |
| 50 mM Histidine | 1.78 | 96.03 | 2.18 | N | 50 mM Histidine | 10.07 | 88.13 | 1.78 | N |
| 200 mM Histidine | 2.1 | 95.7 | 2.19 | N | 200 mM Histidine | 1.32 | 95.56 | 3.11 | N |

*Precipitation indicates if insoluble visible aggregates were observed (Yes or No)

TABLE 17
Impact of Storage of Various DVD-Ig Proteins at Low And High Concentrations
Under Various Histidine Concentrations on IEC

| | | | | | | | |
|---|---|---|---|---|---|---|---|
| DVD-A, pH 5.2, | | | | DVD-A, pH 5.2, | | | |
| 1 mg/ml | Acidic | Main | Basic | 75 mg/ml | Acidic | Main | Basic |

T0

| | | | | | | | |
|---|---|---|---|---|---|---|---|
| 0 Histidine | 15.66 | 49.3 | 35.02 | 0 Histidine | 19.71 | 50.72 | 29.55 |
| 5 mM Histidine | 13.15 | 50.9 | 35.93 | 5 mM Histidine | 19.11 | 50.8 | 30.08 |
| 15 mM Histidine | 15.11 | 46.74 | 38.13 | 15 mM Histidine | 18.58 | 44.52 | 36.88 |
| 50 mM Histidine | 13.27 | 52.38 | 34.33 | 50 mM Histidine | 15.69 | 52.06 | 32.24 |
| 200 mM Histidine | 14.55 | 52.01 | 33.42 | 200 mM Histidine | 16.79 | 45.36 | 37.84 |

40 C., 7 d

| | | | | | | | |
|---|---|---|---|---|---|---|---|
| 0 Histidine | 24.97 | 40.98 | 34.03 | 0 Histidine | 21.7 | 43.6 | 34.68 |
| 5 mM Histidine | 22.21 | 44.35 | 33.43 | 5 mM Histidine | 28.31 | 43.99 | 27.69 |
| 15 mM Histidine | 19.79 | 44.91 | 35.29 | 15 mM Histidine | 21.21 | 41.34 | 37.44 |
| 50 mM Histidine | 18.19 | 46.11 | 35.68 | 50 mM Histidine | 18.42 | 42.84 | 38.73 |
| 200 mM Histidine | 18.08 | 48.08 | 33.83 | 200 mM Histidine | 16.71 | 42.09 | 41.19 |

5 C., 21 d

| 0 Histidine | 15.66 | 46.47 | 37.86 | 0 Histidine | 16.41 | 48.16 | 35.41 |
|---|---|---|---|---|---|---|---|
| 5 mM Histidine | 18.76 | 49.37 | 31.85 | 5 mM Histidine | 16.19 | 49.44 | 34.36 |
| 15 mM Histidine | 14.34 | 52.8 | 32.85 | 15 mM Histidine | 16.91 | 45.77 | 37.3 |
| 50 mM Histidine | 15.06 | 50.33 | 34.59 | 50 mM Histidine | 17.17 | 47.45 | 35.37 |
| 200 mM Histidine | 14.49 | 48.8 | 36.7 | 200 mM Histidine | 15.77 | 46.18 | 38.03 |
| DVD-C, pH 5.4, | | | | DVD-C, pH 5.4, | | | |
| 1 mg/ml | | | | 100 mg/ml | | | |

T0

| | | | | | | | |
|---|---|---|---|---|---|---|---|
| 0 Histidine | 26.28 | 59.4 | 14.3 | 0 Histidine | 24.53 | 66.44 | 9.01 |
| 5 mM Histidine | 23.01 | 69.42 | 7.56 | 5 mM Histidine | 24.17 | 65.96 | 9.86 |
| 15 mM Histidine | 22.05 | 67.79 | 10.14 | 15 mM Histidine | 23.67 | 67.77 | 8.55 |
| 50 mM Histidine | 21.9 | 69.09 | 9 | 50 mM Histidine | 22.71 | 67.65 | 9.63 |
| 200 mM Histidine | 20.99 | 70.74 | 8.26 | 200 mM Histidine | | | |

40 C., 7 d

| | | | | | | | |
|---|---|---|---|---|---|---|---|
| 0 Histidine | 34.05 | 55.74 | 10.2 | 0 Histidine | 28.68 | 61.69 | 9.61 |
| 5 mM Histidine | 31.26 | 59.07 | 9.65 | 5 mM Histidine | 29.05 | 61.57 | 9.37 |
| 15 mM Histidine | 27.8 | 61.86 | 10.33 | 15 mM Histidine | 29.2 | 61.25 | 9.54 |
| 50 mM Histidine | 25.97 | 59.33 | 14.49 | 50 mM Histidine | 27.43 | 61.86 | 10.69 |
| 200 mM Histidine | 26.35 | 62.28 | 11.36 | 200 mM Histidine | 28.04 | 59.58 | 12.37 |

5 C., 21 d

| 0 Histidine | 25.05 | 65.7 | 9.23 | 0 Histidine | 23.43 | 66.85 | 9.71 |
|---|---|---|---|---|---|---|---|
| 5 mM Histidine | 23.66 | 66.17 | 10.15 | 5 mM Histidine | 22.3 | 68.58 | 9.11 |
| 15 mM Histidine | 22.11 | 69.96 | 8.91 | 15 mM Histidine | 23.39 | 67.31 | 9.29 |
| 50 mM Histidine | 21.79 | 68.96 | 9.23 | 50 mM Histidine | 23.67 | 67.96 | 8.35 |
| 200 mM Histidine | 21.96 | 70.23 | 7.8 | 200 mM Histidine | 22.6 | 69.68 | 7.65 |
| IL12IL18, pH 5.4, | | | | IL12IL18, pH 5.4, | | | |
| 1 mg/ml | | | | 150 mg/ml | | | |

T0

| | | | | | | | |
|---|---|---|---|---|---|---|---|
| 0 Histidine | 30.24 | 51.83 | 17.92 | 0 Histidine | 31.05 | 50.74 | 18.2 |
| 5 mM Histidine | 29.11 | 54.71 | 16.16 | 5 mM Histidine | 30.3 | 53.53 | 16.16 |
| 15 mM Histidine | 28.36 | 57.23 | 14.39 | 15 mM Histidine | 30.02 | 53.9 | 16.07 |
| 50 mM Histidine | 29.15 | 53.29 | 17.55 | 50 mM Histidine | 28.42 | 51.31 | 20.26 |
| 200 mM Histidine | 35.59 | 52.31 | 12.09 | 200 mM Histidine | 28.85 | 55.24 | 15.9 |

40 C., 7 d

| | | | | | | | |
|---|---|---|---|---|---|---|---|
| 0 Histidine | 37.24 | 45.3 | 17.45 | 0 Histidine | 34.95 | 44.85 | 20.19 |
| 5 mM Histidine | 36 | 47.57 | 16.42 | 5 mM Histidine | 31.94 | 45.7 | 22.34 |
| 15 mM Histidine | 36.17 | 50.01 | 13.81 | 15 mM Histidine | 32.39 | 45.54 | 22.05 |
| 50 mM Histidine | 35.39 | 49.12 | 15.47 | 50 mM Histidine | 37 | 41.76 | 21.23 |
| 200 mM Histidine | 38.48 | 45.03 | 16.47 | 200 mM Histidine | 30.86 | 47.34 | 21.78 |

5 C., 21 d

| | | | | | | | |
|---|---|---|---|---|---|---|---|
| 0 Histidine | 30.72 | 51.83 | 17.44 | 0 Histidine | 30.91 | 50.49 | 18.59 |
| 5 mM Histidine | 30.25 | 51 | 18.74 | 5 mM Histidine | 29.14 | 50.47 | 20.37 |
| 15 mM Histidine | 30.07 | 56.33 | 13.58 | 15 mM Histidine | 28.45 | 50.95 | 20.58 |
| 50 mM Histidine | 30.51 | 55.5 | 13.97 | 50 mM Histidine | 27.91 | 52.66 | 19.42 |
| 200 mM Histidine | 42.9 | 49 | 8.08 | 200 mM Histidine | 27.96 | 48.84 | 22.19 |

Tables 16 and 17 show that the amount of monomer remaining at different time points and at the formation of insoluble aggregates indicates that histidine concentrations in the range of 5-50 mM provided optimum stability. A concentration of 200 mM histidine resulted in the formation of insoluble aggregates in some cases (see indication of precipitation in Table 16). 0 mM histidine formulations showed enhanced aggregation as indicated by formation of insoluble and soluble aggregates in some cases (in some cases soluble aggregates were higher at 0 than that at 5 mM histidine concentrations). Secondly, the pH is expected to be well maintained for longer storage times in formulations containing histidine.

The error in IEC measurements is usually higher (2-3% variation) compared to SEC measurements with the same formulation. Hence taking that into account, no significant differences were observed within formulations as assayed by IEC.

### Example 8: Example of the Stability of an AS-DVD-Ig Protein (DVD-C, Anti-IL1 Alpha/Beta) in Solution

An anti-IL1 alpha/beta DVD-IgG protein (DVD C) was assessed for stability over time at both 100 mg/ml and 1 mg/ml in different buffers, at different pHs and at different temperatures. Buffers that were tested at 100 mg/ml of DVD C included 15 mM acetate pH 4; 15 mM acetate pH 5; 15 mM histidine pH 5.5; 15 mM succinate pH 5.5; 15 mM histidine pH 6.0; Water (no buffer) pH 6.0; 15 mM citrate pH 6.0; 15 mM histidine pH 6.5; and 15 mM Tris pH 8.0. Buffers that were tested at 1 mg/ml of DVD C included 10 mM citrate+10 mM phosphate buffer at pH 3, 4, 5, 6, 7, and 8.

The samples were stored at 50° C., 40° C., 25° C., and 5° C. At certain time points, samples were pulled and evaluated for stability. Physical stability was evaluated by size exclusion chromatography (SE-HPLC or SEC), including % aggregrate, % monomer, % fragment, and total species recovered were quantitated. Chemical stability was evaluated by weak cation exchange chromatography (IEX-HPLC or IEC), including % acidic, % main, and % basic species quantitated.

Tables 18 and 19 describe stability of DVD-C at 100 mg/ml and 1 mg/ml, respectively, in various buffers and pHs. In both Tables 18 and 19, size exclusion chromatography (SEC) data and ion-exchange chromatography (IEC) data is displayed. Formulation and abbreviation keys are given below each table.

TABLE 18
Stability at Various Temperatures of DVD-C at 100 mg/ml in Different Buffers
and at Different pHs.

| | | | | SEC data | | | | IEX data | | |
|---|---|---|---|---|---|---|---|---|---|---|
| | Temp | | | % | % | % | | % | % | % |
| Time | (° C.) | Formulation | pH | HMW | M | LMW | AUC | Acidic | Main | Basic |
| T0 | — | ace | 4 | 0.88 | 98.03 | 1.09 | 45941 | 19.65 | 71.68 | 8.67 |
| T0 | — | ace | 5 | 0.94 | 98.19 | 0.87 | 45789 | 19.79 | 71.60 | 8.61 |
| T0 | — | his | 5.5 | 0.98 | 98.15 | 0.86 | 48085 | 20.40 | 71.90 | 7.70 |
| T0 | — | succ | 5.5 | 1.04 | 97.96 | 1.00 | 49317 | 19.70 | 71.54 | 8.76 |
| T0 | — | his | 6 | 1.15 | 97.93 | 0.92 | 48468 | 20.68 | 70.94 | 8.38 |
| T0 | — | water | 6 | 1.87 | 97.27 | 0.86 | 48356 | 18.95 | 72.35 | 8.70 |
| T0 | — | citrate | 6 | 1.38 | 97.54 | 1.09 | 44457 | 19.35 | 72.37 | 8.28 |
| T0 | — | his | 6.5 | 1.16 | 97.93 | 0.91 | 47102 | 21.17 | 70.45 | 8.38 |
| T0 | — | tris | 8 | 2.40 | 96.65 | 0.95 | 53889 | 21.71 | 69.12 | 9.17 |
| T7 d | 40 | ace | 4 | 0.92 | 97.03 | 2.05 | 49124 | 19.16 | 71.61 | 9.23 |
| T7 d | 40 | ace | 5 | 1.12 | 97.31 | 1.57 | 49077 | 21.62 | 71.09 | 7.29 |
| T7 d | 40 | his | 5.5 | 1.17 | 97.27 | 1.56 | 49662 | 19.48 | 73.60 | 6.92 |
| T7 d | 40 | succ | 5.5 | 1.46 | 97.06 | 1.48 | 48821 | 23.18 | 69.51 | 7.31 |
| T7 d | 40 | his | 6 | 1.72 | 96.96 | 1.32 | 42872 | 20.99 | 73.36 | 5.64 |
| T7 d | 40 | water | 6 | 2.48 | 96.22 | 1.30 | 37817 | 21.67 | 72.02 | 6.31 |
| T7 d | 40 | citrate | 6 | 1.79 | 96.83 | 1.37 | 43022 | 21.70 | 71.52 | 6.78 |
| T7 d | 40 | his | 6.5 | 2.08 | 96.65 | 1.27 | 48176 | 23.68 | 70.89 | 5.43 |
| T7 d | 40 | tris | 8 | 4.58 | 93.87 | 1.55 | 48306 | 40.86 | 52.70 | 6.44 |
| T1 mo | 40 | ace | 4 | 1.32 | 94.29 | 4.38 | 52518 | 34.78 | 32.07 | 33.16 |
| T1 mo | 40 | ace | 5 | 1.64 | 95.36 | 3.00 | 53762 | 36.48 | 52.11 | 11.41 |
| T1 mo | 40 | his | 5.5 | 1.61 | 95.38 | 3.01 | 52489 | 33.12 | 55.52 | 11.36 |
| T1 mo | 40 | succ | 5.5 | 2.25 | 94.73 | 3.01 | 51508 | 40.94 | 46.88 | 12.19 |
| T1 mo | 40 | his | 6 | 2.81 | 94.53 | 2.66 | 52229 | 34.46 | 56.00 | 9.54 |
| T1 mo | 40 | water | 6 | 3.66 | 93.75 | 2.58 | 52285 | 33.46 | 56.53 | 10.01 |
| T1 mo | 40 | citrate | 6 | 2.67 | 94.91 | 2.42 | 51968 | 36.54 | 52.80 | 10.67 |
| T1 mo | 40 | his | 6.5 | 3.61 | 93.69 | 2.71 | 50276 | 39.06 | 51.20 | 9.74 |
| T1 mo | 40 | tris | 8 | 7.49 | 85.24 | 7.28 | 53081 | 52.90 | 15.51 | 31.58 |
| T1 mo | 25 | ace | 4 | 0.98 | 97.30 | 1.72 | 56026 | 23.39 | 59.02 | 17.59 |
| T1 mo | 25 | ace | 5 | 1.15 | 97.31 | 1.54 | 55264 | 22.44 | 69.01 | 8.55 |
| T1 mo | 25 | his | 5.5 | 1.16 | 97.41 | 1.43 | 54356 | 22.05 | 69.43 | 8.51 |
| T1 mo | 25 | succ | 5.5 | 1.42 | 97.07 | 1.51 | 52417 | 23.00 | 67.63 | 9.37 |
| T1 mo | 25 | his | 6 | 1.75 | 96.74 | 1.51 | 52220 | 23.80 | 67.67 | 8.52 |
| T1 mo | 25 | water | 6 | 2.66 | 95.94 | 1.40 | 51198 | 23.68 | 67.35 | 8.98 |
| T1 mo | 25 | citrate | 6 | 1.91 | 96.59 | 1.49 | 51137 | 22.41 | 68.14 | 9.45 |
| T1 mo | 25 | his | 6.5 | 1.93 | 96.58 | 1.49 | 50594 | 25.93 | 65.50 | 8.56 |
| T1 mo | 25 | tris | 8 | 4.35 | 93.87 | 1.78 | 51644 | 39.99 | 50.14 | 9.87 |
| T3 mo | 40 | ace | 4 | 2.19 | 85.66 | 12.15 | 54546 | 45.24 | 29.61 | 25.15 |
| T3 mo | 40 | ace | 5 | 2.69 | 89.70 | 7.61 | 63493 | 52.09 | 31.52 | 16.39 |
| T3 mo | 40 | his | 5.5 | 2.57 | 89.79 | 7.64 | 64361 | 48.68 | 35.18 | 16.14 |
| T3 mo | 40 | succ | 5.5 | 3.53 | 88.89 | 7.58 | 57855 | 60.00 | 24.73 | 15.28 |
| T3 mo | 40 | his | 6 | 4.08 | 90.70 | 5.21 | 60062 | 50.85 | 37.10 | 12.04 |
| T3 mo | 40 | water | 6 | 4.80 | 90.50 | 4.70 | 58908 | 47.55 | 43.39 | 9.06 |
| T3 mo | 40 | citrate | 6 | 3.87 | 91.38 | 4.75 | 55505 | 56.39 | 30.85 | 12.76 |
| T3 mo | 40 | his | 6.5 | 5.56 | 89.08 | 5.37 | 56243 | 58.77 | 32.28 | 8.95 |
| T3 mo | 40 | tris | 8 | 14.20 | 75.16 | 10.64 | 59362 | 63.76 | 22.12 | 14.12 |
| T3 mo | 25 | ace | 4 | 1.15 | 96.39 | 2.46 | 60821 | 25.24 | 61.82 | 12.94 |
| T3 mo | 25 | ace | 5 | 1.29 | 96.85 | 1.86 | 53513 | 23.62 | 66.47 | 9.91 |
| T3 mo | 25 | his | 5.5 | 1.34 | 96.84 | 1.82 | 56041 | 23.05 | 67.70 | 9.25 |
| T3 mo | 25 | succ | 5.5 | 1.69 | 96.36 | 1.95 | 53657 | 27.27 | 61.25 | 11.48 |
| T3 mo | 25 | his | 6 | 2.04 | 96.23 | 1.73 | 51684 | 26.17 | 63.16 | 10.67 |
| T3 mo | 25 | water | 6 | 2.81 | 95.49 | 1.70 | 52442 | 25.01 | 64.07 | 10.91 |
| T3 mo | 25 | citrate | 6 | 2.08 | 96.11 | 1.80 | 51993 | 24.48 | 64.34 | 11.18 |
| T3 mo | 25 | his | 6.5 | 2.26 | 96.01 | 1.72 | 50988 | 30.52 | 59.06 | 10.42 |
| T3 mo | 25 | tris | 8 | 5.61 | 91.87 | 2.51 | 52070 | 59.46 | 31.54 | 8.99 |
| T3 mo | 5 | ace | 4 | 0.85 | 98.01 | 1.14 | 50571 | 18.16 | 71.76 | 10.09 |
| T3 mo | 5 | ace | 5 | 1.06 | 97.91 | 1.04 | 49173 | 18.44 | 71.54 | 10.02 |
| T3 mo | 5 | his | 5.5 | 1.33 | 97.65 | 1.02 | 51947 | 19.97 | 70.28 | 9.75 |
| T3 mo | 5 | succ | 5.5 | 1.32 | 97.59 | 1.09 | 50358 | 19.12 | 70.88 | 10.00 |
| T3 mo | 5 | his | 6 | 2.18 | 96.79 | 1.03 | 49875 | 22.34 | 67.47 | 10.19 |
| T3 mo | 5 | water | 6 | 8.21 | 90.70 | 1.09 | 48448 | 18.89 | 67.56 | 13.55 |
| T3 mo | 5 | citrate | 6 | 2.20 | 96.65 | 1.15 | 48907 | 19.08 | 70.77 | 10.16 |
| T3 mo | 5 | his | 6.5 | 1.86 | 97.12 | 1.02 | 47895 | 22.91 | 67.10 | 9.99 |
| T3 mo | 5 | tris | 8 | 5.93 | 93.02 | 1.05 | 49015 | 24.67 | 63.29 | 12.04 |

Formulation and abbreviation key:
ace = 15 mM acetate
his = 15 mM histidine
succ = 15 mM succinate
water = formulated in water by dialysis
citrate = 15 mM citrate
tris = 15 mM TRIS
% HMW = percentage of high molecule weight species quantitated by SEC
% M = percentage of monomer quantitated by SEC
% LMW = percentage of low molecule weight species quantitated by SEC
AUC = total integrated area of the SEC curve
% acidic = percentage of acidic species relative to the main species quantitated by IEC
% main = percentage of main species quantitated by IEC
% basic = percentage of basic species relative to the main species quantitated by IEX
T0 = time zero
T7 d = 7 days of storage
T1 mo = 1 month of storage
T3 mo = 3 months of storage

TABLE 19
Stability at Various Temperatures of DVD-C at 1.0 mg/ml in 10 mM Citrate +
10 mM Phosphate Buffer and at Different pHs.
NR = assay not performed

| | | | SEC data | | | | IEX data | | |
|---|---|---|---|---|---|---|---|---|---|
| | Temp | | % | % | % | | % | % | % |
| Time | (° C.) | pH | HMW | M | LMW | AUC | Acidic | Main | Basic |
| 0 | — | 3 | ND | ND | ND | ND | ND | ND | ND |
| 0 | — | 4 | 0.78 | 97.97 | 1.25 | 106805 | 10.14 | 73.38 | 16.48 |
| 0 | — | 5 | 1.07 | 97.71 | 1.21 | 104165 | 9.80 | 73.84 | 16.35 |
| 0 | — | 6 | 1.23 | 97.62 | 1.15 | 99838 | 10.80 | 73.52 | 15.69 |
| 0 | — | 7 | 1.37 | 97.43 | 1.21 | 98566 | 10.45 | 73.79 | 15.76 |
| 0 | — | 8 | 1.48 | 97.30 | 1.22 | 93914 | 10.33 | 74.41 | 15.26 |
| 7 d | 40 | 3 | ND | ND | ND | ND | ND | ND | ND |
| 7 d | 40 | 4 | 0.70 | 94.51 | 4.79 | 99177 | 38.59 | 41.06 | 20.35 |
| 7 d | 40 | 5 | 1.09 | 97.43 | 1.48 | 105735 | 24.92 | 57.07 | 18.01 |
| 7 d | 40 | 6 | 1.30 | 97.16 | 1.54 | 101459 | 21.48 | 62.95 | 15.57 |
| 7 d | 40 | 7 | 1.53 | 96.38 | 2.09 | 94903 | 16.60 | 69.48 | 13.93 |
| 7 d | 40 | 8 | 2.00 | 95.74 | 2.25 | 95090 | 50.88 | 36.05 | 13.07 |
| 7 d | 50 | 3 | ND | ND | ND | ND | ND | ND | ND |
| 7 d | 50 | 4 | ND | ND | ND | ND | ND | ND | ND |
| 7 d | 50 | 5 | 2.72 | 93.91 | 3.37 | 102195 | 49.52 | 32.87 | 17.61 |
| 7 d | 50 | 6 | 2.23 | 95.55 | 2.22 | 99767 | 37.88 | 47.12 | 15.00 |
| 7 d | 50 | 7 | 1.67 | 94.59 | 3.74 | 96751 | 54.67 | 32.09 | 13.24 |
| 7 d | 50 | 8 | 1.82 | 94.13 | 4.05 | 85005 | 61.11 | 13.48 | 25.41 |
| 21 d | 5 | 3 | ND | ND | ND | ND | ND | ND | ND |
| 21 d | 5 | 4 | 0.01 | 96.54 | 3.45 | 105701 | 11.64 | 73.47 | 14.89 |
| 21 d | 5 | 5 | 0.00 | 96.29 | 3.71 | 102947 | 11.40 | 73.19 | 15.41 |
| 21 d | 5 | 6 | 0.01 | 96.33 | 3.66 | 98166 | 11.56 | 73.70 | 14.74 |
| 21 d | 5 | 7 | 0.01 | 96.24 | 3.75 | 96077 | 17.02 | 67.39 | 15.60 |
| 21 d | 5 | 8 | 0.01 | 95.63 | 4.36 | 91507 | 17.87 | 68.99 | 13.15 |
| 21 d | 25 | 3 | ND | ND | ND | ND | ND | ND | ND |
| 21 d | 25 | 4 | 0.08 | 95.89 | 4.02 | 107054 | 32.05 | 49.45 | 18.51 |
| 21 d | 25 | 5 | 0.07 | 96.85 | 3.08 | 104609 | 20.27 | 62.85 | 16.88 |
| 21 d | 25 | 6 | 0.05 | 96.86 | 3.09 | 100308 | 18.61 | 66.21 | 15.18 |
| 21 d | 25 | 7 | 0.08 | 96.40 | 3.52 | 97790 | 18.83 | 67.03 | 14.14 |
| 21 d | 25 | 8 | 0.07 | 95.82 | 4.12 | 92666 | 41.03 | 45.81 | 13.16 |
| 21 d | 40 | 3 | ND | ND | ND | ND | ND | ND | ND |
| 21 d | 40 | 4 | 0.09 | 88.58 | 11.33 | 79690 | 61.27 | 18.98 | 19.74 |
| 21 d | 40 | 5 | 0.20 | 95.13 | 4.67 | 105902 | 46.17 | 37.31 | 16.52 |
| 21 d | 40 | 6 | 0.23 | 95.96 | 3.81 | 101455 | 31.13 | 54.02 | 14.85 |
| 21 d | 40 | 7 | 0.33 | 94.32 | 5.35 | 98972 | 53.39 | 34.34 | 12.27 |
| 21 d | 40 | 8 | 0.37 | 93.05 | 6.57 | 91576 | 59.60 | 15.00 | 25.40 |
| 21 d | 50 | 3 | ND | ND | ND | ND | ND | ND | ND |
| 21 d | 50 | 4 | ND | ND | ND | ND | ND | ND | ND |
| 21 d | 50 | 5 | 0.20 | 91.14 | 8.66 | 90993 | 56.65 | 10.23 | 33.13 |
| 21 d | 50 | 6 | 0.34 | 93.57 | 6.09 | 100229 | 66.40 | 21.90 | 11.70 |
| 21 d | 50 | 7 | 0.54 | 89.92 | 9.54 | 91695 | 50.18 | 26.86 | 22.97 |
| 21 d | 50 | 8 | 0.47 | 87.11 | 12.41 | 67323 | 47.09 | 17.82 | 35.09 |
| 3 mo | 40 | 3 | ND | ND | ND | ND | ND | ND | ND |
| 3 mo | 40 | 4 | 27.59 | 40.51 | 31.91 | 61759 | NR | NR | NR |
| 3 mo | 40 | 5 | 5.12 | 85.68 | 9.19 | 119476 | NR | NR | NR |
| 3 mo | 40 | 6 | 2.79 | 91.55 | 5.67 | 113431 | NR | NR | NR |
| 3 mo | 40 | 7 | 3.40 | 88.33 | 8.27 | 106409 | NR | NR | NR |
| 3 mo | 40 | 8 | 5.41 | 80.86 | 13.73 | 98142 | NR | NR | NR |
| 3 mo | 25 | 3 | ND | ND | ND | ND | ND | ND | ND |
| 3 mo | 25 | 4 | 1.10 | 91.64 | 7.26 | 109681 | 53.69 | 23.99 | 22.32 |
| 3 mo | 25 | 5 | 1.24 | 96.19 | 2.57 | 106230 | 37.33 | 41.02 | 21.65 |
| 3 mo | 25 | 6 | 1.56 | 96.65 | 1.79 | 102674 | 27.46 | 53.25 | 19.29 |
| 3 mo | 25 | 7 | 1.85 | 95.59 | 2.56 | 100526 | 17.69 | 65.85 | 16.46 |
| 3 mo | 25 | 8 | 2.37 | 94.80 | 2.83 | 95500 | 65.32 | 20.08 | 14.59 |
| 3 mo | 5 | 3 | ND | ND | ND | ND | ND | ND | ND |
| 3 mo | 5 | 4 | 0.83 | 97.90 | 1.28 | 104383 | 18.59 | 60.63 | 20.78 |
| 3 mo | 5 | 5 | 1.14 | 97.86 | 1.00 | 101554 | 16.91 | 58.78 | 24.30 |
| 3 mo | 5 | 6 | 1.31 | 97.75 | 0.95 | 97897 | 17.38 | 63.16 | 19.46 |
| 3 mo | 5 | 7 | 1.54 | 97.43 | 1.03 | 96067 | 18.48 | 63.33 | 18.19 |
| 3 mo | 5 | 8 | 1.67 | 97.27 | 1.06 | 92532 | 20.44 | 61.36 | 18.21 |
| 6.5 mo | 25 | 3 | ND | ND | ND | ND | ND | ND | ND |
| 6.5 mo | 25 | 4 | 1.67 | 85.69 | 12.64 | 7753 | 70.60 | 10.11 | 19.28 |
| 6.5 mo | 25 | 5 | 1.35 | 94.61 | 4.04 | 7865 | 50.36 | 28.97 | 20.67 |
| 6.5 mo | 25 | 6 | 1.69 | 95.80 | 2.51 | 7719 | 36.64 | 46.73 | 16.63 |
| 6.5 mo | 25 | 7 | 2.12 | 94.15 | 3.74 | 7523 | 55.68 | 28.89 | 15.44 |
| 6.5 mo | 25 | 8 | 2.71 | 93.02 | 4.26 | 7155 | 68.76 | 16.22 | 15.01 |
| 6.5 mo | 5 | 3 | ND | ND | ND | ND | ND | ND | ND |
| 6.5 mo | 5 | 4 | 0.76 | 97.78 | 1.46 | 7508 | 19.30 | 59.53 | 21.17 |
| 6.5 mo | 5 | 5 | 1.07 | 97.92 | 1.00 | 7315 | 17.96 | 60.34 | 21.70 |
| 6.5 mo | 5 | 6 | 1.38 | 97.75 | 0.87 | 6969 | 19.99 | 60.32 | 19.69 |
| 6.5 mo | 5 | 7 | 1.61 | 97.38 | 1.02 | 6859 | 19.46 | 62.30 | 18.24 |
| 6.5 mo | 5 | 8 | 1.71 | 97.17 | 1.11 | 6501 | 21.06 | 59.64 | 19.30 |

Abbreviation key:
% HMW = percentage of high molecule weight species quantitated by SEC
% M = percentage of monomer quantitated by SEC
% LMW = percentage of low molecule weight species quantitated by SEC
AUC = total integrated area of the SEC curve
% acidic = percentage of acidic species relative to the main species quantitated by IEX
% main = percentage of main species quantitated by IEX
% basic = percentage of basic species relative to the main species quantitated by IEX
ND = no species detected
NR = assay not performed
0 = time zero
7 d = 7 days of storage
21 d = 1 month of storage
3 mo = 3 months of storage
6.5 mo = 6.5 months of storage

The molecule completely degraded during dialysis at pH 3. No species were detected by SEC or IEC after dialysis at this condition at time zero. Also, storage at 50° C. at pH 4 yielded the same result. Both results are indicated by ND.

In addition, the thermal stability of DVD-C was assessed by differential scanning calorimetry (DSC) (see Table 20). The thermal stability was evaluated at 1.0 mg/ml of the molecule formulated in 10 mM citrate+10 mM phosphate buffer at pHs 4, 5, 6, 7, or 8. A higher onset temperature of unfolding or higher domain midpoint temperature of unfolding means greater thermal stability. The thermal stability at different pHs is often correlated with the long-term stability of the molecule formulated at those pHs. Therefore, the DSC data can help identify the pHs at which the molecule is most and least stable.

TABLE 20
Differential Scanning Calorimetry data of DVD-C at 1.0 mg/ml in 10 mM
Citrate + 10 mM Phosphate Buffer and at Different pHs*

| | | | | Tm3 | |
|---|---|---|---|---|---|
| pH | Onset (° C.) | Tm1 (° C.) | Tm2 (° C.) | (° C.) | Tm4 (° C.) |
| 4 | 49.2 | 64.4 | 67.3 | 74.2 | 78.8 |
| 5 | 55.0 | 68.1 | 69.9 | 76.2 | 82.0 |
| 6 | 54.8 | 67.5 | 69.4 | 75.7 | 83.1 |
| 7 | 55.0 | 67.2 | 69.0 | 74.4 | 82.7 |
| 8 | 54.4 | 67.1 | 68.8 | 73.8 | 82.5 |

*Numbered Tm values indicate the midpoint of the unfolding transitions.

As described above, the stability of DVD-C was tested in a number of different buffers and pHs, when stored at three different temperatures (40 C, 25 C, 5 C). The concentration of DVD-C ranged from 1 mg/ml to 100 mg/ml. At 100 mg/ml, buffers included acetate, histidine, succinate, citrate, and Tris. DVD C was also formulated in plain water. The pH of the formulations ranged from 4 to 8. At 1 mg/ml, the protein was formulated in citrate-phosphate buffer with the pH ranging from 3 to 8. Once formulated, the samples of the DVD-IgG proteins were stored at the aforementioned temperatures. At specific time points, aliquots were taken and assessed for physical stability by SEC and chemical stability by weak cation exchange (WCX).

Overall, the data indicated DVD-C is stable except at pHs 3 and 4. Specifically, the data suggest that physical stability of DVD C is greatest at a pH near 5.5 and that chemical stability is highest at a pH near 6.0. Histidine and succinate were determined to be appropriate buffers for these pHs.

In addition, the thermal stability of DVD-C was assessed by differential scanning calorimetry (DSC), as described in Table 20. The thermal stability was evaluated at 1.0 mg/ml of DVD-C formulated in citrate-phosphate buffer at pHs 4 to 8. A higher onset temperature of unfolding (Ton) or higher domain midpoint temperature of unfolding (Tm) means greater thermal stability. Thermal stability is likely correlated with the long-term stability of the DVD-IgG protein. The data indicate similar thermal stability in the pH range of 5 to 8.

### Example 9: Effect of a Polyol on the Stability of AS-DVD-Ig Proteins

Dynamic scanning fluorescence (DSF) was employed to assess the propensity of a protein to unfold. The impact of polyols, e.g., sucrose and sorbitol, was investigated in order to assess the effect of these polyols on the stability of the protein in solution. DVD-A, DVD-C and an IL12/IL18 DVD were used as exemplary AS-DVD-Ig proteins. As shown in Table 21, in general, AS-DVD-Ig proteins are stable with the presence of sucrose (e.g., 40-160 mg/ml) and sorbitol (e.g., 20-80 mg/ml). Moreover, an increase in the concentration of sorbitol and sucrose provided resulted in a slight increased stability (see Table 21). Hence addition of sugars to a buffer (e.g., histidine) containing protein formulation is favored.

TABLE 21
Impact Of Polyols On The Thermosynamic Stability Of
Various AS-DVD-Ig Proteins
As Assessed By Dynamic Scanning Fluorescence At pH 6

| | Onset of |
|---|---|
| IL12IL18 | Unfolding |
| DVD-Ig Protein Conc. | (° C.) |
| 1 mg/ml, No Sorbitol | 62.8 |
| 1 mg/ml, 20 mg/ml Sorbitol | 63.2 |
| 1 mg/ml, 40 mg/ml Sorbitol | 63.1 |
| 1 mg/ml, 80 mg/ml Sorbitol | 63.5 |
| 150 mg/ml, No Sorbitol | 57.5 |
| 150 mg/ml, 20 mg/ml Sorbitol | 57.6 |
| 150 mg/ml, 40 mg/ml Sorbitol | 57.6 |
| 150 mg/ml, 80 mg/ml Sorbitol | 58.5 |
| 1 mg/ml, No Sucrose | 62.8 |
| 1 mg/ml, 40 mg/ml Sucrose | 63.1 |
| 1 mg/ml, 80 mg/ml Sucrose | 64.1 |
| 1 mg/ml, 160 mg/ml Sucrose | 64.3 |
| 150 mg/ml, No Sucrose | 57.5 |
| 150 mg/ml, 40 mg/ml Sucrose | 58.2 |
| 150 mg/ml, 80 mg/ml Sucrose | 58.3 |
| 150 mg/ml, 160 mg/ml Sucrose | 58.3 |
| DVD-C | |
| 1 mg/ml, No Sorbitol | 62 |
| 1 mg/ml, 20 mg/ml Sorbitol | 62.3 |
| 1 mg/ml, 40 mg/ml Sorbitol | 62 |
| 1 mg/ml, 80 mg/ml Sorbitol | 62.1 |
| 100 mg/ml, No Sorbitol | 47 |
| 100 mg/ml, 20 mg/ml Sorbitol | 47.5 |
| 100 mg/ml, 40 mg/ml Sorbitol | 47 |
| 100 mg/ml, 80 mg/ml Sorbitol | 48.1 |
| 1 mg/ml, No Sucrose | 62 |
| 1 mg/ml, 40 mg/ml Sucrose | 62.1 |
| 1 mg/ml, 80 mg/ml Sucrose | 62 |
| 1 mg/ml, 160 mg/ml Sucrose | 62.2 |
| 100 mg/ml, No Sucrose | 47 |
| 100 mg/ml, 40 mg/ml Sucrose | 46.9 |
| 100 mg/ml, 80 mg/ml Sucrose | 48.1 |
| 100 mg/ml, 160 mg/ml Sucrose | 48 |
| DVD-A | |
| 1 mg/ml, No Sorbitol | 55.8 |
| 1 mg/ml, 20 mg/ml Sorbitol | 56 |
| 1 mg/ml, 40 mg/ml Sorbitol | 56 |
| 1 mg/ml, 80 mg/ml Sorbitol | 56.1 |
| 75 mg/ml, No Sorbitol | 49.5 |
| 75 mg/ml, 20 mg/ml Sorbitol | 50 |
| 75 mg/ml, 40 mg/ml Sorbitol | 50.5 |
| 75 mg/ml, 80 mg/ml Sorbitol | 50.2 |
| 1 mg/ml, No Sucrose | 55.8 |
| 1 mg/ml, 40 mg/ml Sucrose | 56 |
| 1 mg/ml, 80 mg/ml Sucrose | 56.4 |
| 1 mg/ml, 160 mg/ml Sucrose | 56.1 |
| 75 mg/ml, No Sucrose | 49.5 |
| 75 mg/ml, 40 mg/ml Sucrose | 49.5 |
| 75 mg/ml, 80 mg/ml Sucrose | 50 |
| 75 mg/ml, 160 mg/ml Sucrose | 51.1 |

As evidenced by the above data in Table 21, polyols (e.g., sorbitaol and sucrose) improved stability of AS-DVD-Ig proteins in solution in a broad range (20 to 160 mg/ml sucrose and 20 to 80/mg/ml sorbitol). Amounts less than 20 mg/ml provided a stabilizing benefit (Table 21).

### V. AS-DVD-Ig Proteins are Stable in Formulations Containing a Buffer and a Polyol at pH Range of 4.5-7.5

### Example 10: Effect of a Buffer on the Storage Stability of DVD-A in a Formulation Containing a Polyol

To assess the effect of a buffer on the storage stability of DVD-A in different buffers, the stability of the formulations was assessed before storage (T0) or after 7 days (7 d) or 21 days (21 d) of storage at 40° C. (accelerated storage). DVD A (85 mg/ml), an AS-DVD-Ig protein, was formulated in various buffers (15 mM citrate, 15 mM histidine, 15 mM arginine, 15 mM acetate, or water) at a pH of 5.2. Samples were filled into sterile vials (approx. 500 μl each) and stored under controlled conditions in temperature chambers and in the absence of light. The samples were analyzed using SEC and the results are provided in Tables 22 and 23.

TABLE 22
Effect Of Buffer Type on Storage Stability
of DVD-A as Measured by SEC

| Formulation | Time Point | Monomer | Aggregates | CFragments |
|---|---|---|---|---|
| 15 mM acetate, | T0 | 95.96 | 2.75 | 1.28 |
| 80 mg/ml | | | | |
| sucrose, pH 4.0 | | | | |
| | 7 d, 40 C. | 71.84 | 24.93 | 3.21 |
| | 21 d, 40 C. | 66.62 | 26.91 | 6.45 |
| 15 mM acetate, | T0 | 96.06 | 2.78 | 1.14 |
| 80 mg/ml | | | | |
| sucrose, pH 5.2 | | | | |
| | 7 d, 40 C. | 89.19 | 8.76 | 2.03 |
| | 21 d, 40 C. | 90.93 | 4.41 | 4.65 |
| 15 mM arginine, | T0 | 95.84 | 3.07 | 1.08 |
| 80 mg/ml | | | | |
| sucrose, pH 5.2 | | | | |
| | 7 d, 40 C. | 89.62 | 8.54 | 1.82 |
| | 21 d, 40 C. | 81.03 | 12.41 | 6.54 |
| 15 mM citrate, | T0 | 95.72 | 3.19 | 1.08 |
| 80 mg/ml | | | | |
| sucrose, pH 5.2 | | | | |
| | 7 d, 40 C. | 90.54 | 6.96 | 2.48 |
| | 21 d, 40 C. | 87.39 | 7.74 | 4.86 |
| 15 mM histidine, | T0 | 96 | 2.9 | 1.09 |
| 80 mg/ml | | | | |
| sucrose, pH 5.2 | | | | |
| | 7 d, 40 C. | 91.04 | 6.91 | 2.03 |
| | 21 d, 40 C. | 87.58 | 8.63 | 3.78 |
| Water, 80 mg/ml | T0 | 94.89 | 4.04 | 1.05 |
| sucrose, pH 5.2 | | | | |
| | 7 d, 40 C. | 88.4 | 9.76 | 1.82 |
| | 21 d, 40 C. | 81.41 | 14.39 | 4.18 |

TABLE 23
Effect of Buffer Type on Storage Stability
of DVD-A as Measured By IEC

| | Time | | | |
|---|---|---|---|---|
| Formulation | Point | Main | Acidic | Basic |
| 15 mM acetate, 80 mg/ml sucrose, | T0 | 56.88 | 9.43 | 33.68 |
| pH 4.0 | | | | |
| | 7 d, 40 C. | 46.91 | 18.74 | 34.33 |
| | 21 d, 40 C. | 34.62 | 24.82 | 40.54 |
| 15 mM acetate, 80 mg/ml sucrose, | T0 | 57.2 | 9.45 | 33.34 |
| pH 5.2 | | | | |
| | 7 d, 40 C. | 47.61 | 18.74 | 33.64 |
| | 21 d, 40 C. | 35.29 | 33.35 | 31.35 |
| 15 mM arginine, 80 mg/ml | T0 | 63.44 | 9.93 | 26.62 |
| sucrose, pH 5.2 | | | | |
| | 7 d, 40 C. | 49.22 | 19.57 | 31.2 |
| | 21 d, 40 C. | 37.87 | 25.3 | 36.81 |
| 15 mM citrate, 80 mg/ml sucrose, | T0 | 59.59 | 7.88 | 35.51 |
| pH 5.2 | | | | |
| | 7 d, 40 C. | 45.92 | 20.82 | 33.24 |
| | 21 d, 40 C. | 31.97 | 31.81 | 36.21 |
| 15 mM histidine, 80 mg/ml | T0 | 58.29 | 8.18 | 33.52 |
| sucrose, pH 5.2 | | | | |
| | 7 d, 40 C. | 44.97 | 16.84 | 38.17 |
| | 21 d, 40 C. | 36.33 | 24.88 | 38.77 |
| Water, 80 mg/ml sucrose, pH 5.2 | T0 | 57.05 | 9.53 | 33.41 |
| | 7 d, 40 C. | 48.23 | 19.96 | 37.15 |
| | 21 d, 40 C. | 37.69 | 25.15 | 37.15 |

SEC results provided in Table 22 show that a pH 5.2 histidine formulation compared to the pH 4 histidine formulation had the lowest level of aggregates, although citrate and acetate buffers showed low levels of aggregates as well. Although citrate and acetate showed less soluble aggregates as measured by SEC, the solutions were visibly turbid indicating formation of insoluble aggregates. The visual turbidity was not significant, however, given the SEC measurements. Overall, citrate and acetate formulations were only slightly less stable than the histidine formulations. Given that the noise/error in IEC measurement is usually higher, no significant differences in the chemical stability were observed within the formulations presented in Table 23. The polyol only formulation was also slightly less stable as compared to the histidine formulation. Given the overall stability characteristics of proteins identified as AS-DVD-Ig proteins, the slight differences observed in the SEC and IEC analysis of Tables 22 and 23 showed that each of the tested buffers at pH 5.2 was stable. Thus, the differences observed between the tested buffers at pH 5.2 indicated that each could be used to provide stable formulations for AS-DVD-Ig proteins, including those at high concentrations.

### VI. AS-DVD-Ig Proteins are Stable in Formulations Containing a Buffer, a Polyol, and a Surfactant at pH Range of 4.5-7.5

### Example 11: AS-DVD-Ig Proteins are Stable in a Range of Histidine Formulations Containing Surfactants and Sugars

The following example describes the impact of pH, buffers, and excipients (including surfactants and polyols) on the physico-chemical stability of DVD-Ig proteins at low and high concentration formulations during accelerated/real time stability testing.

Using size exclusion chromatography (SEC) and ion exchange chromatography (IEC), the storage stability of low concentration (1 mg/ml) and higher concentration (100 mg/ml) AS-DVD-Ig protein formulations was evaluated following three storage conditions: no storage (T0), 1 month at a controlled temperature of 5° C. (1 m, 5 C), and 1 month at a controlled temperature of 40° C. (1 m, 40 C). Formulations with varying pH (a range of 5.25 to 7.2 was selected), buffers, and excipients were tested, according to the following conditions:

1) pH 5.25 and pH 6, 15 mM histidine, 80 mg/ml sucrose, 0.01% Tween 80;

2) pH 6, 15 mM histidine, 40 mg/ml sorbitol, 0.01% Tween 80;

3) PBS (10 mM phosphate, 125 mM NaCl) at pH 6 and 7.2;

4) 20 mM glycine, 26 mg/ml glycerol pH 6; and

5) Water, 0.01% Tween 80 pH 5 and 6.

The results are presented in Tables 24-27 below. The data show that not all DVD-Ig proteins are stable in all tested pH and formulation conditions. DVD B and DVD 5 were observed to form high amounts of aggregates under all the solution conditions tested and were classified as non-AS-DVD-Ig proteins (in accordance with the assay presented in Example 4). All the other DVD-Ig proteins (previously selected as being AS-DVD-Ig proteins) behaved well and were stable.

Sucrose, sorbitol, glycerol, and glycine were used to evaluate the effect of these excipients. Tween 80 (polysorbate 80), a surfactant that provides stabilization against shaking stress, was also used to evaluate its impact on the stability of high concentration solutions. The impact of salt concentration was evaluated by varying the ionic strength using sodium chloride.

In general, a formulation at pH 6 or pH 5.2 in a histidine buffer was effective for all AS-DVD-Ig proteins. Both sorbitol and sucrose each improved stability. Sucrose resulted in the formation of less monomer after defined time points. The presence of salt resulted in increased instability (defined as less monomer remaining). The presence of glycerol and glycine resulted in less monomer remaining following shelf stability as compared to other formulations. For example, according to Table 8, the IL12IL18 DVD-Ig protein was more stable at pH 6 in the presence of sucrose than sorbitol. The data also demonstrate that the formulations with either none or very little ionic strength showed comparable stability over time.

SEC data showed the physical stability of the DVD-Ig proteins, wherein the rate of formation of aggregates and/or fragments was evaluated (see Table 24 and 26). IEC data is an indicator of the chemical stability of a DVD-Ig protein. Deamidation, for example, results in formation of acidic species (conversion of the main species to acidic species). Generation of positively charged variants would lead to an increase in the basic species. Formation of any of the two acidic or basic species indicates instability, as the formation of these two species results in an overall decrease in the % of main species (see Tables 25 and 27).

The results in Tables 24 to 27 suggest that an AS-DVD-Ig protein is stable in formulations comprising a surfactant alone, e.g., 0.01% Tween 80 at pH 5-6.

TABLE 24
100 mg/ml SEC Data For Various Stored Formulations

| | | Time | | | |
|---|---|---|---|---|---|
| | | Point, | | | |
| DVD-Ig | Formulation | temp | Mon. | Agg. | Frag. |
| IL12IL18 | 15 mM Histidine, 80 mg/ml | T0 | 96.53 | 1.1 | 2.36 |
| | Sucrose, 0.01% Tween, pH | | | | |
| | 5.25 | | | | |
| | | 1 m, 5 C. | 95.97 | 1.75 | 2.26 |
| | | 1 m, 40 C. | 92.58 | 2.86 | 4.55 |
| IL12IL18 | 15 mM Histidine, 80 mg/ml | T0 | 96.31 | 1.15 | 2.52 |
| | Sucrose, 0.01% Tween, | | | | |
| | pH 6 | | | | |
| | | 1 m, 5 C. | 96 | 1.71 | 2.27 |
| | | 1 m, 40 C. | 92.11 | 3.5 | 4.38 |
| IL12IL18 | 15 mM Histidine, 40 mg/ml | T0 | 96.61 | 1.25 | 2.13 |
| | Sorbitol, 0.01% Tween, | | | | |
| | pH 6 | | | | |
| | | 1 m, 5 C. | 95.71 | 1.78 | 2.5 |
| | | 1 m, 40 C. | 91.62 | 4.04 | 4.33 |
| 6 | 15 mM Histidine, 80 mg/ml | T0 | 89.61 | 10.38 | 0 |
| | Sucrose, 0.01% Tween, pH | | | | |
| | 5.25 | | | | |
| | | 1 m, 5 C. | 89.48 | 10.51 | 0 |
| | | 1 m, 40 C. | 85.85 | 9.96 | 4.18 |
| 6 | 15 mM Histidine, 80 mg/ml | T0 | 88.58 | 11.41 | 0 |
| | Sucrose, 0.01% Tween, | | | | |
| | pH 6 | | | | |
| | | 1 m, 5 C. | 86.8 | 13.19 | 0 |
| | | 1 m, 40 C. | 83.09 | 12.68 | 4.21 |
| 65 | 15 mM Histidine, 80 mg/ml | T0 | 93.08 | 0.93 | 5.98 |
| | Sucrose, 0.01% Tween, pH | | | | |
| | 5.25 | | | | |
| | | 1 m, 5 C. | 92.59 | 1.67 | 5.72 |
| | | 1 m, 40 C. | 85.25 | 11.47 | 3.27 |
| 65 | 15 mM Histidine, 80 mg/ml | T0 | 93.34 | 0.85 | 5.79 |
| | Sucrose, 0.01% Tween, | | | | |
| | pH 6 | | | | |
| | | 1 m, 5 C. | 92.45 | 1.71 | 5.82 |
| | | 1 m, 40 C. | 86.15 | 10.69 | 3.14 |
| 66 | 15 mM Histidine, 80 mg/ml | T0 | 97.57 | 0.62 | 1.8 |
| | Sucrose, 0.01% Tween, pH | | | | |
| | 5.25 | | | | |
| | | 1 m, 5 C. | 97.6 | 0.92 | 1.47 |
| | | 1 m, 40 C. | 91.65 | 5 | 3.34 |
| 66 | 15 mM Histidine, 80 mg/ml | T0 | 97.41 | 0.71 | 1.87 |
| | Sucrose, 0.01% Tween, | | | | |
| | pH 6 | | | | |
| | | 1 m, 5 C. | 97.69 | 1.07 | 1.22 |
| | | 1 m, 40 C. | 92.68 | 4.3 | 3.01 |
| 66 | 15 mM Histidine, 40 mg/ml | T0 | 97.65 | 0.61 | 1.73 |
| | Sorbitol, 0.01% Tween, | | | | |
| | pH 6 | | | | |
| | | 1 m, 5 C. | 97.52 | 1.04 | 1.42 |
| | | 1 m, 40 C. | 91.53 | 3.98 | 4.47 |
| IL12IL18 | 10 mM Phosphate, 125 mM | T0 | 96.13 | 1.57 | 2.28 |
| | NaCl, pH 6 | | | | |
| | | 1 m, 5 C. | 94.2 | 3.34 | 2.44 |
| | | 1 m, 40 C. | 88.48 | 7.3 | 4.2 |
| IL12IL18 | 10 mM Phosphate, 125 mM | T0 | 96.07 | 1.66 | 2.26 |
| | NaCl, pH 7.2 | | | | |
| | | 1 m, 5 C. | 94.64 | 2.91 | 2.43 |
| | | 1 m, 40 C. | 87.71 | 8.03 | 4.25 |
| DVD B | 10 mM Phosphate, 125 mM | T0 | 96.24 | 2.11 | 1.64 |
| | NaCl, pH 6 | | | | |
| | | 1 m, 5 C. | 94.66 | 3.85 | 1.48 |
| | | 1 m, 40 C. | 41.53 | 53.68 | 4.77 |
| DVD B | 10 mM Phosphate, 125 mM | T0 | 95.75 | 2.45 | 1.79 |
| | NaCl, pH 7.2 | | | | |
| | | 1 m, 5 C. | 95.11 | 3.62 | 1.26 |
| | | 1 m, 40 C. | 33.96 | 60.56 | 5.46 |
| IL12IL18 | 20 mM Glycine, 26 mg/ml | T0 | 96.48 | 1.27 | 2.24 |
| | Glycerol, pH 6.0 | | | | |
| | | 1 m, 5 C. | 95.06 | 2.07 | 2.86 |
| | | 1 m, 40 C. | 90.33 | 4.7 | 4.95 |
| DVD B | 20 mM Glycine, 26 mg/ml | T0 | 96.29 | 1.88 | 1.81 |
| | Glycerol, pH 6.0 | | | | |
| | | 1 m, 5 C. | 95.93 | 2.62 | 1.43 |
| | | 1 m, 40 C. | 23.32 | 73.53 | 3.14 |
| IL12IL18 | Water, 0.01% Tween 80, | T0 | 95.06 | 2.55 | 2.37 |
| | pH 5.0 | | | | |
| | | 1 m, 5 C. | 94.12 | 2.97 | 2.89 |
| | | 1 m, 40 C. | 90.99 | 4.93 | 4.07 |
| IL12IL18 | Water, 0.01% Tween 80, | T0 | 94.59 | 2.88 | 2.52 |
| | pH 6.0 | | | | |
| | | 1 m, 5 C. | 94.22 | 3.28 | 2.48 |
| | | 1 m, 40 C. | 90.9 | 4.82 | 4.26 |
| 5 | Water, 0.01% Tween 80, | T0 | 65.33 | 31.34 | 3.31 |
| | pH 5.0 | | | | |
| | | 1 m, 5 C. | 17.02 | 79.12 | 3.84 |
| | | 1 m, 40 C. | 82.54 | 13.41 | 4.03 |
| 5 | Water, 0.01% Tween 80, | T0 | 43.43 | 53.33 | 3.23 |
| | pH 6.0 | | | | |
| | | 1 m, 5 C. | 17.53 | 79.15 | 3.3 |
| | | 1 m, 40 C. | 74.6 | 21.38 | 4.03 |

TABLE 25
100 mg/ml IEC Data For Various Stored Formulations

| | | Time Point, | | | |
|---|---|---|---|---|---|
| DVD-Ig | Formulation | temp | Main | Acidic | Basic |
| IL12IL18 | 15 mM Histidine, | T0 | 58.32 | 27.98 | 13.68 |
| | 80 mg/ml Sucrose, | | | | |
| | 0.01% Tween, | | | | |
| | pH 5.25 | | | | |
| | | 1 m, 5 C. | 57.54 | 26.72 | 15.73 |
| | | 1 m, 40 C. | 44.91 | 37.65 | 17.43 |
| IL12IL18 | 15 mM Histidine, | T0 | 58.63 | 27.84 | 13.51 |
| | 80 mg/ml Sucrose, | | | | |
| | 0.01% Tween, pH 6 | | | | |
| | | 1 m, 5 C. | 56.89 | 29.15 | 13.94 |
| | | 1 m, 40 C. | 41.47 | 44.38 | 14.14 |
| IL12IL18 | 15 mM Histidine, | T0 | 58.1 | 28.09 | 13.79 |
| | 40 mg/ml Sorbitol, | | | | |
| | 0.01% Tween, pH 6 | | | | |
| | | 1 m, 5 C. | 60.53 | 27.48 | 11.98 |
| | | 1 m, 40 C. | 41.02 | 44.69 | 14.29 |
| 6 | 15 mM Histidine, | T0 | 42.99 | 11.66 | 45.34 |
| | 80 mg/ml Sucrose, | | | | |
| | 0.01% Tween, | | | | |
| | pH 5.25 | | | | |
| | | 1 m, 5 C. | 42.53 | 10.26 | 47.19 |
| | | 1 m, 40 C. | 34.99 | 25.55 | 39.45 |
| 6 | 15 mM Histidine, | T0 | 41.03 | 11.84 | 47.12 |
| | 80 mg/ml Sucrose, | | | | |
| | 0.01% Tween, | | | | |
| | pH 6 | | | | |
| | | 1 m, 5 C. | 41.73 | 10.91 | 47.34 |
| | | 1 m, 40 C. | 36.18 | 25.94 | 37.86 |
| 65 | 15 mM Histidine, | T0 | 49.64 | 39.02 | 11.33 |
| | 80 mg/ml Sucrose, | | | | |
| | 0.01% Tween, pH | | | | |
| | 5.25 | | | | |
| | | 1 m, 5 C. | 45.48 | 38.77 | 15.73 |
| | | 1 m, 40 C. | 51.17 | 30.64 | 18.18 |
| 65 | 15 mM Histidine, | T0 | 50 | 39.1 | 10.88 |
| | 80 mg/ml Sucrose, | | | | |
| | 0.01% Tween, | | | | |
| | pH 6 | | | | |
| | | 1 m, 5 C. | 49.31 | 38.95 | 11.72 |
| | | 1 m, 40 C. | 29.83 | 52.65 | 17.5 |
| 66 | 15 mM Histidine, | T0 | 63.78 | 24.78 | 11.43 |
| | 80 mg/ml Sucrose, | | | | |
| | 0.01% Tween, pH | | | | |
| | 5.25 | | | | |
| | | 1 m, 5 C. | 60.03 | 25.27 | 14.69 |
| | | 1 m, 40 C. | 42.13 | 41.93 | 15.93 |
| 66 | 15 mM Histidine, | T0 | 61.5 | 26.21 | 12.27 |
| | 80 mg/ml Sucrose, | | | | |
| | 0.01% Tween, pH 6 | | | | |
| | | 1 m, 5 C. | 59.06 | 25.85 | 15.07 |
| | | 1 m, 40 C. | 42.31 | 43.47 | 41.2 |
| 66 | 15 mM Histidine, | T0 | 57.18 | 24.54 | 18.26 |
| | 40 mg/ml Sorbitol, | | | | |
| | 0.01% Tween, pH 6 | | | | |
| | | 1 m, 5 C. | 61.9 | 26.5 | 11.59 |
| | | 1 m, 40 C. | 41.24 | 45.29 | 13.46 |
| IL12IL18 | 10 mM Phosphate, | T0 | 58.2 | 28.1 | 13.69 |
| | 125 mM NaCl, pH 6 | | | | |
| | | 1 m, 5 C. | 57.43 | 27.35 | 15.21 |
| | | 1 m, 40 C. | 40.68 | 42.04 | 17.27 |
| IL12IL18 | 10 mM Phosphate, | T0 | 54.51 | 27.99 | 17.48 |
| | 125 mM NaCl, pH 7.2 | | | | |
| | | 1 m, 5 C. | 53.79 | 28.08 | 18.11 |
| | | 1 m, 40 C. | 27.08 | 58.25 | 14.65 |
| DVD B | 10 mM Phosphate, | T0 | 52.94 | 27.32 | 19.72 |
| | 125 mM NaCl, pH 6 | | | | |
| | | 1 m, 5 C. | 54.38 | 27.19 | 18.42 |
| | | 1 m, 40 C. | 29.14 | 38.63 | 32.21 |
| DVD B | 10 mM Phosphate, | T0 | 27.93 | 52.59 | 19.46 |
| | 125 mM NaCl, pH 7.2 | | | | |
| | | 1 m, 5 C. | 54.07 | 26.57 | 19.35 |
| | | 1 m, 40 C. | 19.79 | 48.61 | 31.59 |
| IL12IL18 | 20 mM Glycine, | T0 | 57.95 | 28.99 | 13.05 |
| | 26 mg/ml Glycerol, | | | | |
| | pH 6.0 | | | | |
| | | 1 m, 5 C. | 57.85 | 29.95 | 12.18 |
| | | 1 m, 40 C. | 37.55 | 47.49 | 14.94 |
| DVD B | 20 mM Glycine, | T0 | 30.77 | 51 | 18.22 |
| | 26 mg/ml Glycerol, | | | | |
| | pH 6.0 | | | | |
| | | 1 m, 5 C. | 50 | 33.32 | 16.67 |
| | | 1 m, 40 C. | 27.41 | 58.52 | 14.05 |
| IL12IL18 | Water, 0.01% | T0 | 56.03 | 27.31 | 16.64 |
| | Tween 80, pH 5.0 | | | | |
| | | 1 m, 5 C. | 57.67 | 28.06 | 14.26 |
| | | 1 m, 40 C. | 44.97 | 41.34 | 13.68 |
| IL12IL18 | Water, 0.01% | T0 | 58.4 | 28.57 | 13.02 |
| | Tween 80, pH 6.0 | | | | |
| | | 1 m, 5 C. | 58.93 | 26.36 | 14.7 |
| | | 1 m, 40 C. | 45.37 | 39.4 | 15.21 |
| 5 | Water, 0.01% | T0 | 46.39 | 19.48 | 34.12 |
| | Tween 80, pH 5.0 | | | | |
| | | 1 m, 5 C. | 16.75 | 8.54 | 74.69 |
| | | 1 m, 40 C. | 44.57 | 36.64 | 18.78 |
| 5 | Water, 0.01% | T0 | 32.31 | 14.89 | 52.78 |
| | Tween 80, pH 6.0 | | | | |
| | | 1 m, 5 C. | 16.32 | 8.27 | 75.4 |
| | | 1 m, 40 C. | 39.7 | 35.23 | 25.06 |

TABLE 26
1 mg/ml SEC Data for Various Stored Formulations

| | | Time Point, | | | |
|---|---|---|---|---|---|
| DVD-Ig | Formulation | temp | Mon. | Agg. | Frag. |
| IL12IL18 | 15 mM Histidine, | T0 | 97.87 | 0.73 | 1.39 |
| | 80 mg/ml Sucrose, | | | | |
| | 0.01% Tween, pH 5.25 | | | | |
| | | 1 m, 5 C. | 97.83 | 0.57 | 1.59 |
| | | 1 m, 40 C. | 95.55 | 0.74 | 3.7 |
| IL12IL18 | 15 mM Histidine, | T0 | 98.04 | 0.32 | 1.62 |
| | 80 mg/ml Sucrose, | | | | |
| | 0.01% Tween, pH 6 | | | | |
| | | 1 m, 5 C. | 97.95 | 0.24 | 1.79 |
| | | 1 m, 40 C. | 95.83 | 0.58 | 3.58 |
| IL12IL18 | 15 mM Histidine, | T0 | 96.2 | 0.56 | 3.22 |
| | 40 mg/ml Sorbitol, | | | | |
| | 0.01% Tween, pH 6 | | | | |
| | | 1 m, 5 C. | 96.41 | 0.47 | 3.1 |
| | | 1 m, 40 C. | 94.21 | 0.66 | 5.12 |
| 6 | 15 mM Histidine, | T0 | 92.05 | 7.94 | 0 |
| | 80 mg/ml Sucrose, | | | | |
| | 0.01% Tween, pH 5.25 | | | | |
| | | 1 m, 5 C. | 99.23 | 0.76 | 0 |
| | | 1 m, 40 C. | 95.52 | 0.53 | 3.94 |
| 6 | 15 mM Histidine, | T0 | 93.59 | 6.4 | 0 |
| | 80 mg/ml Sucrose, | | | | |
| | 0.01% Tween, pH 6 | | | | |
| | | 1 m, 5 C. | 99.33 | 0.66 | 0 |
| | | 1 m, 40 C. | 95.3 | 0.59 | 4.1 |
| 65 | 15 mM Histidine, | T0 | 94.74 | 0.51 | 4.73 |
| | 80 mg/ml Sucrose, | | | | |
| | 0.01% Tween, pH 5.25 | | | | |
| | | 1 m, 5 C. | 94.34 | 0.49 | 5.15 |
| | | 1 m, 40 C. | 93.06 | 0.91 | 6.02 |
| 65 | 15 mM Histidine, | T0 | 94.33 | 0.37 | 5.29 |
| | 80 mg/ml Sucrose, | | | | |
| | 0.01% Tween, pH 6 | | | | |
| | | 1 m, 5 C. | 93.98 | 0.36 | 5.65 |
| | | 1 m, 40 C. | 92.71 | 0.87 | 6.41 |
| 66 | 15 mM Histidine, | T0 | 98.76 | 0.51 | 0.72 |
| | 80 mg/ml Sucrose, | | | | |
| | 0.01% Tween, pH 5.25 | | | | |
| | | 1 m, 5 C. | 98.59 | 0.51 | 0.89 |
| | | 1 m, 40 C. | 96.37 | 1.02 | 2.59 |
| 66 | 15 mM Histidine, | T0 | 98.82 | 0.2 | 0.96 |
| | 80 mg/ml Sucrose, | | | | |
| | 0.01% Tween, pH 6 | | | | |
| | | 1 m, 5 C. | 98.9 | 0.16 | 0.92 |
| | | 1 m, 40 C. | 96.82 | 0.66 | 2.51 |
| 66 | 15 mM Histidine, | T0 | 97.87 | 0.29 | 1.83 |
| | 40 mg/ml Sorbitol, | | | | |
| | 0.01% Tween, pH 6 | | | | |
| | | 1 m, 5 C. | 97.3 | 0.23 | 2.45 |
| | | 1 m, 40 C. | 95.5 | 1.02 | 3.48 |
| IL12IL18 | 10 mM Phosphate, | T0 | 95.38 | 1.54 | 3.07 |
| | 125 mM NaCl, pH 6 | | | | |
| | | 1 m, 5 C. | 95.29 | 1.54 | 3.15 |
| | | 1 m, 40 C. | 91.87 | 1.95 | 6.17 |
| IL12IL18 | 10 mM Phosphate, | T0 | 94.36 | 2.73 | 2.89 |
| | 125 mM NaCl, pH 7.2 | | | | |
| | | 1 m, 5 C. | 93.78 | 2.99 | 3.22 |
| | | 1 m, 40 C. | 86.45 | 3.64 | 9.9 |
| DVD B | 10 mM Phosphate, | T0 | 95.82 | 1.66 | 2.5 |
| | 125 mM NaCl, pH 6 | | | | |
| | | 1 m, 5 C. | 95.54 | 1.53 | 2.91 |
| | | 1 m, 40 C. | 91.39 | 2.31 | 6.29 |
| DVD B | 10 mM Phosphate, | T0 | 94.87 | 2.43 | 2.68 |
| | 125 mM NaCl, pH 7.2 | | | | |
| | | 1 m, 5 C. | 95.01 | 2.3 | 2.68 |
| | | 1 m, 40 C. | 88.42 | 3.24 | 8.32 |
| IL12IL18 | 20 mM Glycine, | T0 | 96.48 | 0.36 | 3.14 |
| | 26 mg/ml Glycerol, | | | | |
| | pH 6.0 | | | | |
| | | 1 m, 5 C. | 96.51 | 0.29 | 3.18 |
| | | 1 m, 40 C. | 93.82 | 0.4 | 5.77 |
| DVD B | 20 mM Glycine, | T0 | 96.05 | 1.33 | 2.6 |
| | 26 mg/ml Glycerol, | | | | |
| | pH 6.0 | | | | |
| | | 1 m, 5 C. | 95.47 | 1.15 | 3.37 |
| | | 1 m, 40 C. | 94.14 | 1.22 | 4.63 |
| IL12IL18 | Water, 0.01% Tween 80, | T0 | 89.16 | 2.42 | 8.41 |
| | pH 5.0 | | | | |
| | | 1 m, 5 C. | 95.1 | 1.86 | 3.02 |
| | | 1 m, 40 C. | 89.69 | 4.11 | 6.18 |
| IL12IL18 | Water, 0.01% Tween 80, | T0 | 94.24 | 2.88 | 2.86 |
| | pH 6.0 | | | | |
| | | 1 m, 5 C. | 94.89 | 2.6 | 2.49 |
| | | 1 m, 40 C. | 90.66 | 3.38 | 5.94 |
| 5 | Water, pH 5.0 | T0 | 69.49 | 26.36 | 4.13 |
| | | 1 m, 5 C. | 68.87 | 24.88 | 6.23 |
| | | 1 m, 40 C. | 91.35 | 3.6 | 5.08 |
| 5 | Water, pH 6.0 | T0 | 49.15 | 46.65 | 4.19 |
| | | 1 m, 5 C. | 51.96 | 43.27 | 4.76 |
| | | 1 m, 40 C. | 92.56 | 2.45 | 4.97 |

TABLE 27
1 mg/ml IEC Data for the Various Stored Formulations

| | | Time | | | |
|---|---|---|---|---|---|
| | | Point, | | | |
| DVD-Ig | Formulation | temp | Main | Acidic | Basic |
| IL12IL18 | 15 mM Histidine, | T0 | 61.83 | 24 | 14.14 |
| | 80 mg/ml Sucrose, | | | | |
| | 0.01% Tween, pH 5.25 | | | | |
| | | 1 m, 5 C. | 61.76 | 26.77 | 11.46 |
| | | 1 m, 40 C. | 41.58 | 39.69 | 18.72 |
| IL12IL18 | 15 mM Histidine, | T0 | 61.31 | 26.12 | 12.55 |
| | 80 mg/ml Sucrose, | | | | |
| | 0.01% Tween, pH 6 | | | | |
| | | 1 m, 5 C. | 59.67 | 25.3 | 15.02 |
| | | 1 m, 40 C. | 42.98 | 45.85 | 11.15 |
| IL12IL18 | 15 mM Histidine, | T0 | 60.81 | 25.86 | 13.31 |
| | 40 mg/ml Sorbitol, | | | | |
| | 0.01% Tween, pH 6 | | | | |
| | | 1 m, 5 C. | 59.07 | 27.12 | 13.8 |
| | | 1 m, 40 C. | 38.22 | 47.55 | 14.21 |
| 6 | 15 mM Histidine, | T0 | 41.13 | 10.3 | 48.56 |
| | 80 mg/ml Sucrose, | | | | |
| | 0.01% Tween, pH 5.25 | | | | |
| | | 1 m, 5 C. | 46.96 | 10.38 | 42.64 |
| | | 1 m, 40 C. | 36 | 24.57 | 39.42 |
| 6 | 15 mM Histidine, | T0 | 44.43 | 9.92 | 45.63 |
| | 80 mg/ml Sucrose, | | | | |
| | 0.01% Tween, pH 6 | | | | |
| | | 1 m, 5 C. | 46.33 | 11.55 | 42.11 |
| | | 1 m, 40 C. | 37.38 | 26.09 | 36.52 |
| 65 | 15 mM Histidine, | T0 | 50.6 | 39.58 | 9.81 |
| | 80 mg/ml Sucrose, | | | | |
| | 0.01% Tween, pH 5.25 | | | | |
| | | 1 m, 5 C. | 49.57 | 37.75 | 11.66 |
| | | 1 m, 40 C. | 33.38 | 50.27 | 16.33 |
| 65 | 15 mM Histidine, | T0 | 51.74 | 38.73 | 9.52 |
| | 80 mg/ml Sucrose, | | | | |
| | 0.01% Tween, pH 6 | | | | |
| | | 1 m, 5 C. | 53.21 | 36.58 | 10.2 |
| | | 1 m, 40 C. | 35.11 | 49.96 | 14.92 |
| 66 | 15 mM Histidine, | T0 | 59.89 | 25.96 | 14.14 |
| | 80 mg/ml Sucrose, | | | | |
| | 0.01% Tween, pH 5.25 | | | | |
| | | 1 m, 5 C. | 58.1 | 25.05 | 16.84 |
| | | 1 m, 40 C. | 45.43 | 37.21 | 17.34 |
| 66 | 15 mM Histidine, | T0 | 58.18 | 24.86 | 16.94 |
| | 80 mg/ml Sucrose, | | | | |
| | 0.01% Tween, pH 6 | | | | |
| | | 1 m, 5 C. | 61.69 | 24.94 | 13.36 |
| | | 1 m, 40 C. | 44.83 | 41.39 | 13.77 |
| 66 | 15 mM Histidine, | T0 | 61.49 | 24.96 | 13.53 |
| | 40 mg/ml Sorbitol, | | | | |
| | 0.01% Tween, pH 6 | | | | |
| | | 1 m, 5 C. | 60.34 | 24.96 | 16.69 |
| | | 1 m, 40 C. | 38.32 | 50.92 | 10.75 |
| IL12IL18 | 10 mM Phosphate, | T0 | 58.52 | 26.18 | 15.29 |
| | 125 mM NaCl, pH 6 | | | | |
| | | 1 m, 5 C. | 58.98 | 27.17 | 13.84 |
| | | 1 m, 40 C. | 37.54 | 47.49 | 14.96 |
| IL12IL18 | 10 mM Phosphate, | T0 | 63.15 | 23.13 | 13.71 |
| | 125 mM NaCl, pH 7.2 | | | | |
| | | 1 m, 5 C. | 58.58 | 26.07 | 15.33 |
| | | 1 m, 40 C. | 16.92 | 73.33 | 9.74 |
| DVD B | 10 mM Phosphate, | T0 | 54.38 | 27.35 | 18.25 |
| | 125 mM NaCl, pH 6 | | | | |
| | | 1 m, 5 C. | 56.48 | 27.42 | 16.09 |
| | | 1 m, 40 C. | 30.99 | 50.68 | 18.32 |
| DVD B | 10 mM Phosphate, | T0 | 54.86 | 30 | 15.12 |
| | 125 mM NaCl, pH 7.2 | | | | |
| | | 1 m, 5 C. | 52.66 | 31.63 | 15.69 |
| | | 1 m, 40 C. | 8.64 | 60.51 | 30.84 |
| IL12IL18 | 20 mM Glycine, | T0 | 57.58 | 27.8 | 14.6 |
| | 26 mg/ml Glycerol, | | | | |
| | pH 6.0 | | | | |
| | | 1 m, 5 C. | 58.02 | 29.5 | 12.46 |
| | | 1 m, 40 C. | 28.8 | 60.9 | 10.29 |
| TNFPGE2 | 20 mM Glycine, | T0 | 53.05 | 30.46 | 16.48 |
| | 26 mg/ml Glycerol, | | | | |
| | pH 6.0 | | | | |
| | | 1 m, 5 C. | 44.21 | 33.97 | 21.8 |
| | | 1 m, 40 C. | 5.52 | 81.17 | 13.3 |
| IL12IL18 | Water, 0.01% Tween 80, | T0 | 57.76 | 26.51 | 15.71 |
| | pH 5.0 | | | | |
| | | 1 m, 5 C. | 58.44 | 26.13 | 15.41 |
| | | 1 m, 40 C. | 36.16 | 44.81 | 19.02 |
| IL12IL18 | Water, 0.01% Tween 80, | T0 | 59.24 | 26.09 | 14.66 |
| | pH 6.0 | | | | |
| | | 1 m, 5 C. | 58.66 | 26.96 | 14.36 |
| | | 1 m, 40 C. | 35.97 | 44.16 | 19.85 |
| 5 | Water, 0.01% Tween 80, | T0 | 51.73 | 19.4 | 28.85 |
| | pH 5.0 | | | | |
| | | 1 m, 5 C. | 48.63 | 20.14 | 31.21 |
| | | 1 m, 40 C. | 44.52 | 41.73 | 13.73 |
| 5 | Water, 0.01% Tween 80, | T0 | 34.34 | 14 | 51.62 |
| | pH 6.0 | | | | |
| | | 1 m, 5 C. | 32.27 | 15.38 | 47.33 |
| | | 1 m, 40 C. | 42.03 | 44.28 | 13.67 |

### Example 12. Impact of Storage on the Stability of DVD-Ig Protein (DVD-C) in Various Formulations

The pH and the storage temperature of a protein formulation are two important factors influencing protein stability during accelerated/long-term storage. To assess the impact of these factors, the DVD-Ig protein was exposed to short-term storage at elevated and real time temperatures in order to gain insight into the formulation feasibility of long-term storage at lower temperatures (e.g., 2-8° C.).

The storage stability of DVD-C in solution (100 mg/ml) was evaluated in formulations at 40° C. After defined storage periods, samples were pulled and the impact of storage time on DVD-Ig protein stability was evaluated. Samples were filled into sterile vials (approx. 500 μl each) and stored under controlled conditions, in a temperature chamber and in the absence of light. At predefined points of time, samples of prepared solutions were pulled for analysis according to the sample pull scheme. The resulting data is provided in Tables 28 and 29.

TABLE 28
Impact of Storage of DVD-C at 100 mg/ml Concentrations in Various
Conditions as Measured by SEC

| Formulation | Time Point | Monomer | Aggregates | Fragments |
|---|---|---|---|---|
| 15 mM acetate | T0 | 97.66391 | 1.091349 | 1.201071 |
| 80 mg/ml sucrose | 7 d, 40° C. | 96.32112 | 1.6992 | 1.97968 |
| 0.02% Tween 80 | 1 m, 40° C. | 94.1791 | 2.430244 | 3.390653 |
| pH 5 | | | | |
| 15 mM histidine | T0 | 97.66391 | 1.091349 | 1.201071 |
| 80 mg/ml sucrose | 7 d, 40° C. | 95.92304 | 2.09104 | 1.985924 |
| 0.02% Tween 80 | 1 m, 40° C. | 93.63996 | 2.724012 | 3.636029 |
| pH 6 | | | | |

TABLE 29
Impact of Storage of DVD-C at 100 mg/ml Concentrations in Various
Conditions as Measured By IEC

| Formulation | Time Point | Main | Acidic | Basic |
|---|---|---|---|---|
| 15 mM acetate | T0 | 73.84454 | 9.804467 | 16.35099 |
| 80 mg/ml sucrose | T2m, 5° C. | 69.17591 | 10.87888 | 19.94521 |
| 0.02% Tween 80 pH 5 | | | | |
| 15 mM histidine | T0 | 73.84454 | 9.804467 | 16.35099 |
| 80 mg/ml sucrose | T2m, 5° C. | 67.99154 | 10.58779 | 21.42067 |
| 0.02% Tween 80 pH 6 | | | | |

The data provided in Tables 28 and 29 show that DVD-C was found to be very stable (compared to some unstable DVD-Ig proteins, for example, in Example 5) in terms that only minimal loss in monomer levels occurred during test conditions and hence would be classified as an AS-DVD-Ig protein.

### Example 13: Effect of a Surfactant on the Stability of AS-DVD-Ig Proteins in Buffer and Polyol Containing Formulations

It is generally beneficial to set a formulation pH more than 1 unit from the protein's isoelectric point (pI). The more a formulation pH approximates the pI, generally, the more the overall surface of the protein is regarded uncharged, thus contributing to protein-protein attraction of non-polar groups, and thus enhancing non-covalent aggregation and instability. Shaking and stifling foster physical instability, creating hydrophobic air/water interfaces, which result in alignment of protein molecules at these interfaces, and eventually result in aggregation. Given that air is more hydrophobic than water, the interface between air and liquid is deemed a denaturing surface at which aggregation, especially of (partially) unfolded proteins can originate. The effective air-water interface can be increased by shaking or stirring.

In the following example, the effect of various concentrations of a surfactant (e.g., Tween 80) on the instability of various DVD-Ig proteins was evaluated. The study was done in the absence or presence of the polyol sucrose. Turbidity measurements at 500 nm using UV are listed in Table 30 and the SEC analysis of the samples is listed in Table 31. Ranges of pH were also tested in the formulations described below.

TABLE 30
Effect Of Tween On The Stability Of Various DVD-Ig Proteins As
Assessed By Air/Liquid Interface Denaturation Study/Shaking Study*

| | T0 | T24 h | T96 h |
|---|---|---|---|
| DVD-A, 15 mM Histidine, pH 5.2, | | | |
| 80 mg/ml Sucrose | | | |
| 0 Tween 80 | 0.11 | 0.07 | 0.55 |
| 2 mg/ml Tween 80 | 0.069 | 0.018 | 0.004 |
| 0.5 mg/ml Tween 80 | 0.014 | 0.014 | 0.002 |
| 0.1 mg/ml Tween 80 | 0.061 | 0.02 | 0.001 |
| 0.05 mg/ml Tween 80 | 0.018 | 0.006 | 0.001 |
| 0.01 mg/ml Tween 80 | 0.007 | 0.007 | 1.03 |
| DVD-C, 15 mM Histidine, pH 5.4, | | | |
| 80 mg/ml Sucrose | | | |
| 0 Tween 80 | 0.085 | 0.08 | 0.107 |
| 2 mg/ml Tween 80 | 0.085 | 0.038 | 0.003 |
| 0.5 mg/ml Tween 80 | 0.025 | 0.036 | 0.004 |
| 0.1 mg/ml Tween 80 | 0.043 | 0.007 | 0.002 |
| 0.05 mg/ml Tween 80 | 0.019 | 0.008 | 0.002 |
| 0.01 mg/ml Tween 80 | 0.03 | 0.033 | 0.22 |
| IL12IL18, 15 mM Histidine, pH 5.4, | | | |
| 80 mg/ml Sucrose | | | |
| 0 Tween 80 | 0.051 | 0.051 | 0.051 |
| 2 mg/ml Tween 80 | 0.069 | 0.072 | 0.017 |
| 0.5 mg/ml Tween 80 | 0.09 | 0.006 | 0.008 |
| 0.1 mg/ml Tween 80 | 0.055 | 0.011 | 0.0109 |
| 0.05 mg/ml Tween 80 | 0.022 | 0.008 | 0.0208 |
| 0.01 mg/ml Tween 80 | 0.057 | 0.081 | 0.082 |
| DVD-A, 15 mM Histidine, pH 5.2 | | | |
| 0 Tween 80 | 0.045 | 0.038 | 0.29 |
| 2 mg/ml Tween 80 | 0.049 | 0.024 | 0.004 |
| 0.5 mg/ml Tween 80 | 0.016 | 0.058 | 0.004 |
| 0.1 mg/ml Tween 80 | 0.005 | 0.005 | 0.002 |
| 0.05 mg/ml Tween 80 | 0.004 | 0.011 | 0.002 |
| 0.01 mg/ml Tween 80 | 0.005 | 0.03 | 0.66 |
| DVD-C, 15 mM Histidine, pH 5.4 | | | |
| 0 Tween 80 | 0.015 | 0.045 | 0.038 |
| 2 mg/ml Tween 80 | 0.015 | 0.02 | 0.003 |
| 0.5 mg/ml Tween 80 | 0.03 | 0.019 | 0.003 |
| 0.1 mg/ml Tween 80 | 0.004 | 0.005 | 0.002 |
| 0.05 mg/ml Tween 80 | 0.005 | 0.001 | 0.009 |
| 0.01 mg/ml Tween 80 | 0.01 | 0.015 | 0.022 |
| IL12IL18, 15 mM Histidine, pH 5.4 | | | |
| 0 Tween 80 | 0.046 | 0.046 | 0.038 |
| 2 mg/ml Tween 80 | 0.097 | 0.03 | 0.018 |
| 0.5 mg/ml Tween 80 | 0.049 | 0.031 | 0.013 |
| 0.1 mg/ml Tween 80 | 0.012 | 0.021 | 0.017 |
| 0.05 mg/ml Tween 80 | 0.005 | 0.015 | 0.017 |
| 0.01 mg/ml Tween 80 | 0.05 | 0.022 | 0.041 |

*The OD 500 was measured using UV

TABLE 31
Effect of Tween on the Stability Of Various DVD-Ig Proteins as
Assessed By Air/Liquid Interface Denaturation Study/Shaking Study*

| | Aggregate | Monomer | Fragment | AUC |
|---|---|---|---|---|
| T0 | | | | |
| DVD-A, 15 mM Histidine, | | | | |
| pH 5.2, 80 mg/ml Sucrose | | | | |
| 0 Tween 80 | 1.7 | 97.25 | 1.03 | 73550 |
| 2 mg/ml Tween 80 | 1.67 | 97.58 | 0.74 | 759057 |
| 0.5 mg/ml Tween 80 | 1.76 | 97.43 | 0.8 | 75635 |
| 0.1 mg/ml Tween 80 | 1.65 | 97.66 | 0.68 | 73158 |
| 0.05 mg/ml Tween 80 | 1.66 | 97.61 | 0.71 | 77474 |
| 0.01 mg/ml Tween 80 | 1.68 | 97.5 | 0.81 | 77410 |
| DVD-C, 15 mM Histidine, | | | | |
| pH 5.4, 80 mg/ml Sucrose | | | | |
| 0 Tween 80 | 1.96 | 96.79 | 1.23 | 64166 |
| 2 mg/ml Tween 80 | 2.16 | 96.4 | 1.43 | 72649 |
| 0.5 mg/ml Tween 80 | 2 | 96.66 | 1.33 | 71044 |
| 0.1 mg/ml Tween 80 | 2.09 | 96.43 | 1.47 | 70277 |
| 0.05 mg/ml Tween 80 | 2.01 | 96.56 | 1.41 | 700701 |
| 0.01 mg/ml Tween 80 | 2.11 | 96.54 | 1.33 | 70397 |
| IL12IL18, 15 mM | | | | |
| Histidine, pH 5.4, | | | | |
| 80 mg/ml Sucrose | | | | |
| 0 Tween 80 | 7.15 | 91.18 | 1.66 | 76420 |
| 2 mg/ml Tween 80 | 7.29 | 90.64 | 2.05 | 77867 |
| 0.5 mg/ml Tween 80 | 7.04 | 91.4 | 1.54 | 77164 |
| 0.1 mg/ml Tween 80 | 7.08 | 91.23 | 1.68 | 72875 |
| 0.05 mg/ml Tween 80 | 7.03 | 91.3 | 1.65 | 69962 |
| 0.01 mg/ml Tween 80 | 6.81 | 91.57 | 1.61 | 77682 |
| DVD-A, 15 mM Histidine, | | | | |
| pH 5.2 | | | | |
| 0 Tween 80 | 1.59 | 97.58 | 0.81 | 76481 |
| 2 mg/ml Tween 80 | 1.55 | 97.65 | 0.79 | 85152 |
| 0.5 mg/ml Tween 80 | 1.65 | 97.64 | 0.7 | 88543 |
| 0.1 mg/ml Tween 80 | 1.69 | 97.57 | 0.73 | 85244 |
| 0.05 mg/ml Tween 80 | 1.72 | 97.57 | 0.7 | 86031 |
| 0.01 mg/ml Tween 80 | 1.63 | 97.63 | 0.72 | 81730 |
| DVD-C, 15 mM Histidine, | | | | |
| pH 5.4 | | | | |
| 0 Tween 80 | 2.05 | 96.56 | 1.37 | 71752 |
| 2 mg/ml Tween 80 | 2.08 | 96.63 | 1.28 | 75988 |
| 0.5 mg/ml Tween 80 | 2.1 | 96.44 | 1.45 | 77324 |
| 0.1 mg/ml Tween 80 | 2.11 | 96.33 | 1.55 | 77859 |
| 0.05 mg/ml Tween 80 | 2.21 | 96.28 | 1.5 | 79134 |
| 0.01 mg/ml Tween 80 | 2.08 | 96.44 | 1.47 | 76726 |
| IL12IL18, 15 mM | | | | |
| Histidine, pH 5.4 | | | | |
| 0 Tween 80 | 6.9 | 91.47 | 1.61 | 75651 |
| 2 mg/ml Tween 80 | 6.77 | 91.76 | 1.46 | 75316 |
| 0.5 mg/ml Tween 80 | 6.88 | 91.57 | 1.53 | 75439 |
| 0.1 mg/ml Tween 80 | 7.02 | 91.18 | 1.78 | 74981 |
| 0.05 mg/ml Tween 80 | 7.04 | 91.37 | 1.57 | 76385 |
| 0.01 mg/ml Tween 80 | 6.66 | 91.69 | 1.64 | 65218 |
| T96h | | | | |
| DVD-A, 15 mM Histidine, | | | | |
| pH 5.2, 80 mg/ml Sucrose | | | | |
| 0 Tween 80 | 1.35 | 97.55 | 1.08 | 39881 |
| 2 mg/ml Tween 80 | 2.33 | 96.67 | 0.99 | 74778 |
| 0.5 mg/ml Tween 80 | 2.35 | 96.42 | 1.21 | 74782 |
| 0.1 mg/ml Tween 80 | 3.85 | 95.06 | 1.07 | 72816 |
| 0.05 mg/ml Tween 80 | 18.86 | 80.36 | 0.76 | 76431 |
| 0.01 mg/ml Tween 80 | 9.32 | 83.25 | 7.41 | 11267 |
| DVD-C, 15 mM Histidine, | | | | |
| pH 5.4, 80 mg/ml Sucrose | | | | |
| 0 Tween 80 | 1.64 | 97.26 | 1.08 | 64855 |
| 2 mg/ml Tween 80 | 1.73 | 96.65 | 1.6 | 71235 |
| 0.5 mg/ml Tween 80 | 1.96 | 96.47 | 1.56 | 70435 |
| 0.1 mg/ml Tween 80 | 2.2 | 96.38 | 1.41 | 69349 |
| 0.05 mg/ml Tween 80 | 6.14 | 92.34 | 1.51 | 67875 |
| 0.01 mg/ml Tween 80 | 2.32 | 96.08 | 1.59 | 45758 |
| IL12IL18, 15 mM | | | | |
| Histidine, pH 5.4, | | | | |
| 80 mg/ml Sucrose | | | | |
| 0 Tween 80 | 6.11 | 92.04 | 1.83 | 72538 |
| 2 mg/ml Tween 80 | 6.09 | 91.94 | 1.96 | 76447 |
| 0.5 mg/ml Tween 80 | 6.14 | 91.98 | 1.87 | 75844 |
| 0.1 mg/ml Tween 80 | 6.4 | 90.9 | 2.68 | 71645 |
| 0.05 mg/ml Tween 80 | 8.8 | 86.44 | 4.75 | 72941 |
| 0.01 mg/ml Tween 80 | 12.77 | 85.19 | 2.03 | 70341 |
| DVD-A, 15 mM Histidine, | | | | |
| pH 5.2 | | | | |
| 0 Tween 80 | 1.09 | 98.02 | 0.87 | 60575 |
| 2 mg/ml Tween 80 | 1.63 | 97.49 | 0.87 | 84633 |
| 0.5 mg/ml Tween 80 | | | | |
| 0.1 mg/ml Tween 80 | 1.61 | 97.66 | 0.72 | 87583 |
| 0.05 mg/ml Tween 80 | 2.22 | 96.67 | 1.09 | 86215 |
| 0.01 mg/ml Tween 80 | 28.99 | 69.37 | 1.63 | 15513 |
| DVD-C, 15 mM Histidine, | | | | |
| pH 5.4 | | | | |
| 0 Tween 80 | 1.71 | 97.3 | 0.98 | 67869 |
| 2 mg/ml Tween 80 | 2.03 | 96.58 | 1.37 | 75354 |
| 0.5 mg/ml Tween 80 | 2 | 96.49 | 1.5 | 76488 |
| 0.1 mg/ml Tween 80 | 1.86 | 96.59 | 1.54 | 76809 |
| 0.05 mg/ml Tween 80 | 2.38 | 96.09 | 1.52 | 77153 |
| 0.01 mg/ml Tween 80 | 2.95 | 95.52 | 1.51 | 74056 |
| IL12IL18, 15 mM | | | | |
| Histidine, pH 5.4 | | | | |
| 0 Tween 80 | 6.34 | 91.6 | 2.05 | 73693 |
| 2 mg/ml Tween 80 | 6.43 | 91.61 | 1.95 | 75143 |
| 0.5 mg/ml Tween 80 | 6.12 | 91.84 | 2.03 | 74397 |
| 0.1 mg/ml Tween 80 | 7.06 | 90.81 | 2.11 | 74698 |
| 0.05 mg/ml Tween 80 | 5.61 | 92.44 | 1.93 | 75375 |
| 0.01 mg/ml Tween 80 | 10.76 | 87.29 | 1.93 | 62074 |

*The SEC data corresponds to 0 and 96 h shaking samples of Table 30

The data presented in Tables 30 and 31 compare various surfactant concentrations (0 to 2 mg/ml) in a histidine buffer at pH 5.2 or 5.4 with and without sucrose (80 mg/ml). Results of turbidity measurements show that a surfactant (Tween 80) in a concentration range of 0.05 mg/ml-2 mg/ml provided stability against shear/denaturation stress to AS-DVD-Ig proteins in general. The turbidity increased on lowering the surfactant concentration to 0.01 mg/ml. Similar observations were made for AS-DVD-Ig proteins in the presence of sucrose. All studies were conducted at 15 mM histidine at a DVD-Ig protein concentration of 1 mg/ml.

### Example 14: Effect of Surfactant Concentration on the Stability of IL12IL18 DVD-Ig Protein as Measured in Shaking Studies

The following example describes the effect of different concentrations (a range of concentration) of polysorbate 80 and poloxamer on the shaking stability of an IL12IL18 DVD-Ig protein at concentrations of 1 mg/mL at pH 6 (15 mM histidine+80 mg/mL sucrose) as measured using optical density at two different wavelengths of 350 and 50 nm.

TABLE 32
Effect of Surfactants on Formulation with Buffer and Polyol

| | | IL12IL18 1 mg/ml; 15 mM Histidine + 80 mg/mL sucrose pH 6 | | | | | | | | | |
|---|---|---|---|---|---|---|---|---|---|---|---|
| | | Shake Time (H) | | | | | | | | | |
| Surfactant | | 0 | 24 | 48 | 120 | 240 | 0 | 24 | 48 | 120 | 240 |
| | | OD500 nm | | | | | OD350 nm | | | | |
| Tween 80 | 0 | 0.003 | 0.06 | 0.085 | 0.137 | 0.387 | 0.01 | 0.097 | 0.135 | 0.22 | 0.574 |
| (mg/ml) | 0.01 | 0.0036 | 0.029 | 0.011 | 0.065 | 0.108 | 0.01 | 0.01 | 0.03 | 0.167 | 0.227 |
| | 0.05 | 0.0056 | 0.003 | 0.01 | 0.087 | 0.113 | 0.013 | 0.01 | 0.027 | 0.17 | 0.22 |
| | 0.1 | 0.0036 | 0.002 | 0.0022 | 0.067 | 0.004 | 0.01 | 0.01 | 0.009 | 0.135 | 0.011 |
| | 0.5 | 0.003 | 0.002 | 0.002 | 0.04 | 0.001 | 0.01 | 0.009 | 0.009 | 0.086 | 0.009 |
| | 2 | 0.035 | 0.002 | 0.0025 | 0.0025 | 0.002 | 0.062 | 0.012 | 0.013 | 0.014 | 0.013 |
| | | 500 nM | | | | | 350 nM | | | | |
| Poloxamer | 0 | 0.003 | 0.035 | 0.044 | 0.236 | 0.226 | 0.01 | 0.06 | 0.08 | 0.4 | 0.374 |
| (% w/v) | 0.01 | 0.004 | 0.004 | 0.007 | 0.013 | 0.0126 | 0.01 | 0.012 | 0.019 | 0.031 | 0.034 |
| | 0.05 | 0.003 | 0.014 | 0.002 | 0.027 | 0.002 | 0.01 | 0.02 | 0.011 | 0.009 | 0.011 |
| | 0.1 | 0003 | 0.035 | 0.0037 | 0.0038 | 0.003 | 0.01 | 0.01 | 0.012 | 0.012 | 0.012 |
| | 0.5 | 0.003 | 0.003 | 0.003 | 0.003 | 0.004 | 0.01 | 0.01 | 0.011 | 0.011 | 0.013 |
| | 2 | 0.0036 | 0.003 | 0.003 | 0.005 | 0.004 | 0.011 | 0.01 | 0.01 | 0.013 | 0.013 |

As described in Table 32, the addition of surfactants increased the shaking stability of the IL12/IL18 DVD-Ig protein. Polysorbate concentration in the range of 0.05-2 mg/mL was determined to be the most effective for stability of the IL12/IL18 DVD-Ig protein, and poloxamer was determined to be most effective in the range of 01-2% w/v. DVD-B was also tested and showed similar stability when tested in this particular shaking assay.

### Example 15: Effect of Polyol (Sucrose and Sorbitol) Concentration on the Stability of IL12IL18 DVD-Ig Protein

The following example shows the effect of polyols sucrose and sorbitol in the presence of polysorbate 80 on the stability of the IL12IL18 DVD-Ig protein as measured at 3 mg/mL at pH 6 by intrinsic fluorescence using Optim-1000 instrument.

TABLE 33
Effect of polyols on formulation with buffer and polysorbate

| | | | | | | DSF |
|---|---|---|---|---|---|---|
| | | | | | | (temp |
| | Conc | | Sucrose | Sorbitol | | Deg |
| | (mg/ml) | Buffer | (mg/ml) | (mg/ml) | Tween | C.) |
| IL12/18 | 3 | 15 mM His | 0 | | 0.01% | 60 |
| | | pH = 6 | | | | |
| IL12/18 | 3 | 15 mM His | 10 | | 0.01% | 61 |
| | | pH = 6 | | | | |
| IL12/18 | 3 | 15 mM His | 40 | | 0.01% | 61 |
| | | pH = 6 | | | | |
| IL12/18 | 3 | 15 mM His | 70 | | 0.01% | 60 |
| | | pH = 6 | | | | |
| IL12/18 | 3 | 15 mM His | 100 | | 0.01% | 60 |
| | | pH = 6 | | | | |
| IL12/18 | 3 | 15 mM His | | 0 | 0.01% | 61 |
| | | pH = 6 | | | | |
| IL12/18 | 3 | 15 mM His | | 10 | 0.01% | 62 |
| | | pH = 6 | | | | |
| IL12/18 | 3 | 15 mM His | | 20 | 0.01% | 61 |
| | | pH = 6 | | | | |
| IL12/18 | 3 | 15 mM His | | 40 | 0.01% | 62 |
| | | pH = 6 | | | | |
| IL12/18 | 3 | 15 mM His | | 60 | 0.01% | 62 |
| | | pH = 6 | | | | |

Based on the data, the stability of the DVD-Ig protein increased with an increase in the concentration of either polyol (sucrose or sorbitol). It was determined that 100 mg/mL for sucrose and 60 mg/mL for sorbitol was about the maximum concentrations that would achive osmolality and hence were investigated. Sucrose in the range of 60-100 mg/mL was determined to be effective, while sorbitol in the range of 20-60 mg/mL was effective for stability. DVD-B was also tested and showed similar stability when tested in this particular assay.

### Example 16: Impact of pH and Histidine Concentration (at pH 6) on the Shelf Stability of DVD-B and IL12IL18 DVD-Ig Proteins at 100 mg/ml

The following examples show the effect of histidine concentration (ranging from 0 to 200 mM) on the shelf stability of the DVD B and the IL12IL18 DVD-Ig protein as measured using size exclusion chromatography (SEC). Table 34 also shows the effect of pH range from 4.5-7.4 (pH 4.5 is 15 mM Acetate, pH 6 is 15 mM Histidine and pH 7.4 is 15 mM Phosphate) on the stability of the two DVD-Ig proteins. It is clearly visible that the stability of IL1IL18 DVD-Ig protein is maintained between the pH range of 4.5-7.4, while DVD B is unstable at either 4.5 OR 7.4. The stability of the two DVD-Ig proteins show similar profiles, however, between the 0-200 mM histidine concentration range at pH 6 given the below conditions.

TABLE 34
Effect of pH on stability of IL12 DVD-Ig protein and DVD B

| Deg | | Time | | % | % | % |
|---|---|---|---|---|---|---|
| C. | Sample | (D) | Formulation | agg | mon | frag |
| N/A | IL12-18 | 0 | 0 mM His pH 6 + tween + sucrose | 3.08 | 95.12 | 1.8 |
| N/A | IL12-18 | 0 | 5 mM His pH 6 + tween + sucrose | 2.8 | 95.18 | 2.02 |
| N/A | IL12-18 | 0 | 10 mM His pH 6 + tween + sucrose | 2.92 | 95.06 | 2.02 |
| N/A | IL12-18 | 0 | 50 mM His pH 6 + tween + sucrose | 2.94 | 94.93 | 2.13 |
| N/A | IL12-18 | 0 | 200 mM His pH 6 + tween + sucrose | 3.11 | 94.63 | 2.25 |
| N/A | IL12-18 | 0 | 15 mM Ace pH 4.50 + tween + sucrose | 2.88 | 95.06 | 2.06 |
| N/A | IL12-18 | 0 | 15 mM His ph 6 + tween + sucrose | 2.54 | 95.4 | 2.06 |
| N/A | IL12-18 | 0 | 15 mM Pho pH 7.40 + tween + sucrose | 3.16 | 95.53 | 1.31 |
| N/A | DVDB | 0 | 0 mM His pH 6 + tween + sucrose | 3.96 | 94.2 | 1.84 |
| N/A | DVDB | 0 | 5 mM His pH 6 + tween + sucrose | 4.55 | 93.66 | 1.79 |
| N/A | DVDB | 0 | 10 mM His pH 6 + tween + sucrose | 4.61 | 93.64 | 1.75 |
| N/A | DVDB | 0 | 50 mM His pH 6 + tween + sucrose | 5.52 | 92.6 | 1.88 |
| N/A | DVDB | 0 | 200 mM His pH 6 + tween + sucrose | 4.94 | 93.24 | 1.83 |
| N/A | DVDB | 0 | 15 mM Ace pH 4.50 + tween + sucrose | 11.19 | 86.86 | 1.95 |
| N/A | DVDB | 0 | 15 mM His ph 6 + tween + sucrose | 5.92 | 92.9 | 1.18 |
| N/A | DVDB | 0 | 15 mM Pho pH 7.40 + tween + sucrose | 3.26 | 94.9 | 1.73 |
| 5 | IL12-18 | 7 | 0 mM His pH 6 + tween + sucrose | 2.82 | 94.98 | 2.19 |
| 5 | IL12-18 | 7 | 5 mM His pH 6 + tween + sucrose | 2.53 | 95.36 | 2.1 |
| 5 | IL12-18 | 7 | 10 mM His pH 6 + tween + sucrose | 2.63 | 95.9 | 1.48 |
| 5 | IL12-18 | 7 | 50 mM His pH 6 + tween + sucrose | 2.7 | 95.57 | 1.73 |
| 5 | IL12-18 | 7 | 200 mM His pH 6 + tween + sucrose | 3.03 | 95.08 | 1.9 |
| 5 | IL12-18 | 7 | 15 mM Ace pH 4.50 + tween + sucrose | 2.23 | 95.73 | 2.05 |
| 5 | IL12-18 | 7 | 15 mM His ph 6 + tween + sucrose | 2.38 | 95.46 | 2.16 |
| 5 | IL12-18 | 7 | 15 mM Pho pH 7.40 + tween + sucrose | 2.76 | 94.66 | 2.56 |
| 5 | DVDB | 7 | 0 mM His pH 6 + tween + sucrose | 4.76 | 93.43 | 1.82 |
| 5 | DVDB | 7 | 5 mM His pH 6 + tween + sucrose | 5.41 | 92.75 | 1.85 |
| 5 | DVDB | 7 | 10 mM His pH 6 + tween + sucrose | 5.15 | 93.11 | 1.74 |
| 5 | DVDB | 7 | 50 mM His pH 6 + tween + sucrose | 6.44 | 91.64 | 1.92 |
| 5 | DVDB | 7 | 200 mM His pH 6 + tween + sucrose | 5.99 | 92.27 | 1.74 |
| 5 | DVDB | 7 | 15 mM Ace pH 4.50 + tween + sucrose | 14.69 | 83.4 | 1.91 |
| 5 | DVDB | 7 | 15 mM His ph 6 + tween + sucrose | 7.97 | 90.15 | 1.87 |
| 5 | DVDB | 7 | 15 mM Pho pH 7.40 + tween + sucrose | 4.06 | 94.24 | 1.69 |
| 40 | IL12-18 | 7 | 0 mM His pH 6 + tween + sucrose | 2.56 | 95.01 | 2.44 |
| 40 | IL12-18 | 7 | 5 mM His pH 6 + tween + sucrose | 2.06 | 95.45 | 2.5 |
| 40 | IL12-18 | 7 | 10 mM His pH 6 + tween + sucrose | 1.93 | 95.59 | 2.48 |
| 40 | IL12-18 | 7 | 50 mM His pH 6 + tween + sucrose | 1.82 | 95.3 | 2.92 |
| 40 | IL12-18 | 7 | 200 mM His pH 6 + tween + sucrose | 2.18 | 95.04 | 2.78 |
| 40 | IL12-18 | 7 | 15 mM Ace pH 4.50 + tween + sucrose | 1.97 | 95.27 | 2.76 |
| 40 | IL12-18 | 7 | 15 mM His ph 6 + tween + sucrose | 1.85 | 95.61 | 2.54 |
| 40 | IL12-18 | 7 | 15 mM Pho pH 7.40 + tween + sucrose | 4.35 | 92.57 | 3.08 |
| 40 | DVDB | 7 | 0 mM His pH 6 + tween + sucrose | 44.35 | 54.38 | 1.28 |
| 40 | DVDB | 7 | 5 mM His pH 6 + tween + sucrose | 51.66 | 46.62 | 1.72 |
| 40 | DVDB | 7 | 10 mM His pH 6 + tween + sucrose | 50.76 | 47.45 | 1.79 |
| 40 | DVDB | 7 | 50 mM His pH 6 + tween + sucrose | 53.53 | 44.78 | 1.69 |
| 40 | DVDB | 7 | 200 mM His pH 6 + tween + sucrose | 43.21 | 54.95 | 1.84 |
| 40 | DVDB | 7 | 15 mM Ace pH 4.50 + tween + sucrose | 64.17 | 33.34 | 2.49 |
| 40 | DVDB | 7 | 15 mM His ph 6 + tween + sucrose | 50.51 | 46.87 | 2.5 |
| 40 | DVDB | 7 | 15 mM Pho pH 7.40 + tween + sucrose | 36.56 | 60.79 | 2.65 |
| 5 | IL12-18 | 21 | 0 mM His pH 6 + tween + sucrose | 4.01 | 91.56 | 4.43 |
| 5 | IL12-18 | 21 | 5 mM His pH 6 + tween + sucrose | 3.08 | 92.37 | 4.55 |
| 5 | IL12-18 | 21 | 10 mM His pH 6 + tween + sucrose | 2.95 | 92.69 | 4.35 |
| 5 | IL12-18 | 21 | 50 mM His pH 6 + tween + sucrose | 2.86 | 92.71 | 4.43 |
| 5 | IL12-18 | 21 | 200 mM His pH 6 + tween + sucrose | 3.28 | 92.22 | 4.5 |
| 5 | IL12-18 | 21 | 15 mM Ace pH 4.50 + tween + sucrose | 2.18 | 93.18 | 4.65 |
| 5 | IL12-18 | 21 | 15 mM His ph 6 + tween + sucrose | 2.42 | 92.36 | 5.22 |
| 5 | IL12-18 | 21 | 15 mM Pho pH 7.40 + tween + sucrose | 3.95 | 91.84 | 4.2 |
| 5 | DVDB | 21 | 0 mM His pH 6 + tween + sucrose | 5.21 | 91.17 | 3.62 |
| 5 | DVDB | 21 | 5 mM His pH 6 + tween + sucrose | 6.68 | 89.81 | 3.51 |
| 5 | DVDB | 21 | 10 mM His pH 6 + tween + sucrose | 6.26 | 89.85 | 3.89 |
| 5 | DVDB | 21 | 50 mM His pH 6 + tween + sucrose | 8.13 | 87.88 | 3.99 |
| 5 | DVDB | 21 | 200 mM His pH 6 + tween + sucrose | 8.29 | 87.66 | 4.06 |
| 5 | DVDB | 21 | 15 mM Ace pH 4.50 + tween + sucrose | 19.23 | 77.03 | 3.73 |
| 5 | DVDB | 21 | 15 mM His ph 6 + tween + sucrose | 9.54 | 86.75 | 3.71 |
| 5 | DVDB | 21 | 15 mM Pho pH 7.40 + tween + sucrose | 6.25 | 89.65 | 4.09 |
| 40 | IL12-18 | 21 | 0 mM His pH 6 + tween + sucrose | 4.09 | 90.71 | 5.19 |
| 40 | IL12-18 | 21 | 5 mM His pH 6 + tween + sucrose | 3.16 | 91.26 | 5.58 |
| 40 | IL12-18 | 21 | 10 mM His pH 6 + tween + sucrose | 3.15 | 91.78 | 5.07 |
| 40 | IL12-18 | 21 | 50 mM His pH 6 + tween + sucrose | 2.53 | 91.99 | 5.48 |
| 40 | IL12-18 | 21 | 200 mM His pH 6 + tween + sucrose | 2.88 | 91.52 | 5.61 |
| 40 | IL12-18 | 21 | 15 mM Ace pH 4.50 + tween + sucrose | 2.52 | 91.59 | 5.89 |
| 40 | IL12-18 | 21 | 15 mM His ph 6 + tween + sucrose | 2.29 | 91.97 | 5.74 |
| 40 | IL12-18 | 21 | 15 mM Pho pH 7.40 + tween + sucrose | 5.35 | 88.56 | 6.09 |
| 40 | DVDB | 21 | 0 mM His pH 6 + tween + sucrose | 55.03 | 39.46 | 5.51 |
| 40 | DVDB | 21 | 5 mM His pH 6 + tween + sucrose | 57.67 | 37.17 | 5.16 |
| 40 | DVDB | 21 | 10 mM His pH 6 + tween + sucrose | 55.56 | 39.33 | 5.11 |
| 40 | DVDB | 21 | 50 mM His pH 6 + tween + sucrose | 58.62 | 36.27 | 5.11 |
| 40 | DVDB | 21 | 200 mM His pH 6 + tween + sucrose | 56.6 | 37.74 | 5.66 |
| 40 | DVDB | 21 | 15 mM Ace pH 4.50 + tween + sucrose | 67.47 | 26.81 | 5.71 |
| 40 | DVDB | 21 | 15 mM His ph 6 + tween + sucrose | 63.14 | 31.52 | 5.34 |
| 40 | DVDB | 21 | 15 mM Pho pH 7.40 + tween + sucrose | 49.52 | 44.7 | 5.78 |

### Example 17: Effect of Citrate Concentration at pH 6 on the Shelf Stability of DVD-B and IL12IL18 DVD-Ig Proteins at 100 mg/ml

The following example describes the impact of citrate buffer concentration (ranging from 0 to 100 mM) on the shelf stability of the DVD B and IL12/IL18 DVD-Ig protein. The stability of the two DVD-Ig proteins show similar profiles between the 0-100 mM citrate at pH 6 given the tested conditions.

TABLE 35
Effect of pH on stability of DVD-B and IL12IL18 DVD proteins

| | Time | | % | % | % |
|---|---|---|---|---|---|
| Deg C. | (D) | Formulation | agg | mon | frag |
| N/A | 0 | 0 mM Cit pH 6 | 3.2 | 94.98 | 1.82 |
| N/A | 0 | 5 mM Cit pH 6 | 3.3 | 94.68 | 2.2 |
| N/A | 0 | 10 mM Cit pH 6 | 3.06 | 94.76 | 2.18 |
| N/A | 0 | 50 mM Cit pH 6 | 2.52 | 95.28 | 2.19 |
| N/A | 0 | 100 mM Cit pH 6 | 2.82 | 95.05 | 2.13 |
| N/A | 0 | 0 mM Cit pH 6 + Sucrose + tween | 2.73 | 95.11 | 2.16 |
| N/A | 0 | 5 mM Cit pH 6 + Sucrose + tween | 2.47 | 95.55 | 1.97 |
| N/A | 0 | 10 mM Cit pH 6 + | 2.67 | 95.1 | 2.23 |
| | | Sucrose + tween | | | |
| N/A | 0 | 50 mM Cit pH 6 + | 2.57 | 95.37 | 2.05 |
| | | Sucrose + tween | | | |
| N/A | 0 | 100 mM Cit pH 6 + | 2.55 | 95.24 | 2.21 |
| | | Sucrose + tween | | | |
| 4 | 7 | 0 mM Cit pH 6 | 2.91 | 94.51 | 2.58 |
| 4 | 7 | 5 mM Cit pH 6 | 3.16 | 94.76 | 2.08 |
| 4 | 7 | 10 mM Cit pH 6 | 2.77 | 94.77 | 2.46 |
| 4 | 7 | 50 mM Cit pH 6 | 3.16 | 94.67 | 2.17 |
| 4 | 7 | 100 mM Cit pH 6 | 2.84 | 95.81 | 1.36 |
| 4 | 7 | 0 mM Cit pH 6 + Sucrose + tween | 2.58 | 95.21 | 2.21 |
| 4 | 7 | 5 mM Cit pH 6 + Sucrose + tween | 2.98 | 94.84 | 2.18 |
| 4 | 7 | 10 mM Cit pH 6 + | 2.78 | 94.82 | 2.4 |
| | | Sucrose + tween | | | |
| 4 | 7 | 50 mM Cit pH 6 + | 2.87 | 94.79 | 2.35 |
| | | Sucrose + tween | | | |
| 4 | 7 | 100 mM Cit pH 6 + | 2.77 | 94.97 | 2.25 |
| | | Sucrose + tween | | | |
| 4 | 21 | 0 mM Cit pH 6 | 3.22 | 94.21 | 2.57 |
| 4 | 21 | 5 mM Cit pH 6 | 3.64 | 95.31 | 1.05 |
| 4 | 21 | 10 mM Cit pH 6 | 3.4 | 93.9 | 2.7 |
| 4 | 21 | 50 mM Cit pH 6 | 3.66 | 94.53 | 1.81 |
| 4 | 21 | 100 mM Cit pH 6 | 3.3 | 94.47 | 2.23 |
| 4 | 21 | 0 mM Cit pH 6 + Sucrose + tween | 2.93 | 94.44 | 2.68 |
| 4 | 21 | 5 mM Cit pH 6 + Sucrose + tween | 3.19 | 94.71 | 2.1 |
| 4 | 21 | 10 mM Cit pH 6 + | 3.2 | 94.54 | 2.26 |
| | | Sucrose + tween | | | |
| 4 | 21 | 50 mM Cit pH 6 + | 3.12 | 94.51 | 2.37 |
| | | Sucrose + tween | | | |
| 4 | 21 | 100 mM Cit pH 6 + | 2.88 | 94.94 | 2.19 |
| | | Sucrose + tween | | | |
| 40 | 7 | 0 mM Cit pH 6 | 3.06 | 94.81 | 2.12 |
| 40 | 7 | 5 mM Cit pH 6 | 3.4 | 94.33 | 2.27 |
| 40 | 7 | 10 mM Cit pH 6 | 3.28 | 94.47 | 2.24 |
| 40 | 7 | 50 mM Cit pH 6 | 3.81 | 94.13 | 2.06 |
| 40 | 7 | 100 mM Cit pH 6 | 3.55 | 94.07 | 2.38 |
| 40 | 7 | 0 mM Cit pH 6 + Sucrose + tween | 2.7 | 96.87 | 0.44 |
| 40 | 7 | 5 mM Cit pH 6 + Sucrose + tween | 2.84 | 94.61 | 2.55 |
| 40 | 7 | 10 mM Cit pH 6 + | 2.98 | 94.61 | 2.4 |
| | | Sucrose + tween | | | |
| 40 | 7 | 50 mM Cit pH 6 + | 2.88 | 94.84 | 2.27 |
| | | Sucrose + tween | | | |
| 40 | 7 | 100 mM Cit pH 6 + | 2.96 | 94.64 | 2.4 |
| | | Sucrose + tween | | | |
| 40 | 21 | 0 mM Cit pH 6 | 4.48 | 91.27 | 4.26 |
| 40 | 21 | 5 mM Cit pH 6 | 4.92 | 90.99 | 4.1 |
| 40 | 21 | 10 mM Cit pH 6 | 4.62 | 91.2 | 4.18 |
| 40 | 21 | 50 mM Cit pH 6 | 4.69 | 91.53 | 3.78 |
| 40 | 21 | 100 mM Cit pH 6 | 4.24 | 92.23 | 3.52 |
| 40 | 21 | 0 mM Cit pH 6 + Sucrose + tween | 3.92 | 91.87 | 4.21 |
| 40 | 21 | 5 mM Cit pH 6 + Sucrose + tween | 3.92 | 92.05 | 4.03 |
| 40 | 21 | 10 mM Cit pH 6 + | 4.01 | 91.63 | 4.36 |
| | | Sucrose + tween | | | |
| 40 | 21 | 50 mM Cit pH 6 + | 3.66 | 92.52 | 3.81 |
| | | Sucrose + tween | | | |
| 40 | 21 | 100 mM Cit pH 6 + | 3.78 | 91.89 | 4.33 |
| | | Sucrose + tween | | | |
| DVD-B | 0 | 0 mM Cit pH 6 | 2.6 | 95.63 | 1.77 |
| | 0 | 5 mM Cit pH 6 | 2.6 | 95.49 | 1.87 |
| | 0 | 10 mM Cit pH 6 | 2.41 | 95.88 | 1.72 |
| | 0 | 50 mM Cit pH 6 | 1.76 | 96.62 | 1.62 |
| | 0 | 100 mM Cit pH 6 | 1.34 | 97 | 1.66 |
| | 0 | 0 mM Cit pH 6 + Sucrose + tween | 1.54 | 97.31 | 1.15 |
| | 0 | 5 mM Cit pH 6 + Sucrose + tween | 1.52 | 96.74 | 1.73 |
| | 0 | 10 mM Cit pH 6 + | 1.5 | 96.8 | 1.71 |
| | | Sucrose + tween | | | |
| | 0 | 50 mM Cit pH 6 + | 1.5 | 96.74 | 1.75 |
| | | Sucrose + tween | | | |
| | 0 | 100 mM Cit pH 6 + | 1.11 | 97.16 | 1.73 |
| | | Sucrose + tween | | | |
| 4 | 7 | 0 mM Cit pH 6 | 2.61 | 95.54 | 1.85 |
| 4 | 7 | 5 mM Cit pH 6 | 3.01 | 95.37 | 1.62 |
| 4 | 7 | 10 mM Cit pH 6 | 3.2 | 95.12 | 1.68 |
| 4 | 7 | 50 mM Cit pH 6 | 2.85 | 95.5 | 1.64 |
| 4 | 7 | 100 mM Cit pH 6 | 2.14 | 96.22 | 1.64 |
| 4 | 7 | 0 mM Cit pH 6 + Sucrose + tween | 2.32 | 95.95 | 1.73 |
| 4 | 7 | 5 mM Cit pH 6 + Sucrose + tween | 2.86 | 95.47 | 1.66 |
| 4 | 7 | 10 mM Cit pH 6 + | 2.71 | 95.61 | 1.68 |
| | | Sucrose + tween | | | |
| 4 | 7 | 50 mM Cit pH 6 + | 2.75 | 95.62 | 1.64 |
| | | Sucrose + tween | | | |
| 4 | 7 | 100 mM Cit pH 6 + | 1.97 | 96.35 | 1.68 |
| | | Sucrose + tween | | | |
| 4 | 21 | 0 mM Cit pH 6 | 3.33 | 94.44 | 2.23 |
| 4 | 21 | 5 mM Cit pH 6 | 4.97 | 92.72 | 2.31 |
| 4 | 21 | 10 mM Cit pH 6 | 5.52 | 92.56 | 1.92 |
| 4 | 21 | 50 mM Cit pH 6 | 4.88 | 93.82 | 1.3 |
| 4 | 21 | 100 mM Cit pH 6 | 3.7 | 94.76 | 1.54 |
| 4 | 21 | 0 mM Cit pH 6 + Sucrose + tween | 3 | 95.28 | 1.72 |
| 4 | 21 | 5 mM Cit pH 6 + Sucrose + tween | 4.88 | 93.06 | 2.06 |
| 4 | 21 | 10 mM Cit pH 6 + | 5 | 93.41 | 1.59 |
| | | Sucrose + tween | | | |
| 4 | 21 | 50 mM Cit pH 6 + | 5.01 | 93.14 | 1.84 |
| | | Sucrose + tween | | | |
| 4 | 21 | 100 mM Cit pH 6 + | 3.77 | 94.38 | 1.85 |
| | | Sucrose + tween | | | |
| 40 | 7 | 0 mM Cit pH 6 | 51.75 | 46.63 | 1.62 |
| 40 | 7 | 5 mM Cit pH 6 | 58.47 | 39.22 | 2.31 |
| 40 | 7 | 10 mM Cit pH 6 | 58.39 | 38.8 | 2.8 |
| 40 | 7 | 50 mM Cit pH 6 | 47.99 | 49.69 | 2.32 |
| 40 | 7 | 100 mM Cit pH 6 | 39.78 | 58.14 | 2.09 |
| 40 | 7 | 0 mM Cit pH 6 + Sucrose + tween | 47.61 | 49.83 | 2.56 |
| 40 | 7 | 5 mM Cit pH 6 + Sucrose + tween | 56.88 | 40.48 | 2.64 |
| 40 | 7 | 10 mM Cit pH 6 + | 46.46 | 50.97 | 2.57 |
| | | Sucrose + tween | | | |
| 40 | 7 | 50 mM Cit pH 6 + | 45.57 | 52.03 | 2.4 |
| | | Sucrose + tween | | | |
| 40 | 7 | 100 mM Cit pH 6 + | 34.2 | 63.53 | 2.27 |
| | | Sucrose + tween | | | |
| 40 | 21 | 0 mM Cit pH 6 | 60.28 | 36.18 | 3.54 |
| 40 | 21 | 5 mM Cit pH 6 | | | |
| 40 | 21 | 10 mM Cit pH 6 | | | |
| 40 | 21 | 50 mM Cit pH 6 | | | |
| 40 | 21 | 100 mM Cit pH 6 | | | |
| 40 | 21 | 0 mM Cit pH 6 + Sucrose + tween | 56.73 | 39.59 | 3.67 |
| 40 | 21 | 5 mM Cit pH 6 + Sucrose + tween | 59.87 | 36.82 | 3.3 |
| 40 | 21 | 10 mM Cit pH 6 + | 53.36 | 43.59 | 3.05 |
| | | Sucrose + tween | | | |
| 40 | 21 | 50 mM Cit pH 6 + | 51.27 | 45.72 | 3.01 |
| | | Sucrose + tween | | | |
| 40 | 21 | 100 mM Cit pH 6 + | 44.11 | 52.84 | 3.05 |
| | | Sucrose + tween | | | |

### VIII. Stable Lyophilized DVD-Ig (LS-DVD-Ig) Protein Formulations

Examples 18 and 19 describe the stability of LS-DVD-Ig proteins in the lyophilized form. Example 18 describes surprising results that demonstrate freeze/thaw (F/T) stability of LS-DVD-Ig proteins. Example 19 describes studies showing stable lyophilized formulations containing LS-DVD-Ig proteins. Freezing is the first step in lyophilization and hence molecules that do not have freeze thaw stability are susceptible to instability during lyophilization.

### Example 18: Impact of Solution pH on the Stability of DVD-Ig Proteins Subjected to Repeated Freeze/Thaw Cycles

The freeze thaw behavior of DVD-Ig proteins at a protein concentration of 1 mg/ml in 5 mM citrate/5 mM phosphate buffer was evaluated by cycling the protein solution up to 2 times between the frozen state and the liquid state at pH 4, pH 6, and pH 8. Freezing was performed using temperature controlled −80° C. freezer, and thawing was performed using a 30° C. temperature controlled water bath. Samples were pulled after the second freeze/thaw (F/T) cycle and analyzed by SEC. Table 36 shows the effect of freeze/thaw processing on the amount of monomer (Mon) of remaining and the amount of fragments (Frag) and aggregates (Agg) formed in the samples formulated at these pH levels.

TABLE 36
Numbers Of DVD-Ig Protein Monomers, Aggregates, And Fragments As
Determined By SEC Before And After Repeated Freeze/Thaw Cycles Of
DVD-Ig Protein Formulations With Solution pH Values Of 4, 6, Or 8

| DVD-Ig | pH | Mon/T0 | Agg/T0 | Frag/T0 | Mon/T2 | Agg/T2 | Frag/T2 |
|---|---|---|---|---|---|---|---|
| 5 | 4 | 95.06 | 0.44 | 4.49 | 93.57 | 1.52 | 4.89 |
| 6 | 4 | 95.26 | 1.21 | 3.52 | 94.94 | 1.72 | 3.33 |
| 37 | 4 | 97.32 | 1.89 | 0.78 | 96.64 | 2.48 | 0.86 |
| 38 | 4 | 94.46 | 3.81 | 1.72 | 94.91 | 3.55 | 1.52 |
| 53 | 4 | 97.47 | 1 | 1.52 | 97.41 | 1.06 | 1.52 |
| 54 | 4 | 96.06 | 1.87 | 2.06 | 95.87 | 1.85 | 2.26 |
| 65 | 4 | 94.44 | 1.2 | 4.35 | 93.82 | 1.15 | 5.01 |
| 66 | 4 | 98.01 | 0.91 | 1.07 | 97.78 | 1.03 | 1.18 |
| 165 | 4 | 96.62 | 2.17 | 1.2 | 95.45 | 2.34 | 2.19 |
| 166 | 4 | 97.74 | 0.91 | 1.34 | 97.53 | 0.91 | 1.54 |
| 257 | 4 | 97.63 | 1.8 | 0.56 | 96.73 | 2.73 | 0.53 |
| 258 | 4 | 98.84 | 0.19 | 0.95 | 96.73 | 2.28 | 0.97 |
| 277 | 4 | 95.53 | 1.43 | 3.03 | 95.45 | 1.6 | 2.93 |
| 278 | 4 | 95.75 | 1.56 | 2.68 | 95.4 | 1.99 | 2.59 |
| 281 | 4 | 98.72 | 0.63 | 0.63 | 97.47 | 1.83 | 0.68 |
| 282 | 4 | 98.63 | 0.61 | 0.74 | 97.88 | 1.38 | 0.72 |
| 5 | 6 | 94.8 | 3.13 | 2.06 | 94.96 | 3.37 | 1.67 |
| 6 | 6 | 95.79 | 1.99 | 2.2 | 93.71 | 4.02 | 2.26 |
| 37 | 6 | 96.73 | 2.52 | 0.74 | 94.88 | 4.42 | 0.68 |
| 38 | 6 | 95.01 | 3.74 | 1.23 | 95.5 | 3.29 | 1.2 |
| 53 | 6 | 97.56 | 1 | 1.43 | 97.51 | 1.08 | 1.4 |
| 54 | 6 | 95.86 | 2.07 | 2.06 | 95.68 | 2.18 | 2.13 |
| 65 | 6 | 94.17 | 1.09 | 4.72 | 94.13 | 1.16 | 4.7 |
| 66 | 6 | 97.99 | 0.85 | 1.15 | 98.03 | 0.83 | 1.13 |
| 165 | 6 | 96.86 | 2.12 | 1.01 | 96.03 | 2.38 | 1.57 |
| 166 | 6 | 97.75 | 0.91 | 1.33 | 97.82 | 0.9 | 1.26 |
| 257 | 6 | 97.51 | 1.97 | 0.51 | 97.31 | 2.19 | 0.49 |
| 258 | 6 | 99.07 | 0.18 | 0.73 | 98.52 | 0.76 | 0.7 |
| 277 | 6 | 96.74 | 1.79 | 1.46 | 96.64 | 1.92 | 1.43 |
| 278 | 6 | 97.65 | 1.22 | 1.12 | 97.62 | 1.32 | 1.04 |
| 281 | 6 | 95.67 | 0.92 | 3.4 | 95.7 | 0.98 | 3.3 |
| 282 | 6 | 98.55 | 0.83 | 0.61 | 98.49 | 0.89 | 0.6 |
| 5 | 8 | 93.23 | 3.86 | 2.89 | 93.7 | 3.56 | 2.73 |
| 6 | 8 | 95.35 | 1.93 | 2.7 | 94.3 | 2.94 | 2.74 |
| 37 | 8 | 94.88 | 4.27 | 0.84 | 95.64 | 3.54 | 0.8 |
| 38 | 8 | 95.52 | 3.14 | 1.32 | 96.09 | 2.61 | 1.28 |
| 53 | 8 | 97.56 | 1.04 | 1.39 | 97.66 | 1.02 | 1.31 |
| 54 | 8 | 95.77 | 1.91 | 2.3 | 95.91 | 1.9 | 2.17 |
| 65 | 8 | 94.1 | 1.14 | 4.74 | 94.29 | 1.14 | 4.56 |
| 66 | 8 | 97.84 | 0.95 | 1.2 | 97.74 | 0.95 | 1.3 |
| 165 | 8 | 96.63 | 2.23 | 1.12 | 96.07 | 2.32 | 1.59 |
| 166 | 8 | 97.55 | 0.9 | 1.54 | 97.77 | 0.85 | 1.37 |
| 257 | 8 | 97.12 | 2.19 | 0.67 | 96.62 | 2.7 | 0.67 |
| 258 | 8 | 98.81 | 0.28 | 0.9 | 98.71 | 0.39 | 0.89 |
| 277 | 8 | 95.48 | 2.23 | 2.27 | 95.52 | 2.24 | 2.22 |
| 278 | 8 | 95.94 | 1.83 | 2.21 | 96.08 | 1.67 | 2.23 |
| 281 | 8 | 95.61 | 1 | 3.38 | 95.94 | 0.98 | 3.06 |
| 282 | 8 | 98.28 | 1.04 | 0.67 | 98.26 | 1.05 | 0.67 |

DVD 5, DVD 6, DVD 37, DVD 38, DVD 53, DVD 54, DVD 65, DVD 66, DVD 165, DVD 166, DVD 257, DVD 258, DVD 277, DVD 278, DVD 281, and DVD 282 demonstrated stability after being subjected to repeated freeze thaw cycles. These data indicate that DVD-Ig proteins that are formulated in a pH range of about 4 to about 8 remain stable after repeated F/T processing. The high stability of the DVD-Ig protein formulations tested (all showed greater than 93% monomer content and 11/16 formulations showed greater than 95% monomer content) was unexpected, because DVD-Ig proteins are much more complex than IgGs. Complex molecules such as DVD-Ig proteins would be expected to aggregate and fragment easily when exposed to freezing and thawing.

### Impact of Solution pH on the Stability of DVD-B Subjected to Repeated Freeze/Thaw Cycles

The freeze thaw behavior of DVD-B at a protein concentration of 2 mg/ml in 10 mM citrate/10 mM phosphate buffer was evaluated by cycling the protein solution up to 4 times between the frozen state and the liquid state at pH 4-9. Freezing was performed by means of a temperature controlled −80° C. freezer, and thawing was performed by means of a 30° C. temperature controlled water bath. Samples were pulled after each freeze/thaw (F/T) cycle and analyzed by light obscuration and SEC. Table 37 shows the effect of freeze/thaw processing on the number of sub-visible particles formed as determined using light obscuration measurements at various pH values.

TABLE 37
Numbers of Subvisible Particles Per ml as Determined by Light
Obscuration Assays After 0-4 Freeze/Thaw (F/T) Cycles of DVD-B
Formulations with a pH Value of 4, 5, 6, 7, 8, or 9

| | | | | | | |
|---|---|---|---|---|---|---|
| Number | | | | | | |
| of F/T | | | | | | |
| cycles | pH = 4 | pH = 5 | pH = 6 | pH = 7 | pH = 8 | pH = 9 |

(A) Numbers of particles ≧1 micron in size.

| | | | | | | |
|---|---|---|---|---|---|---|
| 0 | 39.16 | 55 | 44 | 41 | 55 | 46 |
| 1 | 334.5 | 1291 | 38127 | 31896 | 28444 | 25970 |
| 2 | 6728.6 | 7935 | 80064 | 61592 | 58562 | 46863 |
| 3 | 13658 | 18733 | 128448 | 95775 | 89934 | 67225 |
| 4 | 5702 | 37930 | 175024 | 132768 | 120339 | 89389 |

(B) Numbers of particles ≧10 microns in size.

| | | | | | | |
|---|---|---|---|---|---|---|
| 0 | 1.33 | 1.5 | 2.6 | 2.6 | 5.5 | 1.83 |
| 1 | 15.16 | 3.16 | 28 | 45 | 27.16 | 16.83 |
| 2 | 207.5 | 62 | 142 | 165 | 148 | 134 |
| 3 | 342 | 136 | 440 | 740 | 773 | 375 |
| 4 | 572 | 269 | 2418 | 4099 | 4537 | 1425 |

(B) Number of particles ≧25 microns in size.

| | | | | | | |
|---|---|---|---|---|---|---|
| 0 | 0.33 | 0.33 | 0.16 | 0.5 | 1.16 | 0.16 |
| 1 | 3.3 | 0.5 | 3.83 | 2 | 4.33 | 1.33 |
| 2 | 40.16 | 10.83 | 2.33 | 6.33 | 1.16 | 3.16 |
| 3 | 71.16 | 6.67 | 13.5 | 39.83 | 39.33 | 5.33 |
| 4 | 118.83 | 67.63 | 198.67 | 476.16 | 641.5 | 142.83 |

The results of the light obscuration assays show that the numbers of particles formed by DVD-B formulations with a pH of 4 to 9 was low. The numbers of particles formed increased with increasing pH and were at a maximum at around the pI of the molecule (pI 8.5). However, with only one exception, the protein solutions tested satisfied the requirements of the International Conference on Harmonization of Technical Requirements for Registration of Pharmaceuticals for Human Use (ICH) guidelines, which requires less than 600 particles of size 25 microns or higher per ml.

Table 38 shows SEC measurements of the stability of DVD-B after freeze/thaw processing. These measurements include the percentage of monomers, aggregates, and fragments, as well as the area under the curve (AUC).

TABLE 38
Stability of DVD-B as Determined By SEC After 0-4 Freeze/Thaw
Cycles of DVD-B Formulations Wwth Solution pH Values
of 4, 5, 6, 7, 8, or 9

| | | | |
|---|---|---|---|
| Monomer | Aggregate | Fragment | |
| (%) | (%) | (%) | AUC |

(A) SEC measurements of solutions not subjected to F/T cycles

| | | | | |
|---|---|---|---|---|
| pH 4 Vial 1 | 96.38 | 1.44 | 2.16 | 76861 |
| pH 4 Vial 2 | 95.61 | 1.56 | 2.82 | 76123 |
| pH 4 Mean | 95.995 | 1.5 | 2.49 | 76492 |
| pH 5 Vial 1 | 96.1 | 1.57 | 2.32 | 73704 |
| pH 5 Vial 2 | 96.16 | 1.55 | 2.28 | 74196 |
| pH 5 Mean | 96.13 | 1.56 | 2.3 | 73950 |
| pH 6 Vial 2 | 95.09 | 1.69 | 3.2 | 77475 |
| pH 7 Vial 2 | 95.61 | 1.81 | 2.56 | 75863 |
| pH 8 Vial 2 | 95.84 | 1.87 | 2.27 | 74943 |
| pH 9 Vial 1 | 95.66 | 2.11 | 2.21 | 82103 |
| pH 9 Vial 2 | 95.43 | 2.14 | 2.41 | 81843 |
| pH 9 Mean | 95.545 | 2.125 | 2.31 | 81973 |

(B) SEC measurements after one F/T cycle

| | | | | |
|---|---|---|---|---|
| pH 4 Vial 1 | 96.37 | 1.68 | 1.94 | 74694 |
| pH 4 Vial 2 | 95.39 | 1.74 | 2.86 | 76213 |
| pH 4 Mean | 95.88 | 1.71 | 2.4 | 75453.5 |
| pH 5 Vial 1 | 96.18 | 1.82 | 1.98 | 71511 |
| pH 5 Vial 2 | 96.08 | 1.6 | 2.31 | 76345 |
| pH 5 Mean | 96.13 | 1.71 | 2.145 | 73928 |
| pH 6 Vial 1 | 96.37 | 1.6 | 2.02 | 75448 |
| pH 6 Vial 2 | 95.24 | 1.6 | 3.14 | 77009 |
| pH 6 Mean | 95.805 | 1.6 | 2.58 | 76228.5 |
| pH 7 Vial 1 | 95.88 | 1.86 | 2.24 | 75821 |
| pH 7 Vial 2 | 95.28 | 1.96 | 2.74 | 76470 |
| pH 7 Mean | 95.58 | 1.91 | 2.49 | 76145.5 |
| pH 8 Vial 1 | 95.64 | 2.01 | 2.34 | 74757 |
| pH 8 Vial 2 | 95.59 | 2.08 | 2.31 | 74270 |
| pH 8 Mean | 95.81536 | 1.777143 | 2.3925 | 75306.68 |
| pH 9 Vial 1 | 95.62 | 2.15 | 2.22 | 81981 |
| pH 9 Vial 2 | 95.57 | 2.13 | 2.29 | 81516 |
| pH 9 Mean | 95.595 | 2.14 | 2.255 | 81748.5 |

(C) SEC measurements after two F/T cycles

| | | | | |
|---|---|---|---|---|
| pH 4 Vial 1 | 95.75 | 1.98 | 2.25 | 75402 |
| pH 4 Vial 2 | 95.29 | 1.98 | 2.71 | 74567 |
| pH 4 Mean | 95.52 | 1.98 | 2.48 | 74984.5 |
| pH 5 Vial 1 | 96.07 | 1.85 | 2.06 | 71454 |
| pH 5 Vial 2 | 95.92 | 1.88 | 2.19 | 71525 |
| pH 5 Mean | 95.995 | 1.865 | 2.125 | 71489.5 |
| pH 6 Vial 1 | 96.33 | 1.58 | 2.08 | 74666 |
| pH 6 Vial 2 | 95.43 | 1.62 | 2.93 | 75080 |
| pH 6 Mean | 95.88 | 1.6 | 2.505 | 74873 |
| pH 7 Vial 1 | 95.77 | 1.87 | 2.34 | 74813 |
| pH 7 Vial 2 | 95.72 | 1.84 | 2.42 | 74584 |
| pH 7 Mean | 95.745 | 1.855 | 2.38 | 74698.5 |
| pH 8 Vial 1 | 95.72 | 2.02 | 2.24 | 73460 |
| pH 8 Vial 2 | 95.78 | 2 | 2.21 | 68769 |
| pH 8 Mean | 95.75 | 2.01 | 2.225 | 71114.5 |
| pH 9 Vial 1 | 95.58 | 2.1 | 2.3 | 73356 |
| pH 9 Vial 2 | 95.57 | 2.12 | 2.3 | 77337 |
| pH 9 Mean | 95.575 | 2.11 | 2.3 | 75346.5 |

(D) SEC measurements after three F/T cycles

| | | | | |
|---|---|---|---|---|
| pH 4 Vial 1 | 95.62 | 2.14 | 2.22 | 72539 |
| pH 4 Vial 2 | 95.01 | 2.23 | 2.75 | 72480 |
| pH 4 Mean | 95.315 | 2.185 | 2.485 | 72509.5 |
| pH 5 Vial 1 | 95.9 | 1.99 | 2.09 | 68689 |
| pH 5 Vial 2 | 95.8 | 1.98 | 2.21 | 65381 |
| pH 5 Mean | 95.85 | 1.985 | 2.15 | 67035 |
| pH 6 Vial 2 | 95.55 | 1.63 | 2.81 | 71195 |
| pH 7 Vial 1 | 96.03 | 1.84 | 2.12 | 70617 |
| pH 7 Vial 2 | 95.77 | 1.86 | 2.35 | 71405 |
| pH 7 Mean | 95.9 | 1.85 | 2.235 | 71011 |
| pH 8 Vial 1 | 95.86 | 2.04 | 2.08 | 69364 |
| pH 8 Vial 2 | 95.59 | 2.12 | 2.28 | 69308 |
| pH 8 Mean | 95.725 | 2.08 | 2.18 | 69336 |
| pH 9 Vial 1 | 95.03 | 2.42 | 2.54 | 77679 |
| pH 9 Vial 2 | 95.59 | 2.22 | 2.18 | 76113 |
| pH 9 Mean | 95.31 | 2.32 | 2.36 | 76896 |

(E) SEC measurements after four F/T cycles

| | | | | |
|---|---|---|---|---|
| pH 4 Vial 1 | 94.38 | 2.53 | 3.08 | 53902 |
| pH 4 Vial 2 | 94.08 | 2.4 | 3.5 | 72943 |
| pH 4 Mean | 94.23 | 2.465 | 3.29 | 63422.5 |
| pH 5 Vial 1 | 96.01 | 1.88 | 2.1 | 66962 |
| pH 5 Vial 2 | 95.85 | 1.96 | 2.18 | 67786 |
| pH 5 Mean | 95.93 | 1.92 | 2.14 | 67374 |
| pH 6 Vial 1 | 96.26 | 1.62 | 2.11 | 70371 |
| pH 6 Vial 2 | 95.41 | 1.67 | 2.9 | 71086 |
| pH 6 Mean | 95.835 | 1.645 | 2.505 | 70728.5 |
| pH 7 Vial 1 | 95.87 | 1.82 | 2.29 | 67317 |
| pH 7 Vial 2 | 96.03 | 1.81 | 2.15 | 67869 |
| pH 7 Mean | 95.95 | 1.815 | 2.22 | 67593 |
| pH 8 Vial 1 | 95.62 | 2.07 | 2.3 | 69629 |
| pH 8 Vial 2 | 95.31 | 2.15 | 2.52 | 69401 |
| pH 8 Mean | 95.465 | 2.11 | 2.41 | 69515 |
| pH 9 Vial 1 | 95.44 | 2.27 | 2.28 | 77495 |
| pH 9 Vial 2 | 95.58 | 2.18 | 2.22 | 73373 |
| pH 9 Mean | 95.51 | 2.225 | 2.25 | 75434 |

The results in Table 38 indicate that the DVD-Ig protein does not form significant aggregates even after 4 F/T cycles. The good F/T stability is surprising as it was anticipated that the stability might not be as good as observed.

### Example 19: Impact of Lyophilization on the Stability of DVD-Ig Proteins

Aggregates may form during the process of lyophilization as well as later on during shelf stability of the solid protein. The aggregates formed during lyophilization are generally measured following immediate reconstitution.

Storage stability of DVD-Ig proteins was evaluated for prolonged periods of time at controlled temperature conditions. After defined storage periods, samples were pulled and the impact of storage time and storage temperature on the stability of lyophilized DVD-Ig proteins was evaluated by size exclusion chromatography (SEC) and ion exchange chromatography (IEC). Three DVD-Ig proteins were studied: DVD-B (TNF-PGE2), DVD-A (TNF-IL17), and DVD-C (IL1α/IL1β). Of the three, DVD-A and DVD-C are AS-DVD-Ig proteins. DVD-B is a non-AS-DVD-Ig protein, but was identified as an LS-DVD-Ig protein as it (as well as LS-DVD-Ig proteins DVD-A and DVD-C) was found to be stable in a lyophilized formulation. The formulations were lyophilized in solutions as shown in Table 39 in a formulation containing a buffer, a polyol, and a surfactant.

TABLE 39
Composition of Lyophilized Formulations

| | DVD-A | | DVD-B | | DVD-C | |
|---|---|---|---|---|---|---|
| Component | 55 mg/ml | | 55 mg/ml | | 48 mg/ml | |
| Histidine | 2.33 | (15 mM) | 2.33 | (15 mM) | 2.33 | (15 mM) |
| Sucrose | 46 | (4.6%) | 46 | (4.6%) | 46 | (4.6%) |
| Polysorbate | 0.2 | (0.02%) | 0.2 | (0.02%) | 0.1 | (0.01%) |
| 80 | | | | | | |
| 0.1M HCl | q.s. | (pH 5.25) | q.s. | (pH 5.25) | q.s. | (pH 6.0) |

Table 40 describes the percentages of monomers, aggregates, and fragments that were measured using SEC before storage (T0) or following storage of the lyophilized formulations at either 5° C. or 40° C. for the given storage periods.

TABLE 40
Stability Of Stored Lyophilized Formulations As Assessed Using SEC

| DVD-Ig | Storage Condition | Monomer | Aggregate | Fragment |
|---|---|---|---|---|
| DVD A | T0 | 94.22 | 5.09 | 0.67 |
| | 1 Week 40° C. | 94.12 | 5.23 | 0.64 |
| | 4 Week 40° C. | 94.41 | 5.14 | 0.43 |
| | 3 m 40 C. | 93.31 | 6.24 | 0.43 |
| | 3 m 40 C. + 3 h RT | 93.17 | 6.38 | 0.43 |
| DVD B | T0 | 96.59 | 2.11 | 1.28 |
| | 1 Week 40° C. | 96.11 | 2.59 | 1.28 |
| | 4 Week 40° C. | 95.44 | 3.19 | 1.36 |
| | 4 Week 5° C. | 96.47 | 2.2 | 1.31 |
| | 4 Week 40° C. + 3 h RT | 95.35 | 3.29 | 1.34 |
| DVD C | T0 | 96.55 | 2.94 | 0.5 |
| | 1 Week 40° C. | 97.165 | 1.59 | 1.23 |
| | 1 m 40° C. | 96.88 | 1.93 | 1.18 |
| | 3 m 5° C. | 97.42 | 1.36 | 1.21 |

The SEC data in Table 40 shows that lyophilized LS-DVD-Ig proteins remain stable for periods of up to 3 months of storage because they show high percentages of monomers and low percentages of aggregates and fragments. After accelerated storage for 3 months at 40° C. and 3 hours of reconstitution time, lyophilized DVD-A had more than 93% monomers, less than 6.4% aggregates, and only about 0.4% fragments. After 4 weeks of accelerated storage at 40° C., lyophilized DVD B had more than 95% monomers and only about 3.2% aggregates and about 1.4% fragments. After 1 month of accelerated storage at 40° C., lyophilized DVD C had approximately 97% monomers, 2% aggregates, and 1% fragments. Thus, lyophilized formulations of DVD-A, DVD-B, and DVD-C showed long term stability, as assessed by SEC.

Table 41 below provides data regarding the stability of stored formulations measured using IEC. The impact of chemical stability was not significant as observed from formation of acidic and basic species with time.

TABLE 41
Stability Of Stored Lyophilized Formulations As Assessed Using IEC

| DVD-Ig | Storage condition | Main | Acidic | Basic | X |
|---|---|---|---|---|---|
| DVD A | T0 | 73.28 | 3.6 | 20.78 | 2.32 |
| | 1 Week 40° C. | 72.59 | 3.38 | 21.91 | 2.1 |
| | 4 Week 40° C. | 70.73 | 3.6 | 23.44 | 2.21 |
| | 3 m 40° C. | 71.75 | 3.84 | 22 | 2.39 |
| | 3 m 40° C. + 3 h RT | 71.8 | 3.71 | 22.03 | 2.44 |
| DVD B | T0 | 48.84 | 38.54 | 12.6 | |
| | 1 Week 40° C. | 48.18 | 38.72 | 13.09 | |
| | 4 Week 40° C. | 45.87 | 37.6 | 16.53 | |
| | 4 Week 5° C. | 48.17 | 38.16 | 13.56 | |
| | 4 Week 40° C. + 3 h RT | 45.7 | 37.79 | 16.5 | |
| DVD C | 1 Week 40° C. | 54.82 | 28.82 | 16.35 | |
| | 1 m 40° C. | 53.61 | 28.84 | 17.53 | |
| | 3 m 5° C. | 55.43 | 28.57 | 15.99 | |

Table 42 below provides the reconstitution times of the stored lyophilized formulations.

TABLE 42
Reconstitution Time (RT) Of The Lyophilized Formulations
(With Water For Injection)

| | Storage | |
|---|---|---|
| DVD-Ig | condition | RT |
| DVD-A | T0 | 30 s |
| | T1 week 40° C. | 26 s |
| | T4 week 40° C. | 48 s |
| | T3 month 40° C. | 300 s |
| DVD-B | T0 | 48 s |
| | T1 week 40° C. | 60 s |
| | T4 week 5° C. | 69 s |
| | T4 week 40° C. | 89 s |
| DVD-C | T0 | 47 s |
| | T1 week 40° C. | 76 s |
| | 3 m 5° C. | 62 s |
| | 1 m 40° C. | 53 s |

The above experiments show that AS-DVD-Ig proteins (e.g., DVDs A & C) can be formulated as a stable lyophilized formulation. Moreover, the above examples show that LS-DVD-Ig proteins—which are not stable in liquid formulations—can be stabilized using lyophilization (e.g., DVD-B).

### IX. Characterization of Dvd-Ig Proteins

Examples 20-23 provide further characterization of DVD-Ig proteins generally.

### Example 20: Solubility Assessment of DVD-Ig Proteins Using PEG 3000

Polyethylene glycols (PEG) are often used as crowders to assess the true solubility of a protein by utilizing micro amounts of the protein material available at early stages of development. In general, the greater the amount of PEG is required to induce precipitation, the greater is the anticipated true solubility of the protein in solution.

The following studies were carried out using small aliquots of PEG solution (50% w/v) added to a stock solution of protein (0.5 mg/ml) in a buffer (5 mM citrate and 5 mM phosphate) at pH 6. Table 43 shows the data for various DVD-Ig proteins.

TABLE 43
Amount of PEG 3000 Required To Induce Precipitation
in a 0.5 mg/ml Protein Solution of DVD-Ig Proteins

| | % PEG 3000 required to induce |
|---|---|
| DVD | precipitation |
| 5 | 10 |
| 6 | 9.37 |
| 37 | 10 |
| 38 | 9.33 |
| 53 | 8.12 |
| 54 | 9.37 |
| 65 | 7.5 |
| 66 | 7.5 |
| 165 | 10.625 |
| 166 | 11.875 |
| 257 | 9.37 |
| 258 | 10.62 |
| 277 | 12.5 |
| 278 | 13.75 |
| 281 | 8.12 |
| 282 | 7.5 |

These data show that the amount of PEG 3000 required to induce precipitation of DVD-Ig proteins is typical of highly soluble proteins (i.e., those proteins with a true solubility that exceeds about 100 mg/ml). Although the PEG precipitation assay is a standard assay in antibody formulation assessment to provide information about its solubility, it would not be sufficient to predict whether a DVD-Ig protein would be classified as AS or LS or even non LS, indicating the complexity of DVD-Ig proteins and the challenging formulation efforts compared to monoclonal antibodies.

### Example 21: Assessment of the Tertiary Structure of DVD-Ig Proteins Using Near UV CD Scans

The structure of a protein is one of the important factors influencing protein stability during accelerated/long-term storage of protein liquid and lyophilizate formulations. To assess the tertiary structure of the DVD-Ig proteins, near UV CD scan between 250-320 nm was taken on a Jasco Spectrometer with a scan rate of 50 nm/minute at a concentration of 1 mg/ml. An average of 3 scans was taken. The pH used in the study was 6 in 5 mM citrate and 5 mM phosphate conditions. The data presented in Table 44 show that DVD-Ig proteins behave like typical proteins and have a compact folded structure as indicated by significant ellipticity values in 250-320 nm region.

TABLE 44
The Molar Ellipticity in the DVD-Ig Proteins as Measured Using Near UV-CD
Scans Between 250-320 nm Wavelengths

| nm | DVD 5 | DVD 6 | DVD 277 | DVD 278 | DVD 37 | DVD 38 | DVD 165 | DVD 166 |
|---|---|---|---|---|---|---|---|---|
| 320 | 6646.96 | 12702.62 | 3579.666 | 7019.56 | 1218.66 | 1993.26 | 8962.574 | 8114.95 |
| 319 | 7257.04 | 13016.1 | 3662.344 | 6814.7 | 1114.54 | 2078.8 | 9362.45 | 8886.423 |
| 318 | 8145.62 | 14541.52 | 3703.052 | 7513.72 | 872.54 | 2880.56 | 9776.938 | 9277.928 |
| 317 | 9476.46 | 15963.24 | 5070.026 | 7760.452 | 1336.88 | 3421.96 | 10099.72 | 9691.596 |
| 316 | 10185.44 | 16554.02 | 6054.796 | 8935.96 | 2367.68 | 4078.7 | 10729.59 | 10356.54 |
| 315 | 10023.58 | 16536.34 | 6630.815 | 9014.7 | 2548.6 | 3955.04 | 11502.31 | 10968.25 |
| 314 | 10201.96 | 17074.34 | 7202.642 | 9529.62 | 3030.4 | 4855.16 | 12033.56 | 11110.03 |
| 313 | 10907.96 | 18696.88 | 8422.852 | 10622 | 4451.1 | 5342.32 | 12571.84 | 11358.8 |
| 312 | 11824.58 | 19171.1 | 9895.56 | 11913.48 | 4592.9 | 5297.5 | 13507.2 | 12394.13 |
| 311 | 11466.66 | 19059.96 | 10537.74 | 12208.92 | 4720.92 | 5899.76 | 14406.2 | 12631.19 |
| 310 | 11744.26 | 18846.86 | 10694.84 | 12812.48 | 5136.1 | 6597.96 | 15369.48 | 13385.52 |
| 309 | 13150.56 | 20155.96 | 11727.68 | 14628.08 | 5037.88 | 7022.1 | 15918.68 | 13729.84 |
| 308 | 14363.9 | 21871.58 | 13669.32 | 17431.1 | 4898.26 | 7299.36 | 17442.24 | 14465.36 |
| 307 | 14827 | 22449.48 | 15667.88 | 19641.7 | 4348.52 | 6938.18 | 19270.78 | 16158.3 |
| 306 | 15471.46 | 23590.74 | 18448.36 | 23073.2 | 3629.04 | 6811.68 | 21370.84 | 17372.78 |
| 305 | 16128.78 | 24474.14 | 21235.5 | 26813.58 | 2970.78 | 6507.94 | 22937.42 | 18230.84 |
| 304 | 17206.48 | 25912.58 | 24931.98 | 31588.34 | 2601.04 | 6025.44 | 25089.44 | 19314.74 |
| 303 | 17845 | 27053.6 | 29550.96 | 36070.68 | 2684.84 | 5982.68 | 27123.34 | 20000.74 |
| 302 | 18582.12 | 28474.04 | 34999.28 | 41602.88 | 2491.08 | 6500.46 | 30155.74 | 21436.76 |
| 301 | 18984.36 | 29238.66 | 40003.1 | 46542.9 | 3016.84 | 6677.68 | 33531.26 | 22943.42 |
| 300 | 19031.22 | 30795.64 | 44775.24 | 52465.24 | 4192.14 | 7084.12 | 35916.14 | 24073.72 |
| 299 | 18842.08 | 32232.86 | 50531.26 | 58830.46 | 5956.78 | 8385.58 | 39545.44 | 26330.24 |
| 298 | 18310.74 | 34756.84 | 57637.64 | 65440.24 | 7992.56 | 11147.22 | 44240.34 | 29677.12 |
| 297 | 17585.48 | 36848.34 | 65571.74 | 72241.54 | 11037.96 | 13048.21 | 48393.4 | 33419.82 |
| 296 | 13893.14 | 38010.8 | 73072.8 | 79715.4 | 15370.88 | 16061.57 | 52048.38 | 36807.78 |
| 295 | 9789.944 | 37360.08 | 80952.28 | 85191.48 | 19624.23 | 20546.68 | 55017.5 | 39923.5 |
| 294 | 5165.96 | 32790.36 | 89652.96 | 91526.56 | 23669.8 | 26554.56 | 57618.44 | 42225.64 |
| 293 | 1413.2 | 28288.2 | 95485.58 | 95237.78 | 30018.18 | 33581.92 | 59047.16 | 41557.16 |
| 292 | −1473.62 | 22147.68 | 99449.54 | 97813.74 | 34746.56 | 37286.72 | 58765.26 | 37964.46 |
| 291 | −6285.46 | 14384.6 | 101128.1 | 97343.9 | 37414.96 | 40375.22 | 53508.98 | 30940.04 |
| 290 | −13331 | 4297.82 | 101313.8 | 98881.4 | 37024.44 | 39976.88 | 45411.26 | 20001.54 |
| 289 | −21797.7 | −10266.9 | 101312.5 | 99402.86 | 35548.08 | 40677.26 | 36623.7 | 7331.56 |
| 288 | −34293.6 | −22215.8 | 101720 | 100768.6 | 35980.3 | 41280.4 | 24626.54 | −7606.72 |
| 287 | −47656.4 | −33279.2 | 101605 | 98687.64 | 34745.48 | 38341.86 | 11517.6 | −24343.7 |
| 286 | −61219.6 | −48062.2 | 97134.44 | 95381.84 | 27860.52 | 34932.12 | −2030.06 | −40331.5 |
| 285 | −75948.8 | −60752.6 | 93683 | 89838.6 | 21697.61 | 28696.44 | −15666.6 | −53909.6 |
| 284 | 85313.8 | −71219.4 | 86396.76 | 84616.16 | 18783.44 | 24280 | −27030.4 | −65730.2 |
| 283 | −91152 | −80851.2 | 78436.8 | 76356.4 | 9704.06 | 19689.84 | −36635.3 | −76580.9 |
| 282 | −95404.9 | −85162.3 | 71201.54 | 70599.14 | 5073.98 | 12646.96 | −44218.6 | −82657.4 |
| 281 | −97387.2 | −94678.8 | 62561.02 | 64625.02 | −1342.2 | 8017.08 | −47641.5 | −88644.3 |
| 280 | −98015.6 | −98723.8 | 56119.6 | 59977 | −10239 | −494.72 | −51641.2 | −89547.6 |
| 279 | −98171 | −100371 | 48712.98 | 59357.38 | −16922.6 | −6910.34 | −59722.5 | −93925.5 |
| 278 | −97897.4 | −95720.8 | 44356.64 | 56008.04 | −24324.6 | −14277.2 | −63928.8 | −98962.6 |
| 277 | −96345.1 | −99145.7 | 41782.1 | 52264.5 | −30115.4 | −21714.5 | −65494.3 | −98432.5 |
| 276 | −95102.1 | −96707.1 | 38121.48 | 48419.48 | −35745 | −27709.8 | −62437.4 | −97937 |
| 275 | −89955.9 | −91842.9 | 33190.2 | 40597.08 | −42966 | −33181.1 | −62363.9 | −97001.5 |
| 274 | −84500 | −85969.2 | 29553.56 | 35615.88 | −49853.4 | −42771.6 | −63464.5 | −94323.9 |
| 273 | −78084 | −82791.6 | 24659.58 | 26926.88 | −51578.2 | −50168.9 | −58524 | −92889.2 |
| 272 | −72453.3 | −81793.5 | 18882.96 | 20533.34 | −55669.2 | −53055 | −52516 | −88294 |
| 271 | −72395.7 | −84665.9 | 12213.68 | 12167.9 | −62527.2 | −54026.8 | −51326.4 | −86186.2 |
| 270 | −72518.3 | −82144.3 | 5656.34 | 3573.48 | −68542 | −59996.1 | −50200.6 | −82118.2 |
| 269 | −71980.1 | −81188.5 | 171.28 | −2996.52 | −71329.6 | −66527.5 | −49773.5 | −81328.3 |
| 268 | −70389.7 | −79549.9 | −5232.12 | −6204.78 | −73950.2 | −67613.7 | −45568.7 | −76447.9 |
| 267 | −70817 | −78538.2 | −9550.04 | −11210.2 | −76784 | −68726.9 | −43001.8 | −71633.2 |
| 266 | −73340.7 | −78434.3 | −15807.9 | −19018.3 | −81063.8 | −70561.2 | −41993.4 | −68706.8 |
| 265 | −75748.4 | −78150 | −21737.8 | −24832 | −85226.4 | −75652.6 | −41798.7 | −65622.9 |
| 264 | −77113.2 | −80323.4 | −26482.6 | −29822.6 | −86541.6 | −79375.1 | −42935.3 | −64380.3 |
| 263 | −78792.5 | −81903.9 | −30602.5 | −36746.1 | −87244.6 | −81964.6 | −43580.6 | −63965.2 |
| 262 | −81250 | −84495.6 | −37192.2 | −43633.6 | −89128.6 | −84559.9 | −45277.3 | −62478.7 |
| 261 | −83776 | −89228.6 | −44442 | −52471 | −92083.6 | −89257.2 | −46914 | −64373.6 |
| 260 | −86573.9 | −90496.5 | −51107.5 | −58915.1 | −95913.6 | −93213.7 | −50369.2 | −66308.6 |
| 259 | −89539.5 | −93126.1 | −56651.5 | −64341.1 | −97884.6 | −95995.8 | −52277.8 | −66381.4 |
| 258 | −92094.6 | −94878.8 | −62516 | −71983.4 | −98007.4 | −100374 | −52138.4 | −66466.4 |
| 257 | −95753.5 | −94389.1 | −69933.5 | −79216.5 | −99840.8 | −104921 | −53414.7 | −66988.1 |
| 256 | −99615.9 | −96922.3 | −77456.3 | −86799.7 | −106350 | −108982 | −57438.5 | −68269.9 |
| 255 | −104801 | −100722 | −85586.1 | −96349.5 | −112184 | −117285 | −61096.9 | −70194.1 |
| 254 | −111700 | −105908 | −95539.9 | −107715 | −119943 | −126255 | −65197.9 | −72945.7 |
| 253 | −120346 | −111647 | −107838 | −121118 | −126564 | −135675 | −69785 | −74422.6 |
| 252 | −128547 | −116853 | −121815 | −136346 | −136939 | −147233 | −72734.7 | −76593.5 |
| 251 | −138320 | −122715 | −136158 | −151471 | −148183 | −159097 | −76760.8 | −78565.4 |
| 250 | −147423 | −130921 | −153967 | −171349 | −161249 | −173403 | −80181.9 | −80184.1 |
| | 281 | 257 | 258 | 53 | 54 | 65 | 66 | 815 |
| 320 | 12065.92 | 7846.8 | 4097.34 | 4826.54 | 6066.984 | 6067.38 | 9911.934 | 3779.02 |
| 319 | 11847.82 | 7428.24 | 4515.66 | 5556.22 | 7281.7 | 6441.22 | 10218.96 | 4006.72 |
| 318 | 12332.27 | 7805.58 | 4861.12 | 5504.22 | 8071.448 | 6933.64 | 10214.58 | 4494.75 |
| 317 | 12776.91 | 8061.44 | 5074.58 | 5732.32 | 8479.016 | 7348.178 | 10528.19 | 4202.59 |
| 316 | 14142.84 | 8717.78 | 5061.64 | 6523.062 | 9475.612 | 7938.938 | 10988.44 | 4956.7 |
| 315 | 14694.82 | 8798.74 | 5287.42 | 7192.17 | 9958.128 | 8313.312 | 11549.55 | 5549.21 |
| 314 | 14830.16 | 9401.84 | 5649.84 | 8427.526 | 11201.14 | 9177.416 | 12169.2 | 5680.91 |
| 313 | 15740.5 | 10036.5 | 5513.18 | 9076.294 | 11607.36 | 9765.546 | 12648.6 | 6614.77 |
| 312 | 16600.48 | 10508.9 | 5269.74 | 9473.708 | 11558.12 | 11029.02 | 13622.86 | 7529.12 |
| 311 | 17947.58 | 11145.36 | 5854.36 | 11364.37 | 12821.78 | 11602.19 | 14700.16 | 8409.46 |
| 310 | 18647.18 | 11330.56 | 6760.9 | 12940.84 | 13897.06 | 12955.48 | 15438.38 | 9805.26 |
| 309 | 19051.76 | 11874.96 | 7845.46 | 14378.18 | 15412.24 | 13794.56 | 16100.42 | 11138 |
| 308 | 20194.04 | 12901.68 | 9610.94 | 16243.26 | 16708.44 | 15924.24 | 17673.84 | 12709.3 |
| 307 | 21501.32 | 13864.89 | 10389.71 | 18312.92 | 18567.96 | 18969.52 | 19281.56 | 14124 |
| 306 | 22558.46 | 15973.03 | 12652.35 | 20956.14 | 20580.94 | 21496.98 | 22119.52 | 16104.4 |
| 305 | 22955.58 | 18458.64 | 14649.16 | 22956.36 | 22654.68 | 24478.68 | 24957.06 | 17267.1 |
| 304 | 24254.16 | 22225.84 | 17539.24 | 24916.7 | 24910.7 | 28411.44 | 28375.96 | 19980.3 |
| 303 | 25917.86 | 26687.28 | 21594.86 | 28379.08 | 27466.24 | 32104.42 | 32272.3 | 22093.1 |
| 302 | 27687.84 | 31790.54 | 27106.9 | 32466.3 | 30839.18 | 37592.06 | 36915.46 | 24879.4 |
| 301 | 29697.44 | 39105.14 | 33168.46 | 36775.26 | 34289.26 | 42282.94 | 42093.54 | 28566.4 |
| 300 | 32197.92 | 45824.8 | 40252.22 | 41240.42 | 37560.02 | 47196.54 | 47075.94 | 32197.8 |
| 299 | 35759.08 | 53517.4 | 48973.14 | 47424.54 | 42753.74 | 53413.44 | 51815.84 | 34630.8 |
| 298 | 38934.66 | 61028.66 | 59047.34 | 54381.74 | 47954.54 | 60335.34 | 57517.74 | 39145.6 |
| 297 | 42362.58 | 66998.58 | 67954.48 | 62451.48 | 53272.08 | 66398.8 | 63756.6 | 44072.7 |
| 296 | 43671.94 | 74110.54 | 75373.74 | 70486.14 | 61093.74 | 73427.38 | 68884.18 | 49457.2 |
| 295 | 44165.58 | 78735.78 | 82410.6 | 79100.2 | 67392.6 | 79811.3 | 74325.1 | 56396.6 |
| 294 | 42989.48 | 81406.48 | 87185.94 | 87125.34 | 73529.94 | 84681.04 | 77364.84 | 63522.8 |
| 293 | 39734.86 | 86632.46 | 92185.18 | 92039.38 | 75942.38 | 85114.56 | 77161.56 | 69005.4 |
| 292 | 32115.3 | 89147.34 | 94161.72 | 92089.92 | 73611.32 | 83679.86 | 74889.26 | 73476 |
| 291 | 23538.46 | 91692.6 | 94672.62 | 90680.62 | 69166.82 | 80907.98 | 71316.78 | 75368.5 |
| 290 | 12161.46 | 93798.4 | 94973.48 | 86154.68 | 64144.68 | 78362.66 | 68979.86 | 73182.4 |
| 289 | 1548.98 | 93691.6 | 96476.46 | 82341.46 | 60768.66 | 74821.3 | 65818.7 | 71181.6 |
| 288 | −10437.2 | 93939.4 | 94696.2 | 76175.6 | 51123 | 72930.08 | 64672.28 | 66552.3 |
| 287 | −21710.4 | 94069.8 | 92780.26 | 69455.66 | 40023.06 | 70477.3 | 58525.1 | 61655.8 |
| 286 | −36417.6 | 88864.2 | 89536.32 | 60131.32 | 29824.24 | 64280.94 | 49373.74 | 54754.7 |
| 285 | −52204 | 80893.2 | 85038.06 | 52439.46 | 17512.66 | 56298.78 | 42601.38 | 50227.7 |
| 284 | −66884.8 | 72805.4 | 80913.72 | 41558.12 | 3370.34 | 47594.4 | 33624.6 | 45166.8 |
| 283 | −80916 | 68373 | 73927.04 | 29410.2 | −15694.6 | 36812.1 | 22703.88 | 40688.7 |
| 282 | −94698.6 | 60374.2 | 66493.5 | 18565.32 | −31586.9 | 27868.5 | 11283.46 | 35191.8 |
| 281 | −105389 | 52412 | 60397.46 | 6757.02 | −40835.5 | 15774.47 | 1931.42 | 29508.8 |
| 280 | −114357 | 42127.8 | 54220.78 | −927.72 | −49228.4 | 10231.36 | −664.46 | 24412.9 |
| 279 | −118830 | 35497.18 | 48557.46 | −6687.94 | −54286.9 | 8101.54 | −3721.12 | 21517.8 |
| 278 | −124337 | 28584.7 | 41693.96 | −13671.6 | −57605.2 | 3735.14 | −6633.18 | 17817.7 |
| 277 | −125581 | 21975.76 | 34181.04 | −15426.7 | −57275.3 | −291.54 | −8603.12 | 11888.5 |
| 276 | −123254 | 13862.92 | 28423.2 | −18564.2 | −58489.8 | −5107.24 | −12131.8 | 9159.62 |
| 275 | −122231 | 10481.8 | 19792.83 | −21954.7 | −63081.5 | −8052.14 | −15467.9 | 6816.33 |
| 274 | −122022 | 4544.58 | 14228.36 | −25506.2 | −68115 | −12723.1 | −18484.7 | 1898.11 |
| 273 | −119327 | −93 | 7507.14 | −27430.7 | −72064.7 | −18757.6 | −23622.4 | −2457.61 |
| 272 | −118117 | −4741.6 | −898.94 | −31105.6 | −76632.6 | −24065.4 | −27444.4 | −7630.12 |
| 271 | −114873 | −11243 | −8186.58 | −33612.4 | −79479.4 | −26123.6 | −31616.6 | −13317.5 |
| 270 | −110934 | −18278 | −16791.5 | −34326.7 | −81045.7 | −28626.8 | −35511.4 | −19659.7 |
| 269 | −110805 | −24696 | −23278.7 | −35538.1 | −77457.5 | −30895.5 | −36435.1 | −25982.1 |
| 268 | −107896 | −31682.6 | −26968.9 | −34651.5 | −72973.5 | −34416.7 | −38470.5 | −28365 |
| 267 | −104638 | −36057 | −32104.7 | −33289.7 | −68979.1 | −36049.2 | −40023.4 | −30953.2 |
| 266 | −101203 | −43418.8 | −38915.2 | −38202.6 | −68727.4 | −39704.6 | −43716.8 | −36674.6 |
| 265 | −97749.4 | −48343.8 | −46113.6 | −39483.8 | −66502.4 | −43998.7 | −46876.5 | −43499.6 |
| 264 | −97208.6 | −53390.8 | −52130.9 | −40459.1 | −66810.3 | −47957.5 | −49578.5 | −48748.6 |
| 263 | −95943.4 | −59511 | −56187.2 | −44454.4 | −68440.4 | −50835.4 | −52372.8 | −54090.1 |
| 262 | −95072.8 | −65060.4 | −63316.1 | −48747.7 | −70970.1 | −56562.5 | −58150.5 | −60326.8 |
| 261 | −96472.8 | −73045.2 | −69958.6 | −52712.8 | −73444.4 | −60734.8 | −62084.8 | −68172.5 |
| 260 | −98656.6 | −81333.8 | −76318.7 | −56168.7 | −76445.7 | −66930.2 | −66797.6 | −76104.6 |
| 259 | −103382 | −86822.4 | −80926.4 | −59864.4 | −77490.4 | −71080.2 | −70498.6 | −83599.7 |
| 258 | −106107 | −94436.6 | −86234.1 | −63976.9 | −80329.1 | −75652.4 | −74191 | −89179.8 |
| 257 | −110172 | −102156 | −91718.5 | −69322.3 | −82882.1 | −83082.9 | −79564.1 | −94507.5 |
| 256 | −114180 | −111501 | −100340 | −75666.4 | −88183.2 | −92107.3 | −87100.3 | −101872 |
| 255 | −121263 | −120856 | −110048 | −85659.8 | −97907.6 | −99931.1 | −95175.5 | −109970 |
| 254 | −129284 | −130947 | −121464 | −96584.1 | −107518 | −110216 | −106173 | −120889 |
| 253 | −138126 | −144778 | −133496 | −108571 | −117975 | −121301 | −119055 | −132245 |
| 252 | −147461 | −161570 | −146067 | −121456 | −131937 | −134579 | −134207 | −143284 |
| 251 | −160567 | −178374 | −162707 | −137083 | −146618 | −150852 | −152306 | −156392 |
| 250 | −176102 | −199419 | −181217 | −155612 | −165927 | −168321 | −171920 | −174242 |

Molar ellipticity is a standard method to determine unfavorable structures that could lead to stability issues and can be used to predict the stability of proteins. However, the elipticity values presented in Table 44 are comparable to those typically observed for well structured hence, stable monoclonal antibodies. Therefore, surprisingly the stability issues that were observed for LS-DVD-Ig proteins would not be predicted by this method.

### Example 22: Assessment of the Secondary Structure of DVD-Ig Proteins Using ATR-FTIR

To assess the secondary structure of DVD-Ig proteins, the second derivative, area normalized FTIR scans taken on an ATR-FTIR instrument from Bruker (Tensor 27) in the region 1600-1700 cm⁻¹ were curve fitted and the various peaks were analyzed and added up to get total % beta sheet structure in the molecule. Especially, peaks such as that at 1638 cm⁻¹, which are an indicator of the beta sheet arrangement, were taken into account. The studies were done in 5 mM citrate/5 mM phosphate buffer at a pH of 4, 6, or 8. The concentration of the DVD-Ig protein was 1 mg/ml. The total percentage of beta structure was assessed for 16 DVD-Ig proteins (see Table 45).

TABLE 45
The Total % Beta Structure in DVD-Ig Proteins Measured
Using ATR-FTIR

| DVD-Ig | pH 6 | pH 4 | pH 8 |
|---|---|---|---|
| 5 | 89.8 | 86.5 | 95.8 |
| 6 | 94.1 | 78.2 | 92.3 |
| 37 | 89.6 | 85.9 | 90.9 |
| 38 | 96.5 | 92 | 95.6 |
| 53 | NA | NA | 96.4 |
| 54 | 97.4 | 87.7 | 96.8 |
| 65 | 95.2 | 95.8 | 94.8 |
| 66 | 94.6 | 94.2 | 94.2 |
| 165 | NA | NA | 95.2 |
| 166 | 95.1 | 90 | 95.2 |
| 257 | 95.8 | 89.6 | 90.8 |
| 258 | 95.6 | 88.3 | 96.6 |
| 277 | 93.6 | 85.3 | 95 |
| 278 | 93.8 | 77.4 | 94.3 |
| 281 | 94.4 | 84.4 | 85 |
| 282 | 93 | 84.7 | 84.6 |

The results presented in Table 45 show that all of the 16 DVD-Ig proteins studied have a folded secondary structure that is composed primarily of beta elements. The proportion of beta elements ranged from about 85% to about 97%.

### Example 23: Impact of Ionic Strength and pH on the Second Virial Coefficient of DVD-B Formulations

Second virial coefficient (B₂₂) is a thermodyanmic parameter and an indicator of the protein-protein attractive or repulsive interactions in solutions. A positive value indicates repulsive interactions and a negative value indicates attractive interactions. Repulsive interactions usually translate into better long term storage. The scattered light intensity is related to the molecular weight and B₂₂ by the following equation.

$$\frac{Kc}{{R}_{⊖}} = \frac{1}{{M}_{w}} + 2 {B}_{22} c + {B}_{222} {c}^{z} + …$$

Where K is optical constant and is given by

$$K = \frac{{[ 2 π n ( \frac{dn}{d c} ) ]}^{2}}{{N}_{A} {λ}^{4}}$$

R_{θ} is the excess Rayleigh ratio, a measure of light scattered by the solute, n is the solvent refractive index, dn/dc is the refractive index increment of the solute, N_{A} is the Avogadro's number, and λ is the wavelength of the incident light. Since for most dilute solutions, higher order virial coefficients have negligible values, the following equation (Debye) is used to obtain the second virial coefficients.

$$\frac{Kc}{{R}_{θ}} = \frac{1}{{M}_{w}} + 2 {B}_{22} c$$

The scattered instensities were measured on a Malvern Zetasizer Nano. The second virial coefficient values were all positive and indicate that DVD-Ig proteins behave as typical protein molecules with respect to this calculation at least under dilute conditions. The buffers used were acetate for pH 4.5, histidine for pH 6 and Tris for pH 8. 2 mM concentration of buffer was used for 1 mM ionic strength solutions and 10 mM for 20 and 100 mM ionic strength solutions. The rest of the ionic strength was maintained by sodium chloride. The results are shown in Tables 34 and 35. The values of the second virial coefficients were higher on average at pH 4.5 and pH 6.0 than at pH 8.0, suggesting that DVD B would store better at pH 4.5 or pH 6.0 than at pH 8.0. Also, the values of the second virial coefficients were higher at lower ionic strength, suggesting that lower ionic strength may also be associated with stability of TNFPGE2.

Dₛ is the self-diffusion coefficient of the molecule at infinite dilution. k_{d} is a parameter describing the interaction between the molecules in solution. A positive value for k_{d} indicates intermolecular repulsion and vice versa.

TABLE 46
Virial Coefficient Values For DVD B at 0 mm Ionic Strength

| | B₂₂ × 10⁻⁴ | B₂₂₂ × 10⁻² | B₂₂₂₂ × 10⁻¹ | B₂₂₂₂₂ × 10² | M_{w} |
|---|---|---|---|---|---|
| pH | (mol mL/gm²) | (mol mL²/gm³) | (mol mL³/gm⁴) | (mol mL⁴/gm⁵) | (×1000) |
| 6.0 | 136 ± 5.77 | −273.8 ± 16.6 | 3376.1 ± 339 | −221 ± 35.8 | 189 ± 14 |

TABLE 47
Second Virial Coefficients Under Various Conditions for DVD-B

| | Ionic Strength | Dₛ × 10⁻⁷ | | B₂₂ × 10⁻⁴ | M_{w} |
|---|---|---|---|---|---|
| pH | (mM) | (cm²/s)* | k_{d} (mL/gm)^{†} | (mol mL/gm²)^{‡} | (×1000)^{§} |
| 4.5 | 1 | 3.42 ± 0.04 | 434.90 ± 15.31 | 26.09 ± 0.82 | 195 ± 1 |
| | 20 | 3.66 ± 0.004 | 5.96 ± 0.32 | 5.08 ± 0.03 | 168 ± 2.89 |
| | 100 | 3.60 ± 0.03 | −13.33 ± 0.88 | 3.09 ± 0.07 | 160 ± 3.38 |
| 6 | 1 | 3.41 ± 0.06 | 379.28 ± 51.34 | 14.71 ± 1.09 | 183 ± 0.57 |
| | 20 | 3.75 ± 0.02 | −11.04 ± 1.37 | 3.37 ± 0.06 | 163 ± 2.35 |
| | 100 | 3.70 ± .002 | −23.05 ± 0.02 | 2.51 ± 0.19 | 170 ± 3.40 |
| 8 | 1 | 3.81 ± 0.01 | −4.74 ± 0.06 | 3.50 ± 0.05 | 155 ± 0.78 |
| | 20 | 3.73 ± 0.02 | −27.31 ± 0.29 | 2.14 ± 0.03 | 162 ± 5.08 |
| | 100 | 3.66 ± 0.03 | −25.33 ± 0.90 | 2.34 ± 0.07 | 164 ± 4.59 |

### Example 24: Pharmacokinetic Study of DVD-Ig Proteins

The following example describes pharmacokinetic studies of various DVD-Ig proteins.

### Variable Domain Combination and Orientation Impacts Serum Stability

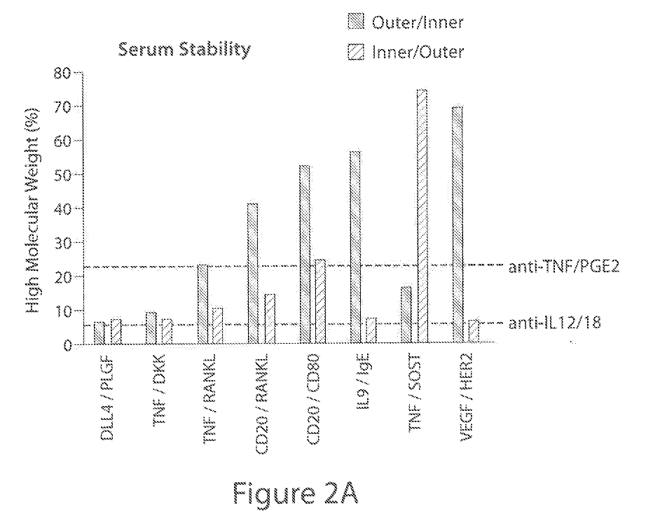

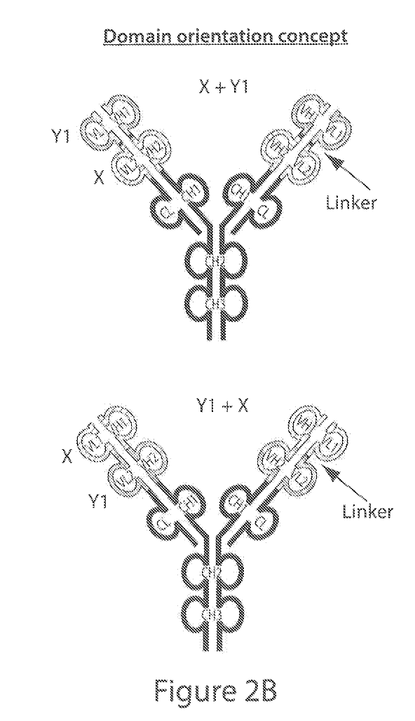

As described in FIG. 2, certain variable domain combinations provide a more stable DVD-Ig protein than other combinations. FIG. 2A describes the serum stability for certain DVD-Ig proteins in two different domain orientations, i.e., “outer/inner” and “inner/outer”. For example, the DVD-Ig protein TNF/SOST has a high molecular weight (HMW) % of about 16% in the “outer/inner” orientation, but has about a 75% HMW in the opposite orientation. The domain orientation concepts are set forth in FIG. 2B. The DVD-Ig proteins in FIG. 2 include short/short linker combinations and are huIgG1 isotypes.

### In Vitro Pharmacokinetic Study

The pharmacokinetic (PK) properties of various biologic therapeutics were assessed following 4 mg/kg single intravenous doses in male Sprague-Dawley rats. Blood samples were collected throughout the 28 day studies. Serum samples were analyzed using an MSD assay employing anti-human Ig capture and Sulfo-Tag labeled goat anti-human IgG antibody for chemiluminescent detection. Pharmacokinetic parameters for each animal were calculated using WinNonlin software by non-compartmental analysis.

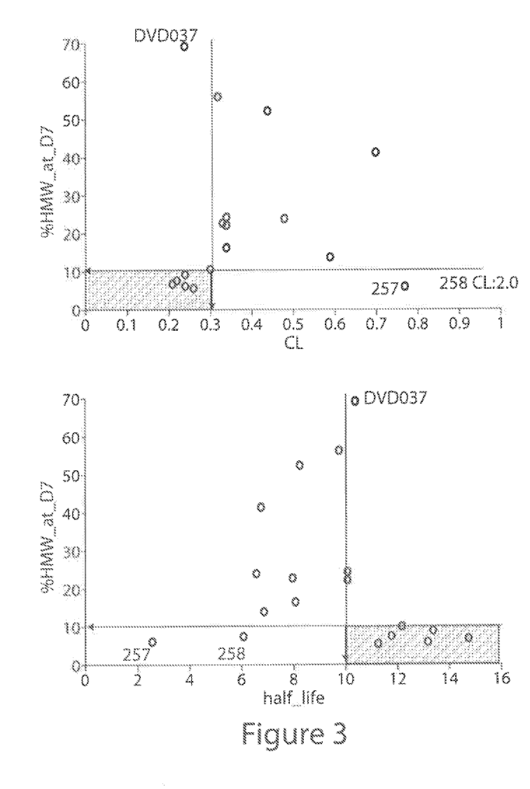

FIG. 3 describes results from a pharmacokinetic study using rats. The study examined the correlation between clearance (CL) and T ½ with high molecular weight (HMW) aggregate formation in vitro in rat serum. As shown in FIG. 3, DVD-Ig proteins with less than 10% HMW aggregate in vitro are more likely to have low (<0.3 ml/hour/kg) clearance and long (>10 days) half life. The outliers included DVD 257 and 258. The DLL4/VEGF DVD-Ig protein recognized the rat target, and the PK was affected by TMD. DVD037 (VEGF/HER2; SEQ ID NOs: 34 and 35) showed bad in vitro properties, but good PK in vivo. The amino acid sequences for the heavy and light chains of DLL4/VEGF DVD are provided in SEQ ID NOs: 68 and 69 of Table 66.

### Example 25: Viscosity Study of DVD-Ig Proteins

The following example describes viscosity studies for an exemplary AS-DVD-Ig protein (DVD-A).

Viscosity was measured on m-VROC low volume viscometer from Rheosense (Redwood, Calif.). m-VROC measure viscosity from the pressure drop of a test liquid as it flows through a rectangular slit. As the test liquid is pumped to flow through the flow channel, pressure is measured at increasing distance from the inlet. Plot of the straight line in the pressure vs. position of the sensor is proportional to the viscosity.

The instrument was evacuated beforehand to minimize the usage of material and susequently recover the material. Air was hence used to clean the instrument before a sample measurement was made. An initial flow rate of 40 μl/minute-200 μl/minute was used to obtain the required pressure differential. Saturation of the pressure chamber quickly stabilizes the viscosity reading, and twenty microliters of sample achieved stabilization. Once the instrument has been primed with the sample, less than 5 microliters of additional sample was enough to give a stable second reading. A total of less than thirty microliters (<35 microliters) of sample was thus enough to give readings in triplicate.

Viscosity of all samples was also measured on a rolling ball viscometer from Anton Paar (X, X). 1.8 mm capillary was used for samples of viscosity range 2-70 cP and 1.6 mm capillary was used for samples of minimal viscosity (less than 2 cP). The instrument was pre-calibrated and run at any of the various possible angles (70°, 50° and/or 40°).

Viscosity of DVDA was determined in histidine formulations having different molarity (i.e., 0 mM to 30 mM histidine) and pH (i.e., pH 4.8 to pH 8.3) in various DVD A concentrations. Results from the measurements are provided below in Tables 48 to 50.

TABLE 48
Viscosity Measurements of 34 mg/ml DVD A at Various
pH And Histidine Molarity
DVDA 34 mg/ml

| | | | | |
|---|---|---|---|---|
| pH 4.8 | pH 5.4 | pH 6 | pH 6.6 | pH 8.3 |

Anton Paar Viscosity readings (mPa · s.)

| | | | | | |
|---|---|---|---|---|---|
| 0 Mm Histidine | 1.83 | 2.9 | 5.22 | na | na |
| 5 Mm Histidine | 1.59 | 1.81 | 2.46 | 3.86 | 1.83 |
| 30 Mm Histidine | 1.33 | 1.71 | 2.03 | 3.5 | 1.97 |

Rheosense Viscosity measurments

| | | | | | |
|---|---|---|---|---|---|
| 0 Mm Histidine | | | | | |
| 1 | 3.07 | 4.11 | 6.03 | na | 2.35 |
| 2 | 3.04 | 4.04 | 5.84 | na | 2.36 |
| 3 | 2.93 | 4.15 | 6.08 | na | 2.40 |
| Average Viscosity | 3.01 | 4.10 | 5.98 | na | 2.37 |
| 5 Mm Histidine | | | | | |
| 1 | 1.58 | 1.73 | 2.56 | 3.87 | 2.22 |
| 2 | 1.52 | 1.71 | 2.55 | 3.87 | 2.12 |
| 3 | 1.59 | 1.72 | 2.53 | 3.86 | 2.13 |
| Average Viscosity | 1.56 | 1.72 | 2.55 | 3.86 | 2.16 |
| 30 Mm Histidine | | | | | |
| 1 | 1.37 | 1.57 | 1.88 | 3.34 | 2.84 |
| 2 | 1.39 | 1.58 | 1.91 | 3.37 | 2.93 |
| 3 | 1.33 | 1.58 | 1.86 | 3.50 | 2.73 |
| Average Viscosity | 1.36 | 1.58 | 1.88 | 3.40 | 2.83 |

TABLE 49
Viscosity Measurements of 15 mg/ml DVD-A At 0 Mm
Histidine at pH 4.8 to 8.3
DVD-A 15 mg/ml 0 mM

| 0 Mm Histidine | pH 4.8 | pH 5.4 | pH 6 | pH 6.6 | pH 8.3 |
|---|---|---|---|---|---|
| 1 | 1.66 | 1.30 | 1.28 | na | 1.85 |
| 2 | 1.64 | 1.34 | 1.31 | na | 1.82 |
| 3 | 1.64 | 1.30 | 1.29 | na | 1.80 |
| Average Viscosity | 1.65 | 1.31 | 1.29 | na | 1.82 |

TABLE 50
Viscosity Measurements of Various Concentrations Of DVD-A
at Various pH and Histidine Molarity
DVD-A

| | | 5 mM | | | |
|---|---|---|---|---|---|
| | | Anton | | 30 mM | |
| | | Paar | Rheosense | Anton Paar | Rheosense |
| pH 4.8 | 1 | 4.63 | 6.924 | 5.29 | 4.179 |
| (95 mg/ml) | 2 | 4.67 | 6.925 | 5.3 | 4.241 |
| | Average | 4.65 | 6.9245 | 5.295 | 4.21 |
| Ph 5.4 | 1 | 10.45 | 10.09 | 6.3 | 6.027 |
| (77 mg/ml) | 2 | 10.5 | 10.1 | 6.29 | 6.016 |
| | Average | 10.475 | 10.095 | 6.295 | 6.0215 |
| pH 6 | 1 | 6.23 | 5.721 | 3.643 | 3.34 |
| (47.8 mg/ml) | 2 | 6.3 | 5.695 | 3.648 | 3.36 |
| | Average | 6.265 | 5.708 | 3.6455 | 3.35 |
| | 5 mM | | 30 mM | | |

The results described above in Tables 48 to 50 show the impact of ionic strength, pH and protein concentration on the viscosity of the DVD-Ig protein solutions. DVD-A (SEQ ID NOs: 62 and 63) is an AS DVD-Ig and the results above show that the viscosity values can be modulated by formulation means to accommodate a syringeable liquid formulation at high concentrations which would be appropriate for pharmaceutical compositions and in vivo use. The results also show that the viscosity values could be accommodated to values that are generally observed for mAbs.

### Example 26: Thermal Stability of DVD-Ig Proteins

The following example describes results from three different tests examining thermal stability of DVD-Ig proteins, including an exemplary AS-DVD-Ig protein and an exemplary LS-DVD-Ig protein, versus monoclonal antibodies, such as Adalimumab.

### Dynamic Scanning Fluorescence

An automated high throughput instrument Optim-1000 from Avacta (York, UK) was used for the study. 9 microliter micro cubic arrays (MCAs) were used for the study. For preparation of stock samples, 3 microliter Sypro orange (Invitrogen, Cambridge, Mass.) was added to 27 microliter sample solution in order to obtain a final 1× concentration of the dye. The dye is available as 5000× commercial product, although any dye would be suitable. Thermal scans were obtained from 26° C. to 95° C. at a scan rate of 60° C./hour. Baseline was fitted for linearity and the first point (the temperature) whose inclusion decreased the R² below 0.95 was taken as the onset temperature. Repeat studies confirmed that the variation in onset temperatures was less than 5%.

### Intrinsic Fluorescence

Tryptophan fluorescence was used to evaluate the unfolding temperatures. Hitachi FL-4500 instrument from Hitachi (Tokyo, Japan) was used for the study. The temperatures were maintained using a water bath. The temperature in the cuvette was monitored using a thermocouple and a temperature monitor CSi32 from Omega Inc. (Stamford, Conn.). A front surface triangular quartz cuvette from VWR (MA) was used as this minimized the inner filter effects and hence resulted in strong emission signals. An excitation wavelength of 295 nm was used. Emission was monitored between 328-338 nm. Although the λₘₐₓ was observed at 332 nm, the intensity was monitored at 335 nm for comparison. The thermal scans were obtained from 30° C. to 70° C. at a scan rate of 78° C./hour. Baseline was fitted for linearity and the first point (the temperature) whose inclusion decreased the R² below 0.95 was taken as the onset temperature. Repeat studies confirmed that the variation in onset temperatures was less than 5%. The increased scan rate did not significantly affect the onset temperatures.

### Differential Scanning Calorimetry (DSC)

DSC was used to characterize the thermodynamic stability of the proteins under various solution conditions. An automated cap DSC instrument from Microcal (Northampton, Mass.) was used. The thermal scans were obtained from 25° C. to 65° C. at a scan rate of 60° C./hour. Since, aggregation and precipitation that follows unfolding in high concentration samples can lead to blocking of the cap DSC cells which than become rather difficult to clean, the scans were obtained only until ≈5° C. beyond the onset temperature to prevent any such occurrence. A prescan equilibration thermostat of 10 minutes was applied before each scan. A corresponding buffer scan was taken immediately following the sample scan. The difference in onset was less than 2° C. between repeat scans. Baseline was fitted for linearity and the first point (the temperature) whose inclusion decreased the R² below 0.95 was taken as the onset temperature. Repeat studies confirmed that the variation in onset temperatures was less than 5%.

Results from the study are provided in Table 51.

TABLE 51
Results from Thermal Stability Studies Comparing DVD-Ig Proteins
to Mabs

| | Intrinsic | | Extrinsic | | | |
|---|---|---|---|---|---|---|
| | fluorescence | | fluorescence | | DSC | |
| | 1 | 75 | 1 | 75 | 1 | 75 |
| | mg/mL | mg/mL | mg/mL | mg/mL | mg/mL | mg/mL |
| mAb1 | 66 | 64 | 63 | 57 | 59 | 57 |
| mAb2 | 74 | 69 | 73 | 68 | 63 | 59 |
| mAb3 | 65 | 62 | 65 | 63 | 59 | 55 |
| mAb4 | 72 | 67 | 64 | 58 | 61 | 59 |
| mAb5 | 71 | 64 | 66 | 61 | 59 | 56 |
| DVD1 | 69 | 67 | 64 | 61 | 62 | 59 |
| (IL12IL18) | | | | | | |
| DVDB | 56 | 53 | 53 | 49 | 54 | 50 |
| TNF/PGE2 | | | | | | |

The results described in Table 51 show the impact of protein concentration on the thermal stability of the protein solution. DVD1 (IL12IL18), an AS DVD and DVD2 (also referred to herein as DVD B), an LS-DVD-Ig protein, and other mAbs all show that protein concentration only has a slight impact on the thermal stability of the protein. So the feasibility of a liquid high concentration formulation may be independent of the impact of protein concentration on the thermal stability of the protein; however, high concentration liquid formulations present other well known types of instabilities, e.g., shelf instabilities. Generally, as described in Examples 1-3, DVD-Ig proteins have a lower melting temperature than antbodies. In some instances, e.g., DVD1, similar melting temperatures are observed, but generally this is not the case.

### X. Anti-DLL4/Anti-VEGF DVD-Ig Formulations (Aqueous and Lyophilized)

### Example 27: Preformulation Characterization of Anti-DLL4/Anti-VEGF DVD-Ig Protein h1A11.1SL-Av

The storage stability (5° C.) and accelerated stability (40° C.) of an anti-DLL4/anti-VEGF DVD (h1A11.1-SL-Av, Table 40) was evaluated in the formulations and protein concentrations listed below. Stability was evaluated by size exclusion chromatography (SEC) and % aggregrate, % monomer, % fragment, and total species recovered were quantitated. Overall, the formulations cover a pH range of 5 to 7 and a protein concentration range of 1.0 to 118 mg/ml.

At 5° C. and 40° C. temperatures and at protein concentrations of 50, 30, and 10 mg/ml, formulations were: 15 mM acetate pH 5; 15 mM phosphate pH 7; 30 mM acetate, 80 mg/ml sucrose, 0.02% Tween 80 at pH 5; 30 mM histidine, 80 mg/ml sucrose, 0.02% Tween 80 at pH 6; PBS (phosphate buffered saline). All formulations contained 0.02% sodium azide to prevent microbial growth during storage. At 5° C. and 40° C. temperatures and at protein concentrations of 60, 50, 30, and 10 mg/ml, the formulation was 15 mM histidine pH 6 (also containing 0.02% sodium azide to prevent microbial growth during storage). At 5° C. and at a protein concentration of 118 mg/ml, the formulation was 15 mM histidine pH 6 (also containing 0.02% sodium azide to prevent microbial growth during storage). At 40° C. and at a protein concentration of 1.0 mg/ml, the formulations were 10 mM citrate and 10 mM phosphate at pHs 5, 6, and 7. Formulations with protein were filtered to remove possible microbes.

Freeze-thaw stability was performed by subjecting the protein in formulation to four cycles of freezing at −80° C. for at least 20 hours and thawing in a 30° C. water bath. The formulations that were tested for freeze-thaw stability are listed below. Stability was evaluated by SE-HPLC and % aggregrate, % monomer, % fragment, and total species recovered were quantitated. The formulations were 15 mM histidine pH 6 at 60 mg/ml protein (also containing 0.02% sodium azide to prevent microbial growth) and 10 mM citrate and 10 mM phosphate at pHs 5, 6, and 7 and 1.0 mg/ml protein (filtered to remove possible microbes).

Finally, differential scanning calorimetry to measure thermal stability was performed on the protein in 10 mM citrate and 10 mM phosphate buffer at pHs 5, 6, and 7 and 1.0 mg/ml protein. The onset temperature of unfolding and the midpoint temperatures of unfolding (Tm) of each protein domain were quantitated.

TABLE 52
Accelerated Stability at 40° C. of H1a11.1-SL-Av At Different Concentrations and in
Different Buffers, Excipients, and pHs

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 2.71 | 96.31 | 0.98 | 53058 |
| | dialysis | | | | | | | |
| 50, 30, 10 | T0 | — | ace | 5 | 2.89 | 96.08 | 1.03 | 48033 |
| 50, 30, 10 | T0 | — | his | 6 | 2.81 | 96.23 | 0.96 | 46995 |
| 50, 30, 10 | T0 | — | phos | 7 | 2.91 | 96.09 | 1.00 | 52571 |
| 50, 30, 10 | T0 | — | ace-suc-tw | 5 | 2.54 | 96.50 | 0.96 | 50185 |
| 50, 30, 10 | T0 | — | his-suc-tw | 6 | 2.37 | 96.62 | 1.01 | 50771 |
| 50, 30, 10 | T0 | — | PBS | 7 | 2.90 | 96.08 | 1.01 | 49170 |
| 50 | T7 d | 40 | ace | 5 | 5.19 | 93.32 | 1.49 | 49028 |
| 30 | T7 d | 40 | ace | 5 | 3.86 | 94.68 | 1.47 | 48171 |
| 10 | T7 d | 40 | ace | 5 | 2.60 | 95.97 | 1.43 | 48379 |
| 50 | T7 d | 40 | his | 6 | 5.25 | 93.46 | 1.29 | 47731 |
| 30 | T7 d | 40 | his | 6 | 4.13 | 94.58 | 1.29 | 46684 |
| 10 | T7 d | 40 | his | 6 | 2.73 | 95.84 | 1.42 | 46877 |
| 50 | T7 d | 40 | phos | 7 | 9.02 | 89.52 | 1.46 | 53429 |
| 30 | T7 d | 40 | phos | 7 | 6.11 | 92.40 | 1.49 | 51923 |
| 10 | T7 d | 40 | phos | 7 | 3.94 | 94.57 | 1.49 | 53098 |
| 50 | T7 d | 40 | ace-suc-tw | 5 | 5.42 | 92.85 | 1.73 | 50373 |
| 30 | T7 d | 40 | ace-suc-tw | 5 | 4.07 | 94.06 | 1.87 | 48768 |
| 10 | T7 d | 40 | ace-suc-tw | 5 | 2.66 | 95.20 | 2.14 | 49396 |
| 50 | T7 d | 40 | his-suc-tw | 6 | 3.44 | 95.02 | 1.54 | 50040 |
| 30 | T7 d | 40 | his-suc-tw | 6 | 4.16 | 94.14 | 1.70 | 48715 |
| 10 | T7 d | 40 | his-suc-tw | 6 | 2.86 | 95.24 | 1.90 | 49871 |
| 50 | T7 d | 40 | PBS | 7 | 8.13 | 90.28 | 1.60 | 49207 |
| 30 | T7 d | 40 | PBS | 7 | 5.82 | 92.55 | 1.63 | 48853 |
| 10 | T7 d | 40 | PBS | 7 | 3.62 | 94.82 | 1.56 | 48166 |
| 50 | T21 d | 40 | ace | 5 | 6.65 | 90.83 | 2.51 | 48536 |
| 30 | T21 d | 40 | ace | 5 | 4.55 | 92.91 | 2.54 | 48520 |
| 10 | T21 d | 40 | ace | 5 | 2.71 | 94.70 | 2.59 | 48395 |
| 50 | T21 d | 40 | his | 6 | 7.01 | 90.71 | 2.27 | 46729 |
| 30 | T21 d | 40 | his | 6 | 4.69 | 93.10 | 2.21 | 46687 |
| 10 | T21 d | 40 | his | 6 | 2.77 | 94.93 | 2.30 | 46866 |
| 50 | T21 d | 40 | phos | 7 | 13.39 | 83.83 | 2.78 | 52244 |
| 30 | T21 d | 40 | phos | 7 | 9.38 | 87.76 | 2.86 | 53556 |
| 10 | T21 d | 40 | phos | 7 | 4.77 | 92.32 | 2.91 | 52536 |
| 50 | T21 d | 40 | ace-suc-tw | 5 | 6.37 | 90.34 | 3.30 | 48268 |
| 30 | T21 d | 40 | ace-suc-tw | 5 | 4.27 | 91.91 | 3.82 | 47211 |
| 10 | T21 d | 40 | ace-suc-tw | 5 | 2.26 | 93.02 | 4.72 | 46322 |
| 50 | T21 d | 40 | his-suc-tw | 6 | 6.84 | 89.82 | 3.34 | 47140 |
| 30 | T21 d | 40 | his-suc-tw | 6 | 4.60 | 91.90 | 3.50 | 47416 |
| 10 | T21 d | 40 | his-suc-tw | 6 | 2.67 | 93.66 | 3.67 | 48166 |
| 50 | T21 d | 40 | PBS | 7 | 12.13 | 84.81 | 3.06 | 49845 |
| 30 | T21 d | 40 | PBS | 7 | 8.09 | 88.78 | 3.13 | 48108 |
| 10 | T21 d | 40 | PBS | 7 | 4.20 | 92.63 | 3.17 | 48803 |

Buffer key (all buffers contain 0.02% sodium azide to prevent microbial growth):
ace = 15 mM acetate pH 5;
his = 15 mM histidine pH 6;
phos = 15 mM phosphate pH 7
ace-suc-tw = 30 mM acetate, 80 mg/ml sucrose, 0.02% Tw80
his-suc-tw = 30 mM histidine, 80 mg/ml sucrose, 0.02% Tw80
PBS = phosphate buffered saline

TABLE 53
Storage Stability At 5° C. of H1a11.1-SL-Av at Different Concentrations and in
Different Buffers, Excipients, and pHs (Buffer Key Same As In Table 52)

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 2.71 | 96.31 | 0.98 | 53058 |
| | dialysis | | | | | | | |
| 50, 30, 10 | T0 | — | ace | 5 | 2.89 | 96.08 | 1.03 | 48033 |
| 50, 30, 10 | T0 | — | his | 6 | 2.81 | 96.23 | 0.96 | 46995 |
| 50, 30, 10 | T0 | — | phos | 7 | 2.91 | 96.09 | 1.00 | 52571 |
| 50, 30, 10 | T0 | — | ace-suc-tw | 5 | 2.54 | 96.50 | 0.96 | 50185 |
| 50, 30, 10 | T0 | — | his-suc-tw | 6 | 2.37 | 96.62 | 1.01 | 50771 |
| 50, 30, 10 | T0 | — | PBS | 7 | 2.90 | 96.08 | 1.01 | 49170 |
| 50 | T7 d | 5 | ace | 5 | 2.96 | 95.99 | 1.05 | 49118 |
| 30 | T7 d | 5 | ace | 5 | 2.74 | 96.21 | 1.06 | 48434 |
| 10 | T7 d | 5 | ace | 5 | 2.62 | 96.23 | 1.15 | 48915 |
| 50 | T7 d | 5 | his | 6 | 2.93 | 95.87 | 1.20 | 47967 |
| 30 | T7 d | 5 | his | 6 | 2.75 | 96.06 | 1.19 | 47182 |
| 10 | T7 d | 5 | his | 6 | 2.55 | 96.31 | 1.13 | 47395 |
| 50 | T7 d | 5 | phos | 7 | 3.15 | 95.64 | 1.21 | 53843 |
| 30 | T7 d | 5 | phos | 7 | 3.10 | 95.76 | 1.14 | 53372 |
| 10 | T7 d | 5 | phos | 7 | 2.91 | 95.96 | 1.13 | 53269 |
| 50 | T7 d | 5 | ace-suc-tw | 5 | 2.75 | 96.13 | 1.12 | 50236 |
| 30 | T7 d | 5 | ace-suc-tw | 5 | 2.62 | 96.11 | 1.27 | 50026 |
| 10 | T7 d | 5 | ace-suc-tw | 5 | 2.56 | 96.18 | 1.26 | 49290 |
| 50 | T7 d | 5 | his-suc-tw | 6 | 2.84 | 96.10 | 1.07 | 50129 |
| 30 | T7 d | 5 | his-suc-tw | 6 | 2.58 | 96.19 | 1.23 | 49272 |
| 10 | T7 d | 5 | his-suc-tw | 6 | 2.64 | 96.08 | 1.28 | 50926 |
| 50 | T7 d | 5 | PBS | 7 | 3.26 | 95.59 | 1.15 | 49502 |
| 30 | T7 d | 5 | PBS | 7 | 3.07 | 95.64 | 1.29 | 49724 |
| 10 | T7 d | 5 | PBS | 7 | 2.83 | 95.87 | 1.29 | 49563 |
| 50 | T21 d | 5 | ace | 5 | 2.57 | 95.76 | 1.67 | 49722 |
| 30 | T21 d | 5 | ace | 5 | 2.37 | 96.03 | 1.60 | 48882 |
| 10 | T21 d | 5 | ace | 5 | 2.22 | 96.09 | 1.69 | 49255 |
| 50 | T21 d | 5 | his | 6 | 2.63 | 95.63 | 1.74 | 44884 |
| 30 | T21 d | 5 | his | 6 | 2.42 | 95.95 | 1.62 | 47510 |
| 10 | T21 d | 5 | his | 6 | 2.19 | 96.08 | 1.73 | 47015 |
| 50 | T21 d | 5 | phos | 7 | 3.06 | 94.96 | 1.98 | 53449 |
| 30 | T21 d | 5 | phos | 7 | 2.69 | 95.46 | 1.85 | 52938 |
| 10 | T21 d | 5 | phos | 7 | 2.35 | 95.84 | 1.81 | 52703 |
| 50 | T21 d | 5 | ace-suc-tw | 5 | 2.25 | 95.76 | 1.99 | 50960 |
| 30 | T21 d | 5 | ace-suc-tw | 5 | 2.08 | 95.90 | 2.02 | 49042 |
| 10 | T21 d | 5 | ace-suc-tw | 5 | 1.97 | 95.84 | 2.19 | 49851 |
| 50 | T21 d | 5 | his-suc-tw | 6 | 2.24 | 95.62 | 2.14 | 49983 |
| 30 | T21 d | 5 | his-suc-tw | 6 | 2.09 | 95.86 | 2.05 | 48813 |
| 10 | T21 d | 5 | his-suc-tw | 6 | 1.97 | 95.83 | 2.19 | 49984 |
| 50 | T21 d | 5 | PBS | 7 | 2.84 | 95.07 | 2.09 | 50641 |
| 30 | T21 d | 5 | PBS | 7 | 2.27 | 95.62 | 2.12 | 48441 |
| 10 | T21 d | 5 | PBS | 7 | 1.99 | 95.94 | 2.07 | 48978 |
| 50 | T10 mo | 5 | his | 6 | 8.05 | 91.04 | 0.91 | 45552 |
| 30 | T10 mo | 5 | his | 6 | 5.81 | 93.29 | 0.90 | 46607 |
| 10 | T10 mo | 5 | his | 6 | 3.62 | 95.46 | 0.92 | 46207 |
| 50 | T10 mo | 5 | his-suc-tw | 6 | 8.08 | 90.26 | 1.67 | 45430 |
| 30 | T10 mo | 5 | his-suc-tw | 6 | 5.98 | 92.43 | 1.58 | 42967 |
| 10 | T10 mo | 5 | his-suc-tw | 6 | 3.95 | 94.25 | 1.80 | 42567 |

TABLE 54
Storage Stability At 5° C., Accelerated Stability At 40° C., and Freeze-Thaw Stability
of H1a11.1-SL-Av at Different Concentrations And In Different Buffers and pHs

| Protein | | | | | | | | |
|---|---|---|---|---|---|---|---|---|
| conc | | temp | | | % | % | % | Total |
| (mg/ml) | time/FT | (° C.) | buffer | pH | Aggregrate | Monomer | Fragment | Area |
| 1 | T0 | — | cit-phos | 5 | 7.07 | 92.14 | 0.80 | 46824 |
| 1 | T8 d | 40 | cit-phos | 5 | 2.23 | 96.39 | 1.38 | 47090 |
| 1 | T22 d | 40 | cit-phos | 5 | 7.10 | 89.62 | 3.28 | 47956 |
| 1 | FT2 | — | cit-phos | 5 | 7.91 | 90.75 | 1.34 | 46502 |
| 1 | FT4 | — | cit-phos | 5 | 7.41 | 92.18 | 0.41 | 52181 |
| 1 | T0 | — | cit-phos | 6 | 7.17 | 92.33 | 0.50 | 45809 |
| 1 | T8 d | 40 | cit-phos | 6 | 2.56 | 96.03 | 1.42 | 46783 |
| 1 | T22 d | 40 | cit-phos | 6 | 5.79 | 91.73 | 2.48 | 47401 |
| 1 | FT2 | — | cit-phos | 6 | 7.14 | 91.48 | 1.38 | 45256 |
| 1 | FT4 | — | cit-phos | 6 | 7.09 | 92.56 | 0.34 | 45004 |
| 1 | T0 | — | cit-phos | 7 | 6.82 | 92.67 | 0.51 | 47025 |
| 1 | T8 d | 40 | cit-phos | 7 | 2.52 | 95.95 | 1.53 | 48080 |
| 1 | T22 d | 40 | cit-phos | 7 | 5.52 | 91.58 | 2.90 | 48706 |
| 1 | FT2 | — | cit-phos | 7 | 7.23 | 91.52 | 1.25 | 46732 |
| 1 | FT4 | — | cit-phos | 7 | 7.15 | 92.49 | 0.36 | 46561 |
| 60 and | T0 | — | his | 6 | 8.03 | 91.15 | 0.82 | 43528 |
| 118 | | | | | | | | |
| 60 | T7 d | 40 | his | 6 | 7.17 | 91.76 | 1.07 | 45333 |
| 60 | T21 d | 40 | his | 6 | 15.77 | 82.13 | 2.10 | 44729 |
| 60 | T7 d | 5 | his | 6 | 3.83 | 95.32 | 0.86 | 46774 |
| 60 | T26 d | 5 | his | 6 | 7.14 | 92.56 | 0.30 | 63982 |
| 118 | T5 mo | 5 | his | 6 | 12.82 | 86.65 | 0.53 | 55869 |
| 60 | T5 mo | 5 | his | 6 | 9.46 | 90.03 | 0.51 | 64573 |
| 60 | FT2 | — | his | 6 | 6.71 | 92.59 | 0.70 | 42259 |
| 60 | FT4 | — | his | 6 | 6.33 | 93.62 | 0.05 | 41054 |

Key:
FT = freeze thaw
FT2 = analysis after two cycles of freeze and thaw; freezing at −80° C. and thawing in a 30° C. water bath
FT4 = analysis after four cycles of freeze and thaw; freezing at −80° C. and thawing in a 30° C. water bath
cit-phos = 10 mM citrate and 10 mM phosphate
his = 15 mM histidine and 0.02% sodium azide (azide for preventing microbial growth)

TABLE 55
Differential Scanning Calorimetry Data Of H1a11.1-SL-Av At
1 mg/ml in 10 mm Citrate + 10 Mm Phosphate At Different pHs

| | | | | | Tm4 |
|---|---|---|---|---|---|
| pH | Onset (° C.) | Tm1 (° C.) | Tm2 (° C.) | Tm3 (° C.) | (° C.) |
| 5 | 55 | 68.2 | 68.86 | 75.56 | 81.18 |
| 6 | 58 | 69.04 | 70.47 | 75.24 | 82.04 |
| 7 | 59 | 69.52 | 70.94 | 74.44 | 82.06 |

### Example 28: Formulation Selection for Anti-DLL4/Anti-VEGF DVD-Ig Protein

### Materials and Methods.

The stability of anti-DLL4/anti-VEGF DVD-Ig h1A11.1-SL-Av protein was evaluated in the five formulations listed in Table 56. All formulations were prepared in 15 mM histidine buffer. Formulations F1 to F4 were prepared at 50 mg/ml protein concentration. In these formulations, the pH ranged from 5.5 to 6.0, polysorbate 80 concentration ranged from 0 to 0.05% w/v, sucrose concentration ranged from 0 to 7.5% w/v, and arginine concentration ranged from 0 to 1% w/v. Formulation F4 was prepared in 15 mM histidine buffer at pH 6.0 without any stabilizers and served as a study control for the 50 mg/ml liquid formulation stability assessment. In addition, one formulation was prepared at 25 mg/ml protein concentration at pH 6.0 (Formulation F5). In this formulation, the polysorbate 80 concentration was 0.025% w/v and sucrose concentration was 3.8% w/v.

TABLE 56
Formulation Composition Description

| | anti-DLL4/ | | | Poly- | | |
|---|---|---|---|---|---|---|
| | anti-VEGF | | | sorbate | | |
| | DVD-Ig | | | 80 | | |
| Formu- | Concen- | | | (Tween | | |
| lation | tration | | | 80) | Sucrose | Arginine |
| Identifier | (mg/mL) | Buffer | pH | (% w/v) | (% w/v) | (% w/v) |
| F1 | 50 | 15 mM | 6.0 | 0.05 | 7.5 | 0 |
| | | Histidine | | | | |
| F2 | 50 | 15 mM | 5.5 | 0.05 | 7.5 | 0 |
| | | Histidine | | | | |
| F3 | 50 | 15 mM | 6.0 | 0.05 | 7.5 | 1 |
| | | Histidine | | | | |
| F4 | 50 | 15 mM | 6.0 | 0 | 0 | 0 |
| | | Histidine | | | | |
| F5 | 25 | 15 mM | 6.0 | 0.025 | 3.8 | 0 |
| | | Histidine | | | | |

In the above formulations, 15 mM histidine buffer was selected because it provides adequate buffering capacity to maintain the target formulation pH. Sucrose was evaluated as a stabilizer against freeze-thaw stress (cryoprotectant) and lyophilization process-induced stress (lyoprotectant). Polysorbate 80 (surfactant) and arginine were added to potentially stabilize the formulation against aggregates and particulates formation.

### Freeze-Thaw and Liquid Formulation Stability Testing.

The stability of all liquid formulations was evaluated after three cycles of freeze/thaw (F/T) stress, and after 1 month storage at −80, 5, 25 and 40° C. Stability was tested by a broad panel of analytical assays including Visual appearance, % Aggregates by Size Exclusion Chromatography (SE-HPLC), Charge heterogeneity by Cation Exchange Chromatography (CEX-HPLC), Fragmentation by reduced SDS-Capillary Electrophoresis (CE-SDS), Sub-visible particles by Micro Flow Imaging (MFI) and DLL4/VEGF binding potency using ELISA.

The freeze/thaw and liquid stability testing results are provided in Table 57. Freeze-thaw stress resulted in the formation of visible particles and significantly higher sub-visible particle counts in Formulation F4 that was formulated without any stabilizers (polysorbate 80, sucrose, arginine). Relative to the other formulations, this formulation also showed a trend of higher sub-visible particle counts after 1 month storage at 25 and 40° C. Formulation F5 with 25 mg/mL protein concentration showed significantly lower aggregation relative to the 50 mg/mL formulations over 1 month storage at 5, 25 and 40° C.

TABLE 57
Freeze-Thaw and Liquid Formulation Stability Results

| | | | | | | | | Sub-visible | | | | |
|---|---|---|---|---|---|---|---|---|---|---|---|---|
| | | | % | CEX-HPLC | | | | Particles by MFI | | | Binding | |
| | | | Aggregates | % | % | % | | ≧2 | ≧10 | ≧25 | Potency | |
| Formulation | Time | Visual | by | Acidic | Main | Basic | % | μm/ | μm/ | μm/ | by ELISA | |
| Identifier | Points | Appearance | SEC-HPLC | region | peak | region | Purity | mL | mL | mL | DLL4 | VEGF |
| F1 | T0 | EFVP | 1.0 | 21.6 | 61.7 | 16.7 | 97.7 | 3333 | 5 | 0 | 93 | 113 |
| | 3FT | EFVP | 1.1 | 21.5 | 61.8 | 16.6 | 97.7 | 2388 | 50 | 5 | NP | NP |
| | 1M at | EFVP | 1.1 | 21.0 | 62.1 | 16.8 | 97.8 | 1364 | 15 | 5 | NP | NP |
| | −80° C. | | | | | | | | | | | |
| | 1M at | EFVP | 2.0 | 21.2 | 62.4 | 16.3 | 97.8 | 1064 | 0 | 0 | NP | NP |
| | 5° C. | | | | | | | | | | | |
| | 1M at | EFVP | 4.3 | 23.5 | 62.2 | 14.2 | 97.4 | 1559 | 5 | 5 | NP | NP |
| | 25° C. | | | | | | | | | | | |
| | 1M at | EFVP | 7.2 | 35.8 | 43.0 | 21.2 | 95.0 | 1219 | 15 | 0 | 94 | 104 |
| | 40° C. | | | | | | | | | | | |
| F2 | T0 | EFVP | 1.3 | 21.4 | 61.8 | 16.8 | 97.5 | 1589 | 15 | 0 | 93 | 113 |
| | 3FT | EFVP | 1.3 | 21.4 | 61.8 | 16.9 | 97.6 | 435 | 5 | 5 | NP | NP |
| | 1M at | EFVP | 1.4 | 21.0 | 62.0 | 17.0 | 97.8 | 315 | 0 | 0 | NP | NP |
| | −80° C. | | | | | | | | | | | |
| | 1M at | EFVP | 2.1 | 20.9 | 62.3 | 16.8 | 97.8 | 230 | 5 | 5 | NP | NP |
| | 5° C. | | | | | | | | | | | |
| | 1M at | EFVP | 4.4 | 22.8 | 61.6 | 15.6 | 97.4 | 1149 | 5 | 0 | NP | NP |
| | 25° C. | | | | | | | | | | | |
| | 1M at | EFVP | 7.9 | 33.8 | 40.9 | 25.3 | 95.1 | 655 | 5 | 0 | 95 | 97 |
| | 40° C. | | | | | | | | | | | |
| F3 | T0 | EFVP | 1.1 | 21.5 | 61.7 | 16.7 | 97.6 | 784 | 0 | 0 | 93 | 113 |
| | 3FT | EFVP | 1.1 | 21.3 | 61.8 | 16.9 | 97.5 | 490 | 10 | 0 | NP | NP |
| | 1M at | EFVP | 1.1 | 21.0 | 61.9 | 17.1 | 97.9 | 250 | 0 | 0 | NP | NP |
| | −80° C. | | | | | | | | | | | |
| | 1M at | EFVP | 2.0 | 20.8 | 62.0 | 17.2 | 97.8 | 225 | 5 | 0 | NP | NP |
| | 5° C. | | | | | | | | | | | |
| | 1M at | EFVP | 4.9 | 21.5 | 58.3 | 20.2 | 97.3 | 834 | 10 | 0 | NP | NP |
| | 25° C. | | | | | | | | | | | |
| | 1M at | EFVP | 11.3 | 32.1 | 42.3 | 25.6 | 95.0 | 1464 | 5 | 0 | 97 | 101 |
| | 40° C. | | | | | | | | | | | |
| F4 | T0 | TMTC | 1.2 | 21.6 | 61.8 | 16.6 | 97.4 | 23707 | 370 | 5 | 93 | 113 |
| | 3FT | TMTC | 1.5 | 21.4 | 61.8 | 16.8 | 97.4 | 105467 | 5906 | 30 | NP | NP |
| | 1M at | TMTC | 1.2 | 21.1 | 62.0 | 16.9 | 97.8 | 42024 | 1329 | 60 | NP | NP |
| | −80° C. | | | | | | | | | | | |
| | 1M at | EFVP | 2.0 | 21.3 | 62.3 | 16.5 | 97.8 | 1189 | 0 | 0 | NP | NP |
| | 5° C. | | | | | | | | | | | |
| | 1M at | EFVP | 4.5 | 23.5 | 61.8 | 14.7 | 97.2 | 89299 | 3053 | 165 | NP | NP |
| | 25° C. | | | | | | | | | | | |
| | 1M at | EFVP | 7.7 | 34.9 | 44.2 | 20.9 | 94.8 | 61754 | 5051 | 670 | 101 | 97 |
| | 40° C. | | | | | | | | | | | |
| F5 | T0 | EFVP | 0.9 | 21.6 | 61.6 | 16.7 | 97.7 | 2808 | 5 | 5 | 93 | 113 |
| | 3FT | EFVP | 1.2 | 21.5 | 61.8 | 16.8 | 97.6 | 1949 | 0 | 0 | NP | NP |
| | 1M at | EFVP | 1.1 | 21.0 | 62.2 | 16.8 | 97.8 | 270 | 5 | 0 | NP | NP |
| | −80° C. | | | | | | | | | | | |
| | 1M at | EFVP | 1.5 | 21.2 | 61.9 | 16.8 | 97.8 | 709 | 10 | 0 | NP | NP |
| | 5° C. | | | | | | | | | | | |
| | 1M at | EFVP | 2.6 | 23.5 | 62.0 | 14.6 | 97.2 | 944 | 15 | 0 | NP | NP |
| | 25° C. | | | | | | | | | | | |
| | 1M at | EFVP | 3.9 | 37.0 | 44.4 | 18.6 | 95.0 | 974 | 20 | 0 | 92 | 95 |
| | 40° C. | | | | | | | | | | | |

Key.
EFVP: Essentially Free of Visible Particles,
TMTC: Too Many To Count,
NP: Not Performed

### Lyophilized Formulation Stability Testing.

The stability of select formulations was also evaluated after the formulations were lyophilized. The lyophilized drug product stability was assessed for all sucrose-containing formulations (F1, F2, F3, and F5). Stability was assessed after 2 weeks storage at 55° C. Stability was tested by a broad panel of analytical assays including Visual appearance (before and after reconstitution), Reconstitution time, % Aggregates by Size Exclusion Chromatography (SE-HPLC), Charge heterogeneity by Cation Exchange Chromatography (CEX-HPLC), Fragmentation by reduced SDS-Capillary Electrophoresis (CE-SDS), Sub-visible particles by Micro Flow Imaging (MFI), and Water Content by Karl Fischer titration.

The lyophilized formulation stability testing results are provided in Table 58. Reconstitution time for all evaluated formulations was approximately 1 minute. A slight increase in aggregation by SEC and % basic region by CEX was observed for all formulations under the stressed storage condition of 55° C. Minimal changes were observed in all other measured product stability attributes.

TABLE 58
Lyophilized Formulation Stability Results

| | | | | % | | | | % | Sub-visible | | |
|---|---|---|---|---|---|---|---|---|---|---|---|
| | | Visual | | Aggreg | CEX-HPLC | | | Purity | Particles by MFI | | |
| | | Appearance | | by | % | % | % | by | ≧2 | ≧10 | ≧25 |
| Formulation | | After | Before | SEC- | Acidic | Main | Basic | RCE- | μm/ | μm/ | μm/ |
| Identifier | Time | Recon | Recon | HPLC | region | peak | region | SDS | mL | mL | mL |
| F1 | T0 | White to | EFVP | 1.1 | 21.3 | 61.9 | 16.8 | 97.6 | 749 | 20 | 10 |
| | | off-white | | | | | | | | | |
| | | cake | | | | | | | | | |
| | 2 weeks | White to | EFVP | 1.6 | 20.6 | 58.1 | 21.3 | 97.5 | 1639 | 15 | 0 |
| | at 55° C. | off-white | | | | | | | | | |
| | | cake | | | | | | | | | |
| F2 | T0 | White to | EFVP | 1.3 | 21.2 | 61.8 | 17.0 | 97.6 | 1254 | 15 | 0 |
| | | off-white | | | | | | | | | |
| | | cake | | | | | | | | | |
| | 2 weeks | White to | EFVP | 2.0 | 20.4 | 57.8 | 21.8 | 97.6 | 1609 | 10 | 0 |
| | at 55° C. | off-white | | | | | | | | | |
| | | cake | | | | | | | | | |
| F3 | T0 | White to | EFVP | 1.1 | 21.2 | 61.9 | 16.9 | 97.6 | 719 | 5 | 0 |
| | | off-white | | | | | | | | | |
| | | cake | | | | | | | | | |
| | 2 weeks | White to | EFVP | 1.4 | 20.8 | 59.5 | 19.8 | 97.5 | 475 | 10 | 5 |
| | at 55° C. | off-white | | | | | | | | | |
| | | cake | | | | | | | | | |
| F5 | T0 | White to | EFVP | 1.0 | 21.3 | 61.8 | 16.9 | 97.5 | 844 | 35 | 5 |
| | | off-white | | | | | | | | | |
| | | cake | | | | | | | | | |
| | 2 weeks | White to | EFVP | 1.5 | 20.5 | 58.0 | 21.5 | 97.7 | 270 | 5 | 0 |
| | at 55° C. | off-white | | | | | | | | | |
| | | cake | | | | | | | | | |

Key.
EFVP = Essentially Free of Visible Particles;
NP = Not Performed

### Example 29: Extended Preformulation Characterization of Second Anti-DLL4/Anti-VEGF DVD-Ig Protein (h1A11.1-LS-Av)

Extended preformulation characterization on anti-DLL4/-antiVEGF DVD-Ig proteins was performed to explore how different formulations conditions impact the stability of the DVD-Ig proteins. Data for h1A11.1-LS-Av is presented in Tables 59 and 60. The storage stability (5° C.) and accelerated stability (40° C.) of the DVD-Ig protein was evaluated in the formulations and protein concentrations listed below. Stability was evaluated by SEC and % aggregrate, % monomer, % fragment, and total species recovered were quantitated. Overall, the formulations cover a pH range of 5 to 7 and a protein concentration range of 10 to 50 mg/ml.

At 5° C. and 40° C. temperatures and at concentrations of 50, 30, and 10 mg/ml the following formulations were evaluated: 15 mM acetate pH 5, 15 mM histidine pH 6, 15 mM phosphate pH 7, 30 mM acetate, 80 mg/ml sucrose, 0.02% Tween 80 at pH 5, 30 mM histidine, 80 mg/ml sucrose, 0.02% Tween 80 at pH 6, and PBS (phosphate buffered saline). All formulations contained 0.02% sodium azide to prevent microbial growth during storage.

TABLE 59
Accelerated Stability At 40° C. of h1A11.1-LS-Av

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 0.21 | 98.42 | 1.36 | 56054 |
| | dialysis | | | | | | | |
| 50, 30, 10 | T0 | — | ace | 5 | 0.28 | 98.41 | 1.31 | 56381 |
| 50, 30, 10 | T0 | — | his | 6 | 0.46 | 98.23 | 1.31 | 54316 |
| 50, 30, 10 | T0 | — | phos | 7 | 0.74 | 97.86 | 1.40 | 53212 |
| 50, 30, 10 | T0 | — | ace-suc-tw | 5 | 0.24 | 98.16 | 1.60 | 56244 |
| 50, 30, 10 | T0 | — | his-suc-tw | 6 | 0.30 | 98.11 | 1.59 | 54076 |
| 50, 30, 10 | T0 | — | PBS | 7 | 0.52 | 98.05 | 1.43 | 50085 |
| 50 | T7 d | 40 | ace | 5 | 1.63 | 96.74 | 1.63 | 55563 |
| 30 | T7 d | 40 | ace | 5 | 1.13 | 97.24 | 1.62 | 55194 |
| 10 | T7 d | 40 | ace | 5 | 0.84 | 97.49 | 1.67 | 55029 |
| 50 | T7 d | 40 | his | 6 | 2.00 | 96.62 | 1.38 | 53566 |
| 30 | T7 d | 40 | his | 6 | 1.17 | 97.46 | 1.38 | 52443 |
| 10 | T7 d | 40 | his | 6 | 0.60 | 98.00 | 1.40 | 53812 |
| 50 | T7 d | 40 | phos | 7 | 4.31 | 94.02 | 1.67 | 52934 |
| 30 | T7 d | 40 | phos | 7 | 2.85 | 95.46 | 1.69 | 52663 |
| 10 | T7 d | 40 | phos | 7 | 1.20 | 97.11 | 1.69 | 52411 |
| 50 | T7 d | 40 | ace-suc-tw | 5 | 1.10 | 96.23 | 2.66 | 54837 |
| 30 | T7 d | 40 | ace-suc-tw | 5 | 0.77 | 96.40 | 2.83 | 52474 |
| 10 | T7 d | 40 | ace-suc-tw | 5 | 0.43 | 96.39 | 3.17 | 50855 |
| 50 | T7 d | 40 | his-suc-tw | 6 | 1.69 | 96.27 | 2.05 | 53017 |
| 30 | T7 d | 40 | his-suc-tw | 6 | 1.14 | 96.84 | 2.02 | 52153 |
| 10 | T7 d | 40 | his-suc-tw | 6 | 0.59 | 97.30 | 2.11 | 52208 |
| 50 | T7 d | 40 | PBS | 7 | 2.77 | 95.30 | 1.93 | 51623 |
| 30 | T7 d | 40 | PBS | 7 | 1.73 | 96.28 | 1.99 | 49973 |
| 10 | T7 d | 40 | PBS | 7 | 0.78 | 97.25 | 1.97 | 50851 |
| 50 | T21 d | 40 | ace | 5 | 3.66 | 94.30 | 2.04 | 55920 |
| 30 | T21 d | 40 | ace | 5 | 2.56 | 95.33 | 2.10 | 54188 |
| 10 | T21 d | 40 | ace | 5 | 1.85 | 96.00 | 2.15 | 55213 |
| 50 | T21 d | 40 | his | 6 | 4.14 | 94.28 | 1.58 | 54807 |
| 30 | T21 d | 40 | his | 6 | 2.67 | 95.79 | 1.54 | 53071 |
| 10 | T21 d | 40 | his | 6 | 1.59 | 96.82 | 1.58 | 54053 |
| 50 | T21 d | 40 | phos | 7 | 8.52 | 89.32 | 2.16 | 53273 |
| 30 | T21 d | 40 | phos | 7 | 5.58 | 92.54 | 1.89 | 53162 |
| 10 | T21 d | 40 | phos | 7 | 3.01 | 94.89 | 2.10 | 52747 |
| 50 | T21 d | 40 | ace-suc-tw | 5 | 4.12 | 93.78 | 2.10 | 56278 |
| 30 | T21 d | 40 | ace-suc-tw | 5 | 2.93 | 94.94 | 2.13 | 55481 |
| 10 | T21 d | 40 | ace-suc-tw | 5 | 1.99 | 95.75 | 2.26 | 54696 |
| 50 | T21 d | 40 | his-suc-tw | 6 | 4.94 | 93.21 | 1.85 | 54034 |
| 30 | T21 d | 40 | his-suc-tw | 6 | n/a | n/a | n/a | n/a |
| 10 | T21 d | 40 | his-suc-tw | 6 | 2.00 | 96.30 | 1.70 | 52686 |
| 50 | T21 d | 40 | PBS | 7 | 8.44 | 89.65 | 1.90 | 51697 |
| 30 | T21 d | 40 | PBS | 7 | 5.54 | 92.43 | 2.03 | 50282 |
| 10 | T21 d | 40 | PBS | 7 | 2.89 | 95.05 | 2.06 | 51580 |

Buffer key (all buffers contain 0.02% sodium azide to prevent microbial growth):ace=15 mM acetate pH 5; his=15 mM histidine pH 6; phos=15 mM phosphate pH 7; ace-suc-tw=30 mM acetate, 80 mg/ml sucrose, 0.02% Tween80; his-suc-tw=30 mM histidine, 80 mg/ml sucrose, 0.02% Tween80; PBS=phosphate buffered saline

TABLE 60
Storage Stability At 5° C. of h1A11.1-LS-Av

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 0.21 | 98.42 | 1.36 | 56054 |
| | dialysis | | | | | | | |
| 50, 30, 10 | T0 | — | ace | 5 | 0.28 | 98.41 | 1.31 | 56381 |
| 50, 30, 10 | T0 | — | his | 6 | 0.46 | 98.23 | 1.31 | 54316 |
| 50, 30, 10 | T0 | — | phos | 7 | 0.74 | 97.86 | 1.40 | 53212 |
| 50, 30, 10 | T0 | — | ace-suc-tw | 5 | 0.24 | 98.16 | 1.60 | 56244 |
| 50, 30, 10 | T0 | — | his-suc-tw | 6 | 0.30 | 98.11 | 1.59 | 54076 |
| 50, 30, 10 | T0 | — | PBS | 7 | 0.52 | 98.05 | 1.43 | 50085 |
| 50 | T7 d | 5 | ace | 5 | 0.18 | 98.17 | 1.64 | 57599 |
| 30 | T7 d | 5 | ace | 5 | 0.16 | 98.21 | 1.64 | 55889 |
| 10 | T7 d | 5 | ace | 5 | 0.13 | 98.17 | 1.70 | 53289 |
| 50 | T7 d | 5 | his | 6 | 0.18 | 98.14 | 1.68 | 55742 |
| 30 | T7 d | 5 | his | 6 | 0.12 | 98.06 | 1.82 | 53603 |
| 10 | T7 d | 5 | his | 6 | 0.13 | 98.07 | 1.80 | 53505 |
| 50 | T7 d | 5 | phos | 7 | 0.23 | 97.72 | 2.05 | 54355 |
| 30 | T7 d | 5 | phos | 7 | 0.18 | 97.77 | 2.04 | 53561 |
| 10 | T7 d | 5 | phos | 7 | 0.13 | 97.72 | 2.15 | 53151 |
| 50 | T7 d | 5 | ace-suc-tw | 5 | 0.09 | 97.40 | 2.51 | 57158 |
| 30 | T7 d | 5 | ace-suc-tw | 5 | 0.08 | 97.43 | 2.49 | 55025 |
| 10 | T7 d | 5 | ace-suc-tw | 5 | 0.08 | 97.34 | 2.58 | 53882 |
| 50 | T7 d | 5 | his-suc-tw | 6 | 0.10 | 97.48 | 2.43 | 55272 |
| 30 | T7 d | 5 | his-suc-tw | 6 | 0.08 | 97.63 | 2.29 | 52763 |
| 10 | T7 d | 5 | his-suc-tw | 6 | 0.05 | 97.41 | 2.53 | 52903 |
| 50 | T7 d | 5 | PBS | 7 | 0.12 | 97.31 | 2.58 | 51698 |
| 30 | T7 d | 5 | PBS | 7 | 0.09 | 97.24 | 2.67 | 50144 |
| 10 | T7 d | 5 | PBS | 7 | 0.08 | 97.28 | 2.64 | 50428 |
| 50 | T21 d | 5 | ace | 5 | 0.87 | 98.45 | 0.68 | 57706 |
| 30 | T21 d | 5 | ace | 5 | 0.80 | 98.55 | 0.65 | 56566 |
| 10 | T21 d | 5 | ace | 5 | 0.83 | 98.47 | 0.70 | 54226 |
| 50 | T21 d | 5 | his | 6 | 1.05 | 98.29 | 0.66 | 55911 |
| 30 | T21 d | 5 | his | 6 | 0.92 | 98.40 | 0.68 | 54225 |
| 10 | T21 d | 5 | his | 6 | 0.90 | 98.41 | 0.70 | 54128 |
| 50 | T21 d | 5 | phos | 7 | 1.25 | 98.09 | 0.66 | 54980 |
| 30 | T21 d | 5 | phos | 7 | 1.20 | 98.11 | 0.69 | 53903 |
| 10 | T21 d | 5 | phos | 7 | 1.01 | 98.29 | 0.69 | 53271 |
| 50 | T21 d | 5 | ace-suc-tw | 5 | 0.92 | 98.36 | 0.72 | 61574 |
| 30 | T21 d | 5 | ace-suc-tw | 5 | 0.89 | 98.39 | 0.72 | 55532 |
| 10 | T21 d | 5 | ace-suc-tw | 5 | 0.83 | 98.46 | 0.71 | 55841 |
| 50 | T21 d | 5 | his-suc-tw | 6 | 1.00 | 98.27 | 0.73 | 55484 |
| 30 | T21 d | 5 | his-suc-tw | 6 | 0.92 | 98.37 | 0.70 | 53335 |
| 10 | T21 d | 5 | his-suc-tw | 6 | 0.82 | 98.49 | 0.69 | 53736 |
| 50 | T21 d | 5 | PBS | 7 | 1.49 | 97.79 | 0.71 | 52405 |
| 30 | T21 d | 5 | PBS | 7 | 1.29 | 98.02 | 0.70 | 51284 |
| 10 | T21 d | 5 | PBS | 7 | 1.12 | 98.18 | 0.70 | 51377 |

The buffer key for Table 60 is the same as in Table 59.

The sequence of the anti-DLL4/anti-VEGF DVD-Ig protein H1A11.1-SL-Av is set forth in Table 61 (DVD-Ig protein described in Examples 27 and 28).

TABLE 61
Full Length Sequence For DVD h1A11.1-SL-Av

| Name | Sequence |
|---|---|
| h1A11A-SL- | DIQMTQSPSSLSASVGDRVTITCRASEDIYSNLAWYQQKPGKAPKLLIYDTNNLADGV |
| Av light chain | PSRFSGSGSGTDFTLTISSLQPEDFATYYCQQYNNYPPTEGQGTKLEIKRTVAAPSVF |
| | IFPPDIQMTQSPSSLSASVGDRVTITCSASQDISNYLNWYQQKPGKAPKVLIYFTSSL |
| | HSGVPSRFSGSGSGTDFTLTISSLQPEDFATYYCQQYSTVPWTFGQGTKVEIKRTVAA |
| | PSVFIFPPSDEQLKSGTASVVCLLNNFYPREAKVQWKVDNALQSGNSQESVTEQDSKD |
| | STYSLSSTLTLSKADYEKHKVYACEVTHQGLSSPVTKSFNRGEC (SEQ ID NO: |
| | 28) |
| h1A11.1-SL- | EVQLVESGGGLVQPGGSLRLSCAASGFTFSNFPMAWVRQAPGKGLEWVATIS |
| Av heavy | SSDGTTYYRDSVKGRFTISRDNAKNSLYLQMNSLRAEDTAVYYCARGYYNSPF |
| chain | AYWGQGTLVTVSSASTKGPEVQLVESGGGLVQPGGSLRLSCAASGYTFTNYG |
| | MNWVRQAPGKGLEWVGWINTYTGEPTYAADFKRRFTFSLDTSKSTAYLQMNS |
| | LRAEDTAVYYCAKYPHYYGSSHWYEDVWGQGTLVTVSSASTKGPSVFPLAPSS |
| | KSTSGGTAALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEAAGGPSV |
| | FLEPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 29) |

Table 61 provides the full-length heavy and light chan sequences for DVD h1A11.1-SL-Av. Linker sequences are underlined, while constant region sequences are in bold.

### Example 30: Formulation Studies of Additional DLL4-VEGF DVD-Ig Proteins h1A11.1.A6-LS-Av and h1A11.1.A6-SL-Av

Extended preformulation characterization on additional DLL4/VEGF DVD-Ig proteins, was performed to explore how different formulations conditions impact the stability. Data for h1A11.1.A6-LS-Av and h1A11.1.A6-SL-Av DLL4/VEGF DVD-Ig proteins are presented below. These DVD-Ig proteins proved to be unstable and failed the screening criteria to be considered AS-DVD-Ig proteins.

The storage stability (5° C.) and accelerated stability (40° C.) of h1A11.1.A6-LS-Av and h1A11.1.A6-SL-Av were evaluated in the formulations and protein concentrations listed below. Stability was evaluated by SEC and % aggregrate, % monomer, % fragment, and total species recovered were quantitated. Overall, the formulations cover a pH range of 5 to 7 and a protein concentration range of 10 to 50 mg/ml.

At 5° C. and 40° C. temperatures and at 50, 30, and 10 mg/ml, the following conditions were tested: 15 mM acetate pH 5; 15 mM histidine pH 6; 15 mM phosphate pH 7; 30 mM acetate, 80 mg/ml sucrose, 0.02% Tween 80 at pH 5; 30 mM histidine, 80 mg/ml sucrose, 0.02% Tween 80 at pH 6; and PBS (phosphate buffered saline). All formulations contained 0.02% sodium azide to prevent microbial growth during storage

Overall, the data provided in Tables 62-64 suggests the two DVD-Ig proteins have an atypical degradation profile not observed for stable monoclonal antibodies. The aggregation rate was actually greater at 5° C. than at 40° C.

At 5° C., there is a rapid increase in aggregation after 21 days of storage. At 40° C., the amount of degradation also increases, but not at the rate observed at 5° C. In both cases, the aggregation is concentration dependent.

Overall, these DVD-Ig proteins failed the screen based on their 5° C. instability and are examples of non-AS-DVD-Ig proteins.

TABLE 62
Accelerated Stability at 40° C. Of H1a11.1.A6-LS-Av at Different Concentrations
And In Different Buffers, Excipients, and pHs

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 0.66 | 98.64 | 0.70 | 48425 |
| | dialysis | | | | | | | |
| 50, 30, 10 | T0 | — | ace | 5 | 1.24 | 98.05 | 0.72 | 50944 |
| 50, 30, 10 | T0 | — | his | 6 | 1.49 | 97.74 | 0.76 | 46462 |
| 50, 30, 10 | T0 | — | phos | 7 | 1.76 | 97.67 | 0.58 | 43322 |
| 50, 30, 10 | T0 | — | ace-suc-tw | 5 | 1.66 | 97.69 | 0.65 | 57038 |
| 50, 30, 10 | T0 | — | his-suc-tw | 6 | 1.62 | 97.87 | 0.52 | 54299 |
| 50, 30, 10 | T0 | — | PBS | 7 | 2.22 | 97.25 | 0.53 | 48116 |
| 50 | T7 d | 40 | ace | 5 | 4.40 | 94.52 | 1.08 | 43246 |
| 30 | T7 d | 40 | ace | 5 | 2.76 | 96.17 | 1.08 | 46266 |
| 10 | T7 d | 40 | ace | 5 | 1.54 | 97.41 | 1.05 | 52178 |
| 50 | T7 d | 40 | his | 6 | 5.11 | 94.02 | 0.87 | 50471 |
| 30 | T7 d | 40 | his | 6 | 3.17 | 95.97 | 0.87 | 40875 |
| 10 | T7 d | 40 | his | 6 | 1.60 | 97.52 | 0.88 | 48759 |
| 50 | T7 d | 40 | phos | 7 | 12.24 | 86.69 | 1.07 | 44327 |
| 30 | T7 d | 40 | phos | 7 | 7.81 | 91.09 | 1.11 | 42284 |
| 10 | T7 d | 40 | phos | 7 | 3.46 | 95.42 | 1.12 | 44023 |
| 50 | T7 d | 40 | ace-suc-tw | 5 | 5.46 | 93.48 | 1.07 | 63100 |
| 30 | T7 d | 40 | ace-suc-tw | 5 | 3.42 | 95.55 | 1.03 | 59243 |
| 10 | T7 d | 40 | ace-suc-tw | 5 | 1.83 | 97.03 | 1.14 | 60361 |
| 50 | T7 d | 40 | his-suc-tw | 6 | 4.90 | 94.14 | 0.96 | 56706 |
| 30 | T7 d | 40 | his-suc-tw | 6 | 3.65 | 95.50 | 0.84 | 53538 |
| 10 | T7 d | 40 | his-suc-tw | 6 | 1.78 | 97.31 | 0.91 | 55447 |
| 50 | T7 d | 40 | PBS | 7 | 14.78 | 84.08 | 1.14 | 58511 |
| 30 | T7 d | 40 | PBS | 7 | 9.20 | 89.56 | 1.24 | 46917 |
| 10 | T7 d | 40 | PBS | 7 | 4.08 | 94.73 | 1.19 | 49978 |
| 50 | T21 d | 40 | ace | 5 | 5.58 | 92.75 | 1.67 | 53248 |
| 30 | T21 d | 40 | ace | 5 | 3.59 | 94.67 | 1.74 | 51038 |
| 10 | T21 d | 40 | ace | 5 | 1.90 | 96.38 | 1.72 | 50617 |
| 50 | T21 d | 40 | his | 6 | 6.25 | 92.37 | 1.37 | 46908 |
| 30 | T21 d | 40 | his | 6 | 3.82 | 94.77 | 1.41 | 46075 |
| 10 | T21 d | 40 | his | 6 | 1.93 | 96.61 | 1.47 | 47827 |
| 50 | T21 d | 40 | phos | 7 | 13.12 | 84.98 | 1.90 | 40311 |
| 30 | T21 d | 40 | phos | 7 | 9.11 | 88.98 | 1.91 | 42037 |
| 10 | T21 d | 40 | phos | 7 | 4.05 | 93.96 | 1.99 | 43009 |
| 50 | T21 d | 40 | ace-suc-tw | 5 | 6.45 | 91.73 | 1.82 | 56752 |
| 30 | T21 d | 40 | ace-suc-tw | 5 | 4.21 | 93.92 | 1.87 | 56330 |
| 10 | T21 d | 40 | ace-suc-tw | 5 | 2.11 | 95.88 | 2.00 | 57757 |
| 50 | T21 d | 40 | ace-suc-tw | 6 | 6.69 | 91.83 | 1.49 | 54091 |
| 30 | T21 d | 40 | ace-suc-tw | 6 | 4.22 | 94.30 | 1.48 | 52932 |
| 10 | T21 d | 40 | ace-suc-tw | 6 | 2.09 | 96.45 | 1.47 | 54118 |
| 50 | T21 d | 40 | PBS | 7 | 15.74 | 82.23 | 2.03 | 52080 |
| 30 | T21 d | 40 | PBS | 7 | 9.98 | 87.82 | 2.19 | 47188 |
| 10 | T21 d | 40 | PBS | 7 | 4.71 | 93.04 | 2.25 | 47400 |

Buffer key (all buffers contain 0.02% sodium azide to prevent microbial growth):
ace = 15 mM acetate pH 5;
his = 15 mM histidine pH 6;
phos = 15 mM phosphate pH 7
ace-suc-tw = 30 mM acetate, 80 mg/ml sucrose, 0.02% Tw80
his-suc-tw = 30 mM histidine, 80 mg/ml sucrose, 0.02% Tw80
PBS = phosphate buffered saline

TABLE 63
Storage Stability At 5° C. Of H1a11.1.A6-LS-Av At Different Concentrations
And In Different Buffers, Excipients, And pHs (Buffer Key Same As In Table 63)

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 0.66 | 98.64 | 0.70 | 48425 |
| | dialysis | | | | | | | |
| 50, 30, 10 | T0 | — | ace | 5 | 1.24 | 98.05 | 0.72 | 50944 |
| 50, 30, 10 | T0 | — | his | 6 | 1.49 | 97.74 | 0.76 | 46462 |
| 50, 30, 10 | T0 | — | phos | 7 | 1.76 | 97.67 | 0.58 | 43322 |
| 50, 30, 10 | T0 | — | ace-suc-tw | 5 | 1.66 | 97.69 | 0.65 | 57038 |
| 50, 30, 10 | T0 | — | his-suc-tw | 6 | 1.62 | 97.87 | 0.52 | 54299 |
| 50, 30, 10 | T0 | — | PBS | 7 | 2.22 | 97.25 | 0.53 | 48116 |
| 50 | T7 d | 5 | ace | 5 | 4.68 | 94.74 | 0.58 | 54446 |
| 30 | T7 d | 5 | ace | 5 | 2.77 | 96.62 | 0.62 | 51286 |
| 10 | T7 d | 5 | ace | 5 | 1.75 | 97.62 | 0.63 | 51444 |
| 50 | T7 d | 5 | his | 6 | 7.46 | 91.88 | 0.66 | 46683 |
| 30 | T7 d | 5 | his | 6 | 4.83 | 94.49 | 0.68 | 46282 |
| 10 | T7 d | 5 | his | 6 | 2.36 | 96.95 | 0.69 | 47844 |
| 50 | T7 d | 5 | phos | 7 | 1.84 | 97.46 | 0.71 | 34008 |
| 30 | T7 d | 5 | phos | 7 | 1.89 | 97.48 | 0.63 | 36992 |
| 10 | T7 d | 5 | phos | 7 | 2.05 | 97.30 | 0.65 | 41962 |
| 50 | T7 d | 5 | ace-suc-tw | 5 | 4.74 | 94.65 | 0.61 | 57937 |
| 30 | T7 d | 5 | ace-suc-tw | 5 | N/A | N/A | N/A | N/A |
| 10 | T7 d | 5 | ace-suc-tw | 5 | 1.95 | 97.41 | 0.64 | 58618 |
| 50 | T7 d | 5 | his-suc-tw | 6 | 7.97 | 91.45 | 0.59 | 54735 |
| 30 | T7 d | 5 | his-suc-tw | 6 | 4.85 | 94.49 | 0.66 | 53379 |
| 10 | T7 d | 5 | his-suc-tw | 6 | 2.34 | 97.01 | 0.65 | 54187 |
| 50 | T7 d | 5 | PBS | 7 | 5.68 | 93.65 | 0.67 | 46544 |
| 30 | T7 d | 5 | PBS | 7 | 5.20 | 94.13 | 0.67 | 45219 |
| 10 | T7 d | 5 | PBS | 7 | 3.56 | 95.76 | 0.68 | 47653 |
| 50 | T21 d | 5 | ace | 5 | 9.33 | 89.97 | 0.70 | 52020 |
| 30 | T21 d | 5 | ace | 5 | 4.25 | 95.04 | 0.70 | 51223 |
| 10 | T21 d | 5 | ace | 5 | 1.95 | 97.36 | 0.69 | 50950 |
| 50 | T21 d | 5 | his | 6 | 19.35 | 79.71 | 0.94 | 44105 |
| 30 | T21 d | 5 | his | 6 | 9.91 | 89.29 | 0.80 | 46096 |
| 10 | T21 d | 5 | his | 6 | 3.11 | 96.15 | 0.73 | 47777 |
| 50 | T21 d | 5 | phos | 7 | 1.65 | 97.49 | 0.86 | 25059 |
| 30 | T21 d | 5 | phos | 7 | 1.49 | 97.75 | 0.76 | 29723 |
| 10 | T21 d | 5 | phos | 7 | 2.00 | 97.09 | 0.90 | 38517 |
| 50 | T21 d | 5 | ace-suc-tw | 5 | 9.41 | 89.79 | 0.79 | 56438 |
| 30 | T21 d | 5 | ace-suc-tw | 5 | 4.72 | 94.50 | 0.79 | 56230 |
| 10 | T21 d | 5 | ace-suc-tw | 5 | 2.13 | 97.01 | 0.86 | 58579 |
| 50 | T21 d | 5 | his-suc-tw | 6 | 20.57 | 78.32 | 1.11 | 53114 |
| 30 | T21 d | 5 | his-suc-tw | 6 | 10.17 | 88.99 | 0.85 | 53155 |
| 10 | T21 d | 5 | his-suc-tw | 6 | 3.20 | 95.94 | 0.86 | 54028 |
| 50 | T21 d | 5 | PBS | 7 | 5.65 | 93.38 | 0.98 | 34294 |
| 30 | T21 d | 5 | PBS | 7 | 2.99 | 96.04 | 0.96 | 36457 |
| 10 | T21 d | 5 | PBS | 7 | 5.14 | 93.95 | 0.91 | 46566 |

TABLE 64
Accelerated Stability At 40° C. Of H1a11.1.A6-SL-Av At Different Concentrations
And In Different Buffers, Excipients, And pHs

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 1.19 | 97.75 | 1.06 | 58988 |
| | dialysis | | | | | | | |
| 50, 30, 10 | T0 | — | ace | 5 | 1.23 | 97.85 | 0.92 | 47138 |
| 50, 30, 10 | T0 | — | his | 6 | 1.30 | 97.84 | 0.87 | 44711 |
| 50, 30, 10 | T0 | — | phos | 7 | 0.86 | 98.00 | 1.14 | 57373 |
| 50, 30, 10 | T0 | — | ace-suc-tw | 5 | 1.30 | 97.85 | 0.85 | 52129 |
| 50, 30, 10 | T0 | — | his-suc-tw | 6 | 1.28 | 97.85 | 0.87 | 47563 |
| 6.6 | T0 | — | PBS | 7 | 1.35 | 97.76 | 0.89 | 71146 |
| 50 | T7 d | 40 | ace | 5 | 7.66 | 90.88 | 1.46 | 48331 |
| 30 | T7 d | 40 | ace | 5 | 4.05 | 94.43 | 1.52 | 45731 |
| 10 | T7 d | 40 | ace | 5 | 1.45 | 97.05 | 1.51 | 47455 |
| 50 | T7 d | 40 | his | 6 | 8.68 | 90.23 | 1.09 | 45897 |
| 30 | T7 d | 40 | his | 6 | 4.74 | 94.15 | 1.11 | 44807 |
| 10 | T7 d | 40 | his | 6 | 1.62 | 97.07 | 1.31 | 45567 |
| 3.4 | T7 d | 40 | phos | 7 | 2.04 | 96.51 | 1.45 | 56002 |
| 50 | T7 d | 40 | ace-suc-tw | 5 | 9.10 | 89.27 | 1.62 | 52696 |
| 30 | T7 d | 40 | ace-suc-tw | 5 | 5.23 | 93.14 | 1.64 | 50591 |
| 10 | T7 d | 40 | ace-suc-tw | 5 | 1.91 | 96.09 | 2.00 | 51790 |
| 50 | T7 d | 40 | his-suc-tw | 6 | 8.55 | 90.18 | 1.27 | 48458 |
| 30 | T7 d | 40 | his-suc-tw | 6 | 4.82 | 93.19 | 1.99 | 46963 |
| 10 | T7 d | 40 | his-suc-tw | 6 | 1.78 | 96.78 | 1.45 | 46676 |
| 6.6 | T7 d | 40 | PBS | 7 | 4.53 | 93.83 | 1.64 | 70277 |
| 50 | T21 d | 40 | ace | 5 | 8.27 | 89.20 | 2.53 | 47139 |
| 30 | T21 d | 40 | ace | 5 | 4.41 | 93.03 | 2.56 | 45779 |
| 10 | T21 d | 40 | ace | 5 | 1.60 | 95.68 | 2.72 | 46794 |
| 50 | T21 d | 40 | his | 6 | 9.26 | 88.56 | 2.18 | 44423 |
| 30 | T21 d | 40 | his | 6 | 5.19 | 92.86 | 1.96 | 43874 |
| 10 | T21 d | 40 | his | 6 | 1.74 | 95.71 | 2.55 | 45249 |
| 3.4 | T21 d | 40 | phos | 7 | 2.43 | 95.14 | 2.42 | 54476 |
| 50 | T21 d | 40 | ace-suc-tw | 5 | 9.45 | 87.66 | 2.89 | 51846 |
| 30 | T21 d | 40 | ace-suc-tw | 5 | 5.35 | 91.28 | 3.37 | 50456 |
| 10 | T21 d | 40 | ace-suc-tw | 5 | 1.94 | 94.29 | 3.78 | 50414 |
| 50 | T21 d | 40 | his-suc-tw | 6 | 8.72 | 89.02 | 2.26 | 46410 |
| 30 | T21 d | 40 | his-suc-tw | 6 | 5.08 | 92.63 | 2.29 | 43210 |
| 10 | T21 d | 40 | his-suc-tw | 6 | 1.95 | 95.68 | 2.37 | 46539 |
| 6.6 | T21 d | 40 | PBS | 7 | 4.71 | 92.43 | 2.86 | 68811 |

Buffer key (all buffers contain 0.02% sodium azide to prevent microbial growth):
ace = 15 mM acetate pH 5;
his = 15 mM histidine pH 6;
phos = 15 mM phosphate pH 7
ace-suc-tw = 30 mM acetate, 80 mg/ml sucrose, 0.02% Tw80
his-suc-tw = 30 mM histidine, 80 mg/ml sucrose, 0.02% Tw80
PBS = phosphate buffered saline

TABLE 65
Storage Stability At 5° C. Of H1a11.1.A6-SL-Av At Different Concentrations
And In Different Buffers, Excipients, And pHs

| Protein | | | | | | | | |
|---|---|---|---|---|---|---|---|---|
| conc | | temp | | | % | % | % | Total |
| (mg/ml) | time | (° C.) | buffer | pH | Aggregrate | Monomer | Fragment | Area |
| — | pre- | — | — | — | 1.19 | 97.75 | 1.06 | 58988 |
| | dialysis | | | | | | | |
| 50, 30, 10 | T0 | — | ace | 5 | 1.23 | 97.85 | 0.92 | 47138 |
| 50, 30, 10 | T0 | — | his | 6 | 1.30 | 97.84 | 0.87 | 44711 |
| 50, 30, 10 | T0 | — | phos | 7 | 0.86 | 98.00 | 1.14 | 57373 |
| 50, 30, 10 | T0 | — | ace-suc-tw | 5 | 1.30 | 97.85 | 0.85 | 52129 |
| 50, 30, 10 | T0 | — | his-suc-tw | 6 | 1.28 | 97.85 | 0.87 | 47563 |
| 6.6 | T0 | — | PBS | 7 | 1.35 | 97.76 | 0.89 | 71146 |
| 50 | T7 d | 5 | ace | 5 | 3.52 | 95.40 | 1.08 | 46507 |
| 30 | T7 d | 5 | ace | 5 | 2.35 | 96.55 | 1.10 | 40267 |
| 10 | T7 d | 5 | ace | 5 | 1.46 | 97.48 | 1.07 | 47761 |
| 50 | T7 d | 5 | his | 6 | 3.48 | 95.24 | 1.27 | 46306 |
| 30 | T7 d | 5 | his | 6 | 3.63 | 95.34 | 1.03 | 46581 |
| 10 | T7 d | 5 | his | 6 | 2.01 | 97.01 | 0.98 | 45843 |
| 3.4 | T7 d | 5 | phos | 7 | 1.42 | 97.41 | 1.17 | 52150 |
| 50 | T7 d | 5 | ace-suc-tw | 5 | 3.97 | 94.99 | 1.04 | 53439 |
| 30 | T7 d | 5 | ace-suc-tw | 5 | 2.65 | 96.35 | 1.00 | 52119 |
| 10 | T7 d | 5 | ace-suc-tw | 5 | 1.60 | 97.23 | 1.17 | 52681 |
| 50 | T7 d | 5 | his-suc-tw | 6 | 4.81 | 94.10 | 1.09 | 48136 |
| 30 | T7 d | 5 | his-suc-tw | 6 | 3.38 | 95.49 | 1.13 | 48266 |
| 10 | T7 d | 5 | his-suc-tw | 6 | 1.88 | 96.90 | 1.22 | 47966 |
| 6.6 | T7 d | 5 | PBS | 7 | 2.25 | 96.78 | 0.97 | 68563 |
| 50 | T21 d | 5 | ace | 5 | 7.82 | 92.06 | 0.11 | 47402 |
| 30 | T21 d | 5 | ace | 5 | 4.22 | 95.68 | 0.10 | 45736 |
| 10 | T21 d | 5 | ace | 5 | 1.77 | 98.13 | 0.09 | 47900 |
| 50 | T21 d | 5 | his | 6 | 7.36 | 92.48 | 0.17 | 47111 |
| 30 | T21 d | 5 | his | 6 | 6.51 | 93.34 | 0.15 | 43911 |
| 10 | T21 d | 5 | his | 6 | 2.92 | 96.98 | 0.10 | 45446 |
| 3.4 | T21 d | 5 | phos | 7 | 0.05 | 99.53 | 0.42 | 45856 |
| 50 | T21 d | 5 | ace-suc-tw | 5 | 9.13 | 90.73 | 0.14 | 52424 |
| 30 | T21 d | 5 | ace-suc-tw | 5 | 4.79 | 95.07 | 0.15 | 51575 |
| 10 | T21 d | 5 | ace-suc-tw | 5 | 1.98 | 97.87 | 0.15 | 51870 |
| 50 | T21 d | 5 | his-suc-tw | 6 | 9.50 | 90.30 | 0.20 | 46588 |
| 30 | T21 d | 5 | his-suc-tw | 6 | 6.20 | 93.64 | 0.16 | 46023 |
| 10 | T21 d | 5 | his-suc-tw | 6 | 2.61 | 97.27 | 0.12 | 47285 |
| 6.6 | T21 d | 5 | PBS | 7 | 3.12 | 96.76 | 0.13 | 62856 |

Buffer key (all buffers contain 0.02% sodium azide to prevent microbial growth):
ace = 15 mM acetate pH 5;
his = 15 mM histidine pH 6;
phos = 15 mM phosphate pH 7
ace-suc-tw = 30 mM acetate, 80 mg/ml sucrose, 0.02% Tw80
his-suc-tw = 30 mM histidine, 80 mg/ml sucrose, 0.02% Tw80
PBS = phosphate buffered saline

### Sequences

Amino acid sequences of the heavy and light chains for the DVD-Ig proteins described herein are provided below in Table 66.

TABLE 66
DVD-Ig protein sequences

| DVD Heavy | Outer | Inner | |
|---|---|---|---|
| or Light | Variable | Variable | Sequence (Line Break Between |
| Chain | Domain | Domain | Variable and Constant Regions) |
| Name | Name | Name | 12345678901234567890123456789012345 |
| DVD005H | AB001VH | AB007VH | QVQLQQPGAELVKPGASVKMSCKASGYTFTSYNMH |
| | | | WVKQTPGRGLEWIGAIYPGNGDTSYNQKFKGKATL |
| | | | TADKSSSTAYMQLSSLTSEDSAVYYCARSTYYGGD |
| | | | WYFNVWGAGTTVTVSAASTKGPQVQLQESGPGLVK |
| | | | PSETLSLTCAVSGGSISGGYGWGWIRQPPGKGLEW |
| | | | IGSFYSSSGNTYYNPSLKSQVTISTDTSKNQFSLK |
| | | | LNSMTAADTAVYYCVRDRLFSVVGMVYNNWFDVWG |
| | | | PGVLVTVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 30) |
| DVD005L | AB001VL | AB007VL | QIVLSQSPAILSPSPGEKVTMTCRASSSVSYIHWF |
| | | | QQKPGSSPKPWIYATSNLASGVPVRFSGSGSGTSY |
| | | | SLTISRVEAEDAATYYCQQWTSNPPTFGGGTKLEI |
| | | | KRTVAAPESALTQPPSVSGAPGQKVTISCTGSTSN |
| | | | IGGYDLHWYQQLPGTAPKLLIYDINKRPSGISDRF |
| | | | SGSKSGTAASLAITGLQTEDEADYYCQSYDSSLNA |
| | | | QVFGGGTRLTVLG |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 31) |
| DVD006H | AB007VH | AB001VH | QVQLQESGPGLVKPSETLSLTCAVSGGSISGGYGW |
| | | | GWIRQPPGKGLEWIGSFYSSSGNTYYNPSLKSQVT |
| | | | ISTDTSKNQFSLKLNSMTAADTAVYYCVRDRLFSV |
| | | | VGMVYNNWFDVWGPGVLVTVSSASTKGPQVQLQQP |
| | | | GAELVKPGASVKMSCKASGYTFTSYNMHWVKQTPG |
| | | | RGLEWIGAIYPGNGDTSYNQKFKGKATLTADKSSS |
| | | | TAYMQLSSLTSEDSAVYYCARSTYYGGDWYFNVWG |
| | | | AGTTVTVSA |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 32) |
| DVD006L | AB007VL | AB001VL | ESALTQPPSVSGAPGQKVTISCTGSTSNIGGYDLH |
| | | | WYQQLPGTAPKLLIYDINKRPSGISDRFSGSKSGT |
| | | | AASLAITGLQTEDEADYYCQSYDSSLNAQVFGGGT |
| | | | RLTVLGQPKAAPQIVLSQSPAILSPSPGEKVTMTC |
| | | | RASSSVSYIHWFQQKPGSSPKPWIYATSNLASGVP |
| | | | VRFSGSGSGTSYSLTISRVEAEDAATYYCQQWTSN |
| | | | PPTFGGGTKLEIKR |
| | | | QPKAAPSVTLFPPSSEELQANKATLVCLISDFYPG |
| | | | AVTVAWKADSSPVKAGVETTTPSKQSNNKYAASSY |
| | | | LSLTPEQWKSHRSYSCQVTHEGSTVEKTVAPTECS |
| | | | (SEQ ID NO: 33) |
| DVD037H | AB014VH | AB004VH | EVQLVESGGGLVQPGGSLRLSCAASGYTFTNYGMN |
| (VEGF/HER | | | WVRQAPGKGLEWVGWINTYTGEPTYAADFKRRFTF |
| 2) | | | SLDTSKSTAYLQMNSLRAEDTAVYYCAKYPHYYGS |
| | | | SHWYFDVWGQGTLVTVSSASTKGPEVQLVESGGGL |
| | | | VQPGGSLRLSCAASGFNIKDTYIHWVRQAPGKGLE |
| | | | WVARIYPTNGYTRYADSVKGRFTISADTSKNTAYL |
| | | | QMNSLRAEDTAVYYCSRWGGDGFYAMDYWGQGTLV |
| | | | TVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 34) |
| DVD037L | AB014VL | AB004VL | DIQMTQSPSSLSASVGDRVTITCSASQDISNYLNW |
| (VEGF/HER | | | YQQKPGKAPKVLIYFTSSLHSGVPSRFSGSGSGTD |
| 2) | | | FTLTISSLQPEDFATYYCQQYSTVPWTFGQGTKVE |
| | | | IKRTVAAPDIQMTQSPSSLSASVGDRVTITCRASQ |
| | | | DVNTAVAWYQQKPGKAPKLLIYSASFLYSGVPSRF |
| | | | SGSRSGTDFTLTISSLQPEDFATYYCQQHYTTPPT |
| | | | FGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 35) |
| DVD038H | AB004VH | AB014VH | EVQLVESGGGLVQPGGSLRLSCAASGFNIKDTYIH |
| | | | WVRQAPGKGLEWVARIYPTNGYTRYADSVKGRFTI |
| | | | SADTSKNTAYLQMNSLRAEDTAVYYCSRWGGDGFY |
| | | | AMDYWGQGTLVTVSSASTKGPEVQLVESGGGLVQP |
| | | | GGSLRLSCAASGYTFTNYGMNWVRQAPGKGLEWVG |
| | | | WINTYTGEPTYAADFKRRFTFSLDTSKSTAYLQMN |
| | | | SLRAEDTAVYYCAKYPHYYGSSHWYFDVWGQGTLV |
| | | | TVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 36) |
| DVD038L | AB004VL | AB014VL | DIQMTQSPSSLSASVGDRVTITCRASQDVNTAVAW |
| | | | YQQKPGKAPKLLIYSASFLYSGVPSRFSGSRSGTD |
| | | | FTLTISSLQPEDFATYYCQQHYTTPPTFGQGTKVE |
| | | | IKRTVAAPDIQMTQSPSSLSASVGDRVTITCSASQ |
| | | | DISNYLNWYQQKPGKAPKVLIYFTSSLHSGVPSRF |
| | | | SGSGSGTDFTLTISSLQPEDFATYYCQQYSTVPWT |
| | | | FGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 37) |
| DVD053H | AB017VH | AB018VH | EVQLVESGGGLVQPGRSLRLSCAASGFTFDDYAMH |
| | | | WVRQAPGKGLEWVSAITWNSGHIDYADSVEGRFTI |
| | | | SRDNAKNSLYLQMNSLRAEDTAVYYCAKVSYLSTA |
| | | | SSLDYWGQGTLVTVSSASTKGPEVQLLESGGGLVQ |
| | | | PGGSLRLSCAASGFTFSSYAMSWVRQAPGKGLEWV |
| | | | SGITGSGGSTYYADSVKGRFTISRDNSKNTLYLQM |
| | | | NSLRAEDTAVYYCAKDPGTTVIMSWFDPWGQGTLV |
| | | | TVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 38) |
| DVD053L | AB017VL | AB018VL | DIQMTQSPSSLSASVGDRVTITCRASQGIRNYLAW |
| | | | YQQKPGKAPKLLIYAASTLQSGVPSRFSGSGSGTD |
| | | | FTLTISSLQPEDVATYYCQRYNRAPYTFGQGTKVE |
| | | | IKRTVAAPEIVLTQSPGTLSLSPGERATLSCRASQ |
| | | | SVRGRYLAWYQQKPGQAPRLLIYGASSRATGIPDR |
| | | | FSGSGSGTDFTLTISRLEPEDFAVFYCQQYGSSPR |
| | | | TFGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 39) |
| DVD054H | AB018VH | AB017VH | EVQLLESGGGLVQPGGSLRLSCAASGFTFSSYAMS |
| | | | WVRQAPGKGLEWVSGITGSGGSTYYADSVKGRFTI |
| | | | SRDNSKNTLYLQMNSLRAEDTAVYYCAKDPGTTVI |
| | | | MSWFDPWGQGTLVTVSSASTKGPEVQLVESGGGLV |
| | | | QPGRSLRLSCAASGFTFDDYAMHWVRQAPGKGLEW |
| | | | VSAITWNSGHIDYADSVEGRFTISRDNAKNSLYLQ |
| | | | MNSLRAEDTAVYYCAKVSYLSTASSLDYWGQGTLV |
| | | | TVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 40) |
| DVD054L | AB018VL | AB017VL | EIVLTQSPGTLSLSPGERATLSCRASQSVRGRYLA |
| | | | WYQQKPGQAPRLLIYGASSRATGIPDRFSGSGSGT |
| | | | DFTLTISRLEPEDFAVFYCQQYGSSPRTFGQGTKV |
| | | | EIKRTVAAPDIQMTQSPSSLSASVGDRVTITCRAS |
| | | | QGIRNYLAWYQQKPGKAPKLLIYAASTLQSGVPSR |
| | | | FSGSGSGTDFTLTISSLQPEDVATYYCQRYNRAPY |
| | | | TFGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 41) |
| DVD065H | AB017VH | AB023VH | EVQLVESGGGLVQPGRSLRLSCAASGFTFDDYAMH |
| | | | WVRQAPGKGLEWVSAITWNSGHIDYADSVEGRFTI |
| | | | SRDNAKNSLYLQMNSLRAEDTAVYYCAKVSYLSTA |
| | | | SSLDYWGQGTLVTVSSASTKGPEVQLVESGGGLVQ |
| | | | PANSLKLSCAASGFTFSDYAMAWVRQSPKKGLEWV |
| | | | ATIIYDGSSTYYRDSVKGRFTISRDNAKSTLYLQM |
| | | | DSLRSEDTATYYCATGLGIATDYFDYWGQGVLVTV |
| | | | SS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 42) |
| DVD065L | AB017VL | AB023VL | DIQMTQSPSSLSASVGDRVTITCRASQGIRNYLAW |
| | | | YQQKPGKAPKLLIYAASTLQSGVPSRFSGSGSGTD |
| | | | FTLTISSLQPEDVATYYCQRYNRAPYTFGQGTKVE |
| | | | IKRTVAAPDIRMTQSPASLSASLGETVNIECLASE |
| | | | DIYSDLAWYQQKPGKSPQLLIYNANSLQNGVPSRF |
| | | | SGSGSGTQYSLKINSLQSEDVATYFCQQYNNYPPT |
| | | | FGGGTKLELKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 43) |
| DVD066H | AB023VH | AB017VH | EVQLVESGGGLVQPANSLKLSCAASGFTFSDYAMA |
| | | | WVRQSPKKGLEWVATIIYDGSSTYYRDSVKGRFTI |
| | | | SRDNAKSTLYLQMDSLRSEDTATYYCATGLGIATD |
| | | | YFDYWGQGVLVTVSSASTKGPEVQLVESGGGLVQP |
| | | | GRSLRLSCAASGFTFDDYAMHWVRQAPGKGLEWVS |
| | | | AITWNSGHIDYADSVEGRFTISRDNAKNSLYLQMN |
| | | | SLRAEDTAVYYCAKVSYLSTASSLDYWGQGTLVTV |
| | | | SS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 44) |
| DVD066L | AB023VL | AB017VL | DIRMTQSPASLSASLGETVNIECLASEDIYSDLAW |
| | | | YQQKPGKSPQLLIYNANSLQNGVPSRFSGSGSGTQ |
| | | | YSLKINSLQSEDVATYFCQQYNNYPPTFGGGTKLE |
| | | | LKRTVAAPDIQMTQSPSSLSASVGDRVTITCRASQ |
| | | | GIRNYLAWYQQKPGKAPKLLIYAASTLQSGVPSRF |
| | | | SGSGSGTDFTLTISSLQPEDVATYYCQRYNRAPYT |
| | | | FGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 45) |
| DVD165H | AB001VH | AB018VH | QVQLQQPGAELVKPGASVKMSCKASGYTFTSYNMH |
| | | | WVKQTPGRGLEWIGAIYPGNGDTSYNQKFKGKATL |
| | | | TADKSSSTAYMQLSSLTSEDSAVYYCARSTYYGGD |
| | | | WYFNVWGAGTTVTVSAASTKGPEVQLLESGGGLVQ |
| | | | PGGSLRLSCAASGFTFSSYAMSWVRQAPGKGLEWV |
| | | | SGITGSGGSTYYADSVKGRFTISRDNSKNTLYLQM |
| | | | NSLRAEDTAVYYCAKDPGTTVIMSWFDPWGQGTLV |
| | | | TVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 46) |
| DVD165L | AB001VL | AB018VL | QIVLSQSPAILSPSPGEKVTMTCRASSSVSYIHWF |
| | | | QQKPGSSPKPWIYATSNLASGVPVRFSGSGSGTSY |
| | | | SLTISRVEAEDAATYYCQQWTSNPPTFGGGTKLEI |
| | | | KRTVAAPEIVLTQSPGTLSLSPGERATLSCRASQS |
| | | | VRGRYLAWYQQKPGQAPRLLIYGASSRATGIPDRF |
| | | | SGSGSGTDFTLTISRLEPEDFAVFYCQQYGSSPRT |
| | | | FGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 47) |
| DVD166H | AB018VH | AB001VH | EVQLLESGGGLVQPGGSLRLSCAASGFTFSSYAMS |
| | | | WVRQAPGKGLEWVSGITGSGGSTYYADSVKGRFTI |
| | | | SRDNSKNTLYLQMNSLRAEDTAVYYCAKDPGTTVI |
| | | | MSWFDPWGQGTLVTVSSASTKGPQVQLQQPGAELV |
| | | | KPGASVKMSCKASGYTFTSYNMHWVKQTPGRGLEW |
| | | | IGAIYPGNGDTSYNQKFKGKATLTADKSSSTAYMQ |
| | | | LSSLTSEDSAVYYCARSTYYGGDWYFNVWGAGTTV |
| | | | TVSA |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 48) |
| DVD166L | AB018VL | AB001VL | EIVLTQSPGTLSLSPGERATLSCRASQSVRGRYLA |
| | | | WYQQKPGQAPRLLIYGASSRATGIPDRFSGSGSGT |
| | | | DFTLTISRLEPEDFAVFYCQQYGSSPRTFGQGTKV |
| | | | EIKRTVAAPQIVLSQSPAILSPSPGEKVTMTCRAS |
| | | | SSVSYIHWFQQKPGSSPKPWIYATSNLASGVPVRF |
| | | | SGSGSGTSYSLTISRVEAEDAATYYCQQWTSNPPT |
| | | | FGGGTKLEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 49) |
| DVD257H | DLL4H | P1GFH | EVQLVESGGGLVQPGGSLRLSCAASGFTFTDNWIS |
| | | | WVRQAPGKGLEWVGYISPNSGFTYYADSVKGRFTI |
| | | | SADTSKNTAYLQMNSLRAEDTAVYYCARDNFGGYF |
| | | | DYWGQGTLVTVSSASTKGPQVQLQQSGAELVKPGA |
| | | | SVKISCKASGYTFTDYYINWVKLAPGQGLEWIGWI |
| | | | YPGSGNTKYNEKFKGKATLTIDTSSSTAYMQLSSL |
| | | | TSEDTAVYFCVRDSPFFDYWGQGTLLTVSS |
| | | | (SEQ ID NO: 50) |
| DVD257L | DLL4L | P1GFL | DIQMTQSPSSLSASVGDRVTITCRASQDVSTAVAW |
| | | | YQQKPGKAPKLLIYSASFLYSGVPSRFSGSGSGTD |
| | | | FTLTISSLQPEDFATTYYCQQSYTGTVTFGQGTKV |
| | | | EIKRTVAAPDIVLTQSPDSLAVSLGERVTMNCKSS |
| | | | QSLLNSGMRKSFLAWYQQKPGQSPKLLIYWASTRE |
| | | | SGVPDRFTGSGSGTDFTLTISSVQAEDVAVYYCKQ |
| | | | SYHLFTFGSGTKLEIKR |
| | | | (SEQ ID NO: 51) |
| DVD258H | P1GFH | DLL4H | QVQLQQSGAELVKPGASVKISCKASGYTFTDYYIN |
| | | | WVKLAPGQGLEWIGWIYPGSGNTKYNEKFKGKATL |
| | | | TIDTSSSTAYMQLSSLTSEDTAVYFCVRDSPFFDY |
| | | | WGQGTLLTVSSASTKGPEVQLVESGGGLVQPGGSL |
| | | | RLSCAASGFTFTDNWISWVRQAPGKGLEWVGYISP |
| | | | NSGFTYYADSVKGRFTISADTSKNTAYLQMNSLRA |
| | | | EDTAVYYCARDNFGGYFDYWGQGTLVTVSS |
| | | | (SEQ ID NO: 52) |
| DVD258L | P1GFL | DLL4L | DIVLTQSPDSLAVSLGERVTMNCKSSQSLLNSGMR |
| | | | KSFLAWYQQKPGQSPKLLIYWASTRESGVPDRFTG |
| | | | SGSGTDFTLTISSVQAEDVAVYYCKQSYHLFTFGS |
| | | | GTKLEIKRTVAAPDIQMTQSPSSLSASVGDRVTIT |
| | | | CRASQDVSTAVAWYQQKPGKAPKLLIYSASFLYSG |
| | | | VPSRFSGSGSGTDFTLTISSLQPEDFATTYYCQQS |
| | | | YTGTVTFGQGTKVEIKR |
| | | | (SEQ ID NO: 53) |
| DVD277H | AB017VH | AB050VH | EVQLVESGGGLVQPGRSLRLSCAASGFTFDDYAMH |
| | | | WVRQAPGKGLEWVSAITWNSGHIDYADSVEGRFTI |
| | | | SRDNAKNSLYLQMNSLRAEDTAVYYCAKVSYLSTA |
| | | | SSLDYWGQGTLVTVSSASTKGPEVQLQQSGPELMK |
| | | | PGASVKMSCKASGYTFTDYNMHWMKQNQGKSLEWI |
| | | | GEINPNSGGSGYNQKFKGKATLTVDKSSSTAYMEL |
| | | | RSLTSEDSAVYYCARLGYYGNYEDWYFDVWGAGTT |
| | | | VTVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 54) |
| DVD277L | AB017VL | AB050VL | DIQMTQSPSSLSASVGDRVTITCRASQGIRNYLAW |
| | | | YQQKPGKAPKLLIYAASTLQSGVPSRFSGSGSGTD |
| | | | FTLTISSLQPEDVATYYCQRYNRAPYTFGQGTKVE |
| | | | IKRTVAAPDLQMTQTTSSLSASLGDRVTISCRASQ |
| | | | DISNYLNWYQQKPDGTVKLLIFYTSTLQSGVPSRF |
| | | | SGSGSGTNYSLTITNLEQDDAATYFCQQGDTLPYT |
| | | | FGGGTKLEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 55) |
| DVD278H | AB050VH | AB017VH | EVQLQQSGPELMKPGASVKMSCKASGYTFTDYNMH |
| | | | WMKQNQGKSLEWIGEINPNSGGSGYNQKFKGKATL |
| | | | TVDKSSSTAYMELRSLTSEDSAVYYCARLGYYGNY |
| | | | EDWYFDVWGAGTTVTVSSASTKGPEVQLVESGGGL |
| | | | VQPGRSLRLSCAASGFTFDDYAMHWVRQAPGKGLE |
| | | | WVSAITWNSGHIDYADSVEGRFTISRDNAKNSLYL |
| | | | QMNSLRAEDTAVYYCAKVSYLSTASSLDYWGQGTL |
| | | | VTVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 56) |
| DVD278L | AB050VL | AB017VL | DLQMTQTTSSLSASLGDRVTISCRASQDISNYLNW |
| | | | YQQKPDGTVKLLIFYTSTLQSGVPSRFSGSGSGTN |
| | | | YSLTITNLEQDDAATYFCQQGDTLPYTFGGGTKLE |
| | | | IKRTVAAPDIQMTQSPSSLSASVGDRVTITCRASQ |
| | | | GIRNYLAWYQQKPGKAPKLLIYAASTLQSGVPSRF |
| | | | SGSGSGTDFTLTISSLQPEDVATYYCQRYNRAPYT |
| | | | FGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 57) |
| DVD281H | AB056VH | AB053VH | QVQLQQSGAELMKPGASVKLSCKATGYTFTGSWIE |
| | | | WIKQRPGHGLEWIGQILPGSGSAYYNEKFKGKATF |
| | | | TADTSSKTVYIQLISLTTEDSAIYYCAREDNYGSS |
| | | | SLAYWGQGTLLTVSAASTKGPEVQLVESGGGLVQP |
| | | | GGSLRLSCAVSGYSITSGYSWNWIRQAPGKGLEWV |
| | | | ASITYDGSTNYNPSVKGRITISRDDSKNTFYLQMN |
| | | | SLRAEDTAVYYCARGSHYFGHWHFAVWGQGTLVTV |
| | | | SS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 58) |
| DVD281L | AB056VL | AB053VL | DILLTQSPAILSVSPGERVSFSCRASQSIGTNIHW |
| | | | YQQRTNGSPRLLIKYASESISGIPSRFSGGGSGTD |
| | | | FTLSINSVESEDIADYYCQQSNNWPLTFGAGTKLE |
| | | | LKRTVAAPDIQLTQSPSSLSASVGDRVTITCRASQ |
| | | | SVDYDGDSYMNWYQQKPGKAPKLLIYAASYLESGV |
| | | | PSRFSGSGSGTDFTLTISSLQPEDFATYYCQQSHE |
| | | | DPYTFGQGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 59) |
| DVD282H | AB053VH | AB056VH | EVQLVESGGGLVQPGGSLRLSCAVSGYSITSGYSW |
| | | | NWIRQAPGKGLEWVASITYDGSTNYNPSVKGRITI |
| | | | SRDDSKNTFYLQMNSLRAEDTAVYYCARGSHYFGH |
| | | | WHFAVWGQGTLVTVSSASTKGPQVQLQQSGAELMK |
| | | | PGASVKLSCKATGYTFTGSWIEWIKQRPGHGLEWI |
| | | | GQILPGSGSAYYNEKFKGKATFTADTSSKTVYIQL |
| | | | ISLTTEDSAIYYCAREDNYGSSSLAYWGQGTLLTV |
| | | | SA |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 60) |
| DVD282L | AB053VL | AB056VL | DIQLTQSPSSLSASVGDRVTITCRASQSVDYDGDS |
| | | | YMNWYQQKPGKAPKLLIYAASYLESGVPSRFSGSG |
| | | | SGTDFTLTISSLQPEDFATYYCQQSHEDPYTFGQG |
| | | | TKVEIKRTVAAPDILLTQSPAILSVSPGERVSFSC |
| | | | RASQSIGTNIHWYQQRTNGSPRLLIKYASESISGI |
| | | | PSRFSGGGSGTDFTLSINSVESEDIADYYCQQSNN |
| | | | WPLTFGAGTKLELKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 61) |
| TNF/IL- | TNFH | IL-17H | EVQLVESGGGLVQPGRSLRLSCAASGFTFDDYAMH |
| 17H | | | WVRQAPGKGLEWVSAITWNSGHIDYADSVEGRFTI |
| (DVD A) | | | SRDNAKNSLYLQMNSLRAEDTAVYYCAKVSYLSTA |
| | | | SSLDYWGQGTLVTVSSGGGGSGGGGSEVQLVQSGA |
| | | | EVKKPGSSVKVSCKASGGSFGGYGIGWVRQAPGQG |
| | | | LEWMGGITPFFGFADYAQKFQGRVTITADESTTTA |
| | | | YMELSGLTSDDTAVYYCARDPNEFWNGYYSTHDFD |
| | | | SWGQGTTVTVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPELLGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK |
| | | | (SEQ ID NO: 62) |
| TNF/IL- | TNFL | IL-17L | DIQMTQSPSSLSASVGDRVTITCRASQGIRNYLAW |
| 17L | | | YQQKPGKAPKLLIYAASTLQSGVPSRFSGSGSGTD |
| (DVD A) | | | FTLTISSLQPEDVATYYCQRYNRAPYTFGQGTKVE |
| | | | IKRGGSGGGGSGSEIVLTQSPDFQSVTPKEKVTIT |
| | | | CRASQDIGSELHWYQQKPDQPPKLLIKYASHSTSG |
| | | | VPSRFSGSGSGTDFTLTINGLEAEDAGTYYCHQTD |
| | | | SLPYTFGPGTKVDIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 63) |
| TNF/PGE2H | TNFH | PGE2 | EVQLVESGGGLVQPGRSLRLSCAASGFTFDDYAMH |
| (DVD B) | | | WVRQAPGKGLEWVSAITWNSGHIDYADSVEGRFTI |
| | | | SRDNAKNSLYLQMNSLRAEDTAVYYCAKVSYLSTA |
| | | | SSLDYWGQGTLVTVSSASTKGPEVQLVQSGAEVKK |
| | | | PGASVKVSCKASGYTFTKYWLGWVRQAPGQGLEWM |
| | | | GDIYPGYDYTHYNEKFKDRVTLTTDTSTSTAYMEL |
| | | | RSLRSDDTAVYYCARSDGSSTYWGQGTLVTVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPELLGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK |
| | | | (SEQ ID NO: 64) |
| TNF/PGE2L | TNF | PGE2 | DIQMTQSPSSLSASVGDRVTITCRASQGIRNYLAW |
| (DVD B) | | | YQQKPGKAPKLLIYAASTLQSGVPSRFSGSGSGTD |
| | | | FTLTISSLQPEDVATYYCQRYNRAPYTFGQGTKVE |
| | | | IKRTVAAPDVLMTQTPLSLPVTPGEPASISCTSSQ |
| | | | NIVHSNGNTYLEWYLQKPGQSPQLLIYKVSNRFSG |
| | | | VPDRFSGSGSGTDFTLKISRVEAEDVGVYYCFQVS |
| | | | HVPYTFGGGTKVEIKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 65) |
| IL-1a/IL- | IL-1aH | IL-1bH | EVQLVESGGGVVQPGRSLRLSCSASGFIFSRYDMS |
| 1bH | | | WVRQAPGKGLEWVAYISHGGAGTYYPDSVKGRFTI |
| (DVD C) | | | SRDNSKNTLFLQMDSLRPEDTGVYFCARGGVTKGY |
| | | | FDVWGQGTPVTVSSASTKGPQVQLVESGGGVVQPG |
| | | | RSLRLSCTASGFTFSMFGVHWVRQAPGKGLEWVAA |
| | | | VSYDGSNKYYAESVKGRFTISRDNSKNILFLQMDS |
| | | | LRLEDTAVYYCARGRPKVVIPAPLAHWGQGTLVTF |
| | | | SS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK (SEQ ID NO: 66) |
| IL-1a/IL- | IL-1aL | IL-1bL | DIQMTQSPSSLSASVGDRVTITCRASGNIHNYLTW |
| 1bL | | | YQQTPGKAPKLLIYNAKTLADGVPSRFSGSGSGTD |
| (DVD C) | | | YTFTISSLQPEDIATYYCQHFWSIPYTFGQGTKLQ |
| | | | ITRTVAAPDIQMTQSPSSVSASVGDRVTITCRASQ |
| | | | GISSWLAWYQQKPGKAPKLLIYEASNLETGVPSRF |
| | | | SGSGSGSDFTLTISSLQPEDFATYYCQQTSSFLLS |
| | | | FGGGTKVEHKR |
| | | | TVAAPSVFIFPPSDEQLKSGTASVVCLLNNFYPRE |
| | | | AKVQWKVDNALQSGNSQESVTEQDSKDSTYSLSST |
| | | | LTLSKADYEKHKVYACEVTHQGLSSPVTKSFNRGE |
| | | | C (SEQ ID NO: 67) |
| DLL4/VEGF | | | EVQLVESGGGLVQPGGSLRLSCAASGFTFRHFPMA |
| HC | | | WVRQAPGKGLEWVATISSSDAWPSYRDSVKGRFTI |
| (h1A11.1. | | | SRDNAKNSLYLQMNSLRAEDTAVYYCSRGYYNSPF |
| A6-LS-Av) | | | AYWGQGTLVTVSSASTKGPSVFPLAPEVQLVESGG |
| | | | GLVQPGGSLRLSCAASGYTFTNYGMNWVRQAPGKG |
| | | | LEWVGWINTYTGEPTYAADFKRRFTFSLDTSKSTA |
| | | | YLQMNSLRAEDTAVYYCAKYPHYYGSSHWYFDVWG |
| | | | QGTLVTVSS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK* (SEQ ID NO: 68) |
| DLL4/VEGF | | | DIQMTQSPSSLSASVGDRVTITCRASEDIYSNLAW |
| LC | | | YQQKPGKAPKLLIYDTNNLADGVPSRFSGSGSGTD |
| (h1A11.1. | | | FTLTISSLQPEDFATYYCQQYNNYPPTFGQGTKLE |
| A6-LS-Av) | | | IKR |
| | | | TVAAPDIQMTQSPSSLSASVGDRVTITCSASQDIS |
| | | | NYLNWYQQKPGKAPKVLIYFTSSLHSGVPSRFSGS |
| | | | GSGTDFTLTISSLQPEDFATYYCQQYSTVPWTFGQ |
| | | | GTKVEIKRTVAAPSVFIFPPSDEQLKSGTASVVCL |
| | | | LNNFYPREAKVQWKVDNALQSGNSQESVTEQDSKD |
| | | | STYSLSSTLTLSKADYEKHKVYACEVTHQGLSSPV |
| | | | TKSFNRGEC*(SEQ ID NO: 69) |
| DLL4/VEGF | | | EVQLVESGGGLVQPGGSLRLSCAASGFTFRHFPMA |
| HC | | | WVRQAPGKGLEWVATISSSDAWPSYRDSVKGRFTI |
| (h1A11.1. | | | SRDNAKNSLYLQMNSLRAEDTAVYYCSRGYYNSPF |
| A6-SL-Av) | | | AYWGQGTLVTVSSASTKGPEVQLVESGGGLVQPGG |
| | | | SLRLSCAASGYTFTNYGMNWVRQAPGKGLEWVGWI |
| | | | NTYTGEPTYAADFKRRFTFSLDTSKSTAYLQMNSL |
| | | | RAEDTAVYYCAKYPHYYGSSHWYFDVWGQGTLVTV |
| | | | SS |
| | | | ASTKGPSVFPLAPSSKSTSGGTAALGCLVKDYFPE |
| | | | PVTVSWNSGALTSGVHTFPAVLQSSGLYSLSSVVT |
| | | | VPSSSLGTQTYICNVNHKPSNTKVDKKVEPKSCDK |
| | | | THTCPPCPAPEAAGGPSVFLFPPKPKDTLMISRTP |
| | | | EVTCVVVDVSHEDPEVKFNWYVDGVEVHNAKTKPR |
| | | | EEQYNSTYRVVSVLTVLHQDWLNGKEYKCKVSNKA |
| | | | LPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEAL |
| | | | HNHYTQKSLSLSPGK |
| | | | (SEQ ID NO: 74) |
| DLL4/VEGF | | | DIQMTQSPSSLSASVGDRVTITCRASEDIYSNLAW |
| LC | | | YQQKPGKAPKLLIYDTNNLADGVPSRFSGSGSGTD |
| (h1A11.1. | | | FTLTISSLQPEDFATYYCQQYNNYPPTFGQGTKLE |
| A6-SL-Av) | | | IKRTVAAPSVFIFPPDIQMTQSPSSLSASVGDRVT |
| | | | ITCSASQDISNYLNWYQQKPGKAPKVLIYFTSSLH |
| | | | SGVPSRFSGSGSGTDFTLTISSLQPEDFATYYCQQ |
| | | | YSTVPWTFGQGTKVEIKR |
| | | | TVAAPSVFIFPPSDEQLKSGTASVVCLLNLNYPRE |
| | | | AKVQWKVDNALQSGNSQESVTEQDSKDSTYSLSST |
| | | | LTLSKADYEKHKVYACEVTHQGLSSPVTKSFNRGE |
| | | | C |
| | | | (SEQ ID NO: 75) |
| IL12/IL18 | | | EVTLRESGPALVKPTQTLTLTCTFSGFSLSKSVMG |
| HC | | | VSWIRQPPGKALEWLAHIYWDDDKYYNPSLKSRLT |
| | | | ISKDTSKNQV VLTMTNMDPVDTATYYCARR |
| | | | GIRSAMDYWGQGTTVTVSSASTKGPEVQLVQSGTE |
| | | | VKKPG ESLKISCKGSGYTVTSYWIG |
| | | | WVRQMPGKGLEWMGFIYPGDSETRYSPTFQGQVTI |
| | | | SADKS FNTAFLQWSSLKASDTAMYY |
| | | | CARVGSGWYPYTFDIWGQGTMVTVSS |
| | | | ASTKGPSVFPLAPSSKSTSGGTAALGCLVKDYFPE |
| | | | PVTVS WNSGALTSGVHTFPAVLQSS |
| | | | GLYSLSSVVTVPSSSLGTQTYICNVNHKPSNTKVD |
| | | | KKVEP KSCDKTHTCPPCPAPEAAGG |
| | | | PSVFLFPPKPKDTLMISRTPEVTCVVVDVSHEDPE |
| | | | VKFNW YVDGVEVHNAKTKPREEQYN |
| | | | STYRVVSVLTVLHQDWLNGKEYKCKVSNKALPAPI |
| | | | EKTIS KAKGQPREPQVYTLPPSREE |
| | | | MTKNQVSLTCLVKGFYPSDIAVEWESNGQPENNYK |
| | | | TTPPV LDSDGSFFLYSKLTVDKSRW |
| | | | QQGNVFSCSVMHEALHNHYTQKSLSLSPGK |
| | | | (SEQ ID NO: 70) |
| IL12/IL18 | | | DIVMTQSPDSLAVSLGERATINCKASQSVSNDVAW |
| LC | | | YQQKPGQPPKLLIYYASNRYTGVPD |
| | | | RFSGSGSGTDFTLTISSLQAEDVAVYYCQQDYNSP |
| | | | WTFGG GTKVEIKRTVAAPEIVMTQS |
| | | | PATLSVSPGERATLSCRASESISSNLAWYQQKPGQ |
| | | | APRLF IYTASTRATDIPARFSGSGS |
| | | | GTEFTLTISSLQSEDFAVYYCQQYNNWPSITFGQG |
| | | | TRLEIKR |
| | | | TVAAPSVFIFPPSDEQLKSGTASVVCLLNNFYPRE |
| | | | AKVQW KVDNALQSGNSQESVTEQDS |
| | | | KDSTYSLSSTLTLSKADYEKHKVYACEVTHQGLSS |
| | | | PVTKSFNRGEC |
| | | | (SEQ ID NO: 71) |
| DLL4/VEGF | | | EVQLVESGGGLVQPGGSLRLSCAASGFTFSNFP |
| HC | | | MAWVRQAPGKGLEWVATISSSDGTTYYRDSV |
| h1A11.1- | | | KGRFTISRDNAKNSLYLQMNSLRAEDTAVYYC |
| LS-AV | | | ARGYYNSPFAYWGQGTLVTVSSASTKGPSVFP |
| | | | LAPEVQLVESGGGLVQPGGSLRLSCAASGYTF |
| | | | TNYGMNWVRQAPGKGLEWVGWINTYTGEPT |
| | | | YAADFKRRFTFSLDTSKSTAYLQMNSLRAEDT |
| | | | AVYYCAKYPHYYGSSHWYFDVWGQGTLVTV |
| | | | SS |
| | | | ASTKGPSVFPLAPSSKSTSGGTAALGCLVKDYF |
| | | | PEPVTVSWNSGALTSGVHTFPAVLQSSGLYSLS |
| | | | SVVTVPSSSLGTQTYICNVNHKPSNTKVDKKV |
| | | | EPKSCDKTHTCPPCPAPEAAGGPSVFLFPPKPK |
| | | | DTLMISRTPEVTCVVVDVSHEDPEVKFNWYVD |
| | | | GVEVHNAKTKPREEQYNSTYRVVSVLTVLHQ |
| | | | DWLNGKEYKCKVSNKALPAPIEKTISKAKGQP |
| | | | REPQVYTLPPSREEMTKNQVSLTCLVKGFYPS |
| | | | DIAVEWESNGQPENNYKTTPPVLDSDGSFFLYS |
| | | | KLTVDKSRWQQGNVFSCSVMHEALHNHYTQK |
| | | | SLSLSPGK |
| | | | (SEQ ID NO: 72) |
| DLL 4 /VEGF | | | DIQMTQSPSSLSASVGDRVTITCRASEDIYSNLA |
| LC | | | WYQQKPGKAPKLLIYDTNNLADGVPSRFSGSG |
| h1A11.1- | | | SGTDFTLTISSLQPEDFATYYCQQYNNYPPTFG |
| LS-AV | | | QGTKLEIKRTVAAPDIQMTQSPSSLSASVGDRV |
| | | | TITCSASQDISNYLNWYQQKPGKAPKVLIYFTS |
| | | | SLHSGVPSRFSGSGSGTDFTLTISSLQPEDFATY |
| | | | YCQQYSTVPWTFGQGTKVEIKR |
| | | | TVAAPSVFIFPPSDEQLKSGTASVVCLLNNFYP |
| | | | REAKVQWKVDNALQSGNSQESVTEQDSKDST |
| | | | YSLS STLTLSKADYEKHKVYACEVTHQGLSSP |
| | | | VTKSFNRGEC |
| | | | (SEQ ID NO: 73) |

### INCORPORATION BY REFERENCE

The contents of all cited references (including literature references, patents, patent applications, and websites) that may be cited throughout this application are hereby expressly incorporated by reference in their entirety, as are the references cited therein. The practice of the present invention will employ, unless otherwise indicated, conventional techniques of immunology, molecular biology and cell biology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. An aqueous formulation comprising an Aqueous Stable DVD-Ig (AS-DVD-Ig) protein and a buffer having a molarity of 5 to 50 mM, wherein the formulation has a pH of 4.5 to 7.5.

2. The aqueous formulation of claim 1, further comprising a surfactant, a polyol, or combinations thereof.

3. The aqueous formulation of claim 2, wherein the surfactant is a polysorbate.

4-7. (canceled)

8. The aqueous formulation of claim 2, wherein the polyol is selected from the group consisting of sorbitol, mannitol, and sucrose.

9-22. (canceled)

23. The aqueous formulation of claim 1, wherein the buffer is selected from the group consisting of acetate, histidine, glycine, arginine, phosphate, and citrate.

24-30. (canceled)

31. The aqueous formulation of claim 1, wherein the AS-DVD-Ig protein has a binding specificity selected from the group consisting of IL4/IL13, IL1α/IL1β and TNFα/IL17.

32. The aqueous formulation of claim 31, wherein the IL1α/IL1β specific AS-DVD-Ig protein is DVD-C(SEQ ID NOs: 66 and 67).

33-36. (canceled)

37. The aqueous formulation of claim 1, wherein
a) the AS-DVD-Ig protein is characterized as a DVD-Ig protein having less than 6% aggregation as determined by SEC when formulated in a citrate phosphate buffer at a concentration of 60 mg/ml, following 14 days storage at 40 degrees C.;
b) the AS-DVD-Ig protein is characterized as a DVD-Ig protein having a 10% relative (rel.) peak area or less change in monomers at about 40° C. after 21 days of storage at a concentration of 100 mg/ml in an aqueous formulation at a pH between about 5.5 to 6.5; and/or
c) the AS-DVD-Ig protein is characterized as a DVD-Ig protein having a 1% rel. peak area or less change in monomers at about 5° C. after 21 days of storage at a concentration of 100 mg/ml at a pH between about 5.5 to 6.5 in an aqueous formulation.

38-42. (canceled)

43. A lyophilized formulation comprising a Lyophilized-Stable DVD-Ig (LS-DVD-Ig) protein, wherein when said formulation is reconstituted, and comprises about 1-100 mg/ml of the LS-DVD-Ig protein, about 10-50 mM of a buffer, a polyol, about 0.01-0.2 mg/ml of a polysorbate, and has a pH of about 5-7.

44-45. (canceled)

46. A lyophilized formulation comprising an LS-DVD-Ig protein prepared by lyophilizing an aqueous formulation comprising a buffer have a molarity of 5 to 50 mM, a surfactant, and a polyol, wherein the formulation has a pH of 4.5 to 7.5.

47-56. (canceled)

57. The formulation of claim 1, wherein the DVD-Ig protein comprises a variable light or heavy chain amino acid sequence as set forth in SEQ ID NOs: 28 to 75.

58. (canceled)

59. The formulation of claim 1, wherein the DVD-Ig protein has a binding specificity selected from the group consisting of CD20/CD80, VEGF/Her2, TNF/RANKL, TNF/DKK, CD20/RANKL, DLL4/PLGF, TNF/SOST (S2), IL-9(S2)/IgE, IL-12/IL-18, TNF/IL-17, TNF/PGE2, IL1α/IL1β or DLL4/VEGF.

60-61. (canceled)

62. The formulation of claim 1, wherein the formulation is a pharmaceutical formulation.

63. A method of treating a disorder, comprising administering the pharmaceutical formulation of claim 62, such that the disorder is treated.

64. The aqueous formulation of claim 1 comprising about 50 to about 150 mg/ml DVD C DVD-Ig protein (SEQ ID NOs: 66 and 67), about 10 to about 20 mM of histidine buffer, about 30 to about 50 mg/ml sorbitol, and about 0.005% to about 0.02% polysorbate 80, wherein said formulation has a pH of about 5 to about 7.

65. The formulation of claim 64 comprising about 15 mM of histidine buffer, about 35 to about 45 mg/ml sorbitol, and about 0.01% polysorbate 80, wherein said formulation has a pH of about 5.5 to about 6.5.

66. The formulation of claim 64 comprising about 70 to about 110 mg/ml DVD C DVD-Ig protein.

67. The formulation of claim 64 comprising about 100 mg/ml DVD C DVD-Ig protein.
